(19) United States
(12) Patent Application Publication
(10) Pub. No.: US 2022/0169639 A1
(43) Pub. Date: Jun. 2, 2022

(54) N-CONTAINING CHROMEN-4-ONE DERIVATIVES FOR THE TREATMENT AND PROPHYLAXIS OF HEPATITIS B VIRUS INFECTION

(71) Applicant: Hoffmann-La Roche Inc., Little Falls, NJ (US)

(72) Inventors: Song FENG, Shanghai (CN); Jiamin ZHENG, Shanghai (CN); Yongfu LIU, Shanghai (CN); Dongdong CHEN, Shanghai (CN); Hong SHEN, Shanghai (CN); Xuefei TAN, Shanghai (CN)

(73) Assignee: Hoffmann-La Roche Inc., Little Falls, NJ (US)

(21) Appl. No.: 17/413,425

(22) PCT Filed: Dec. 11, 2019
(86) PCT No.: PCT/EP2019/084552

§ 371 Date: Jun. 11, 2021

(30) Foreign Application Priority Data
Dec. 14, 2018 (CN) PCT/CN2018/121081

(51) Int. Cl. C07D 413/12; C07D 405/12; C07D 407/12; C07D 311/30; C07D 411/12; C07D 417/12; C07D 407/14

(52) U.S. Cl. CPC C07D 413/12; C07D 405/12; C07D 407/12; C07D 407/14; C07D 411/12; C07D 417/12; C07D 311/30

(57) ABSTRACT

The present invention provides the compounds having the general formula: (I) wherein R¹ to R⁸, and X are as described herein, compositions including the compounds and methods of using the compounds.

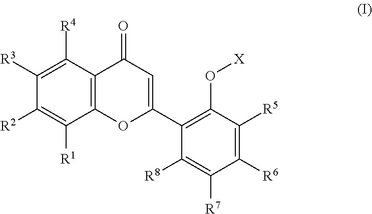

The present invention relates to organic compounds useful for therapy and/or prophylaxis of HBV infection in a mammal, and in particular to cccDNA (covalently closed circular DNA) inhibitors useful for treating HBV infection.

### FIELD OF THE INVENTION

The present invention relates to N-containing chromen-4-one derivatives having pharmaceutical activity, their manufacture, pharmaceutical compositions containing them and their potential use as medicaments.

The present invention relates to compounds of formula (I)

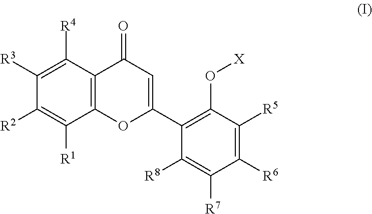

wherein R¹ to R⁸ and X are as described below, or a pharmaceutically acceptable salt thereof.

Hepatitis B virus (HBV) infection is one of the most prevalent viral infections and is a leading cause of chronic hepatitis. It is estimated that worldwide, around 2 billion people have evidence of past or present infection with HBV. Over 250 million individuals are currently chronically infected with HBV and are therefore at high risk to develop liver fibrosis, cirrhosis and hepatocellular carcinoma (HCC). There are data to indicate ˜800,000 deaths per year are directly linked to HBV infection (Lozano, R. et al., Lancet (2012), 380 (9859), 2095-2128; Goldstein, S. T. et al., Int J Epidemiol (2005), 34 (6), 1329-1339).

Many countries in the world administer hepatitis B immunization starting at birth or in early childhood, which has greatly reduced the incidence and prevalence of hepatitis B in most endemic regions over the past few decades. However, the vaccination has no impact on people who were infected before the widespread use of the vaccine in developing end-stage liver disease or HCC (Chen, D. S., J Hepatol (2009), 50 (4), 805-816). Vaccination at birth of infants born to HBV positive mothers is usually not sufficient for protecting vertical transmission and combination with hepatitis B immune globulin is needed (Li, X. M. et al., World J Gastroenterol (2003), 9 (7), 1501-1503).

Currently FDA-approved treatments for chronic hepatitis B include two type 1 interferons (IFN) which are IFNalfa-2b and pegylated IFN alfa-2a and six nucleos(t)ide analogues (NAs) which are lamivudine (3TC), tenofovir disoproxil fumarate (TDF), adefovir (ADV), telbivudine (LdT), entecavir (ETV), and vemlidy (tenofovir alafenamide (TAF)). IFN treatment is finite, but it is known to have severe side effects, and only a small percentage of patients showed a sustained virological response, measured as loss of hepatitis B surface antigen (HBsAg). NAs are inhibitors of the HBV reverse transcriptase, profoundly reduce the viral load in vast majority of treated patients, and lead to improvement of liver function and reduced incidence of liver failure and hepatocellular carcinoma. However, the treatment of NAs is infinite (Ahmed, M. et al., Drug Discov Today (2015), 20 (5), 548-561; Zoulim, F. and Locarnini, S., Gastroenterology (2009), 137 (5), 1593-1608 e1591-1592).

HBV chronic infection is caused by persistence of covalently closed circular (ccc)DNA, which exists as an episomal form in hepatocyte nuclei. cccDNA serves as the template for viral RNA transcription and subsequent viral DNA generation. Only a few copies of cccDNA per liver cell can establish or re-initiate viral replication. Therefore, a complete cure of chronic hepatitis B will require elimination of cccDNA or permanently silencing of cccDNA. However, cccDNA is intrinsically very stable and currently available therapeutics could not eliminate cccDNA or permanently silence cccDNA (Nassal, M., Gut (2015), 64 (12), 1972-1984; Gish, R. G. et al., Antiviral Res (2015), 121, 47-58; Levrero, M. et al., J Hepatol (2009), 51 (3), 581-592.). The current SoC could not eliminate the cccDNA which are already present in the infected cells. There is an urgent need to discover and develop new anti-HBV reagents to eliminate or permanently silence cccDNA, the source of chronicity (Ahmed, M. et al., Drug Discov Today (2015), 20 (5), 548-561; Nassal, M., Gut (2015), 64 (12), 1972-1984).

### SUMMARY OF THE INVENTION

Objects of the present invention are compounds of formula (I), their manufacture, medicaments based on a compound in accordance with the invention and their production as well as the use of compounds of formula (I) as cccDNA inhibitors and for the treatment or prophylaxis of HBV infection. The compounds of formula (I) show superior anti-HBV activity. In addition, the compounds of formula (I) also show good PK profiles.

The present invention relates to a compound of formula (I)

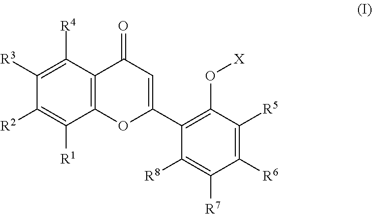

wherein
R¹ is halogen;
R² is selected from H, OH, halogen, C₁₋₆alkyl, haloC₁₋₆alkyl and C₁₋₆alkoxy;
R³ is selected from H, OH, halogen, C₁₋₆alkyl, haloC₁₋₆alkyl and C₁₋₆alkoxy;
R⁴ is selected from H, OH, halogen, C₁₋₆alkyl, haloC₁₋₆alkyl and C₁₋₆alkoxy;
R⁵ is selected from H, OH, halogen, C₁₋₆alkyl, haloC₁₋₆alkyl and C₁₋₆alkoxy;
R⁶ is selected from H, OH, halogen, C₁₋₆alkyl, haloC₁₋₆alkyl and C₁₋₆alkoxy;
R⁷ is selected from H, OH, halogen, C₁₋₆alkyl, haloC₁₋₆alkyl and C₁₋₆alkoxy;
R⁸ is selected from H, OH, halogen, C₁₋₆alkyl, haloC₁₋₆alkyl and C₁₋₆alkoxy;

### X is

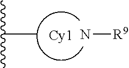

or -L-Y; wherein
Cy1 is N-containing heterocyclyl;
R⁹ is selected from C₃₋₇cycloalkylsulfonyl, carboxycarbonyl, carboxyphenylC₁₋₆alkyl and carboxyC₃₋₇cycloalkyl;
L is selected from C₁₋₆alkyl, C₁₋₆alkoxyC₁₋₆alkyl, C₃₋₇cycloalkyl, C₃₋₇cycloalkylC₁₋₆alkyl and C₁₋₆alkylheterocyclylC₁₋₆alkyl; wherein C₁₋₆alkyl is unsubstituted or substituted by OH;
Y is —NR¹⁰R¹¹ or

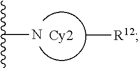

wherein
Cy2 is N-containing heterocyclyl; wherein N-containing heterocyclyl is unsubstituted or substituted by one or two or three substituents independently selected from halogen, C₁₋₆alkyl and OH;
R¹⁰ is selected from H, C₁₋₆alkyl, C₃₋₇cycloalkyl, C₁₋₆alkylsulfonyl, C₃₋₇ cycloalkylsulfonyl, carboxyC₁₋₆alkyl, phenylsulfonyl and C₁₋₆alkylcarbonyl;
R¹¹ is selected from carboxyC₃₋₇cycloalkyl, carboxyC₁₋₆alkyl, carboxycarbonyl, carboxyheterocyclyl, carboxyC₃₋₇cycloalkylC₁₋₆alkyl, heterocyclyl, C₁₋₆alkylsulfonylC₁₋₆alkyl, aminosulfonylC₁₋₆alkyl, C₃₋₇ cycloalkylsulfonyl, C₁₋₆alkoxycarbonylcarbonyl, phenylsulfonyl, C₁₋₆alkylcarbonyl, C₁₋₆alkoxycarbonyl, C₁₋₆alkylaminosulfonyl, aminocarbonylcarbonyl, C₃₋₇cycloalkylaminocarbonylcarbonyl, C₃₋₇ cycloalkylsulfonylaminocarbonylcarbonyl, hydroxyheterocyclylcarbonylcarbonyl, carboxyC₁₋₆alkylaminocarbonyl, C₁₋₆alkylphenylsulfonylaminocarbonyl, aminocarbonyl, aminosulfonyl and aminocarbonylC₁₋₆alkyl;
R¹² is selected from H, carboxy, carboxyC₁₋₆alkyl, C₁₋₆alkylsulfonylaminocarbonyl, C₃₋₇cycloalkylsulfonylaminocarbonyl, C₁₋₆alkyl(C₁₋₆alkylsulfonyl)amino, C₁₋₆alkylsulfonyl, C₁₋₆alkylcarbonyl, C₁₋₆alkylsulfinyl, C₁₋₆alkylsulfonimidoyl, aminocarbonyl, carboxycarbonyl and heterocyclyl;
   or a pharmaceutically acceptable salt thereof.

### DETAILED DESCRIPTION OF THE INVENTION

### Definitions

As used herein, the term “C₁₋₆alkyl” alone or in combination signifies a saturated, linear- or branched chain alkyl group containing 1 to 6, particularly 1 to 4 carbon atoms, for example methyl, ethyl, propyl, isopropyl, butyl, isobutyl, tert-butyl and the like. Particular “C₁₋₆alkyl” groups are methyl, ethyl, propyl, isopropyl and isobutyl. Most particular “C₁₋₆alkyl” group is methyl.

The term “C₁₋₆alkoxy” alone or in combination signifies a group C₁₋₆alkyl-O—, wherein the “C₁₋₆alkyl” is as defined above; for example methoxy, ethoxy, propoxy, iso-propoxy, n-butoxy, iso-butoxy, 2-butoxy, tert-butoxy, pentoxy, hexyloxy and the like. Particular “C₁₋₆alkoxy” groups are methoxy, ethoxy and iso-butoxy.

The term “C₃₋₇cycloalkyl” denotes to a saturated carbon mono or bicyclic ring or a saturated spiro-linked bicyclic carbon ring or a bridged carbon ring, containing from 3, 4, 5, 6, or 7 carbon atoms, particularly from 3 to 6 carbon atoms, for example, cyclopropyl, cyclobutyl, cyclopentyl, cyclohexyl, cycloheptyl, bicyclo[1.1.1]pentanyl and the like. Particular “C₃₋₇cycloalkyl” group is cyclopropyl or cyclobutyl.

The term “halogen” and “halo” are used interchangeably herein and denote fluoro, chloro, bromo, or iodo.

The term “haloC₁₋₆alkyl” denotes an alkyl group wherein at least one of the hydrogen atoms of the alkyl group is replaced by same or different halogen atoms, particularly fluoro atoms. Examples of haloC₁₋₆alkyl include monochloro-, difluoro- or trifluoro-methyl, -ethyl or -propyl, for example difluoromethyl and trifluoromethyl.

The term “haloC₁₋₆alkoxy” denotes a C₁₋₆alkoxy group wherein at least one of the hydrogen atoms of the C₁₋₆alkoxy group is replaced by same or different halogen atoms, particularly fluoro atoms. Examples of haloC₁₋₆alkoxy include monofluoro-, difluoro- or trifluoro-methoxy, -ethoxy or -propoxy, for example trifluoromethoxy.

The term “heterocyclyl” refers to any mono-, bi-, tricyclic or spiro, saturated or unsaturated, aromatic (heteroaryl) or non-aromatic (e.g., heterocycloalkyl), ring system, having 3 to 20 ring atoms, where the ring atoms are carbon, and at least one atom in the ring or ring system is a heteroatom selected from nitrogen, sulfur or oxygen. If any ring atom of a cyclic system is a heteroatom, that system is a heterocyclyl, regardless of the point of attachment of the cyclic system to the rest of the molecule. In one example, heterocyclyl includes 3-11 ring atoms (“members”) and includes monocycles, bicycles, tricycles and spiro ring systems, wherein the ring atoms are carbon, where at least one atom in the ring or ring system is a heteroatom selected from nitrogen, sulfur or oxygen. In one example, heterocyclyl includes 3- to 7-membered monocycles having 1, 2, 3 or 4 heteroatoms selected from nitrogen, sulfur or oxygen. In another example, heterocyclyl includes 4-, 5- or 6-membered monocycles having 1, 2, 3 or 4 heteroatoms selected from nitrogen, sulfur or oxygen. In one example, heterocyclyl includes 8- to 12-membered bicycles having 1, 2, 3, 4, 5 or 6 heteroatoms selected from nitrogen, sulfur or oxygen. In another example, heterocyclyl includes 9- or 10-membered bicycles having 1, 2, 3, 4, 5 or 6 heteroatoms selected from nitrogen, sulfur or oxygen. Exemplary heterocyclyls are oxetanyl, pyrrolidinyl, morpholinyl, azetidinyl, piperidyl, azabicyclo[3.2.1]octanyl, oxopyrrolidinyl, piperazinyl, thiomorpholinyl, dioxothiazinanyl, oxoimidazolidinyl, dioxoimidazolidinyl, tetrahydropyranyl and 2H-tetrazolyl.

The term “carbonyl” alone or in combination refers to the group —C(O)—.

The term “sulfonyl” alone or in combination refers to the group —S(O)₂—.

The term “sulfonimidoyl” alone or in combination refers to the group —S(O)(NH)—, whose formula is

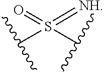 [SMILES: *S(*)(=N)=O]

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of formula (I) and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin R. J., et al., Organic Process Research & Development 2000, 4, 427-435. Particular are the sodium salts of the compounds of formula (I).

Compounds of the general formula (I) which contain one or several chiral centers can either be present as racemates, diastereomeric mixtures, or optically active single isomers. The racemates can be separated according to known methods into the enantiomers. Particularly, diastereomeric salts which can be separated by crystallization are formed from the racemic mixtures by reaction with an optically active acid such as e.g. D- or L-tartaric acid, mandelic acid, malic acid, lactic acid or camphorsulfonic acid.

HBV cccDNA Inhibitors

The present invention provides (i) a compound having the general formula (I):

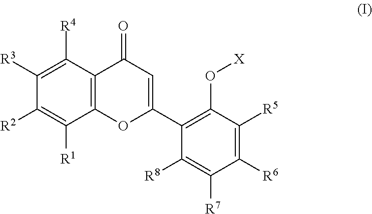

wherein
R¹ is halogen;
R² is selected from H, OH, halogen, C₁₋₆alkyl, haloC₁₋₆alkyl and C₁₋₆alkoxy;
R³ is selected from H, OH, halogen, C₁₋₆alkyl, haloC₁₋₆alkyl and C₁₋₆alkoxy;
R⁴ is selected from H, OH, halogen, C₁₋₆alkyl, haloC₁₋₆alkyl and C₁₋₆alkoxy;
R⁵ is selected from H, OH, halogen, C₁₋₆alkyl, haloC₁₋₆alkyl and C₁₋₆alkoxy;
R⁶ is selected from H, OH, halogen, C₁₋₆alkyl, haloC₁₋₆alkyl and C₁₋₆alkoxy;
R⁷ is selected from H, OH, halogen, C₁₋₆alkyl, haloC₁₋₆alkyl and C₁₋₆alkoxy;
R⁸ is selected from H, OH, halogen, C₁₋₆alkyl, haloC₁₋₆alkyl and C₁₋₆alkoxy;

### X is

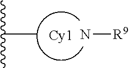

or -L-Y; wherein
Cy1 is N-containing heterocyclyl;
R⁹ is selected from C₃₋₇cycloalkylsulfonyl, carboxycarbonyl, carboxyphenylC₁₋₆alkyl and carboxyC₃₋₇cycloalkyl;
L is selected from C₁₋₆alkyl, C₁₋₆alkoxyC₁₋₆alkyl, C₃₋₇cycloalkyl, C₃₋₇cycloalkylC₁₋₆alkyl and C₁₋₆alkylheterocyclylC₁₋₆alkyl; wherein C₁₋₆alkyl is unsubstituted or substituted by OH;
Y is —NR¹⁰R¹¹ or

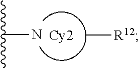

wherein
Cy2 is N-containing heterocyclyl; wherein N-containing heterocyclyl is unsubstituted or substituted by one or two or three substituents independently selected from halogen, C₁₋₆alkyl and OH;
R¹⁰ is selected from H, C₁₋₆alkyl, C₃₋₇cycloalkyl, C₁₋₆alkylsulfonyl, C₃₋₇ cycloalkylsulfonyl, carboxyC₁₋₆alkyl, phenylsulfonyl and C₁₋₆alkylcarbonyl;
R¹¹ is selected from carboxyC₃₋₇cycloalkyl, carboxyC₁₋₆alkyl, carboxycarbonyl, carboxyheterocyclyl, carboxyC₃₋₇cycloalkylC₁₋₆alkyl, heterocyclyl, C₁₋₆alkylsulfonylC₁₋₆alkyl, aminosulfonylC₁₋₆alkyl, C₃₋₇cycloalkylsulfonyl, C₁₋₆alkoxycarbonylcarbonyl, phenylsulfonyl, C₁₋₆alkylcarbonyl, C₁₋₆alkoxycarbonyl, C₁₋₆alkylaminosulfonyl, aminocarbonylcarbonyl, C₃₋₇cycloalkylaminocarbonylcarbonyl, C₃₋₇ cycloalkylsulfonylaminocarbonylcarbonyl, hydroxyheterocyclylcarbonylcarbonyl, carboxyC₁₋₆alkylaminocarbonyl, C₁₋₆alkylphenylsulfonylaminocarbonyl, aminocarbonyl, aminosulfonyl and aminocarbonylC₁₋₆alkyl;
R¹² is selected from H, carboxy, carboxyC₁₋₆alkyl, C₁₋₆alkylsulfonylaminocarbonyl, C₃₋₇cycloalkylsulfonylaminocarbonyl, C₁₋₆alkyl(C₁₋₆alkylsulfonyl)amino, C₁₋₆alkylsulfonyl, C₁₋₆alkylcarbonyl, C₁₋₆alkylsulfinyl, C₁₋₆alkylsulfonimidoyl, aminocarbonyl, carboxycarbonyl and heterocyclyl;
   or a pharmaceutically acceptable salt thereof.

A further embodiment of the present invention is (ii) a compound of formula (I) according to (i), wherein

R¹ is halogen;

### R² is H;

R³ is selected from H, halogen and C₁₋₆alkoxy;
R⁴ is selected from H and haloC₁₋₆alkyl;

### R⁵ is H;

R⁶ is selected from H, halogen, C₁₋₆alkyl, haloC₁₋₆alkyl and C₁₋₆alkoxy;
R⁷ is selected from H, halogen, C₁₋₆alkyl and C₁₋₆alkoxy;
R⁸ is selected from H, halogen and C₁₋₆alkoxy;

### X is

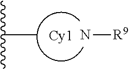

or -L-Y; wherein
Cy1 is selected from piperidyl, pyrrolidinyl and azetidinyl;
R⁹ is selected from C₃₋₇cycloalkylsulfonyl, carboxycarbonyl, carboxyphenylC₁₋₆alkyl and carboxyC₃₋₇cycloalkyl;
L is selected from C₁₋₆alkyl, C₁₋₆alkoxyC₁₋₆alkyl, C₃₋₇cycloalkyl, C₃₋₇cycloalkylC₁₋₆alkyl and C₁₋₆alkyloxetanylC₁₋₆alkyl; wherein C₁₋₆alkyl is unsubstituted or substituted by OH;
Y is —NR¹⁰R¹¹ or

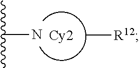

wherein
Cy2 is selected from pyrrolidinyl, morpholinyl, azetidinyl, piperidyl, azabicyclo[3.2.1]octanyl, oxopyrrolidinyl, piperazinyl, thiomorpholinyl, dioxothiazinanyl, oxoimidazolidinyl and dioxoimidazolidinyl; wherein pyrrolidinyl is unsubstituted or substituted by one or two substituents independently selected from halogen, C₁₋₆alkyl and OH;
R¹⁰ is selected from H, C₁₋₆alkyl, C₃₋₇cycloalkyl, C₁₋₆alkylsulfonyl, C₃₋₇ cycloalkylsulfonyl, carboxyC₁₋₆alkyl, phenylsulfonyl and C₁₋₆alkylcarbonyl;
R¹¹ is selected from carboxyC₃₋₇cycloalkyl, carboxyC₁₋₆alkyl, carboxycarbonyl, carboxytetrahydropyranyl, carboxyC₃₋₇cycloalkylC₁₋₆alkyl, dioxothiazinanyl, C₁₋₆alkylsulfonylC₁₋₆alkyl, aminosulfonylC₁₋₆alkyl, C₃₋₇cycloalkylsulfonyl, C₁₋₆alkoxycarbonylcarbonyl, phenylsulfonyl, C₁₋₆alkylcarbonyl, C₁₋₆alkoxycarbonyl, C₁₋₆ alkylaminosulfonyl, aminocarbonylcarbonyl, C₃₋₇cycloalkylaminocarbonylcarbonyl, C₃₋₇ cycloalkylsulfonylaminocarbonylcarbonyl, hydroxypyrrolidinylcarbonylcarbonyl, carboxyC₁₋₆alkylaminocarbonyl, C₁₋₆alkylphenylsulfonylaminocarbonyl, aminocarbonyl, aminosulfonyl and aminocarbonylC₁₋₆alkyl;
R¹² is selected from H, carboxy, carboxyC₁₋₆alkyl, C₁₋₆alkylsulfonylaminocarbonyl, C₃₋₇cycloalkylsulfonylaminocarbonyl, C₁₋₆alkyl(C₁₋₆alkylsulfonyl)amino, C₁₋₆alkylsulfonyl, C₁₋₆alkylcarbonyl, C₁₋₆alkylsulfinyl, C₁₋₆alkylsulfonimidoyl, aminocarbonyl, carboxycarbonyl and 2H-tetrazolyl;
   or a pharmaceutically acceptable salt thereof.

A further embodiment of the present invention is (iii) a compound of formula (I) according to (i), wherein

### R¹ is Cl;

### R² is H;

R³ is selected from H, F and methoxy;
R⁴ is selected from H and CF₃;

### R⁵ is H;

R⁶ is selected from H, Cl, Br, methyl, CF₃, methoxy, ethoxy and isobutoxy;
R⁷ is selected from H, Br, methyl and methoxy;
R⁸ is selected from H, F and methoxy;

### X is

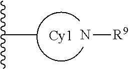

or -L-Y; wherein
Cy1 is selected from piperidyl, pyrrolidinyl and azetidinyl;
R⁹ is selected from cyclopropylsulfonyl, carboxycarbonyl, carboxyphenylmethyl and carboxycyclobutyl;
L is selected from ethyl, propyl, isobutyl, ethoxyethyl, cyclobutyl, cyclopropylmethyl and methyloxetanylmethyl; wherein propyl is unsubstituted or substituted one time by OH;
Y is —NR¹⁰R¹¹ or

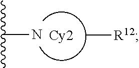

wherein
Cy2 is selected from pyrrolidinyl, morpholinyl, azetidinyl, piperidyl, azabicyclo[3.2.1]octanyl, oxopyrrolidinyl, piperazinyl, thiomorpholinyl, dioxothiazinanyl, oxoimidazolidinyl and dioxoimidazolidinyl; wherein pyrrolidinyl is unsubstituted or substituted by one or two substituents independently selected from F, methyl, isopropyl and OH;
R¹⁰ is selected from H, methyl, cyclopropyl, ethylsulfonyl, cyclopropylsulfonyl, carboxymethyl, phenylsulfonyl and methylcarbonyl;
R¹¹ is selected from carboxycyclobutyl, carboxycyclopentyl, carboxyisobutyl, carboxycarbonyl, carboxydimethylbutyl, carboxybicyclo[1.1.1]pentanyl, carboxycyclohexyl, carboxyethyl, carboxyisopropyl, carboxytetrahydropyranyl, carboxycyclohexylethyl, dioxothiazinanyl, methylsulfonylethyl, aminosulfonylethyl, cyclopropylsulfonyl, ethoxycarbonylcarbonyl, phenylsulfonyl, methylcarbonyl, ethylcarbonyl, methoxycarbonyl, ethoxycarbonyl, methylaminosulfonyl, carboxymethyl, ethylaminosulfonyl, aminocarbonylcarbonyl, cyclopropylaminocarbonylcarbonyl, cyclopropylsulfonylaminocarbonylcarbonyl, hydroxypyrrolidinylcarbonylcarbonyl, carboxyethylaminocarbonyl, carboxymethylaminocarbonyl, methylphenylsulfonylaminocarbonyl, aminocarbonyl, aminosulfonyl, aminocarbonylmethyl and aminocarbonylethyl;
R¹² is selected from H, carboxy, carboxymethyl, methylsulfonylaminocarbonyl, cyclopropylsulfonylaminocarbonyl, methyl(methylsulfonyl)amino, methylsulfonyl, methylcarbonyl, methylsulfinyl, methylsulfonimidoyl, aminocarbonyl, carboxycarbonyl and 2H-tetrazolyl;
   or a pharmaceutically acceptable salt thereof.

A further embodiment of the present invention is (iv) a compound of formula (I) according to (i), or a pharmaceutically acceptable salt thereof, wherein R³ is selected from H.

A further embodiment of the present invention is (v) a compound of formula (I) according to (i), or a pharmaceutically acceptable salt thereof, wherein R⁶ is selected from halogen, C₁₋₆ alkyl and haloC₁₋₆alkyl.

A further embodiment of the present invention is (vi) a compound of formula (I) according to (i), or a pharmaceutically acceptable salt thereof, wherein R⁶ is selected from Br, methyl and CF₃.

A further embodiment of the present invention is (vii) a compound of formula (I) according to (i), or a pharmaceutically acceptable salt thereof, R⁷ is selected from H and C₁₋₆alkoxy.

A further embodiment of the present invention is (viii) a compound of formula (I) according to (i), or a pharmaceutically acceptable salt thereof, wherein R⁷ is selected from H and methoxy.

A further embodiment of the present invention is (ix) a compound of formula (I) according to (viii), or a pharmaceutically acceptable salt thereof, wherein X is -L-Y.

A further embodiment of the present invention is (x) a compound of formula (I) according to (i), or a pharmaceutically acceptable salt thereof, wherein L is selected from C₁₋₆alkyl and C₁₋₆alkoxyC₁₋₆alkyl; wherein C₁₋₆alkyl is unsubstituted or substituted by OH.

A further embodiment of the present invention is (xi) a compound of formula (I) according to (i), or a pharmaceutically acceptable salt thereof, wherein L is selected from ethyl, propyl and ethoxyethyl; wherein propyl is unsubstituted or substituted one time by OH.

A further embodiment of the present invention is (xii) a compound of formula (I) according to (i), or a pharmaceutically acceptable salt thereof, wherein Y is

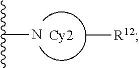

wherein Cy2 is selected from pyrrolidinyl and morpholinyl.

A further embodiment of the present invention is (xiii) a compound of formula (I) according to (i), or a pharmaceutically acceptable salt thereof, wherein R¹² is selected from carboxy, C₁₋₆alkylsulfonylaminocarbonyl and C₁₋₆alkylsulfonyl.

A further embodiment of the present invention is (xiv) a compound of formula (I) according to (i), or a pharmaceutically acceptable salt thereof, wherein R¹² is selected from carboxy, methylsulfonylaminocarbonyl and methylsulfonyl.

A further embodiment of the present invention is (xv) a compound of formula (I) according to (i), or a pharmaceutically acceptable salt thereof, wherein Y is —NHR¹¹.

A further embodiment of the present invention is (xvi) a compound of formula (I) according to (i), or a pharmaceutically acceptable salt thereof, wherein R¹¹ is selected from carboxyC₃₋₇cycloalkyl, carboxycarbonyl, carboxyC₁₋₆alkylaminocarbonyl, aminocarbonyl and aminosulfonyl.

A further embodiment of the present invention is (xvii) a compound of formula (I) according to (i), or a pharmaceutically acceptable salt thereof, wherein R¹¹ is selected from carboxycyclobutyl, carboxycyclopentyl, carboxycarbonyl, carboxyethylaminocarbonyl, aminocarbonyl and aminosulfonyl.

A further embodiment of the present invention is (xviii) a compound of formula (II) according to (i), or a pharmaceutically acceptable salt thereof,

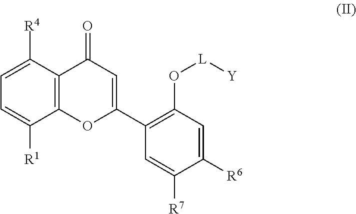

R¹ is halogen;
R⁴ is selected from H and haloC₁₋₆alkyl;
R⁶ is selected from halogen, C₁₋₆alkyl and haloC₁₋₆alkyl;
R⁷ is selected from H and C₁₋₆alkoxy;
L is selected from C₁₋₆alkyl and C₁₋₆alkoxyC₁₋₆alkyl; wherein C₁₋₆alkyl is unsubstituted or substituted by OH;
Y is —NHR¹¹ or

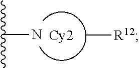

wherein
Cy2 is selected from pyrrolidinyl and morpholinyl;
R¹¹ is selected from carboxyC₃₋₇cycloalkyl, carboxycarbonyl, carboxyC₁₋₆alkylaminocarbonyl, aminocarbonyl and aminosulfonyl;
R¹² is selected from carboxy, C₁₋₆alkylsulfonylaminocarbonyl and C₁₋₆alkylsulfonyl.

A further embodiment of the present invention is (xix) a compound of formula (II) according to (i), or a pharmaceutically acceptable salt thereof, wherein
R¹ is Cl;
R⁴ is selected from H and CF₃;
R⁶ is selected from Br, methyl and CF₃;
R⁷ is selected from H and methoxy;
L is selected from ethyl, propyl and ethoxyethyl; wherein propyl is unsubstituted or substituted one time by OH;
Y is —NHR¹¹ or

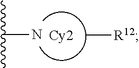

wherein
Cy2 is selected from pyrrolidinyl and morpholinyl;
R¹¹ is selected from carboxycyclobutyl, carboxycyclopentyl, carboxycarbonyl, carboxyethylaminocarbonyl, aminocarbonyl and aminosulfonyl;
R¹² is selected from carboxy, methylsulfonylaminocarbonyl and methylsulfonyl.

In another embodiment (xx) of the present invention, particular compounds of the present invention are selected from:
1-[3-[5-bromo-2-(8-chloro-4-oxo-chromen-2-yl)phenoxy]propyl]pyrrolidine-3-carboxylic acid;
4-[3-[5-bromo-2-(8-chloro-4-oxo-chromen-2-yl)phenoxy]propyl]morpholine-2-carboxylic acid;
(2R)-1-[3-[5-bromo-2-(8-chloro-4-oxo-chromen-2-yl)phenoxy]propyl]pyrrolidine-2-carboxylic acid;
(2S)-1-[3-[5-bromo-2-(8-chloro-4-oxo-chromen-2-yl)phenoxy]propyl]pyrrolidine-2-carboxylic acid;
(3R)-1-[3-[5-bromo-2-(8-chloro-4-oxo-chromen-2-yl)phenoxy]propyl]pyrrolidine-3-carboxylic acid;
3-[3-[2-(8-chloro-4-oxo-chromen-2-yl)-5-(trifluoromethyl)phenoxy]propylamino]cyclobutanecarboxylic acid;
3-[3-[2-(8-chloro-4-oxo-chromen-2-yl)-5-(trifluoromethyl)phenoxy]propylamino]cyclopentanecarboxylic acid;
2-[1-[3-[2-(8-chloro-4-oxo-chromen-2-yl)-5-(trifluoromethyl)phenoxy]propyl]pyrrolidin-3-yl]acetic acid;
(2R)-2-[3-[2-(8-chloro-4-oxo-chromen-2-yl)-5-(trifluoromethyl)phenoxy]propylamino]-3-methyl-butanoic acid;
2-[[3-[[2-(8-chloro-4-oxo-chromen-2-yl)-5-(trifluoromethyl)phenoxy]methyl]oxetan-3-yl]methylamino]-2-oxo-acetic acid;
2-[3-[2-(8-chloro-4-oxo-chromen-2-yl)-5-(trifluoromethyl)phenoxy]propylamino]-4,4-dimethyl-pentanoic acid;
1-[3-[2-(8-chloro-4-oxo-chromen-2-yl)-5-(trifluoromethyl)phenoxy]propyl]pyrrolidine-3-carboxylic acid;
(3R)-1-[3-[2-(8-chloro-4-oxo-chromen-2-yl)-5-(trifluoromethyl)phenoxy]propyl]pyrrolidine-3-carboxylic acid;
(3S)-1-[3-[2-(8-chloro-4-oxo-chromen-2-yl)-5-(trifluoromethyl)phenoxy]propyl]pyrrolidine-3-carboxylic acid;
(3S)-1-[3-[2-(8-chloro-4-oxo-chromen-2-yl)-5-ethoxy-phenoxy]propyl]pyrrolidine-3-carboxylic acid;
(3R)-1-[3-[5-bromo-2-(8-chloro-4-oxo-chromen-2-yl)-4-methoxy-phenoxy]propyl]pyrrolidine-3-carboxylic acid;
(3S)-1-[3-[5-bromo-2-(8-chloro-4-oxo-chromen-2-yl)-4-methoxy-phenoxy]propyl]pyrrolidine-3-carboxylic acid;
4-[3-[2-(8-chloro-4-oxo-chromen-2-yl)-5-(trifluoromethyl)phenoxy]propyl]morpholine-2-carboxylic acid;
1-[2-[5-bromo-2-(8-chloro-4-oxo-chromen-2-yl)phenoxy]ethyl]azetidine-3-carboxylic acid;
1-[2-[5-bromo-2-(8-chloro-4-oxo-chromen-2-yl)phenoxy]ethyl]pyrrolidine-2-carboxylic acid;
1-[2-[5-bromo-2-(8-chloro-4-oxo-chromen-2-yl)phenoxy]ethyl]pyrrolidine-3-carboxylic acid;
1-[2-[5-bromo-2-(8-chloro-4-oxo-chromen-2-yl)phenoxy]ethyl]piperidine-4-carboxylic acid;
4-[2-[5-bromo-2-(8-chloro-4-oxo-chromen-2-yl)phenoxy]ethyl]morpholine-2-carboxylic acid;
(3S,4S)-1-[2-[5-bromo-2-(8-chloro-4-oxo-chromen-2-yl)phenoxy]ethyl]-4-methyl-pyrrolidine-3-carboxylic acid;
8-[2-[5-bromo-2-(8-chloro-4-oxo-chromen-2-yl)phenoxy]ethyl]-8-azabicyclo[3.2.1]octane-3-carboxylic acid;
3-[2-[5-bromo-2-(8-chloro-4-oxo-chromen-2-yl)phenoxy]ethylamino]bicyclo[1.1.1]pentane-1-carboxylic acid;
1-[2-[5-bromo-2-(8-chloro-4-oxo-chromen-2-yl)phenoxy]ethyl]-4,4-difluoro-pyrrolidine-2-carboxylic acid;
1-[2-[2-(8-chloro-4-oxo-chromen-2-yl)phenoxy]ethyl]piperidine-4-carboxylic acid;
(2R)-1-[2-[2-(8-chloro-4-oxo-chromen-2-yl)phenoxy]ethyl]pyrrolidine-2-carboxylic acid;
1-[2-[2-(8-chloro-4-oxo-chromen-2-yl)phenoxy]ethyl]pyrrolidine-3-carboxylic acid;
(2S)-1-[2-[2-(8-chloro-4-oxo-chromen-2-yl)phenoxy]ethyl]pyrrolidine-2-carboxylic acid;
1-[2-[2-(8-chloro-4-oxo-chromen-2-yl)phenoxy]ethyl]azetidine-3-carboxylic acid;
(3S)-1-[2-[2-(8-chloro-4-oxo-chromen-2-yl)-5-(trifluoromethyl)phenoxy]ethyl]pyrrolidine-3-carboxylic acid;
4-[2-[2-(8-chloro-4-oxo-chromen-2-yl)-5-(trifluoromethyl)phenoxy]ethyl]morpholine-2-carboxylic acid;
(2R)-2-[2-[2-(8-chloro-4-oxo-chromen-2-yl)-5-(trifluoromethyl)phenoxy]ethylamino]-3-methyl-butanoic acid;
(2S)-2-[2-[2-(8-chloro-4-oxo-chromen-2-yl)-5-(trifluoromethyl)phenoxy]ethylamino]-3-methyl-butanoic acid;
3-[2-[2-(8-chloro-4-oxo-chromen-2-yl)-5-(trifluoromethyl)phenoxy]ethylamino]cyclobutanecarboxylic acid;
(3R)-1-[2-[2-(8-chloro-4-oxo-chromen-2-yl)-5-isobutoxy-phenoxy]ethyl]pyrrolidine-3-carboxylic acid;
(3S)-1-[2-[2-(8-chloro-4-oxo-chromen-2-yl)-5-isobutoxy-phenoxy]ethyl]pyrrolidine-3-carboxylic acid;
(3R)-1-[2-[5-bromo-2-[8-chloro-4-oxo-5-(trifluoromethyl)chromen-2-yl]phenoxy]ethyl]pyrrolidine-3-carboxylic acid;
(3S)-1-[2-[5-bromo-2-[8-chloro-4-oxo-5-(trifluoromethyl)chromen-2-yl]phenoxy]ethyl]pyrrolidine-3-carboxylic acid;
1-[2-[2-(8-chloro-6-fluoro-4-oxo-chromen-2-yl)phenoxy]ethyl]pyrrolidine-3-carboxylic acid;
(2R)-1-[2-[5-bromo-2-(8-chloro-4-oxo-chromen-2-yl)-4-methyl-phenoxy]ethyl]pyrrolidine-2-carboxylic acid;
(2S)-1-[2-[5-chloro-2-(8-chloro-4-oxo-chromen-2-yl)-4-methyl-phenoxy]ethyl]pyrrolidine-2-carboxylic acid;
3-[3-[5-chloro-2-(8-chloro-4-oxo-chromen-2-yl)-4-methyl-phenoxy]propylamino]cyclobutanecarboxylic acid;
(3R)-1-[2-[2-[5-bromo-2-(8-chloro-4-oxo-chromen-2-yl)phenoxy]ethoxy]ethyl]pyrrolidine-3-carboxylic acid;
(2R)-1-[2-[2-[5-bromo-2-(8-chloro-4-oxo-chromen-2-yl)-4-methyl-phenoxy]ethoxy]ethyl]pyrrolidine-2-carboxylic acid;
4-[2-[2-[5-bromo-2-(8-chloro-4-oxo-chromen-2-yl)phenoxy]ethoxy]ethyl]morpholine-2-carboxylic acid;
(3R)-1-[2-[2-[2-(8-chloro-4-oxo-chromen-2-yl)-5-methoxy-4-methyl-phenoxy]ethoxy]ethyl]pyrrolidine-3-carboxylic acid;
(3S)-1-[2-[2-[2-(8-chloro-4-oxo-chromen-2-yl)-5-methoxy-4-methyl-phenoxy]ethoxy]ethyl]pyrrolidine-3-carboxylic acid;
4-[2-[5-bromo-2-(8-chloro-4-oxo-chromen-2-yl)phenoxy]ethyl]morpholine-3-carboxylic acid;
3-[2-[2-(8-chloro-4-oxo-chromen-2-yl)-5-methyl-phenoxy]ethylamino]cyclobutanecarboxylic acid;
1-[2-[2-(8-chloro-4-oxo-chromen-2-yl)-5-methyl-phenoxy]ethyl]azetidine-3-carboxylic acid;
cis-4-[2-[5-bromo-2-(8-chloro-4-oxo-chromen-2-yl)phenoxy]ethylamino]cyclohexanecarboxylic acid;
trans-4-[2-[5-bromo-2-(8-chloro-4-oxo-chromen-2-yl)phenoxy]ethylamino]cyclohexanecarboxylic acid;
2-[2-[2-(8-chloro-4-oxo-chromen-2-yl)-5-(trifluoromethyl)phenoxy]ethylamino]propanoic acid;
2-[2-[2-(8-chloro-4-oxo-chromen-2-yl)-5-methyl-phenoxy]ethylamino]-2-methyl-propanoic acid;
3-[3-[2-(8-chloro-4-oxo-chromen-2-yl)-5-methyl-phenoxy]propylamino]cyclobutanecarboxylic acid;
cis-4-[3-[5-bromo-2-(8-chloro-4-oxo-chromen-2-yl)phenoxy]propylamino]tetrahydropyran-2-carboxylic acid;
1-[2-[5-chloro-2-(8-chloro-4-oxo-chromen-2-yl)-4-methyl-phenoxy]ethyl]azetidine-3-carboxylic acid;
3-[3-[2-(8-chloro-4-oxo-chromen-2-yl)-5-(trifluoromethyl)phenoxy]propylamino]butanoic acid;
2-[3-[2-(8-chloro-4-oxo-chromen-2-yl)-5-(trifluoromethyl)phenoxy]propylamino]-3-cyclohexyl-propanoic acid;
(3R)-1-[2-[5-bromo-2-(8-chloro-4-oxo-chromen-2-yl)phenoxy]ethyl]-N-methylsulfonyl-pyrrolidine-3-carboxamide;
(3R)-1-[3-[2-(8-chloro-4-oxo-chromen-2-yl)-5-(trifluoromethyl)phenoxy]propyl]-N-methylsulfonyl-pyrrolidine-3-carboxamide;
(3S)-1-[3-[2-(8-chloro-4-oxo-chromen-2-yl)-5-(trifluoromethyl)phenoxy]propyl]-N-methylsulfonyl-pyrrolidine-3-carboxamide;
(3R)-1-[3-[2-(8-chloro-4-oxo-chromen-2-yl)-5-(trifluoromethyl)phenoxy]propyl]-N-cyclopropylsulfonyl-pyrrolidine-3-carboxamide;
4-[2-[2-(8-chloro-4-oxo-chromen-2-yl)-5-(trifluoromethyl)phenoxy]ethyl]-N-methylsulfonyl-morpholine-2-carboxamide;
(3S)-1-[2-[5-bromo-2-(8-chloro-4-oxo-chromen-2-yl)phenoxy]ethyl]-N-methylsulfonyl-pyrrolidine-3-carboxamide;
(3R)-1-[2-[2-(8-chloro-4-oxo-chromen-2-yl)-5-(trifluoromethyl)phenoxy]ethyl]-N-methylsulfonyl-pyrrolidine-3-carboxamide;
(3S)-1-[2-[2-(8-chloro-4-oxo-chromen-2-yl)-5-(trifluoromethyl)phenoxy]ethyl]-N-methylsulfonyl-pyrrolidine-3-carboxamide;
1-[2-[2-(8-chloro-4-oxo-chromen-2-yl)-5-(trifluoromethyl)phenoxy]ethyl]-N-cyclopropylsulfonyl-pyrrolidine-2-carboxamide;
1-[2-[2-(8-chloro-4-oxo-chromen-2-yl)-5-(trifluoromethyl)phenoxy]ethyl]-N-methylsulfonyl-pyrrolidine-2-carboxamide;
1-[2-[5-bromo-2-(8-chloro-4-oxo-chromen-2-yl)phenoxy]ethyl]-N-methylsulfonyl-pyrrolidine-2-carboxamide;
1-[2-[5-bromo-2-(8-chloro-4-oxo-chromen-2-yl)phenoxy]ethyl]pyrrolidin-2-one;
N-[1-[2-[5-bromo-2-(8-chloro-4-oxo-chromen-2-yl)phenoxy]ethyl]pyrrolidin-3-yl]-N-methyl-methanesulfonamide;
8-chloro-2-[2-[3-[(3R,4S)-3,4-dihydroxypyrrolidin-1-yl]propoxy]-4-(trifluoromethyl)phenyl]chromen-4-one;
8-chloro-2-[2-[3-[(1,1-dioxothian-4-yl)amino]propoxy]-4-(trifluoromethyl)phenyl]chromen-4-one;
8-chloro-2-[2-[3-(2-methylsulfonylethylamino)propoxy]-4-(trifluoromethyl)phenyl]chromen-4-one;
2-[3-[2-(8-chloro-4-oxo-chromen-2-yl)-5-(trifluoromethyl)phenoxy]propylamino]ethanesulfonamide;
8-chloro-2-[2-[3-(4-methylsulfonylpiperazin-1-yl)propoxy]-4-(trifluoromethyl)phenyl]chromen-4-one;
2-[2-[2-(4-acetylpiperazin-1-yl)ethoxy]-4-bromo-phenyl]-8-chloro-chromen-4-one;
1-[2-[5-bromo-2-(8-chloro-4-oxo-chromen-2-yl)phenoxy]ethyl]pyrrolidine-3-carboxamide;
2-[4-bromo-2-[2-(3-methylsulfonylpyrrolidin-1-yl)ethoxy]phenyl]-8-chloro-chromen-4-one;
4-[2-[5-bromo-2-(8-chloro-4-oxo-chromen-2-yl)phenoxy]ethyl]thiomorpholine-3-carboxamide;
N-[3-[2-(8-chloro-4-oxo-chromen-2-yl)-5-(trifluoromethyl)phenoxy]-2-hydroxy-propyl]cyclopropanesulfonamide;
8-chloro-2-[2-[2-[4-(1H-tetrazol-5-yl)-1-piperidyl]ethoxy]-4-(trifluoromethyl)phenyl]chromen-4-one;
8-chloro-2-[2-[3-(3-methylsulfonylpyrrolidin-1-yl)propoxy]-4-(trifluoromethyl)phenyl]chromen-4-one;
2-[4-bromo-2-[2-(1,1-dioxo-1,4-thiazinan-4-yl)ethoxy]phenyl]-8-chloro-chromen-4-one;
2-[4-bromo-2-[2-(2-morpholinoethoxy)ethoxy]phenyl]-8-chloro-chromen-4-one;
1-[2-[2-(8-chloro-4-oxo-chromen-2-yl)-5-methyl-phenoxy]ethyl]imidazolidin-2-one;
1-[2-[2-(8-chloro-4-oxo-chromen-2-yl)-5-methyl-phenoxy]ethyl]-3-methyl-imidazolidin-2-one;
8-chloro-2-[2-[2-(4-methylsulfinyl-1-piperidyl)ethoxy]-4-(trifluoromethyl)phenyl]chromen-4-one;
8-chloro-2-[2-[2-[4-(methylsulfonimidoyl)-1-piperidyl]ethoxy]-4-(trifluoromethyl)phenyl]chromen-4-one;
ethyl 2-[3-[2-(8-chloro-4-oxo-chromen-2-yl)-5-methyl-phenoxy]propylamino]-2-oxo-acetate;
N-[2-[2-(8-chloro-4-oxo-chromen-2-yl)-5-(trifluoromethyl)phenoxy]ethyl]cyclopropanesulfonamide;
N-[3-[2-(8-chloro-4-oxo-chromen-2-yl)-5-(trifluoromethyl)phenoxy]propyl]benzenesulfonamide;
N-[3-[2-(8-chloro-4-oxo-chromen-2-yl)-5-(trifluoromethyl)phenoxy]propyl]acetamide;
N-[3-[2-(8-chloro-4-oxo-chromen-2-yl)-5-(trifluoromethyl)phenoxy]propyl]propanamide;
ethyl 2-[3-[2-(8-chloro-4-oxo-chromen-2-yl)-5-(trifluoromethyl)phenoxy]propylamino]-2-oxo-acetate;
ethyl 2-[2-[2-(8-chloro-4-oxo-chromen-2-yl)-5-(trifluoromethyl)phenoxy]ethylamino]-2-oxo-acetate;
ethyl 2-[2-[4-bromo-2-(8-chloro-4-oxo-chromen-2-yl)-5-methoxy-phenoxy]ethylamino]-2-oxo-acetate;
ethyl 2-[2-[2-(8-chloro-6-methoxy-4-oxo-chromen-2-yl)-5-methyl-phenoxy]ethylamino]-2-oxo-acetate;
ethyl 2-[2-[2-(8-chloro-4-oxo-chromen-2-yl)-5-methoxy-phenoxy]ethylamino]-2-oxo-acetate;
methyl N-[2-[5-bromo-2-(8-chloro-4-oxo-chromen-2-yl)phenoxy]ethyl]carbamate;
ethyl N-[2-[5-bromo-2-(8-chloro-4-oxo-chromen-2-yl)phenoxy]ethyl]carbamate;
8-chloro-2-[4-methyl-2-[3-(methylsulfamoylamino)propoxy]phenyl]chromen-4-one;
2-[4-bromo-2-[(1-cyclopropylsulfonyl-4-piperidyl)oxy]phenyl]-8-chloro-chromen-4-one;
ethyl 2-[2-[2-(8-chloro-4-oxo-chromen-2-yl)-5-methyl-phenoxy]ethylamino]-2-oxo-acetate;
2-[[1-[[2-(8-chloro-4-oxo-chromen-2-yl)-5-(trifluoromethyl)phenoxy]methyl]cyclopropyl]amino]-2-oxo-acetic acid;
2-[2-[2-(8-chloro-4-oxo-chromen-2-yl)-5-methoxy-phenoxy]ethylamino]-2-oxo-acetic acid;
2-[2-[2-(8-chloro-4-oxo-chromen-2-yl)-3-fluoro-5-methyl-phenoxy]ethylamino]-2-oxo-acetic acid;
2-[2-[5-bromo-2-(8-chloro-4-oxo-chromen-2-yl)phenoxy]ethylamino]-2-oxo-acetic acid;
2-[2-[2-(8-chloro-4-oxo-chromen-2-yl)-4,5-dimethoxy-phenoxy]ethylamino]-2-oxo-acetic acid;
2-[[2-[2-(8-chloro-4-oxo-chromen-2-yl)-5-(trifluoromethyl)phenoxy]-1,1-dimethyl-ethyl]amino]-2-oxo-acetic acid;
2-[3-[2-(8-chloro-6-methoxy-4-oxo-chromen-2-yl)-5-(trifluoromethyl)phenoxy]propylamino]-2-oxo-acetic acid;
2-[3-[2-(8-chloro-4-oxo-chromen-2-yl)-5-(trifluoromethyl)phenoxy]propylamino]-2-oxo-acetic acid;
2-[2-[2-(8-chloro-4-oxo-chromen-2-yl)-3-methoxy-5-methyl-phenoxy]ethylamino]-2-oxo-acetic acid;
2-[2-[2-(8-chloro-4-oxo-chromen-2-yl)-5-(trifluoromethyl)phenoxy]ethylamino]-2-oxo-acetic acid;
2-[3-[2-(8-chloro-4-oxo-chromen-2-yl)-5-methyl-phenoxy]propylamino]-2-oxo-acetic acid;
2-[2-[2-(8-chloro-4-oxo-chromen-2-yl)-5-methyl-phenoxy]ethylamino]-2-oxo-acetic acid;
2-[2-[4-bromo-2-(8-chloro-4-oxo-chromen-2-yl)-5-methoxy-phenoxy]ethylamino]-2-oxo-acetic acid;
2-[2-[2-(8-chloro-4-oxo-chromen-2-yl)-5-(trifluoromethyl)phenoxy]ethylamino]acetic acid;
3-[2-[2-(8-chloro-4-oxo-chromen-2-yl)-5-(trifluoromethyl)phenoxy]ethylamino]propanoic acid;
2-[3-[2-(8-chloro-4-oxo-chromen-2-yl)-5-(trifluoromethyl)phenoxy]propylamino]acetic acid;
2-oxo-2-[(3S)-3-[2-(8-chloro-4-oxo-chromen-2-yl)-5-(trifluoromethyl)phenoxy]pyrrolidin-1-yl]acetic acid;
2-[3-[2-(8-chloro-4-oxo-chromen-2-yl)-5-(trifluoromethyl)phenoxy]azetidin-1-yl]-2-oxo-acetic acid;
3-[[4-[5-bromo-2-(8-chloro-4-oxo-chromen-2-yl)phenoxy]-1-piperidyl]methyl]benzoic acid;
2-[4-[2-(8-chloro-4-oxo-chromen-2-yl)-5-(trifluoromethyl)phenoxy]-1-piperidyl]-2-oxo-acetic acid;
2-oxo-2-[(3R)-3-[2-(8-chloro-4-oxo-chromen-2-yl)-5-(trifluoromethyl)phenoxy]pyrrolidin-1-yl]acetic acid;
3-[4-[5-bromo-2-(8-chloro-4-oxo-chromen-2-yl)phenoxy]-1-piperidyl]cyclobutanecarboxylic acid;
2-[(3S)-3-[2-(8-chloro-4-oxo-chromen-2-yl)-5-methyl-phenoxy]pyrrolidin-1-yl]-2-oxo-acetic acid;
2-[[3-[2-(8-chloro-4-oxo-chromen-2-yl)-5-(trifluoromethyl)phenoxy]cyclobutyl]amino]-2-oxo-acetic acid;
2-[3-[2-(8-chloro-4-oxo-chromen-2-yl)-5-(trifluoromethyl)phenoxy]propyl-methyl-amino]acetic acid;
2-[2-[2-(8-chloro-4-oxo-chromen-2-yl)-5-methyl-phenoxy]ethyl-cyclopropyl-amino]-2-oxo-acetic acid;
8-chloro-2-[2-[2-[ethylsulfamoyl(methyl)amino]ethoxy]-4-methyl-phenyl]-4-oxo-chromene;
ethyl 2-[2-[2-(8-chloro-4-oxo-chromen-2-yl)-5-methyl-phenoxy]ethyl-methyl-amino]-2-oxo-acetate;
ethyl 2-[3-[2-(8-chloro-4-oxo-chromen-2-yl)-5-(trifluoromethyl)phenoxy]propyl-ethylsulfonyl-amino]-2-oxo-acetate;
3-[3-[2-(8-chloro-4-oxo-chromen-2-yl)-5-(trifluoromethyl)phenoxy]propyl-cyclopropylsulfonyl-amino]cyclobutanecarboxylic acid;
2-[carboxymethyl-[3-[2-(8-chloro-4-oxo-chromen-2-yl)-5-(trifluoromethyl)phenoxy]propyl]amino]acetic acid;
cis-3-[benzenesulfonyl-[3-[2-(8-chloro-4-oxo-chromen-2-yl)-5-(trifluoromethyl)phenoxy]propyl]amino]cyclobutanecarboxylic acid;
trans-3-[benzenesulfonyl-[3-[2-(8-chloro-4-oxo-chromen-2-yl)-5-(trifluoromethyl)phenoxy]propyl]amino]cyclobutanecarboxylic acid;
2-[benzenesulfonyl-[3-[2-(8-chloro-4-oxo-chromen-2-yl)-5-(trifluoromethyl)phenoxy]propyl]amino]acetic acid;
2-[acetyl-[3-[2-(8-chloro-4-oxo-chromen-2-yl)-5-(trifluoromethyl)phenoxy]propyl]amino]acetic acid;
2-[3-[2-(8-chloro-4-oxo-chromen-2-yl)-5-(trifluoromethyl)phenoxy]propyl-cyclopropylsulfonyl-amino]acetic acid;
N′-[2-[2-(8-chloro-4-oxo-chromen-2-yl)-5-(trifluoromethyl)phenoxy]ethyl]oxamide;
N′-[2-[2-(8-chloro-4-oxo-chromen-2-yl)-5-methoxy-phenoxy]ethyl]oxamide;
N-[2-[2-(8-chloro-4-oxo-chromen-2-yl)-5-(trifluoromethyl)phenoxy]ethyl]-N′-cyclopropyl-oxamide;
N-[3-[2-(8-chloro-4-oxo-chromen-2-yl)-5-(trifluoromethyl)phenoxy]propyl]-N′-cyclopropylsulfonyl-oxamide;
N-[2-[2-(8-chloro-4-oxo-chromen-2-yl)-5-methyl-phenoxy]ethyl]-2-[(3S)-3-hydroxypyrrolidin-1-yl]-2-oxo-acetamide;
(2S)-2-[2-[2-(8-chloro-4-oxo-chromen-2-yl)-5-methyl-phenoxy]ethylcarbamoylamino]propanoic acid;
(5S)-3-[2-[2-(8-chloro-4-oxo-chromen-2-yl)-5-methyl-phenoxy]ethyl]-5-isopropyl-imidazolidine-2,4-dione;
(5S)-3-[2-[2-(8-chloro-4-oxo-chromen-2-yl)-5-(trifluoromethyl)phenoxy]ethyl]-5-isopropyl-imidazolidine-2,4-dione;
2-[2-[2-(8-chloro-4-oxo-chromen-2-yl)-5-methyl-phenoxy]ethylcarbamoylamino]acetic acid;
(5S)-3-[2-[2-(8-chloro-4-oxo-chromen-2-yl)-5-(trifluoromethyl)phenoxy]ethyl]-5-methyl-imidazolidine-2,4-dione;
1-[3-[2-(8-chloro-4-oxo-chromen-2-yl)-5-methyl-phenoxy]propyl]-3-(p-tolylsulfonyl)urea;
3-[2-(8-chloro-4-oxo-chromen-2-yl)-5-(trifluoromethyl)phenoxy]propylurea;
1-[2-[2-(8-chloro-4-oxo-chromen-2-yl)-5-(trifluoromethyl)phenoxy]ethyl]-3-(p-tolylsulfonyl)urea;
8-chloro-4-oxo-2-[2-[2-(sulfamoylamino)ethoxy]-4-(trifluoromethyl)phenyl]chromene;
8-chloro-4-oxo-2-[2-[3-(sulfamoylamino)propoxy]-4-(trifluoromethyl)phenyl]chromene;
8-chloro-2-[4-methyl-2-[2-(sulfamoylamino)ethoxy]phenyl]-4-oxo-chromene;
2-[3-[2-[2-(8-chloro-4-oxo-chromen-2-yl)-5-(trifluoromethyl)phenoxy]ethyl]-2-oxo-imidazolidin-1-yl]-2-oxo-acetic acid;
(3S)-1-[3-[2-(8-chloro-4-oxo-chromen-2-yl)-5-(trifluoromethyl)phenoxy]-2-hydroxy-propyl]pyrrolidine-3-carboxylic acid;
(3S)-1-[3-[5-bromo-2-(8-chloro-4-oxo-chromen-2-yl)phenoxy]-2-hydroxy-propyl]pyrrolidine-3-carboxylic acid;
(3R)-1-[3-[5-bromo-2-(8-chloro-4-oxo-chromen-2-yl)phenoxy]-2-hydroxy-propyl]pyrrolidine-3-carboxylic acid;
2-[[3-[2-(8-chloro-4-oxo-chromen-2-yl)-5-(trifluoromethyl)phenoxy]-2-hydroxy-propyl]amino]-2-oxo-acetic acid;
2-[2-[2-(8-chloro-4-oxo-chromen-2-yl)-5-(trifluoromethyl)phenoxy]ethylamino]acetamide;
2-[2-[2-(8-chloro-4-oxo-chromen-2-yl)-5-(trifluoromethyl)phenoxy]ethylamino]propanamide;
and
1-[3-[5-bromo-2-(8-chloro-4-oxo-chromen-2-yl)phenoxy]propyl]-5-oxo-pyrrolidine-3-carboxylic acid;
   or a pharmaceutically acceptable salt thereof.

In another embodiment (xxi) of the present invention, particular compounds of the present invention are selected from:
1-[3-[5-bromo-2-(8-chloro-4-oxo-chromen-2-yl)phenoxy]propyl]pyrrolidine-3-carboxylic acid;
4-[3-[5-bromo-2-(8-chloro-4-oxo-chromen-2-yl)phenoxy]propyl]morpholine-2-carboxylic acid;
3-[3-[2-(8-chloro-4-oxo-chromen-2-yl)-5-(trifluoromethyl)phenoxy]propylamino]cyclobutanecarboxylic acid;
3-[3-[2-(8-chloro-4-oxo-chromen-2-yl)-5-(trifluoromethyl)phenoxy]propylamino]cyclopentanecarboxylic acid;
(3R)-1-[3-[2-(8-chloro-4-oxo-chromen-2-yl)-5-(trifluoromethyl)phenoxy]propyl]pyrrolidine-3-carboxylic acid;
(3S)-1-[3-[2-(8-chloro-4-oxo-chromen-2-yl)-5-(trifluoromethyl)phenoxy]propyl]pyrrolidine-3-carboxylic acid;
(3S)-1-[3-[5-bromo-2-(8-chloro-4-oxo-chromen-2-yl)-4-methoxy-phenoxy]propyl]pyrrolidine-3-carboxylic acid;
4-[3-[2-(8-chloro-4-oxo-chromen-2-yl)-5-(trifluoromethyl)phenoxy]propyl]morpholine-2-carboxylic acid;
1-[2-[5-bromo-2-(8-chloro-4-oxo-chromen-2-yl)phenoxy]ethyl]pyrrolidine-2-carboxylic acid;
4-[2-[5-bromo-2-(8-chloro-4-oxo-chromen-2-yl)phenoxy]ethyl]morpholine-2-carboxylic acid;
(3R)-1-[2-[5-bromo-2-[8-chloro-4-oxo-5-(trifluoromethyl)chromen-2-yl]phenoxy]ethyl]pyrrolidine-3-carboxylic acid;
(3R)-1-[2-[2-[5-bromo-2-(8-chloro-4-oxo-chromen-2-yl)phenoxy]ethoxy]ethyl]pyrrolidine-3-carboxylic acid;
4-[2-[5-bromo-2-(8-chloro-4-oxo-chromen-2-yl)phenoxy]ethyl]morpholine-3-carboxylic acid;
(3R)-1-[2-[5-bromo-2-(8-chloro-4-oxo-chromen-2-yl)phenoxy]ethyl]-N-methylsulfonyl-pyrrolidine-3-carboxamide;
1-[2-[2-(8-chloro-4-oxo-chromen-2-yl)-5-(trifluoromethyl)phenoxy]ethyl]-N-methylsulfonyl-pyrrolidine-2-carboxamide;
2-[4-bromo-2-[2-(3-methylsulfonylpyrrolidin-1-yl)ethoxy]phenyl]-8-chloro-chromen-4-one;
(2S)-2-[2-[2-(8-chloro-4-oxo-chromen-2-yl)-5-methyl-phenoxy]ethylcarbamoylamino]propanoic acid;
3-[2-(8-chloro-4-oxo-chromen-2-yl)-5-(trifluoromethyl)phenoxy]propylurea;
8-chloro-4-oxo-2-[2-[3-(sulfamoylamino)propoxy]-4-(trifluoromethyl)phenyl]chromene;
(3R)-1-[3-[5-bromo-2-(8-chloro-4-oxo-chromen-2-yl)phenoxy]-2-hydroxy-propyl]pyrrolidine-3-carboxylic acid; and
2-[[3-[2-(8-chloro-4-oxo-chromen-2-yl)-5-(trifluoromethyl)phenoxy]-2-hydroxy-propyl]amino]-2-oxo-acetic acid;
   or a pharmaceutically acceptable salt thereof.

### Synthesis

The compounds of the present invention can be prepared by any conventional means. Suitable processes for synthesizing these compounds as well as their starting materials are provided in the schemes below and in the subsequent examples. All substituents, in particular, R¹ to R¹², L, Cy1, Cy2, X and Y are defined above unless otherwise indicated. Furthermore, and unless explicitly otherwise stated, all reactions, reaction conditions, abbreviations and symbols have the meanings well known to a person of ordinary skill in organic chemistry.

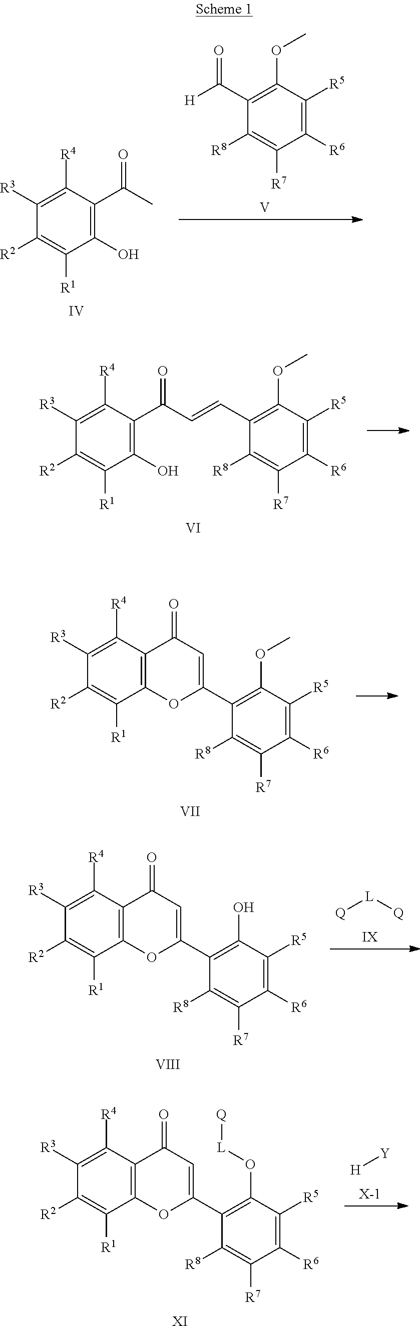

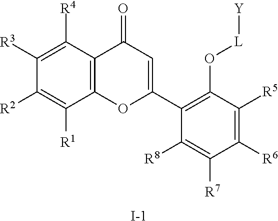

wherein Q is halogen or OMs.

Condensation of ketone IV with aldehyde V in the presence of a base, such as KOH, in a suitable solvent, such as ethanol, affords α,β-unsaturated carbonyl intermediate VI. Cyclization of α,β-unsaturated carbonyl intermediate VI in the presence of a suitable Lewis acid, such as I₂, KI or NaI, in a suitable solvent, such as DMSO, affords flavone derivative VII. Demethylation of flavone derivative VII with a suitable Lewis acid, such as BBr₃, in a suitable solvent, such as dichloromethane, affords compound of formula VIII. Substitution of compounds of formula VIII with compounds of formula IX in the presence of a suitable base, such as K₂CO₃, in a suitable solvent, such as DMF, affords compounds of formula XI. Substitution of compounds of formula XI with compounds of formula X-1 in the presence of a suitable base, such as K₂CO₃, in a suitable solvent, such as DMF, affords compounds of formula I-1.

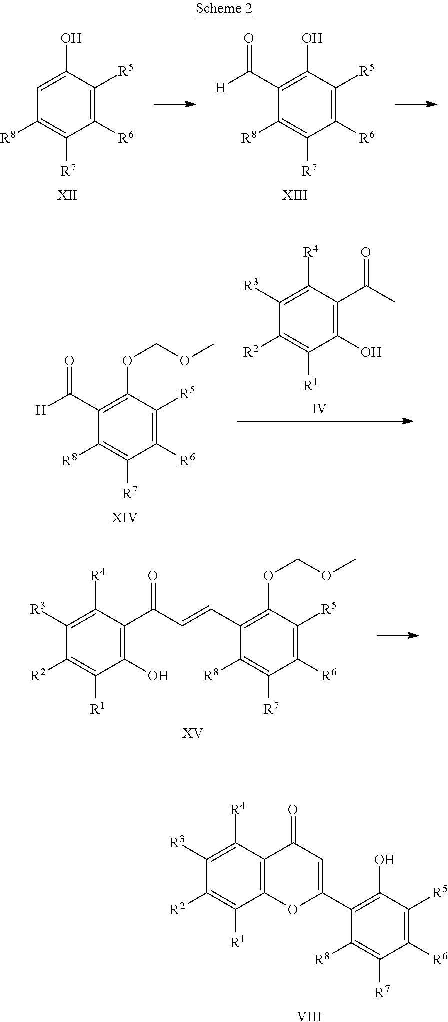

The compounds of formula VIII can also be prepared according to the Scheme 2. Formylation of compounds of formula XII with formaldehyde in the presence of a suitable base, such as TEA, with a suitable Lewis acid, such as MgCl₂, in a suitable solvent, such as ACN, affords aldehyde derivative XIII. Protection of aldehyde derivative XIII with bromo(methoxy)methane in the presence of a suitable base, such as NaH, in a suitable solvent, such as THF, affords aldehyde derivative XIV. Condensation of compounds of formula XIV with substituted ketone IV in the presence of a base, such as KOH, in a suitable solvent, such as ethanol, affords α,β-unsaturated carbonyl intermediate XV. Cyclization of intermediate XV in the presence of a suitable Lewis acid, such as I₂, KI or NaI, in a suitable solvent, such as DMSO, affords compounds of formula VIII.

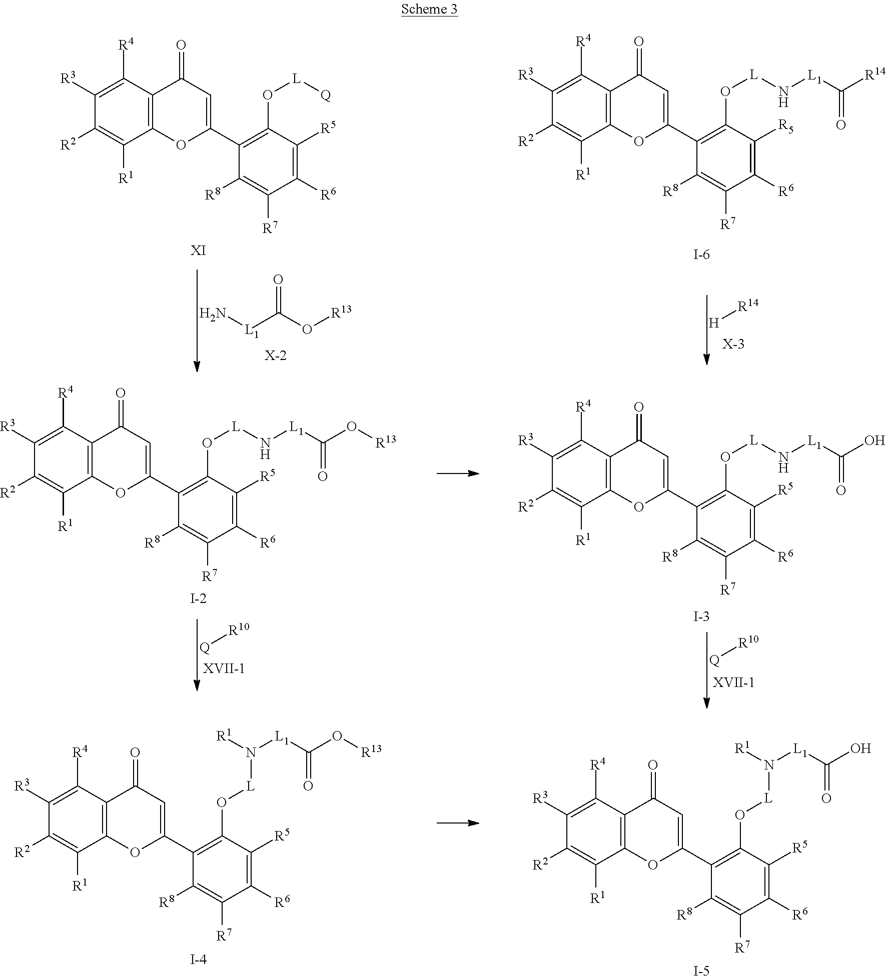

wherein Q is halogen or OMs; L₁ is C₁₋₆alkyl, carbonyl or C₃₋₇cycloalkyl; R¹³ is C₁₋₆alkyl; R¹⁴ is C₁₋₆alkylsulfonylamino, C₃₋₇cycloalkylsulfonylamino, C₃₋₇cycloalkylamino or hydroxyheterocyclyl.

Substitution of compounds of formula XI with amine X-2 in the presence of a suitable base, such as NaHCO₃, in a suitable solvent, such as DMF, affords compounds of formula I-2.

Hydrolysis of compounds of formula I-2 in the presence of a suitable base, such as LiOH, in a suitable solvent, such as THF/water; or a suitable Lewis acid, such as TFA, in a suitable solvent, such as dichloromethane, affords compounds of formula I-3.

Treatment of compounds of formula I-2 with compounds of formula XVII-1 in the presence of a suitable base, such as TEA, in a suitable solvent, such as dichloromethane, affords compounds of formula I-4. The following hydrolysis of compounds of formula I-4 in the presence of a suitable base, such as LiOH, in a suitable solvent, such as THF and water, affords compounds of formula I-5. The compounds of formula I-5 can also be prepared in the other route in scheme 3 via I-3. Treatment of compounds of formula I-3 with compounds of formula XVII-1 in the presence of a suitable base, such as DIPEA, in a suitable solvent, such as dichloromethane, affords compounds of formula I-5.

Condensation of compounds of formula I-3 with compounds of formula X-3 in the presence of a condensation reagent, such as HATU, in a suitable solvent, such as dichloromethane, affords compound of formula I-6.

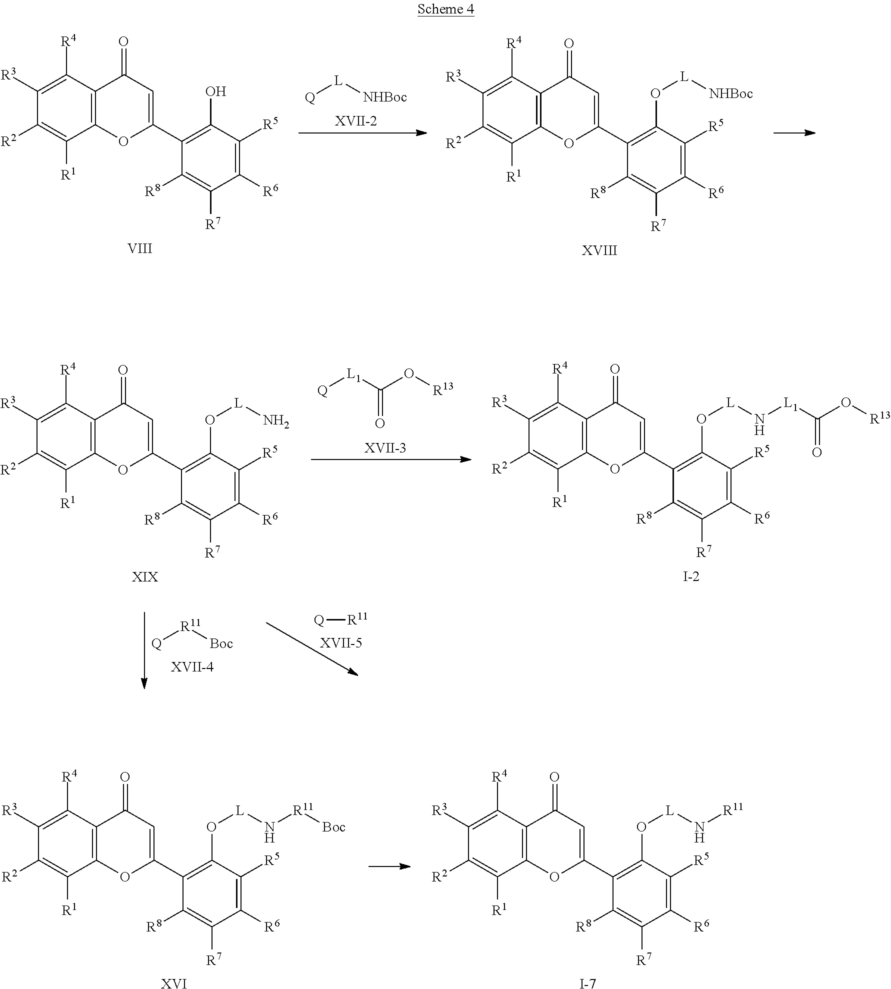

wherein Q is halogen or OMs; L₁ is C₁₋₆alkyl, carbonyl or C₃₋₇cycloalkyl; R¹³ is C₁₋₆alkyl. The compounds of formula I-2 can also be prepared according to the Scheme 4.

Substitution of compounds of formula VIII with compounds of formula XVII-2 in the presence of a suitable base, such as K₂CO₃, in a suitable solvent, such as DMF, affords compounds of formula XVIII. Boc deprotection of compounds of formula XVIII with a suitable Lewis acid, such as TFA, in a suitable solvent, such as dichloromethane, affords compounds of formula XIX.

Treatment of compounds of formula XIX with compounds of formula XVII-3 in the presence of a suitable base, such as DIPEA, in a suitable solvent, such as dichloromethane, affords compounds of formula I-2.

Substitution of compounds of formula XIX with compounds of formula XVII-4 in the presence of a suitable base, such as DIPEA, in a suitable solvent, such as dichloromethane, affords compounds of formula XVI. Deprotection of compounds of formula XVI in the presence of a Lewis acid, such as TFA, in a suitable solvent, such as dichloromethane, affords compounds of formula I-7. Compounds of formula I-7 can also be prepared from the substitution of compounds of formula XIX with halide XVII-5 in the presence of a suitable base, such as DIPEA, in a suitable solvent, such as dichloromethane.

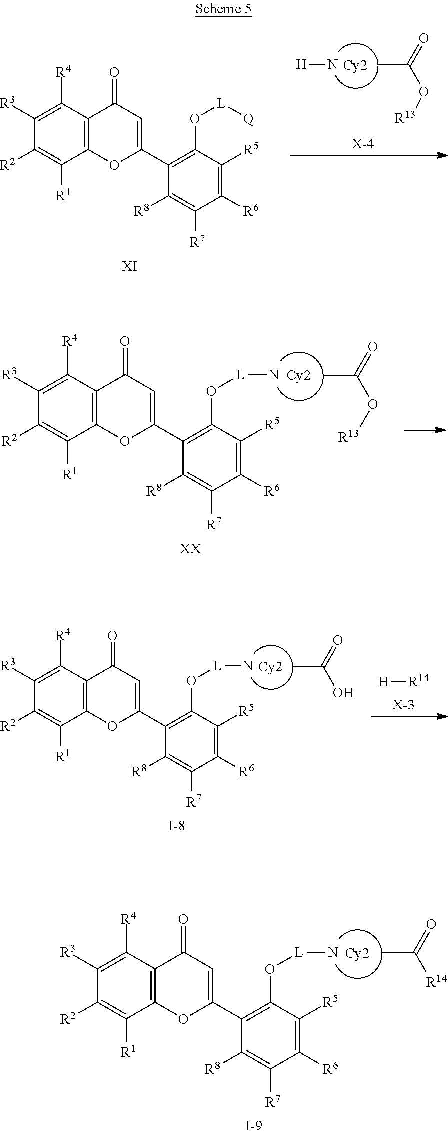

Wherein Q is halogen or OMs; R¹⁴ is C₁₋₆alkylsulfonylamino, C₃₋₇cycloalkylsulfonylamino, C₃₋₇cycloalkylamino or hydroxyheterocyclyl.

Substitution of compounds of formula XI with compounds of formula X-4 in the presence of a suitable base, such as NaHCO₃, in a suitable solvent, such as DMF, affords flavone derivative XX. Hydrolysis of the compounds of formula XX in the presence of a suitable base, such as LiOH, in a suitable solvent, such as THF/water, or a suitable Lewis acid, such as TFA, in a suitable solvent, such as dichloromethane, affords carboxylic acid I-8. Condensation of compounds of formula I-8 with compounds of formula X-3 in the presence of a suitable condensation reagent, such as HATU, with a suitable base, such as NaH, in a suitable solvent, such as THF, affords amide derivative of formula I-9.

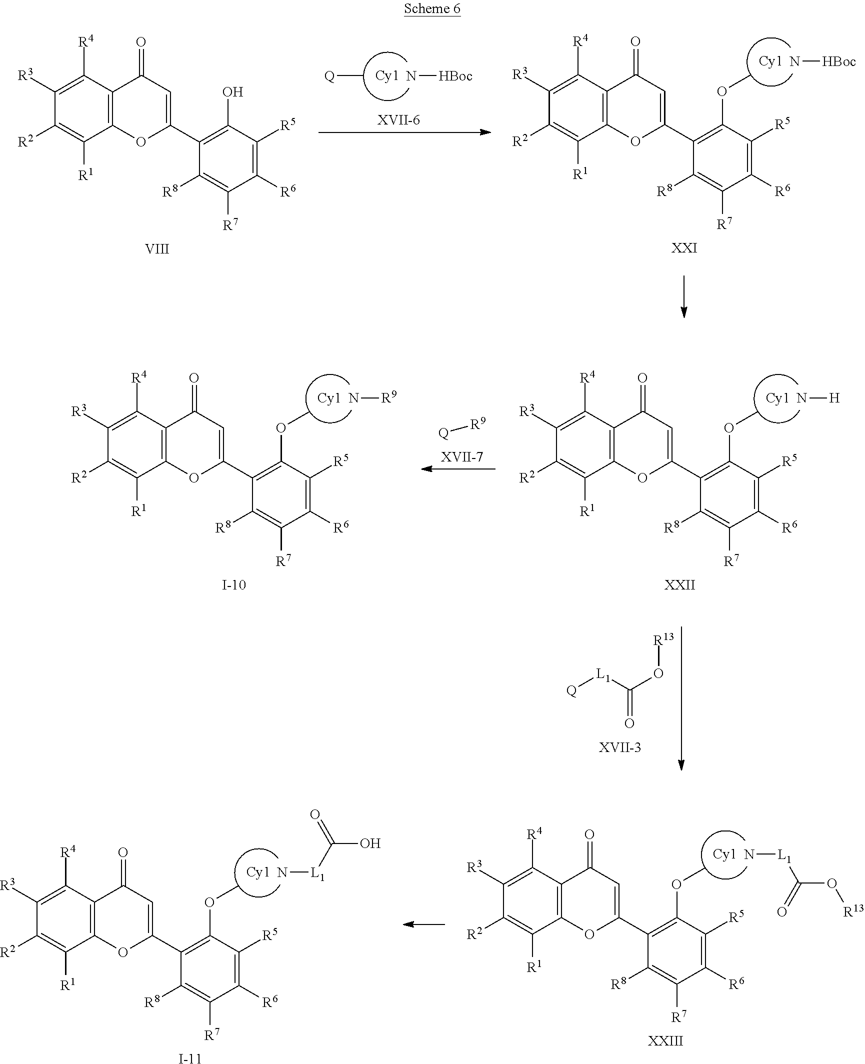

wherein Q is halogen or OMs; L₁ is C₁₋₆alkyl, carbonyl or C₃₋₇cycloalkyl; R¹³ is C₁₋₆alkyl.

Substitution of compounds of formula VIII with compounds of formula XVII-6 in the presence of a suitable base, such as K₂CO₃, in a suitable solvent, such as DMF, affords compounds of formula XXI. Deprotection of compounds of formula XXI with a suitable Lewis acid, such as TFA, in a suitable solvent, such as dichloro methane, affords compounds of formula XXII. Treatment of compounds of formula XXII with compounds of formula XVII-7 in the presence of a suitable base, such as DIPEA, in a suitable solvent, such as dichloro methane, affords compounds of formula I-10.

Treatment of compounds of formula XXII with compounds of formula XVII-3 in the presence of a suitable base, such as DIPEA, in a suitable solvent, such as dichloromethane, affords compounds of formula XXIII. Hydrolysis of compounds of formula XXIII in the presence of a suitable base, such as LiOH, in a suitable solvent, such as THF/water, affords compounds of formula I-11.

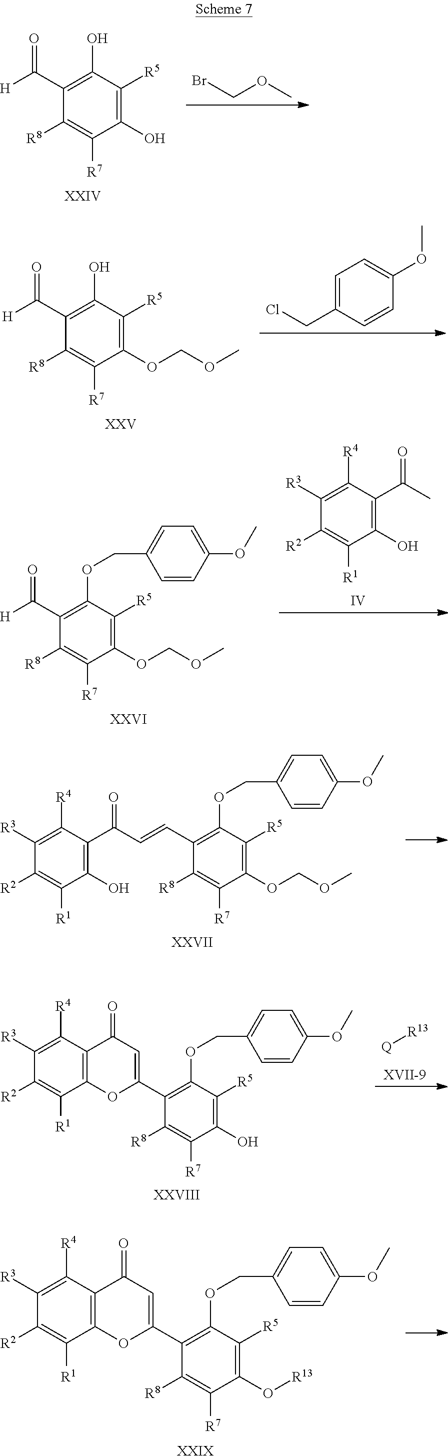

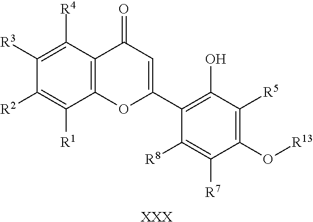

Wherein Q is halogen or OMs; R¹³ is C₁₋₆alkyl.

Protection of 4-hydroxy group of compounds of formula XXIV with bromomethyl methyl ether in the presence of a suitable base, such as DIPEA, in a suitable solvent, such as dichloromethane, affords compounds of formula XXV. Protection of 2-hydroxy group of compounds of formula XXV with 4-methoxybenzyl chloride in the presence of a suitable base, such as K₂CO₃, in a suitable solvent, such as DMF, affords compounds of formula XXVI.

Condensation of ketone IV with aldehyde XXVI in the presence of a base, such as KOH, in a suitable solvent, such as ethanol, affords α,β-unsaturated carbonyl intermediate XXVII. Cyclization of α,β-unsaturated carbonyl intermediate XXVII in the presence of a suitable Lewis acid, such as I₂, KI or NaI, in a suitable solvent, such as DMSO, affords flavone derivative XXVIII. Alkylation of compounds of formula XXVIII with compounds of formula XVII-9 in the presence of a base, such as KOH, in a suitable solvent, such as ethanol, affords compounds of formula XXIX. Deprotection of compounds of formula XXIX in the presence of Lewis acid, such as TFA, affords phenol XXX.

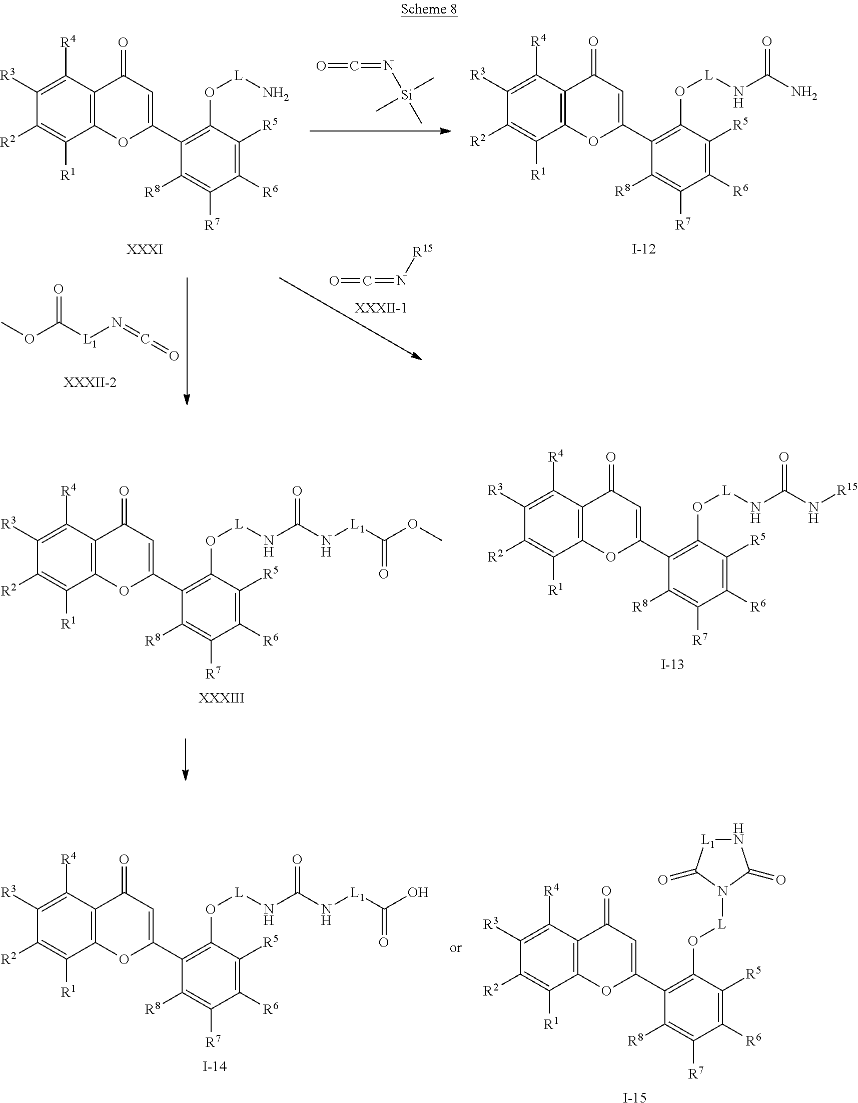

wherein Q is halogen or OMs; L₁ is C₁₋₆alkyl, carbonyl or C₃₋₇cycloalkyl; R¹⁵ is C₁₋₆alkylphenylsulfonyl.

Treatment of compounds of formula XXXI with isocyanato(trimethyl)silane in the presence of a suitable base, such as DIPEA, in a suitable solvent, such as dichloromethane, affords compounds of formula I-12. Treatment of compounds of formula XXXI with compounds of formula XXXII-1 in the presence of a suitable base, such as DIPEA, in a suitable solvent, such as dichloromethane, affords compounds of formula I-13. Treatment of compounds of formula XXXI with compounds of formula XXXII-2 in the presence of a suitable base, such as DIPEA, in a suitable solvent, such as dichloromethane, affords compounds of formula XXXIII.

Deprotection of compounds of formula XXXIII in the presence of a base, such as sodium hydroxide, in a suitable solvent, such as THF, affords compounds of formula I-14 and in situ-cyclization compounds of formula I-15.

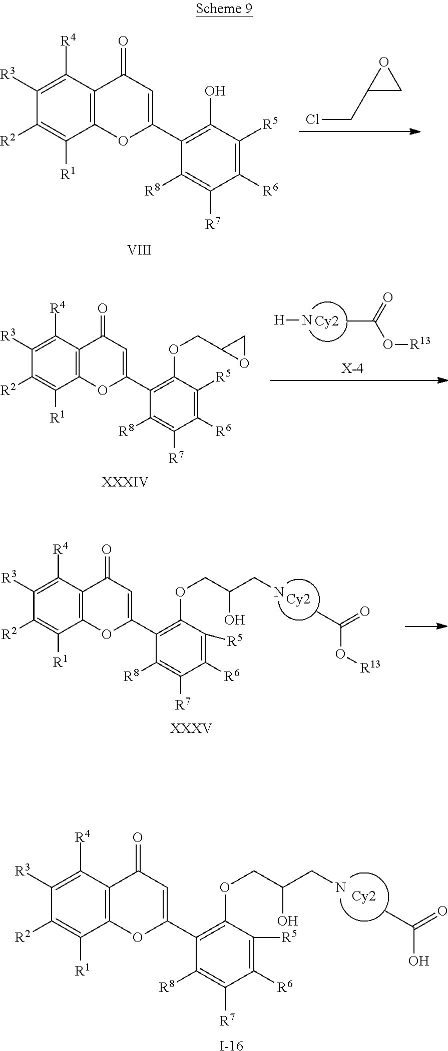

wherein R¹³ is C₁₋₆alkyl.

Substitution of compounds of formula VIII with 2-(chloromethyl)oxirane in the presence of a suitable base, such as K₂CO₃, in a suitable solvent, such as DMF, affords compounds of formula XXXIV. Ring open of the epoxide of compounds of formula XXXIV in the presence of a suitable base, such as K₂CO₃, in a suitable solvent, such as DMF, affords compounds of formula XXXV. Hydrolysis of compounds of formula XXXV in the presence of a suitable base, such as LiOH, in a suitable solvent, such as THF/water, affords compounds of formula I-16.

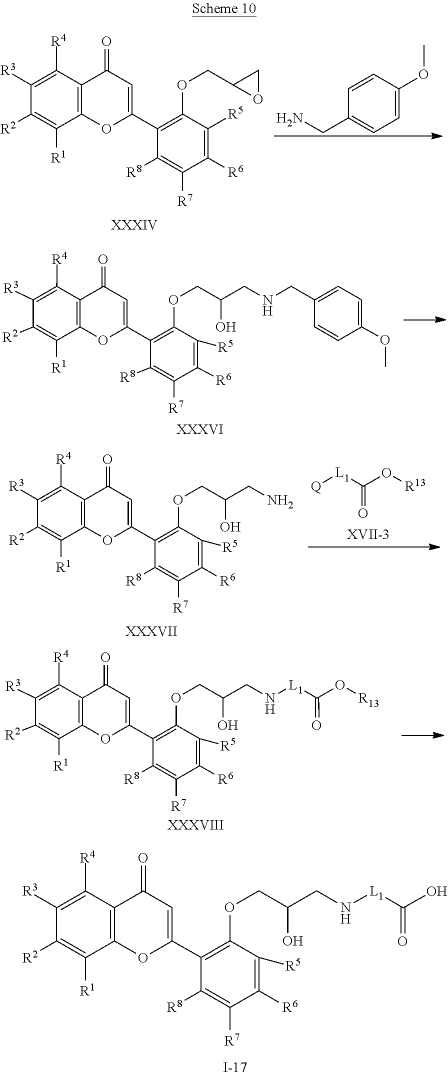

wherein L₁ is C₁₋₆alkyl, carbonyl or C₃₋₇cycloalkyl; R¹³ is C₁₋₆alkyl. Ring open of compounds of formula XXXIV with (4-methoxyphenyl)methanamine in the presence of a suitable base, such as K₂CO₃, in a suitable solvent, such as DMF, affords compounds of formula XXXVI. Deprotection of compounds of formula XXXVI in the presence of suitable Lewis acid, such as TFA, in a suitable solvent, such as dichloromethane, affords compounds of formula XXXVII. Substitution of compounds of formula XXXVII with compounds of formula XVII-3 in the presence of a suitable base, such as K₂CO₃, in a suitable solvent, such as DMF, affords compounds of formula XXXVIII. Hydrolysis of compounds of formula XXXVIII in the presence of a suitable base, such as LiOH, in a suitable solvent, such as THF/water, affords compounds of formula I-17.

This invention also relates to a process for the preparation of a compound of formula (I) comprising at least one of the following steps:

(a) Substitution of a compound of formula (XI),

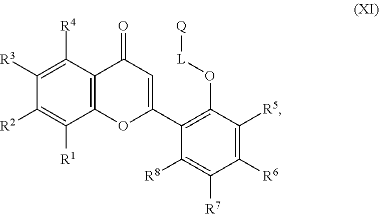

with a compound of formula (X-1),

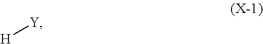

in the presence of a base;
(b) Substitution of a compound of formula (XI) with amine (X-2),

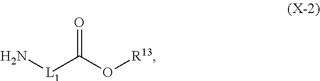

in the presence of a base;
(c) Hydrolysis of a compound of formula (I-2),

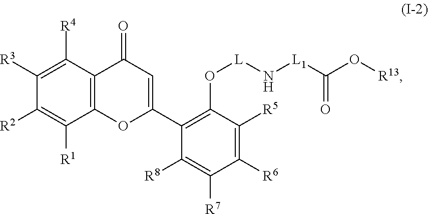

in the presence of a base;
(d) Substitution of a compound of formula (I-2) with a compound of formula (XVII-1),

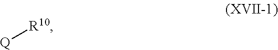

in the presence of a base;
(e) Hydrolysis of a compound of formula (I-4),

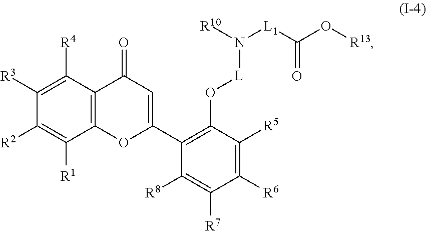

in the presence of a base or a Lewis acid;
(f) Treatment of a compound of formula (I-3),

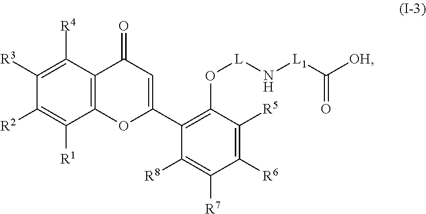

with a compound of formula (XVII-1) in the presence of a base;
(g) Condensation of a compound of formula (I-3), with a compound of formula (X-3),

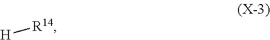

in the presence of a condensation reagent;
(h) Substitution of a compound of formula (XIX),

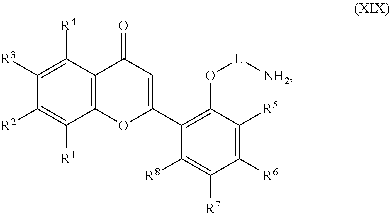

with a compound of formula (XVII-3),

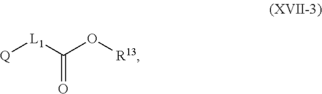

in the presence of a base;
(i) Substitution of a compound of formula (XIX), with a compound of formula (XVII-5), Q-R¹¹ (XVII-5), in the presence of a base;
(j) Deprotection of a compound of formula (XVI),

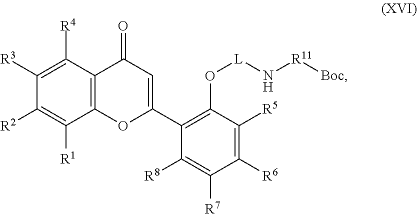

in the presence of a Lewis acid;
(k) Hydrolysis of a compound of formula (XX),

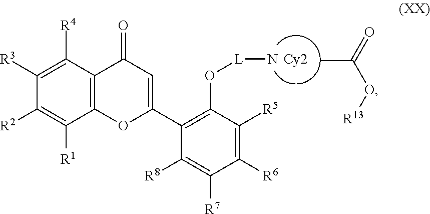

in the presence of a base or a Lewis acid;
(l) Condensation of a compound of formula (I-8),

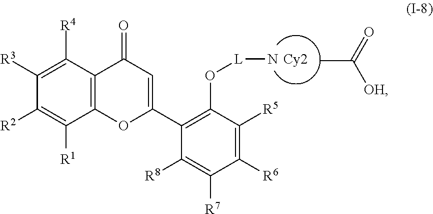

with a compound of formula (X-3), in the presence of a condensation reagent and a base;
(m) Treatment of a compound of formula (XXII),

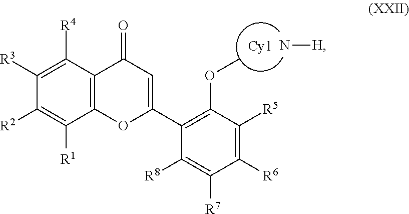

with a compound of formula (XVII-7);

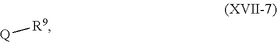

in the presence of a base;
(n) Hydrolysis of a compound of formula (XXIII),

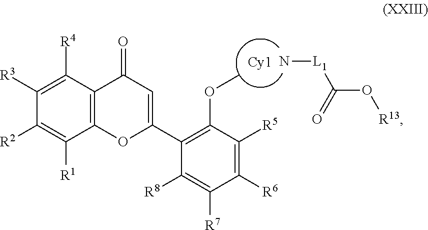

in the presence of a base;
(o) Treatment of a compound of formula (XXXI),

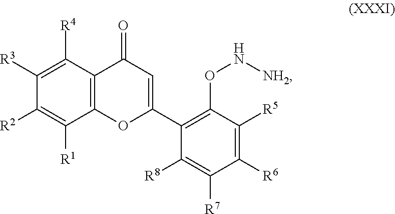

with isocyanato(trimethyl)silane in the presence of a base;
(p) Treatment of a compound of formula (XXXI) with a compound of formula (XXXII-1);

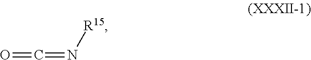

in the presence of a base;
(q) Deprotection of a compounds of formula (XXXIII),

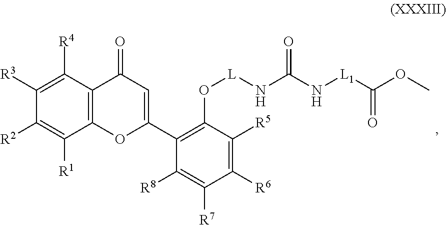

in the presence of a base;
(r) Hydrolysis of a compound of formula (XXXV),

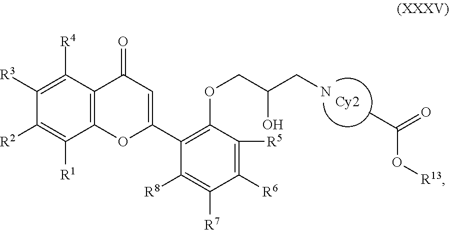

in the presence of a base;
(s) Hydrolysis of a compound of formula (XXXVIII),

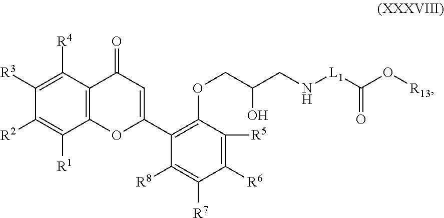

in the presence of a base:
wherein R¹ to R¹², L, Cy1, Cy2 and Y are defined above; wherein Q is halogen or Oms; L₁ is C₁₋₆alkyl, carbonyl or C₃₋₇cycloalkyl; R¹³ is C₁₋₆alkyl; R¹⁴ is C₁₋₆alkylsulfonylamino, C₃₋₇ cycloalkylsulfonylamino, C₃₋₇cycloalkylamino or hydroxyheterocyclyl; R¹⁵ is C₁₋₆alkylphenylsulfonyl.

The base in step (a) can be for example K₂CO₃;

The base in step (b) can be for example NaHCO₃;
The base in step (c) can be for example LiOH;
The base in step (d) can be for example TEA;
The base in step (e) can be for example LiOH;
The Lewis acid in step (e) can be for example TFA;
The base in step (f) can be for example DIPEA;
The condensation reagent in step (g) can be for example HATU;
The base in step (h) can be for example DIPEA;
The base in step (i) can be for example DIPEA;
The Lewis acid in step (j) can be for example TFA;
The base in step (k) can be for example LiOH;
The Lewis acid in step (k) can be for example TFA;
The condensation reagent in step (l) can be for example HATU;
The base in step (l) can be for example NaH;
The condensation reagent in step (m) can be for example LiOH;
The base in step (m) can be for example DIPEA;
The base in step (n) can be for example LiOH;
The base in step (o) can be for example DIPEA;
The base in step (p) can be for example DIPEA;
The base in step (q) can be for example sodium hydroxide;
The base in step (r) can be for example LiOH;
The base in step (s) can be for example LiOH.

A compound of formula (I) or (II) when manufactured according to the above process is also an object of the invention.

The compound of this invention also shows good safety and PK profile.

### Pharmaceutical Compositions and Administration

The invention also relates to a compound of formula (I) or (II) for use as therapeutically active substance. Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) or (II) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) or (II) is formulated in an acetate buffer, at pH 5. In another embodiment, the compounds of formula (I) or (II) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit cccDNA in HBV patients, consequently lead to the reduction of HBsAg and HBeAg (HBV e antigen) in serum. For example, such amount may be below the amount that is toxic to normal cells, or the mammal as a whole.

In one example, the pharmaceutically effective amount of the compound of the invention administered parenterally per dose will be in the range of about 0.1 to 100 mg/kg, alternatively about 0.1 to 50 mg/kg of patient body weight per day, with the typical initial range of compound used being 0.3 to 15 mg/kg/day. In another embodiment, oral unit dosage forms, such as tablets and capsules, preferably contain from about 25 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 25 to 500 mg of the compound of the invention compounded with about 90 to 30 mg anhydrous lactose, about 5 to 40 mg sodium croscarmellose, about 5 to 30 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of Formula (I) or (II), or pharmaceutically acceptable salt or enantiomer or diastereomer thereof.

In a further embodiment includes a pharmaceutical composition comprising a compound of formula (I) or (II), or pharmaceutically acceptable salt or enantiomer or diastereomer thereof, together with a pharmaceutically acceptable carrier or excipient.

Another embodiment includes a pharmaceutical composition comprising a compound of formula (I) or (II), or pharmaceutically acceptable salt or enantiomer or diastereomer thereof for use in the treatment of HBV infection.

### Indications and Methods of Treatment

The compounds of the invention can inhibit cccDNA and have anti-HBV activity.

Accordingly, the compounds of the invention are useful for the treatment or prophylaxis of HBV infection.

The invention relates to the use of a compound of formula (I) or (II) for the inhibition of cccDNA.

The invention also relates to the use of a compound of formula (I) or (II) for the inhibition of HBeAg.

The invention further relates to the use of a compound of formula (I) or (II) for the inhibition of HBsAg.

The invention relates to the use of a compound of formula (I) or (II) for the inhibition of HBV DNA.

The invention relates to the use of a compound of formula (I) or (II) for use in the treatment or prophylaxis of HBV infection.

The use of a compound of formula (I) or (II) for the preparation of medicaments useful in the treatment or prophylaxis diseases that are related to HBV infection is an object of the invention.

The invention relates in particular to the use of a compound of formula (I) or (II) for the preparation of a medicament for the treatment or prophylaxis of HBV infection.

Another embodiment includes a method for the treatment or prophylaxis of HBV infection, which method comprises administering an effective amount of a compound of Formula (I) or (II), or enantiomers, diastereomers, prodrugs or pharmaceutically acceptable salts thereof.

### EXAMPLES

The invention will be more fully understood by reference to the following examples.

They should not, however, be construed as limiting the scope of the invention.

Abbreviations used herein are as follows:
ACN: acetonitrile
BBr₃: boron tribromide
DMAP: 4-dimethylaminopyridine
DMF: N,N-dimethylformamide
IC₅₀: the molar concentration of an inhibitor, which produces 50% of the maximum possible response for that inhibitor.
FBS: fetal bovine serum
H₂O₂: hydrogen peroxide
HPLC: high performance liquid chromatography
MS (ESI): mass spectroscopy (electron spray ionization)
Ms: methylsulfonyl
obsd.: observed
PE: petroleum ether
DCM: dichloromethane
EtOAc: ethyl acetate
AcOH: acetic acid
THF: tetrahydrofuran
TFA: trifluoroacetic acid
DIPEA: N, N-Diisopropylethylamine
TEA: Triethylamine
HATU: 1-[Bis(dimethylamino)methylene]-1H-1,2,3-triazolo[4,5-b]pyridinium 3-oxid hexafluorophosphate
δ: chemical shift

### General Experimental Conditions

Intermediates and final compounds were purified by flash chromatography using one of the following instruments: i) Biotage SP1 system and the Quad 12/25 Cartridge module, ii) column chromatography on silica gel combi-flash chromatography instrument. Silica gel Brand and pore size: i) KP-STL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using X Bridge™ Perp C₁₈ (5 μm, OBD™ 30×100 mm) column or SunFire™ Perp C₁₈ (5 μm, OBD™ 30×100 mm) column.

LC/MS spectra were obtained using a Waters UPLC-SQD Mass. Standard LC/MS conditions were as follows (running time 3 minutes):

Acidic condition: A: 0.1% formic acid and 1% acetonitrile in H₂O; B: 0.1% formic acid in acetonitrile;

Basic condition: A: 0.05% NH₃—H₂O in H₂O; B: acetonitrile.

Mass spectra (MS): generally only ions which indicate the parent mass are reported, and unless otherwise stated the mass ion quoted is the positive mass ion (M+H)⁺.

NMR Spectra were obtained using Bruker Avance 400 MHz.

All reactions involving air-sensitive reagents were performed under an argon atmosphere.

Reagents were used as received from commercial suppliers without further purification unless otherwise noted.

### PREPARATIVE EXAMPLES

### Intermediate 1: 8-chloro-2-[2-hydroxy-4-(trifluoromethyl)phenyl]chromen-4-one

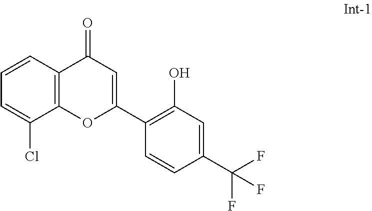 [SMILES: O=c1cc(-c2ccc(C(F)(F)F)cc2O)oc2c(Cl)cccc12]

### Step 1: Preparation of (E)-1-(3-chloro-2-hydroxy-phenyl)-3-[2-methoxy-4-(trifluoromethyl)phenyl]prop-2-en-1-one

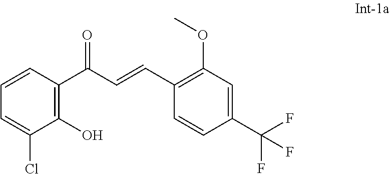 [SMILES: COc1cc(C(F)(F)F)ccc1/C=C/C(=O)c1cccc(Cl)c1O]

A mixture of 1-(3-chloro-2-hydroxy-phenyl)ethanone (2.5 g, 14.7 mmol, CAS registry number: 3226-34-4, Vendor: Bide Pharmatech, Catalog number: BD11027), 2-methoxy-4-(trifluoromethyl)benzaldehyde (2 g, 14.7 mmol, CAS registry number: 132927-09-4, Vendor: Alfa Aesar, Catalog number: H26797) and KOH (1.64 g, 29.3 mmol) in EtOH (25 mL) was stirred at 100° C. for 3 hours. After the reaction was completed, the mixture was adjusted to pH-4 by addition of 2N HCl and the resulting suspension was filtered. The solid was collected and concentrated in vacuo to give (E)-1-(3-chloro-2-hydroxy-phenyl)-3-[2-methoxy-4-(trifluoromethyl)phenyl]prop-2-en-1-one (3.3 g, yield: 78%) as a yellow solid, which was used in the next step without further purification. MS obsd. (ESI⁺) [(M+H)⁺]: 357.2.

### Step 2: Preparation of 8-chloro-2-[2-methoxy-4-(trifluoromethyl)phenyl]chromen-4-one

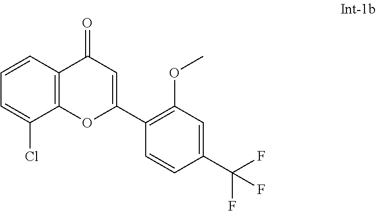 [SMILES: COc1cc(C(F)(F)F)ccc1-c1cc(=O)c2cccc(Cl)c2o1]

To a solution of (E)-1-(3-chloro-2-hydroxy-phenyl)-3-[2-methoxy-4-(trifluoromethyl)phenyl]prop-2-en-1-one (5.3 g, 18.4 mmol) in DMSO (60 mL) was added Iodine (466 mg, 1.84 mmol). The reaction mixture was stirred at 140° C. for 3 hours. After the reaction was completed, the reaction mixture was cooled to room temperature, quenched with sat. NaHSO₃ solution (10 mL). The mixture was diluted with water (100 mL) and the resulting suspension was filtered. The solid was collected and dried in vacuo to give 8-chloro-2-[2-methoxy-4-(trifluoromethyl)phenyl]chromen-4-one (5 g, yield: 94.9%) as a yellow solid, which was used in the next step without further purification. MS obsd. (ESI⁺) [(M+H)⁺]: 355.1.

### Step 3: Preparation of 8-chloro-2-[2-hydroxy-4-(trifluoromethyl)phenyl]chromen-4-one

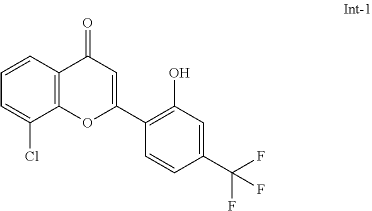 [SMILES: O=c1cc(-c2ccc(C(F)(F)F)cc2O)oc2c(Cl)cccc12]

To a solution of 8-chloro-2-[2-methoxy-4-(trifluoromethyl)phenyl]chromen-4-one (5 g, 14.0 mmol) in dichloromethane (40 mL) was added BBr₃ (1M solution in dichloromethane, 69.8 mL, 69.8 mmol) at room temperature. The mixture was stirred at room temperature overnight. After the reaction was completed, the mixture was concentrated in vacuo and then the residue was suspended in sat. NH₄Cl solution (30 mL). The solid was collected by filtration and dried in vacuo to give 8-chloro-2-[2-hydroxy-4-(trifluoromethyl)phenyl]chromen-4-one (4.1 g, yield: 86%) as a yellow solid, which was used in the next step without further purification. MS obsd. (ESI⁺) [(M+H)⁺]: 341.2.

### Intermediate 2: 8-chloro-2-(2-hydroxy-4-methyl-phenyl)chromen-4-one

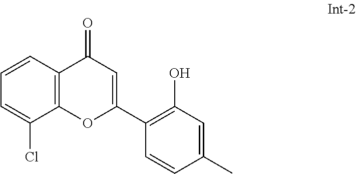 [SMILES: Cc1ccc(-c2cc(=O)c3cccc(Cl)c3o2)c(O)c1]

Int-2 was prepared in analogy to the procedure described for the preparation of compound Int-1 by using 2-methoxy-4-methyl-benzaldehyde as the starting material instead of 2-methoxy-4-(trifluoromethyl)benzaldehyde in Step 1. MS obsd. (ESI⁺) [(M+H)⁺]: 287.1.

### Intermediate 3: 8-chloro-2-(2-hydroxyphenyl)chromen-4-one

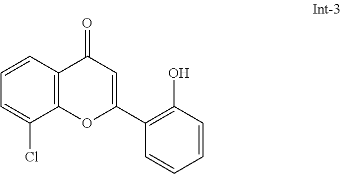 [SMILES: O=c1cc(-c2ccccc2O)oc2c(Cl)cccc12]

Int-3 was prepared in analogy to the procedure described for the preparation of compound Int-1 by using 2-hydroxybenzaldehyde as the starting material instead of 2-methoxy-4-(trifluoromethyl)benzaldehyde in Step 1. MS obsd. (ESI⁺) [(M+H)⁺]: 273.2.

### Intermediate 4: 2-(4-bromo-2-hydroxy-phenyl)-8-chloro-chromen-4-one

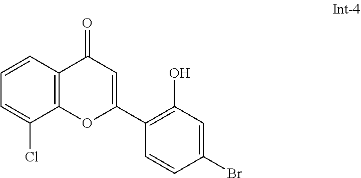 [SMILES: O=c1cc(-c2ccc(Br)cc2O)oc2c(Cl)cccc12]

Int-4 was prepared in analogy to the procedure described for the preparation of compound Int-1 by using 4-bromo-2-hydroxy-benzaldehyde as the starting material instead of 2-methoxy-4-(trifluoromethyl)benzaldehyde in Step 1. MS obsd. (ESI⁺) [(M+H)⁺]: 351.2.

### Intermediate 5: 8-chloro-2-(4-chloro-2-hydroxy-5-methyl-phenyl)chromen-4-one

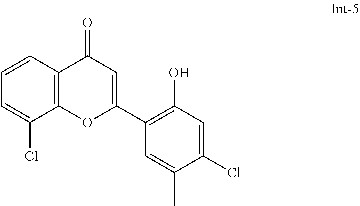 [SMILES: Cc1cc(-c2cc(=O)c3cccc(Cl)c3o2)c(O)cc1Cl]

### Step 1: Preparation of 4-chloro-2-hydroxy-5-methyl-benzaldehyde

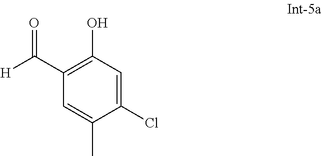

To a solution of 3-chloro-4-methyl-phenol (10.0 g, 70.1 mmol, CAS registry number: 615-62-3, Vendor: Bide Pharmatech, Catalog number: BD85862) in ACN (200 mL) were added formaldehyde (8.42 g, 280.54 mmol), TEA (39.1 mL, 280.5 mmol) and magnesium chloride (27.0 mL, 210.4 mmol) and the mixture was stirred at 80° C. for 16 hours. After the reaction was completed, the reaction was quenched with 1M HCl (500 mL) and extracted with EtOAc (150 mL) three times. The combined organic layer was washed with brine, dried over Na₂SO₄ and concentrated in vacuo to give 4-chloro-2-hydroxy-5-methyl-benzaldehyde (11.3 g, yield: 94.5%) as brown oil, which was used in the next step without further purification.

### Step 2: Preparation of 4-chloro-2-(methoxymethoxy)-5-methyl-benzaldehyde

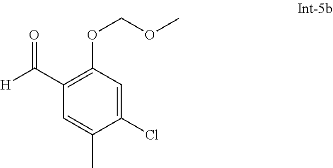

To a solution of 4-chloro-2-hydroxy-5-methyl-benzaldehyde (9.6 g, 56.28 mmol) in THF (100 mL) cooled at 0° C. was added sodium hydride (3.38 g, 84.41 mmol) in small portions. After addition, the mixture was stirred at 0° C. for 30 minutes and then to the resulting mixture was added bromomethyl methyl ether (10.55 g, 84.41 mmol) dropwise. The reaction mixture was stirred at 0° C. for another 2 hours. After the reaction was complete, the mixture was poured into ice-water (200 mL) slowly and extracted with EtOAc (80 mL) three times. The combined organic layer was washed with brine, dried over Na₂SO₄ and concentrated in vacuo to give 4-chloro-2-(methoxymethoxy)-5-methyl-benzaldehyde (12 g, yield: 99.3%) as a white solid, which was used in the next step directly. MS obsd. (ESI⁺)[(M+H)⁺]: 215.1.

### Step 3: Preparation of (E)-1-(3-chloro-2-hydroxy-phenyl)-3-[4-chloro-2-(methoxymethoxy)-5-methyl-phenyl]prop-2-en-1-one

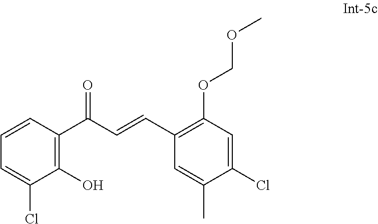 [SMILES: COCOc1cc(Cl)c(C)cc1/C=C/C(=O)c1cccc(Cl)c1O]

To a solution of 4-chloro-2-(methoxymethoxy)-5-methyl-benzaldehyde (6.0 g, 27.95 mmol) and 1-(3-chloro-2-hydroxy-phenyl)ethanone (4.77 g, 27.95 mmol) in EtOH (300 mL) was added KOH (15.68 g, 279.52 mmol). The mixture was stirred at 35° C. for 16 hours. After the reaction was completed, the mixture was poured into 0.5M HCl (200 mL) and the resulting suspension was filtered. The filter cake was collected and dried in vacuo to give (E)-1-(3-chloro-2-hydroxy-phenyl)-3-[4-chloro-2-(methoxymethoxy)-5-methyl-phenyl]prop-2-en-1-one (10 g, 27.23 mmol, yield: 97.4%) as a yellow solid, which was used in the next step without further purification. MS obsd. (ESI⁺)[(M+H)⁺]: 367.0.

### Step 4: Preparation of 8-chloro-2-[4-chloro-2-(methoxymethoxy)-5-methyl-phenyl]chromen-4-one

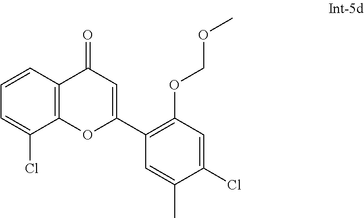 [SMILES: COCOc1cc(Cl)c(C)cc1-c1cc(=O)c2cccc(Cl)c2o1]

To a solution of (E)-1-(3-chloro-2-hydroxy-phenyl)-3-[4-chloro-2-(methoxymethoxy)-5-methyl-phenyl]prop-2-en-1-one (10.0 g, 27.23 mmol) in DMSO (250 mL) was added iodine (345.58 mg, 1.36 mmol). The reaction mixture was stirred at 140° C. under N₂ for 2 hours. After the reaction was complete, the mixture was poured into ice-water and the resulting suspension was filtered. The solid was washed with water and then dried to give 8-chloro-2-[4-chloro-2-(methoxymethoxy)-5-methyl-phenyl]chromen-4-one (8.7 g, yield: 87.5%) as a yellow solid, which was used in the next step without further purification. MS obsd. (ESI⁺)[(M+H)⁺]: 365.0.

### Step 5: Preparation of 8-chloro-2-(4-chloro-2-hydroxy-5-methyl-phenyl)chromen-4-one

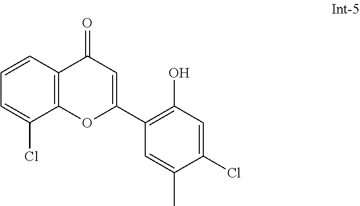 [SMILES: Cc1cc(-c2cc(=O)c3cccc(Cl)c3o2)c(O)cc1Cl]

To a solution of 8-chloro-2-[4-chloro-2-(methoxymethoxy)-5-methyl-phenyl]chromen-4-one (3.4 g, 9.31 mmol) in dichloromethane (10 mL) was added trifluoroacetic acid (10.0 mL, 129.8 mmol). The reaction mixture was stirred at room temperature for 16 hours. After the reaction was completed, the reaction mixture was concentrated in vacuo to give 8-chloro-2-(4-chloro-2-hydroxy-5-methyl-phenyl)chromen-4-one (2.7 g, yield: 90.3%) as a brown solid, which was used in the next step directly. MS obsd. (ESI⁺) [(M+H)⁺]: 320.9.

### Intermediate 6: 2-(4-bromo-2-hydroxy-5-methyl-phenyl)-8-chloro-chromen-4-one

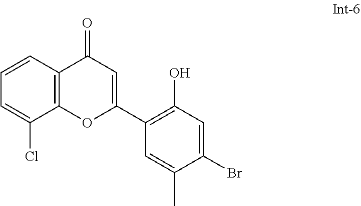 [SMILES: Cc1cc(-c2cc(=O)c3cccc(Cl)c3o2)c(O)cc1Br]

Int-6 was prepared in analogy to the procedure described for the preparation of compound Int-5 by using 3-bromo-4-methyl-phenol as the starting material instead of 3-chloro-4-methyl-phenol in Step 1.

### Intermediate 7: 2-(4-bromo-2-hydroxy-5-methoxy-phenyl)-8-chloro-chromen-4-one

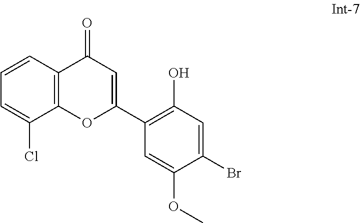 [SMILES: COc1cc(-c2cc(=O)c3cccc(Cl)c3o2)c(O)cc1Br]

Int-7 was prepared in analogy to the procedure described for the preparation of compound Int-5 by using 4-bromo-2-hydroxy-5-methoxy-benzaldehyde as the starting material instead of 4-chloro-2-hydroxy-5-methyl-benzaldehyde in Step 2. MS obsd. (ESI⁺) [(M+H)⁺]: 381.1.

### Intermediate 8: 8-chloro-2-(2-hydroxy-4,5-dimethoxy-phenyl)chromen-4-one

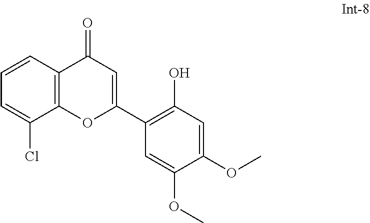 [SMILES: COc1cc(O)c(-c2cc(=O)c3cccc(Cl)c3o2)cc1OC]

Int-8 was prepared in analogy to the procedure described for the preparation of compound Int-5 by using 2-hydroxy-4,5-dimethoxy-benzaldehyde (CAS registry number: 14382-91-3, Vendor: Accela ChemBio Inc., Catalog number: SY025559) as the starting material instead of 4-chloro-2-hydroxy-5-methyl-benzaldehyde in Step 2. MS obsd. (ESI⁺) [(M+H)⁺]: 333.2.

### Intermediate 9: 8-chloro-2-(2-hydroxy-4-methoxy-5-methyl-phenyl)chromen-4-one

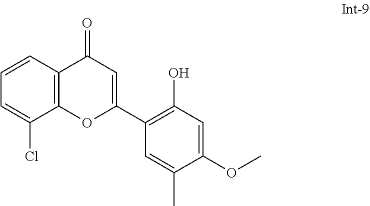 [SMILES: COc1cc(O)c(-c2cc(=O)c3cccc(Cl)c3o2)cc1C]

Int-9 was prepared in analogy to the procedure described for the preparation of compound Int-5 by using 3-methoxy-4-methyl-phenol as the starting material instead of 3-chloro-4-methyl-phenol in Step 1.

### Intermediate 10: 8-chloro-2-(2-hydroxy-4-methoxy-phenyl)chromen-4-one

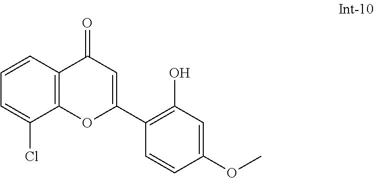 [SMILES: COc1ccc(-c2cc(=O)c3cccc(Cl)c3o2)c(O)c1]

Int-10 was prepared in analogy to the procedure described for the preparation of compound Int-5 by using 2-hydroxy-4-methoxy-benzaldehyde (CAS registry number: 673-22-3, Vendor: Accela ChemBio Inc., Catalog number: SY012912) as the starting material instead of 4-chloro-2-hydroxy-5-methyl-benzaldehyde in Step 2. MS obsd. (ESI⁺) [(M+H)⁺]: 302.9.

### Intermediate 11: 2-(5-bromo-2-hydroxy-4-methoxy-phenyl)-8-chloro-chromen-4-one

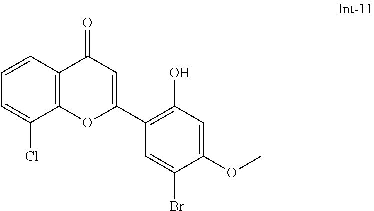 [SMILES: COc1cc(O)c(-c2cc(=O)c3cccc(Cl)c3o2)cc1Br]

Int-11 was prepared in analogy to the procedure described for the preparation of compound Int-5 by using 5-bromo-2-hydroxy-4-methoxybenzaldehyde as the starting material instead of 4-chloro-2-hydroxy-5-methyl-benzaldehyde in Step 2. MS obsd. (ESI⁺)[(M+H)⁺]: 381.1.

### Intermediate 12: 8-chloro-2-(2-hydroxy-5-methoxy-4-methyl-phenyl)chromen-4-one

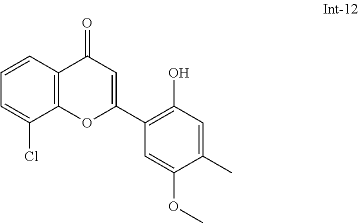 [SMILES: COc1cc(-c2cc(=O)c3cccc(Cl)c3o2)c(O)cc1C]

Int-12 was prepared in analogy to the procedure described for the preparation of compound Int-5 by using 4-methoxy-3-methyl-phenol as the starting material instead of 3-chloro-4-methyl-phenol in Step 1. MS obsd. (ESI⁺) [(M+H)]⁺: 317.2.

### Intermediate 13: 8-chloro-2-(4-ethoxy-2-hydroxy-phenyl)chromen-4-one

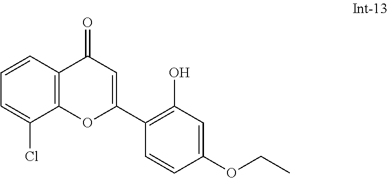 [SMILES: CCOc1ccc(-c2cc(=O)c3cccc(Cl)c3o2)c(O)c1]

### Step 1: Preparation of 2-hydroxy-4-(methoxymethoxy)benzaldehyde

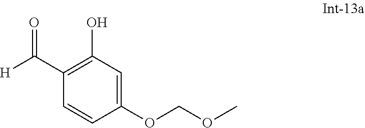

A 500 mL of flask fitted with magnetic stirrer was charged with 2,4-dihydroxybenzaldehyde (5.52 g, 39.97 mmol, CAS registry number: 95-01-2, Vendor: TCI Shanghai, Catalog number: 0564) and DIPEA (10.3 g, 79.93 mmol) in THF (40 mL) and DCM (240 mL). The solution was cooled to 0° C. and then bromomethyl methyl ether (5.0 g, 39.97 mmol) was added. The reaction mixture was allowed to warm to room temperature and stirred for 16 hours. After the starting material was consumed, the reaction mixture was concentrated in vacuo to afford 2-hydroxy-4-(methoxymethoxy)benzaldehyde (4.8 g, yield: 65.9%) as a white solid. MS obsd. (ESI⁺)[(M+H)⁺]: 183.1.

Step 2: Preparation of 4-(methoxymethoxy)-2-[(4-methoxyphenyl)methoxy]benzaldehyde

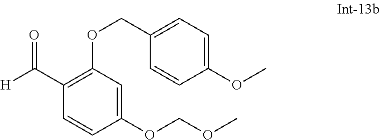 [SMILES: COCOc1ccc(C=O)c(OCc2ccc(OC)cc2)c1]

To a solution of 2-hydroxy-4-(methoxymethoxy)benzaldehyde (4.8 g, 26.35 mmol) and 4-methoxybenzylchloride (4.29 mL, 31.62 mmol) in DMF (100 mL) was added K₂CO₃ (7.27 g, 52.7 mmol). The reaction mixture was stirred at 70° C. for 3 hours. After completion, the reaction was concentrated in vacuo and purified by flash column (eluting with EtOAc:PE=0 to 25%) to give 4-(methoxymethoxy)-2-[(4-methoxyphenyl)methoxy]benzaldehyde (7.0 g, yield: 84.4%) as a white solid. MS obsd. (ESI⁺)[(M+Na)⁺]: 325.1.

### Step 3: Preparation of (E)-1-(3-chloro-2-hydroxy-phenyl)-3-[4-(methoxymethoxy)-2-[(4-methoxyphenyl)methoxy]phenyl]prop-2-en-1-one

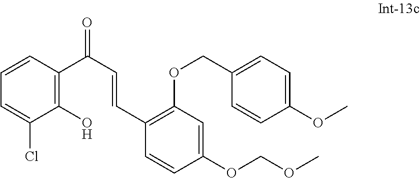 [SMILES: COCOc1ccc(/C=C/C(=O)c2cccc(Cl)c2O)c(OCc2ccc(OC)cc2)c1]

To a solution of 1-(3-chloro-2-hydroxy-phenyl)ethanone (2.82 g, 16.54 mmol) and KOH (7.41 g, 132.31 mmol) in ethanol (500 mL) was added 5-(methoxymethoxy)-2-[(4-methoxyphenyl)methoxy]benzaldehyde (5.0 g, 16.54 mmol). The reaction mixture was stirred at 50° C. for 16 hours. After the starting material was consumed, the reaction mixture was acidified to pH=2 by adding 1N HCl (6 mL) to form a suspension. The suspension was filtered and the filter cake was collected and dried to give (E)-1-(3-chloro-2-hydroxy-phenyl)-3-[4-(methoxymethoxy)-2-[(4-methoxyphenyl)methoxy]phenyl]prop-2-en-1-one (6.0 g, yield: 79.8%) as a yellow solid. MS obsd. (ESI⁺)[(M+Na)⁺]: 477.1.

### Step 4: Preparation of 8-chloro-2-[4-hydroxy-2-[(4-methoxyphenyl)methoxy]phenyl]chromen-4-one

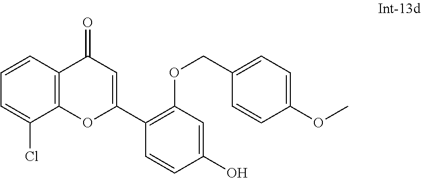 [SMILES: COc1ccc(COc2cc(O)ccc2-c2cc(=O)c3cccc(Cl)c3o2)cc1]

To a solution of (E)-1-(3-chloro-2-hydroxy-phenyl)-3-[4-(methoxymethoxy)-2-[(4-methoxyphenyl)methoxy]phenyl]prop-2-en-1-one (6.0 g, 13.19 mmol) in DMSO (200 mL) was added iodine (167 mg, 0.66 mmol). The reaction mixture was stirred at 140° C. for 3 hours. Then, the reaction mixture was poured into water (100 mL) and the solid was precipitated out. The mixture was filtered and the filter cake was washed with aq. Na₂SO₃ solution (10 mL) and dried to give 8-chloro-2-[4-hydroxy-2-[(4-methoxyphenyl)methoxy]phenyl]chromen-4-one (5.0 g, yield: 92.7% as a dark brown solid. MS obsd. [(M+H)⁺]: (ESI⁺): 409.2.

### Step 5: Preparation of 8-chloro-2-[4-ethoxy-2-[(4-methoxyphenyl)methoxy]phenyl]chromen-4-one

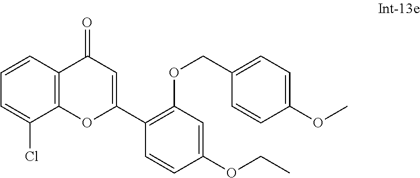 [SMILES: CCOc1ccc(-c2cc(=O)c3cccc(Cl)c3o2)c(OCc2ccc(OC)cc2)c1]

To a solution of 8-chloro-2-[4-hydroxy-2-[(4-methoxyphenyl)methoxy]phenyl]chromen-4-one (3.0 g, 7.34 mmol) and iodoethane (1.17 mL, 14.68 mmol) in DMF (25 mL) was added K₂CO₃ (2.03 g, 14.68 mmol). The reaction mixture was stirred at room temperature. After stirring for 3 hours, the reaction was quenched by adding brine (40 mL). The mixture was extracted with EtOAc (40 mL) twice. The organic layer was combined and concentrated in vacuo to give the crude product. The crude product was purified by flash column (eluting with EtOAc:PE=0 to 40%) to give 8-chloro-2-[4-ethoxy-2-[(4-methoxyphenyl)methoxy]phenyl]chromen-4-one (1.5 g, yield: 46.8%) as a yellow solid. MS obsd. (ESI⁺)[(M+H)⁺]: 437.1.

### Step 6: Preparation of 8-chloro-2-(4-ethoxy-2-hydroxy-phenyl)chromen-4-one

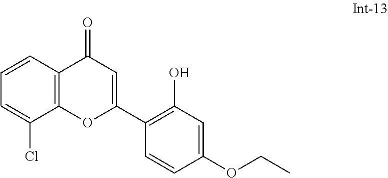 [SMILES: CCOc1ccc(-c2cc(=O)c3cccc(Cl)c3o2)c(O)c1]

A solution of 8-chloro-2-[4-ethoxy-2-[(4-methoxyphenyl)methoxy]phenyl]chromen-4-one (1.5 g, 3.43 mmol) in TFA (10.0 mL, 3.43 mmol) was stirred at 110° C. for 2 hours. After completion, the reaction mixture was cooled in the ice/water bath and then concentrated in vacuo to give 8-chloro-2-(4-ethoxy-2-hydroxy-phenyl)chromen-4-one (1.0 g, yield: 92.0%) as a yellow solid. MS obsd. (ESI⁺)[(M+H)⁺]: 317.0.

### Intermediate 14: 8-chloro-2-(4-ethoxy-2-hydroxy-phenyl)chromen-4-one

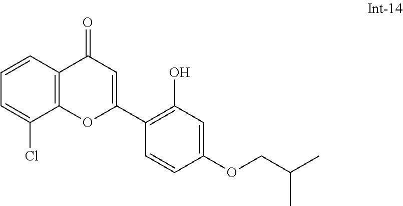 [SMILES: CC(C)COc1ccc(-c2cc(=O)c3cccc(Cl)c3o2)c(O)c1]

Int-14 was prepared in analogy to the procedure described for the preparation of compound Int-13 by using 1-iodo-2-methyl-propane as the reagent instead of iodoethane in Step 5. MS obsd. (ESI⁺) [(M+H)⁺]: 345.2.

### Intermediate 15: 8-chloro-6-fluoro-2-(2-hydroxyphenyl)chromen-4-one

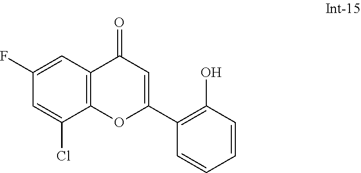 [SMILES: O=c1cc(-c2ccccc2O)oc2c(Cl)cc(F)cc12]

### Step 1: Preparation of 1-(3-chloro-5-fluoro-2-hydroxy-phenyl)ethanone

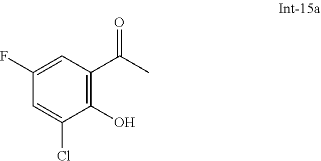

A solution of 2-chloro-4-fluorophenol (4.5 g, 30.71 mmol, CAS registry number: 1996-41-4, Vendor: Bide Pharmatech, Catalog number: BD19192), acetic anhydride (4.7 g, 46.06 mmol) and sulfuric acid (1.51 g, 15.35 mmol) was stirred at room temperature for 2 hours. After completion, the reaction mixture was diluted with water (50 mL). The aqueous layer was extracted by EtOAc (50 mL) twice, and the combined organic layer was concentrated in vacuo. Then, aluminum chloride (6.1 g, 46.06 mmol) was added to the resulting residue and stirred at 120° C. for 8 hours. The reaction mixture was diluted with water (150 mL), and the aqueous layer was extracted by EtOAc (100 mL) twice. The combined organic layer was concentrated in vacuo to give the crude product. The crude product was purified by flash column (eluting with EtOAc:PE=3% to 10%) to give 1-(3-chloro-5-fluoro-2-hydroxy-phenyl)ethanone (3.8 g, yield: 65.62%) as a light yellow solid. MS obsd. (ESI⁺) [(M−H)⁻]: 187.0.

### Step 2-5: Preparation of 8-chloro-6-fluoro-2-(2-hydroxyphenyl)chromen-4-one

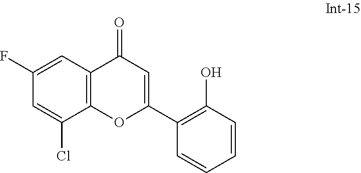 [SMILES: O=c1cc(-c2ccccc2O)oc2c(Cl)cc(F)cc12]

Int-15 was prepared in analogy to the procedure described for the preparation of compound Int-5 by using 2-methoxybenzaldehyde as the starting materials instead of 4-chloro-2-hydroxy-5-methyl-benzaldehyde in Step 2 and using Int-15a instead of 1-(3-chloro-2-hydroxy-phenyl)ethanone in Step 3. MS obsd. (ESI⁺)[(M+H)⁺]: 291.2.

### Intermediate 16: 8-chloro-2-(2-hydroxy-4-methyl-phenyl)-6-methoxy-chromen-4-one

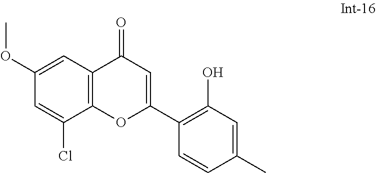 [SMILES: COc1cc(Cl)c2oc(-c3ccc(C)cc3O)cc(=O)c2c1]

### Step 1: Preparation of 1-(3-chloro-2-hydroxy-5-methoxy-phenyl)ethanone

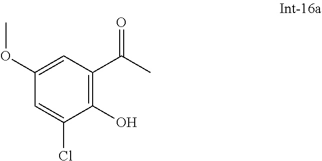

To a solution of 2-hydroxy-5-methoxyacetophenone (2.0 g, 12.04 mmol, CAS registry number: 705-15-7, Vendor: Bide Pharmatech, Catalog number: BD11251) in DMF (15 mL) was added N-chlorosuccinimide (2.41 g, 18.05 mmol). Then, the reaction mixture was stirred for 2 hours. After the starting material was consumed, the reaction mixture was poured into ice water (100 mL) and extracted with EtOAc (50 mL) three times. The combined organic layer was washed with brine (50 mL) twice, dried over anhydrous MgSO₄, filtered and concentrated in vacuo to give the crude product. The crude product was purified by flash column (eluting with EtOAc:PE=10%) to give 1-(3-chloro-2-hydroxy-5-methoxy-phenyl)ethanone (2.3 g, yield: 95.3%) as a light brown solid. (ESI⁺)[(M+H)⁺]: 201.0.

### Step 2-5: Preparation of 8-chloro-2-(2-hydroxy-4-methyl-phenyl)-6-methoxy-chromen-4-one

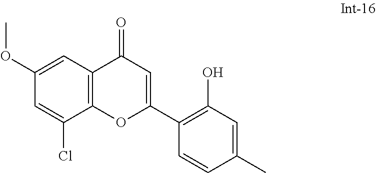 [SMILES: COc1cc(Cl)c2oc(-c3ccc(C)cc3O)cc(=O)c2c1]

Int-16 was prepared in analogy to the procedure described for the preparation of compound Int-5 by using 2-hydroxy-4-methyl-benzaldehyde as the starting materials instead of 4-chloro-2-hydroxy-5-methyl-benzaldehyde in Step 2 and using Int-16a instead of 1-(3-chloro-2-hydroxy-phenyl)ethanone in Step 3. MS obsd. (ESI⁺)[(M+H)⁺]: 291.2.

### Intermediate 17: 2-(4-bromo-2-hydroxy-phenyl)-8-chloro-5-(trifluoromethyl)chromen-4-one

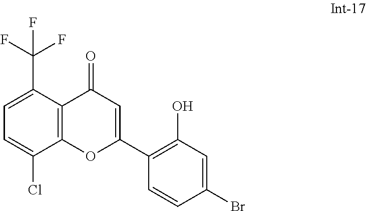 [SMILES: O=c1cc(-c2ccc(Br)cc2O)oc2c(Cl)ccc(C(F)(F)F)c12]

### Step 1: Preparation of 1-(6-bromo-3-chloro-2-hydroxy-phenyl)ethanone

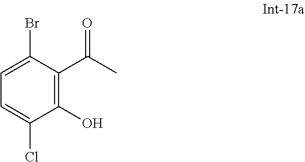

To a mixture of 5-bromo-2-chlorophenol (6.0 g, 28.92 mmol) and acetyl acetate (4.1 mL, 43.38 mmol) was added four drops of concentrated sulfuric acid (0.2 mL) at room temperature.

The reaction was stirred at room temperature for 2 hours. Then, the reaction mixture was poured into water (50 mL) under stirring and layers were separated. The aqueous layer was extracted with EtOAc (50 mL) three times. The combined organic layer was washed with brine, dried over anhydrous Na₂SO₄ and concentrated in vacuo. Into the above residue cooled in an ice-water bath was then added with powdered anhydrous trichloroalumane (5.78 g, 43.38 mmol). The resulting mixture was heated to 120° C. and stirred at this temperature for 5 hours. After completion, the reaction mixture was quenched with crushed ice and extracted with EtOAc (90 mL) three times. The combined organic phase was concentrated in vacuo to give the crude product as light brown solid, which was purified by flash chromatography to give 1-(6-bromo-3-chloro-2-hydroxy-phenyl)ethanone (623 mg, yield: 8.63%) as light yellow liquid. MS obsd. (ESI⁺)[(M+H)⁺]: 248.0.

### Step 2: Preparation of 1-[6-bromo-3-chloro-2-[(4-methoxyphenyl)methoxy]phenyl]ethanone

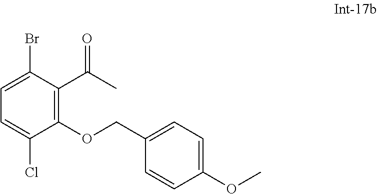 [SMILES: COc1ccc(COc2c(Cl)ccc(Br)c2C(C)=O)cc1]

To a mixture of 1-(6-bromo-3-chloro-2-hydroxy-phenyl)ethanone (300 mg, 1.2 mmol) and 4-methoxybenzylchloride (0.2 mL, 1.44 mmol) in DMF (20 mL) was added potassium carbonate (415 mg, 3.01 mmol). The reaction mixture was stirred at 80° C. for 3 hours. Then, the reaction mixture was quenched with water and extracted with EtOAc (50 mL) three times. The combined organic layer was concentrated in vacuo to give the crude product. The crude product was purified by flash column (eluting with PE:EtOAc=10:1) to give 1-[6-bromo-3-chloro-2-[(4-methoxyphenyl)methoxy]phenyl]ethanone (303 mg, yield: 68.17%) as a white solid. MS obsd. (ESI⁺)[(M+Na)⁺]: 391.0.

### Step 3: Preparation of 1-[3-chloro-2-hydroxy-6-(trifluoromethyl)phenyl]ethanone

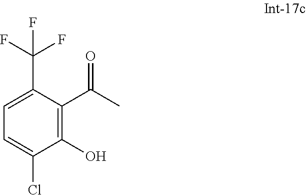

To a solution of 1-[6-bromo-3-chloro-2-[(4-methoxyphenyl)methoxy]phenyl]ethanone (300 mg, 0.810 mmol) and iodocopper (773 mg, 4.06 mmol) in DMF (8 mL) was added methyl 2,2-difluoro-2-fluorosulfinyl-acetate (715 mg, 4.06 mmol). The reaction mixture was stirred at 110° C. for 18 hours. The reaction mixture was quenched with water (10 mL) to form a suspension. The suspension was filtered, and the filter cake was washed with EtOAc (10 mL) three times. The filtrate was collected and layers were separated. The aqueous layer was extracted with EtOAc (10 mL) twice. The organic layer was combined, dried over anhydrous Na₂SO₄, filtered and concentrated in vacuo. The crude product was purified by flash chromatography (eluting with EtOAc:PE=10%) to give 1-[3-chloro-2-hydroxy-6-(trifluoromethyl)phenyl]ethanone (135 mg, yield: 69.7%) as a colorless oil. MS obsd. (ESI⁺)[(M+Na)⁺]: 391.0.

### Step 4-7: Preparation of 2-(4-bromo-2-hydroxy-phenyl)-8-chloro-5-(trifluoromethyl)chromen-4-one

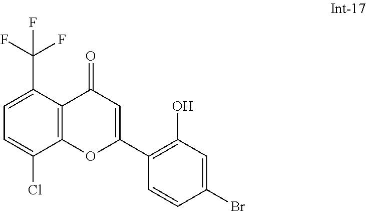 [SMILES: O=c1cc(-c2ccc(Br)cc2O)oc2c(Cl)ccc(C(F)(F)F)c12]

Int-17 was prepared in analogy to the procedure described for the preparation of compound Int-5 by using 2-hydroxy-4-bromo-benzaldehyde as the starting materials instead of 4-chloro-2-hydroxy-5-methyl-benzaldehyde in Step 2 and using Int-17c instead of 1-(3-chloro-2-hydroxy-phenyl)ethanone in Step 3. MS obsd. (ESI⁺)[(M+H)⁺]: 419.0.

### Intermediate 18: 8-chloro-2-(2-fluoro-6-hydroxy-4-methyl-phenyl)chromen-4-one

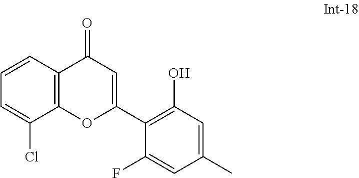 [SMILES: Cc1cc(O)c(-c2cc(=O)c3cccc(Cl)c3o2)c(F)c1]

### Step 1: Preparation of 2-(4-bromo-2-fluoro-6-methoxy-phenyl)-8-chloro-chromen-4-one

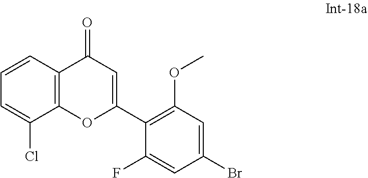 [SMILES: COc1cc(Br)cc(F)c1-c1cc(=O)c2cccc(Cl)c2o1]

Int-18a was prepared in analogy to the procedure described for the preparation of compound Int-1b by using 4-bromo-2-fluoro-6-methoxybenzaldehyde (CAS registry number: 856767-09-4, Vendor: Bide Pharmatech, Catalog number: BD259901) as the starting material instead of 2-methoxy-4-(trifluoromethyl)benzaldehyde in Step 1. MS obsd. (ESI⁺) [(M+H)⁺]: 382.9.

### Step 2: Preparation of 8-chloro-2-(2-fluoro-6-methoxy-4-methyl-phenyl)chromen-4-one

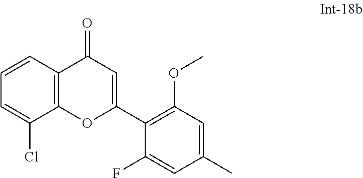 [SMILES: COc1cc(C)cc(F)c1-c1cc(=O)c2cccc(Cl)c2o1]

To a solution of 2-(4-bromo-2-fluoro-6-methoxy-phenyl)-8-chloro-chromen-4-one (300.0 mg, 0.8 mmol), trimethylboroxine (196.4 mg, 1.56 mmol) and K₂CO₃ (324.3 mg, 2.35 mmol) in dioxane (10 mL) under N₂ was added Pd(dppf)₂Cl₂ (57.8 mg, 0.08 mmol). The reaction was stirred at 110° C. under N₂ for 1 hour. After the reaction was completed, the mixture was cooled to room temperature and concentrated in vacuo. The crude product was purified by flash column (eluting with EtOAc:PE=0 to 30%) to give 8-chloro-2-(2-fluoro-6-methoxy-4-methyl-phenyl)chromen-4-one (150 mg, yield: 57.2%) as an off-white solid. MS obsd. (ESI⁺) [(M+H)⁺]: 319.0.

### Step 3: Preparation of 8-chloro-2-(2-fluoro-6-hydroxy-4-methyl-phenyl)chromen-4-one

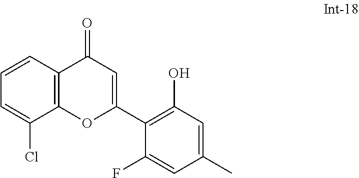 [SMILES: Cc1cc(O)c(-c2cc(=O)c3cccc(Cl)c3o2)c(F)c1]

To a solution of 8-chloro-2-(2-fluoro-6-methoxy-4-methyl-phenyl)chromen-4-one (0.15 g, 0.47 mmol) in dichloromethane (10 mL) was added BBr₃ (1M solution in dichloromethane, 7 mL, 7 mmol) at room temperature. The mixture was stirred at room temperature overnight. After the reaction was completed, the mixture was concentrated in vacuo and then the residue was suspended in sat. NH₄Cl solution (10 mL). The solid was collected by filtration and dried in vacuo to give 8-chloro-2-[2-hydroxy-4-(trifluoromethyl)phenyl]chromen-4-one (0.124 g, yield: 86%) as a yellow solid, which was used in the next step without further purification. MS obsd. (ESI⁺) [(M+H)]⁺: 305.0.

### Intermediate 19: 8-chloro-2-(2-hydroxy-6-methoxy-4-methyl-phenyl)chromen-4-one

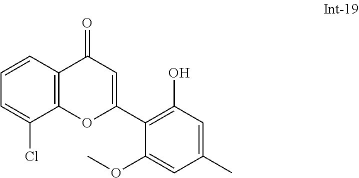 [SMILES: COc1cc(C)cc(O)c1-c1cc(=O)c2cccc(Cl)c2o1]

### Step 1: Preparation of 4-bromo-2-methoxy-6-[(4-methoxyphenyl)methoxy]benzaldehyde

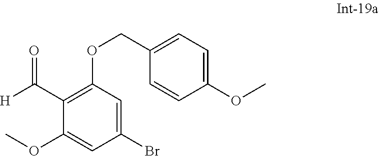 [SMILES: COc1ccc(COc2cc(Br)cc(OC)c2C=O)cc1]

To a solution of 4-bromo-2-hydroxy-6-methoxy-benzaldehyde (1.4 g, 6.06 mmol) in 4-methoxybenzylchloride (0.99 mL, 7.27 mmol) was added potassium carbonate (2.5 g, 18.18 mmol). The reaction was stirred at 80° C. for 2 hours. After completion, the reaction was quenched by adding water (50 mL) and extracted with EtOAc (50 mL) three times. The combined organic layer was washed with brine, dried over anhydrous sodium sulfate and concentrated in vacuo to give the crude product. The crude product was purified by flash column (EtOAc:PE=15%) to give 4-bromo-2-methoxy-6-[(4-methoxyphenyl)methoxy]benzaldehyde (1.9 g, yield: 93%) as a yellow solid. MS obsd. (ESI⁺): 373.0.

### Step 2: Preparation of 2-methoxy-6-[(4-methoxyphenyl)methoxy]-4-methyl-benzaldehyde

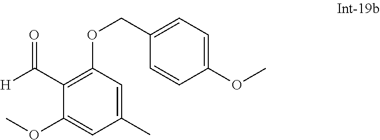 [SMILES: COc1ccc(COc2cc(C)cc(OC)c2C=O)cc1]

To a solution of 4-bromo-2-methoxy-6-[(4-methoxyphenyl)methoxy]benzaldehyde (1.2 mg, 3.42 mmol) in 1,4-dioxane (16.79 mL) was added trimethylboroxine (857 mg, 6.83 mmol) and [1,1′-bis(diphenylphosphino)ferrocene]dichloropalladium(II) (250 mg, 0.340 mmol) under N₂ at room temperature. Then, the reaction was heated at 110° C. for 16 hours. After the reaction was completed, the reaction was quenched by adding ice-water (50 mL) and extracted with EtOAc (50 mL) three times. The combined organic layer was washed with brine, dried over anhydrous sodium sulfate and concentrated in vacuo to give the crude product. The crude product was purified by flash column (EA:PE=50%) to give 2-methoxy-6-[(4-methoxyphenyl)methoxy]-4-methyl-benzaldehyde (700 mg, yield: 67.9%) as a yellow solid. MS obsd. (ESI⁺)[(M+H)⁺]: 287.1.

### Step 3: Preparation of 2-hydroxy-6-methoxy-4-methyl-benzaldehyde

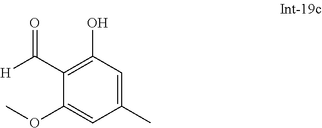

To a solution of 2-methoxy-6-[(4-methoxyphenyl)methoxy]-4-methyl-benzaldehyde (650 mg, 2.27 mmol) in DCM (9 mL) was added sodium hydroxide (181 mg, 4.54 mmol). The reaction mixture was stirred at room temperature for 30 minutes, then trifluoroacetic acid (0.17 mL, 2.27 mmol) was added. After stirring for another 1 hour, the reaction was quenched by adding water (50 mL) and extracted with EtOAc (50 mL) three times. The combined organic layer was washed with brine, dried over anhydrous sodium sulfate and concentrated in vacuo to give the crude product. The crude product was purified by flash column (EtOAc:PE=20%) to give 2-hydroxy-6-methoxy-4-methyl-benzaldehyde (290 mg, yield: 76.87%) as a white solid. MS obsd. (ESI⁺)[(M+H)⁺]: 167.1.

### Step 4: Preparation of 2-methoxy-6-(methoxymethoxy)-4-methyl-benzaldehyde

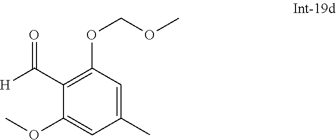

A solution of 2-hydroxy-6-methoxy-4-methyl-benzaldehyde (293 mg, 1.77 mmol) in THF (135 mL) was added bromomethyl methyl ether (441 mg, 3.53 mmol) and sodium hydride (51 mg, 2.12 mmol) at 0° C. for 30 minutes. Then, the reaction solution was diluted with water (50 mL) and extracted with EtOAc (50 mL) three times. The combined organic layer was washed with brine, dried over anhydrous sodium sulfate and concentrated in vacuo to give the crude product. The crude product was purified by flash column (EtOAc:PE=10%) to give 2-methoxy-6-(methoxymethoxy)-4-methyl-benzaldehyde (250 mg, yield: 67.3%) as colorless liquid. MS obsd. (ESI⁺) [(M+H)⁺]: 211.1.

### Step 4: Preparation of (E)-1-(3-chloro-2-hydroxy-phenyl)-3-[2-methoxy-6-(methoxymethoxy)-4-methyl-phenyl]prop-2-en-1-one

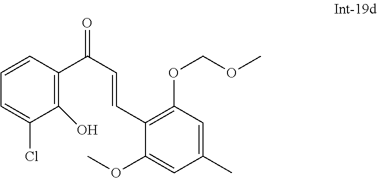 [SMILES: COCOc1cc(C)cc(OC)c1/C=C/C(=O)c1cccc(Cl)c1O]

A solution of 1-(3-chloro-2-hydroxy-phenyl)ethanone (203 mg, 1.19 mmol) in THF (50 mL) was added potassium hydroxide (667 mg, 11.89 mmol) and 2-methoxy-6-(methoxymethoxy)-4-methyl-benzaldehyde (250 mg, 1.19 mmol) at 35° C. for 16 hours. After the reaction was completed, the reaction was adjusted to pH 6-7 by adding 1M HCl. Then, the reaction mixture was partitioned between water (50 mL) and EtOAc (50 mL). The aqueous layer was extracted with EtOAc (50 mL) twice and the combined organic layer was washed with brine, dried over anhydrous sodium sulfate and concentrated in vacuo to give the crude product. The crude product was purified by flash column (EtOAc:PE=10%) to give (E)-1-(3-chloro-2-hydroxy-phenyl)-3-[2-methoxy-6-(methoxymethoxy)-4-methyl-phenyl]prop-2-en-1-one (320 mg, yield: 70.8%) as yellow oil. MS obsd. (ESI⁺)[(M+H)⁺]: 363.1.

### Step 5: Preparation of 8-chloro-2-(2-hydroxy-6-methoxy-4-methyl-phenyl)chromen-4-one

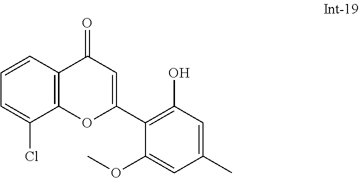 [SMILES: COc1cc(C)cc(O)c1-c1cc(=O)c2cccc(Cl)c2o1]

A solution of (E)-1-(3-chloro-2-hydroxy-phenyl)-3-[2-methoxy-6-(methoxymethoxy)-4-methyl-phenyl]prop-2-en-1-one (320 mg, 0.880 mmol) and iodine (11.19 mg, 0.040 mmol) in DMSO (2.32 mL) was stirred at 140° C. for 4 hours. Then, the reaction mixture was diluted with water (20 mL) to form a suspension. The suspension was filtered and the filter cake was washed with water (50 mL) and dried to give 8-chloro-2-(2-hydroxy-6-methoxy-4-methyl-phenyl)chromen-4-one (130 mg, yield: 44.2%) as a white solid. MS obsd. [(M+H)⁺] (ESI⁺): 317.1.

### Example 001: 1-[3-[5-bromo-2-(8-chloro-4-oxo-chromen-2-yl)phenoxy]propyl]pyrrolidine-3-carboxylic acid

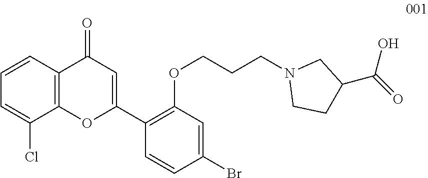 [SMILES: O=C(O)C1CCN(CCCOc2cc(Br)ccc2-c2cc(=O)c3cccc(Cl)c3o2)C1]

### Step 1: Preparation of 2-[4-bromo-2-(3-bromopropoxy)phenyl]-8-chloro-chromen-4-one

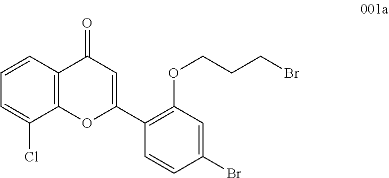 [SMILES: O=c1cc(-c2ccc(Br)cc2OCCCBr)oc2c(Cl)cccc12]

To a solution of 2-(4-bromo-2-hydroxy-phenyl)-8-chloro-chromen-4-one (300 mg, 0.853 mmol, as the “CORE” in Table 1) and 1,3-dibromopropane (861 mg, 4.27 mmol, as the “LINKER” in Table 1) in DMF was added K₂CO₃ (236 mg, 1.71 mmol). The reaction mixture was stirred at 70° C. for 2 hours. After the reaction was completed, the mixture was diluted by EtOAc and partitioned between EtOAc (10 mL) and water (30 mL). The organic layer was separated out and the aqueous layer was extracted with EtOAc (30 mL) twice. The combined organic layer was concentrated in vacuo and the resulting residue was purified by ISCO (eluting with EtOAc:PE=0 to 20%) to give 2-[4-bromo-2-(3-bromopropoxy)phenyl]-8-chloro-chromen-4-one (200 mg, yield: 49.6%) as a yellow solid. MS obsd. (ESI⁺) [(M+H)]⁺: 471.3.

### Step 2: Preparation of methyl 1-[3-[5-bromo-2-(8-chloro-4-oxo-chromen-2-yl)phenoxy]propyl]pyrrolidine-3-carboxylate

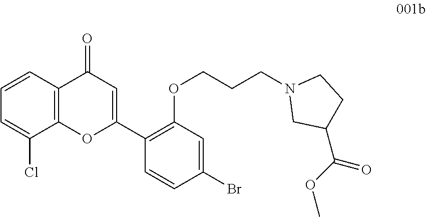 [SMILES: COC(=O)C1CCN(CCCOc2cc(Br)ccc2-c2cc(=O)c3cccc(Cl)c3o2)C1]

To a solution of 2-[4-bromo-2-(3-bromopropoxy)phenyl]-8-chloro-chromen-4-one (300 mg, 0.635 mmol) and methyl pyrrolidine-3-carboxylate hydrochloride (105 mg, 0.635 mmol, as the “AMINE” in Table 1) in DMF was added NaHCO₃ (53.3 mg, 0.635 mmol). The mixture was stirred at 50° C. overnight. After the reaction was completed, the reaction mixture was diluted with EtOAc and partitioned between EtOAc (30 mL) and water (20 mL). The organic layer was separated out and the aqueous phase was extracted with EtOAc (30 mL) twice. The combined organic layer was concentrated in vacuo to give methyl 1-[3-[5-bromo-2-(8-chloro-4-oxo-chromen-2-yl)phenoxy]propyl]pyrrolidine-3-carboxylate (100 mg, yield: 30.2%) as a yellow solid. (ESI⁺) [(M+H)]⁺: 521.9.

### Step 3: Preparation of 1-[3-[5-bromo-2-(8-chloro-4-oxo-chromen-2-yl)phenoxy]propyl]pyrrolidine-3-carboxylic acid

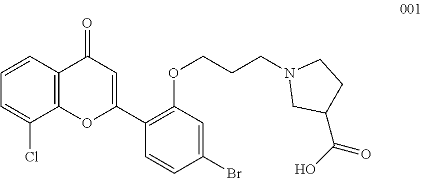 [SMILES: O=C(O)C1CCN(CCCOc2cc(Br)ccc2-c2cc(=O)c3cccc(Cl)c3o2)C1]

To the solution of methyl 1-[3-[5-bromo-2-(8-chloro-4-oxo-chromen-2-yl)phenoxy]propyl]pyrrolidine-3-carboxylate (100 mg, 0.192 mmol) in DMF was added LiOH (13.8 mg, 0.576 mmol). The reaction mixture was stirred at room temperature till completion. Then, the reaction mixture was neutralized by acetic acid and then concentrated in vacuo. The crude material was purified by prep-HPLC to give 1-[3-[5-bromo-2-(8-chloro-4-oxo-chromen-2-yl)phenoxy]propyl]pyrrolidine-3-carboxylic acid (33 mg, yield: 31.2%) as a white powder. (ESI⁺) [(M+H)]⁺: 508.0. Example 001: ¹H NMR (DMSO-d₆, 400 MHz) δ ppm 8.02 (dd, J=7.9, 1.5 Hz, 2H), 7.87 (d, J=8.4 Hz, 1H), 7.39-7.55 (m, 3H), 7.01 (s, 1H), 4.28 (br t, J=6.1 Hz, 2H), 2.64-2.84 (m, 4H), 2.54-2.60 (m, 2H), 2.32-2.42 (m, 1H), 1.87-1.98 (m, 4H).

The following Examples 002 to 061 were prepared in analogy to the procedure described for the preparation of Example 001, replacing 2-(4-bromo-2-hydroxy-phenyl)-8-chloro-chromen-4-one with “CORE”, 1,3-dibromopropane with “LINKER” in Step 1, methyl pyrrolidine-3-carboxylate hydrochloride with “AMINE” by the reagent indicated in Table 1.

TABLE 1
Compounds synthesis and characterization

| Ex- | | CORE, | |
|---|---|---|---|
| ample | | LINKER and | |
| No. | Compounds Name and Structure | AMINE | ¹H NMR and (ESI⁺) |
| 002 | 4-[3-[5-bromo-2-(8-chloro-4- | CORE: Int-4 | ¹H NMR (DMSO-d₆, 400 MHz) |
| | oxo-chromen-2- | LINKER: 1,3- | δ ppm 8.01 (d, J = 7.9 Hz, 2H), |
| | yl)phenoxy]propyl]morpholine- | dibromopropane | 7.88 (d, J = 8.4 Hz, 1H), 7.42- |
| | 2-carboxylic acid | AMINE: | 7.55 (m, 3H), 7.03 (s, 1H), 4.18- |
| | 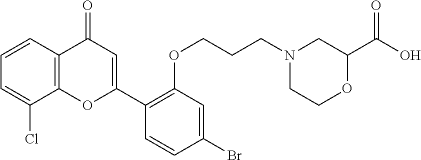 [SMILES: O=C(O)C1CN(CCCOc2cc(Br)ccc2-c2cc(=O)c3cccc(Cl)c3o2)CCO1] | methyl morpholine-2- carboxylate | 4.38 (m, 3H), 3.85-4.08 (m, 2H), 3.47-3.81 (m, 2H), 2.87-3.16 (m, 2H), 1.99-2.26 (m, 2H), 1.17- 1.33 ppm (m, 2H). MS obsd. (ESI⁺) [(M + H)⁺]: 521.9. |
| 003 | (2R)-1-[3-[5-bromo-2-(8- | CORE: Int-4 | ¹H NMR (DMSO-d₆, 400 MHz) |
| | chloro-4-oxo-chromen-2- | LINKER: 1,3- | δ ppm 8.15-8.20 (m, 1H), 7.98- |
| | yl)phenoxy]propyl]pyrrolidine- | dibromopropane | 8.04 (m, 1H), 7.82-7.90 (m, 1H), |
| | 2-carboxylic acid | AMINE: | 7.41-7.55 (m, 2H), 6.96-7.06 (m, |
| | 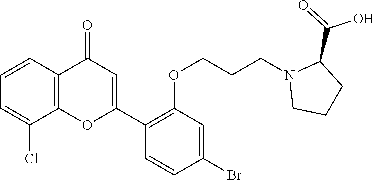 [SMILES: O=C(O)[C@H]1CCCN1CCCOc1cc(Br)ccc1-c1cc(=O)c2cccc(Cl)c2o1] | methyl (2R)- pyrrolidine-2- carboxylate | 1H), 6.48-6.61 (m, 1H), 4.13- 4.34 (m, 3H), 3.60-3.70 (m, 1H, 2.63-2.89 (m, 3H), 2.27-2.38 (m, 1H) 2.08-2.17 (m, 1H), 1.85- 1.98 (m, 1H), 1.52-1.74 (m, 3H). MS obsd. (ESI⁺) [(M + H)⁺]: 506.0. |
| 004 | (2S)-1-[3-[5-bromo-2-(8- | CORE: Int-4 | ¹H NMR (DMSO-d₆, 400 MHz) |
| | chloro-4-oxo-chromen-2- | LINKER: 1,3- | δ ppm 8.20-8.24 (m, 1H), 7.98- |
| | yl)phenoxy]propyl]pyrrolidine- | dibromopropane | 8.03 (m, 2H), 7.85-7.89 (m, 1H), |
| | 2-carboxylic acid | AMINE: | 7.40-7.55 (m, 3H), 7.02 (s, 1H), |
| | 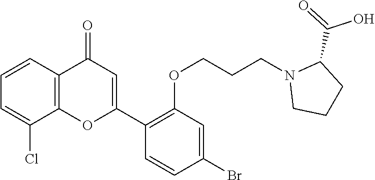 [SMILES: O=C(O)[C@@H]1CCCN1CCCOc1cc(Br)ccc1-c1cc(=O)c2cccc(Cl)c2o1] | methyl (2S)- pyrrolidine-2- carboxylate | 4.16-4.33 (m, 4H), 2.69-2.87 (m, 2H), 2.08-2.17 (m, 2H), 1.85- 2.04 (m, 2H), 1.53-1.73 (m, 2H). MS obsd. (ESI⁺) [(M + H)⁺]: 506.0. |
| 005 | (3R)-1-[3-[5-bromo-2-(8- | CORE: Int-4 | ¹H NMR (DMSO-d₆, 400 MHz) |
| | chloro-4-oxo-chromen-2- | LINKER: 1,3- | δ ppm 8.01 (dq, J = 7.8, 1.5 Hz, |
| | yl)phenoxy]propyl]pyrrolidine- | dibromopropane | 2H), 7.88 (d, J = 8.4 Hz, 1H), |
| | 3-carboxylic acid | AMINE: | 7.47-7.56 (m, 2H), 7.42 (dd, J = |
| | 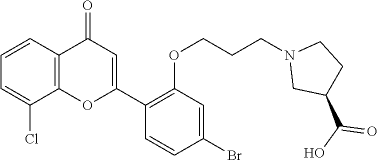 [SMILES: O=C(O)[C@@H]1CCN(CCCOc2cc(Br)ccc2-c2cc(=O)c3cccc(Cl)c3o2)C1] | methyl (3R)- pyrrolidine-3- carboxylate | 8.4, 1.7 Hz, 1H), 7.01-7.10 (m, 1H), 4.24 (t, J = 6.2 Hz, 2H), 2.64-2.84 (m, 4H), 2.54-2.60 (m, 2H), 2.32-2.42 (m, 1H), 1.87- 1.98 (m, 4H). MS obsd. (ESI⁺) [(M + H)⁺]: 505.9. |
| 006 | 3-[3-[2-(8-chloro-4-oxo- | CORE: Int-1 | ¹H NMR (DMSO-d₆, 400 MHz) |
| | chromen-2-yl)-5- | LINKER: 1,3- | δ ppm 12.19-12.69 (br s, 1H), |
| | (trifluoromethyl)phenoxy] | dibromopropane | 8.65-8.99 (m, 1H), 7.90-8.18 (m, |
| | propylamino]cyclobutane- | AMINE: | 3H), 7.44-7.65 (m, 3H), 6.94- |
| | carboxylic acid | methyl 3- | 7.05 (m, 1H), 4.26-4.46 (m, 2H), |
| | 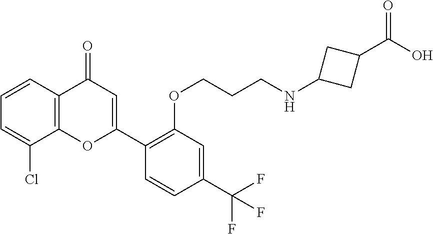 [SMILES: O=C(O)C1CC(NCCCOc2cc(C(F)(F)F)ccc2-c2cc(=O)c3cccc(Cl)c3o2)C1] | amino- cyclobutane- carboxylate hydrochloride | 3.71-3.94 (m, 1H), 2.74-3.20 (m, 4H), 2.33-2.45 (m, 3H), 2.00-2.14 (m, 2H). MS obsd. (ESI⁺) [(M + H)⁺]: 495.9. |
| 007 | 3-[3-[2-(8-chloro-4-oxo- | CORE: Int-1 | ¹H NMR (DMSO-d₆, 400 MHz) |
| | chromen-2-yl)-5- | LINKER: 1,3- | δ ppm 12.26-12.53 (br s, 1H), |
| | (trifluoromethyl)phenoxy] | dibromopropane | 8.64 (br s, 1H), 8.02-8.13 (m, |
| | propylamino]cyclopentane- | AMINE: | 3H), 7.51-7.63 (m, 2H), 7.02 (s, |
| | carboxylic acid | methyl 3- | 1H), 4.31-4.43 (m, 2H), 3.76- |
| | 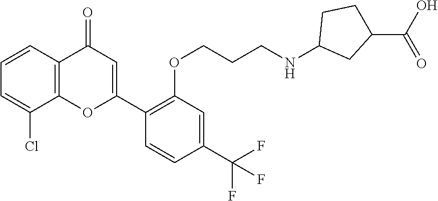 [SMILES: O=C(O)C1CCC(NCCCOc2cc(C(F)(F)F)ccc2-c2cc(=O)c3cccc(Cl)c3o2)C1] | amino- cyclopentane- carboxylate hydrochloride | 3.98 (m, 1H), 3.00-3.11 (m, 2H), 2.70-2.81 (m, 1H), 2.06- 2.29 (m, 3H), 1.90-2.01 (m, 1H), 1.80-1.87 (m, 2H), 1.69-1.78 (m, 1H), 1.56-1.70 (m, 1H). MS obsd. (ESI⁺) [(M + H)⁺]: 509.9. |
| 008 | 2-[1-[3-[2-(8-chloro-4-oxo- | CORE: Int-1 | ¹H NMR (METHANOL-d4, |
| | chromen-2-yl)-5- | LINKER: 1,3- | 400 MHz) δ ppm 8.24 (d, J = 8.6 |
| | (trifluoromethyl)phenoxy] | dibromopropane | Hz, 1H), 8.10 (dd, J = 7.9, 1.3 |
| | propyl]pyrrolidin-3-yl]acetic | AMINE: | Hz, 1H), 7.94 (dd, J = 7.8, 1.3 |
| | acid | methyl 2- | Hz, 1H), 7.46-7.55 (m, 3H), 7.27 |
| | 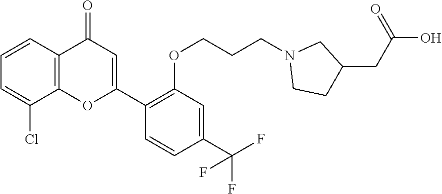 [SMILES: O=C(O)CC1CCN(CCCOc2cc(C(F)(F)F)ccc2-c2cc(=O)c3cccc(Cl)c3o2)C1] | pyrrolidin-3- ylacetate hydrochloride | (s, 1H), 4.30-4.42 (m, 4H), 3.52 (dd, J = 11.6, 7.6 Hz, 1H), 3.35- 3.44 (m, 1H), 3.16-3.31 (m, 1H), 2.93 (dd, J = 11.4, 8.7 Hz, 1H), 2.49-2.73 (m, 3H), 2.17-2.37 (m, 3H), 1.67 (dq, J = 13.2, 8.8 Hz, 1H). MS obsd. (ESI⁺) [(M + H)⁺]: 510.2. |
| 009 | (2R)-2-[3-[2-(8-chloro-4-oxo- | CORE: Int-1 | ¹H NMR (DMSO-d₆, 400 MHz) |
| | chromen-2-yl)-5- | LINKER: 1,3- | δ ppm 8.09-8.16 (m, 1H), 8.04 |
| | (trifluoromethyl)phenoxy] | dibromopropane | (s, 2H), 7.57 (s, 3H), 7.05 (s, |
| | propylamino]-3-methyl- | AMINE: | 1H), 4.31-4.39 (m, 2H), 2.70- |
| | butanoic acid | methyl (2R)-2- | 2.95 (m, 2H), 1.87-2.11 (m, 3H), |
| | 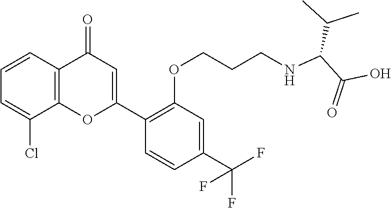 [SMILES: CC(C)[C@@H](NCCCOc1cc(C(F)(F)F)ccc1-c1cc(=O)c2cccc(Cl)c2o1)C(=O)O] | amino-3-methyl- butanoate | 1.19-1.29 (m, 1H), 0.80-0.92 (m, 6H). MS obsd. (ESI⁺) [(M + H)⁺]: 498.1. |
| 010 | 2-[[3-[[2-(8-chloro-4-oxo- | CORE: Int-1 | ¹H NMR (DMSO-d₆, 400 MHz) |
| | chromen-2-yl)-5- | LINKER: 3,3- | δ ppm 8.55-8.79 (m, 1H), 8.10 |
| | (trifluoromethyl)phenoxy] | bis(bromomethyl) | (d, J = 8.3 Hz, 1H), 7.96-8.05 (m, |
| | methyl]oxetan-3- | oxetane | 2H), 7.56-7.67 (m, 2H), 7.51 (t, |
| | yl]methylamino]-2-oxo-acetic | AMINE: | J = 7.9 Hz, 1H), 7.06 (s, 2H), |
| | acid | ethyl 2-amino-2- | 4.43 (d, J = 7.1 Hz, 4H), 4.35 (s, |
| | 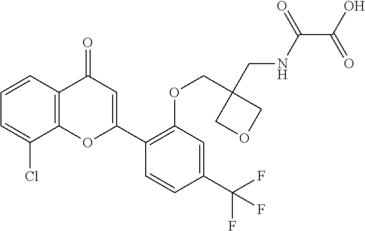 [SMILES: O=C(O)C(=O)NCC1(COc2cc(C(F)(F)F)ccc2-c2cc(=O)c3cccc(Cl)c3o2)COC1] | oxo-acetate | 2H), 3.52 (br d, J = 6.1 Hz, 2H). (ESI⁺) [(M + H)⁺]: 512.0. |
| 011 | 2-[3-[2-(8-chloro-4-oxo- | CORE: Int-1 | ¹H NMR (DMSO-d₆, 400 MHz) |
| | chromen-2-yl)-5- | LINKER: 1,3- | δ ppm 7.96-8.23 (m, 3H), 7.44- |
| | (trifluoromethyl)phenoxy] | dibromopropane | 7.65 (m, 3H), 7.04 (s, 1H), 4.35 |
| | propylamino]-4,4-dimethyl- | AMINE: | (br s, 2H), 2.90 (br d, J = 0.7 Hz, |
| | pentanoic acid | methyl 2-amino- | 2H), 2.07 (s, 4H), 1.58-1.73 (m, |
| | 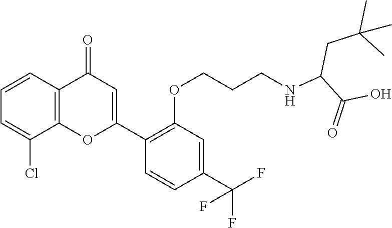 [SMILES: CC(C)(C)CC(NCCCOc1cc(C(F)(F)F)ccc1-c1cc(=O)c2cccc(Cl)c2o1)C(=O)O] | 4,4-dimethyl- pentanoate | 1H), 0.87 (s, 9H). MS obsd. (ESI⁺) [(M + H)⁺]: 526.2. |
| 012 | 1-[3-[2-(8-chloro-4-oxo- | CORE: Int-1 | ¹H NMR (DMSO-d6, 400 MHz) |
| | chromen-2-yl)-5- | LINKER: 1,3- | δ ppm 8.09-8.16 (m, 1H), 7.98- |
| | (trifluoromethyl)phenoxy] | dibromopropane | 8.07 (m, 2H), 7.47-7.64 (m, 3H), |
| | propyl]pyrrolidine-3-carboxylic | AMINE: | 7.02-7.13 (m, 1H), 4.26-4.40 (m, |
| | acid | methyl | 2H), 2.72-3.16 (m, 7H), 1.93- |
| | 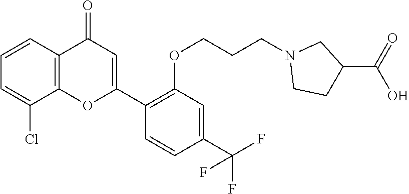 [SMILES: O=C(O)C1CCN(CCCOc2cc(C(F)(F)F)ccc2-c2cc(=O)c3cccc(Cl)c3o2)C1] | pyrrolidine-3- carboxylate hydrochloride | 2.16 (m, 4H). MS obsd. (ESI+) [(M + H)+]: 496.1 |
| 013 | (3R)-1-[3-[2-(8-chloro-4-oxo- | CORE: Int-1 | ¹H NMR (DMSO-d6, 400 MHz) |
| | chromen-2-yl)-5- | LINKER: 1,3- | δ ppm 8.09-8.16 (m, 1H), 7.98- |
| | (trifluoromethyl)phenoxy] | dibromopropane | 8.07 (m, 2H), 7.47-7.64 (m, 3H), |
| | propyl]pyrrolidine-3-carboxylic | AMINE: | 7.02-7.13 (m, 1H), 4.26-4.40 (m, |
| | acid | methyl (3R)- | 2H), 2.72-3.16 (m, 7H), 1.93- |
| | 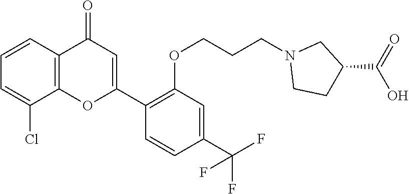 [SMILES: O=C(O)[C@@H]1CCN(CCCOc2cc(C(F)(F)F)ccc2-c2cc(=O)c3cccc(Cl)c3o2)C1] | pyriolidine-3- carboxylate | 2.16 (m, 4H). MS obsd. (ESI+) [(M + H)+]: 496.1 |
| 014 | (3S)-1-[3-[2-(8-chloro-4-oxo- | CORE: Int-1 | ¹H NMR (DMSO-d6, 400 MHz) |
| | chromen-2-yl)-5- | LINKER: 1,3- | δ ppm 8.07-8.15 (m, 1H), 7.98- |
| | (trifluoromethyl)phenoxy] | dibromopropane | 8.06 (m, 2H), 7.47-7.60 (m, 3H), |
| | propyl]pyrrolidine-3-carboxylic | AMINE: | 7.05-7.11 (m, 1H), 4.25-4.36 (m, |
| | acid | methyl (3S)- | 2H), 2.83-2.93 (m, 1H), 2.67- |
| | 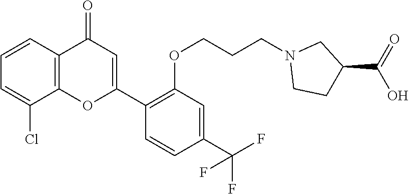 [SMILES: O=C(O)[C@H]1CCN(CCCOc2cc(C(F)(F)F)ccc2-c2cc(=O)c3cccc(Cl)c3o2)C1] | pyrrolidine-3- carboxylate | 2.74 (m, 1H), 2.52-2.59 (m, 4H), 2.37-2.45 (m, 1H), 1.86-2.02 (m, 4H). MS obsd. (ESI+) [(M + H)+]: 496.1 |
| 015 | (3S)-1-[3-[2-(8-chloro-4-oxo- | CORE: Int-13 | ¹H NMR (DMSO-d₆, 400 MHz) |
| | chromen-2-yl)-5-ethoxy- | LINKER: 1,3- | δ ppm 7.97 (s, 3H), 7.43-7.51 |
| | phenoxy]propyl]pyrrolidine- | dibromopropane | (m, 1H), 7.02-7.09 (m, 1H), |
| | 3-carboxylic acid | AMINE: | 6.72-6.81 (m, 2H), 4.10-4.25 (m, |
| | 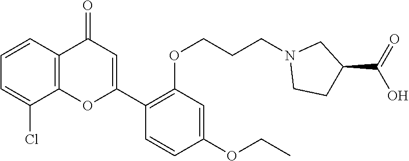 [SMILES: CCOc1ccc(-c2cc(=O)c3cccc(Cl)c3o2)c(OCCCN2CC[C@H](C(=O)O)C2)c1] | methyl (3S)- pyrrolidine-3- carboxylate | 4H), 2.86-2.97 (m, 1H), 2.70- 2.78 (m, 1H), 2.53-2.64 (m, 5H), 1.87-2.02 (m, 4H), 1.31-1.42 (m, 3H). MS obsd. (ESI⁺) [(M + H)+]: 472.1. |
| 016 | (3R)-1-[3-[5-bromo-2-(8- | CORE: Int-7 | ¹H NMR (DMSO-d₆, 400 MHz) |
| | chloro-4-oxo-chromen-2-yl)- | LINKER: 1,3- | δ ppm 7.97-8.04 (m, 2H), 7.58- |
| | 4-methoxy- | dibromopropane | 7.64 (m, 1H), 7.45-7.57 (m, 2H), |
| | phenoxy]propyl]pyrrolidine- | AMINE: | 7.03-7.09 (m, 1H), 4.16-4.24 (m, |
| | 3-carboxylic acid | methyl (3R)- | 2H), 3.90 (s, 3H), 2.75-3.11 (m, |
| | 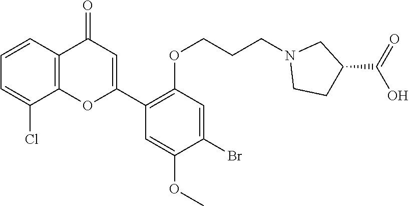 [SMILES: COc1cc(-c2cc(=O)c3cccc(Cl)c3o2)c(OCCCN2CC[C@@H](C(=O)O)C2)cc1Br] | pyrrolidine-3- carboxylate | 7H), 1.95-2.10 (m, 4H). MS obsd. (ESI⁺) [(M + H)+]: 536.1. |
| 017 | (3S)-1-[3-[5-bromo-2-(8- | CORE: Int-7 | ¹H NMR (DMSO-d₆, 400 MHz) |
| | chloro-4-oxo-chromen-2-yl)- | LINKER: 1,3- | δ ppm 7.96-8.05 (m, 2H), 7.60- |
| | 4-methoxy- | dibromopropane | 7.63 (m, 1H), 7.45-7.55 (m, 2H), |
| | phenoxy]propyl]pyrrolidine- | AMINE: | 7.04-7.11 (m, 1H), 4.13-4.21 (m, |
| | 3-carboxylic acid | methyl (3S)- | 2H), 3.88 (s, 3H), 2.65-2.94 (m, |
| | 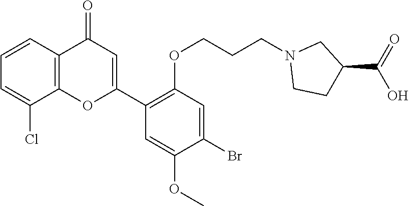 [SMILES: COc1cc(-c2cc(=O)c3cccc(Cl)c3o2)c(OCCCN2CC[C@H](C(=O)O)C2)cc1Br] | pyrrolidine-3- carboxylate | 3H), 2.31-2.46 (m, 3H), 1.84- 1.98 (m, 5H), MS obsd. (ESI⁺) [(M + H)⁺]: 536.1. |
| 018 | 4-[3-[2-(8-chloro-4-oxo- | CORE: Int-1 | ¹H NMR (DMSO-d₆, 400 MHz) |
| | chromen-2-yl)-5- | LINKER: 1,3- | δ ppm 8.13 (d, J = 7.8 Hz, 1H), |
| | (trifluoromethyl)phenoxy] | dibromopropane | 8.04 (d, J = 7.8 Hz, 2H), 7.45- |
| | propyl]morpholine-2- | AMINE: | 7.64 (m, 3H), 7.02-7.10 (m, 1H), |
| | carboxylic acid | methyl | 4.21-4.46 (m, 3H), 3.89-4.16 (m, |
| | 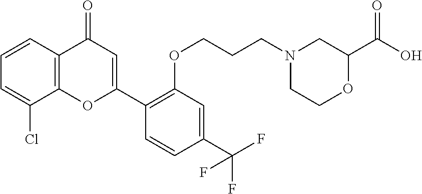 [SMILES: O=C(O)C1CN(CCCOc2cc(C(F)(F)F)ccc2-c2cc(=O)c3cccc(Cl)c3o2)CCO1] | morpholine-3- carboxylate | 2H), 3.63-3.84 (m, 2H), 3.21- 3.22 (m, 2H), 2.89-3.15 (m, 2H), 2.09-2.26 (m, 2H). MS obsd. (ESI⁺) [(M + H)⁺]: 512.0. |
| 019 | 1-[2-[5-bromo-2-(8-chloro-4- | CORE: Int-4 | ¹H NMR (DMSO-d₆, 400 MHz) |
| | oxo-chromen-2- | LINKER: 1,2- | δ ppm 7.98 (dd, J = 8.0, 1.5 Hz, |
| | yl)phenoxy]ethyl]azetidine-3- | dibromoethane | 1H), 7.80-7.94 (m, 2H), 7.39- |
| | carboxylic acid | AMINE: | 7.49 (m, 2H), 7.35 (dd, J = 8.4, |
| | 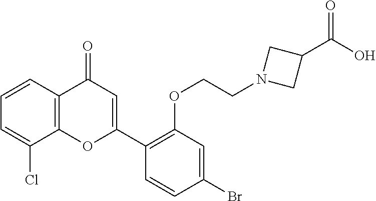 [SMILES: O=C(O)C1CN(CCOc2cc(Br)ccc2-c2cc(=O)c3cccc(Cl)c3o2)C1] | methyl azetidine-3- carboxylate hydrochloride | 1.8 Hz, 1H), 7.09 (s, 1H), 3.79- 3.93 (m, 2H), 3.60-3.71 (m, 2H), 3.42-3.55 (m, 2H), 3.10-3.15 (m, 1H), 3.02-3.07 (m, 2H). MS obsd. (ESI⁺) [(M + H)⁺]: 478.3. |
| 020 | 1-[2-[5-bromo-2-(8-chloro-4- | CORE: Int-4 | ¹H NMR (METHANOL-d₄, |
| | oxo-chromen-2- | LINKER: 1,2- | 400 MHz) δ ppm 7.96-8.02 (m, |
| | yl)phenoxy]ethyl]pyrrolidine- | dibromoethane | 1H), 7.79-7.86 (m, 2H), 7.28- |
| | 2-carboxylic acid | AMINE: | 7.41 (m, 3H), 7.07-7.13 (m, 1H), |
| | 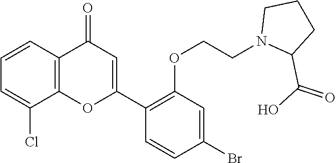 [SMILES: O=C(O)C1CCCN1CCOc1cc(Br)ccc1-c1cc(=O)c2cccc(Cl)c2o1] | methyl pyrrolidine-2- carboxylate hydrochloride | 4.33-4.48 (m, 2H), 3.49-3.92 (m, 4H), 2.97-3.12 (m, 1H), 2.24- 2.36 (m, 1H), 1.68-2.03 (m, 3H). MS obsd. (ESI⁺) [(M + H)⁺]: 492.1. |
| 021 | 1-[2-[5-bromo-2-(8-chloro-4- | CORE: Int-4 | ¹H NMR (DMSO-d₆, 400 MHz) |
| | oxo-chromen-2- | LINKER: 1,2- | δ ppm 8.02 (d, J = 7.8 Hz, 2H), |
| | yl)phenoxy]ethyl]pyrrolidine- | dibromoethane | 7.84 (d, J = 8.4 Hz, 1H), 7.60 (d, |
| | 3-carboxylic acid | AMINE: | J = 1.7 Hz, 1H), 7.45-7.54 (m, |
| | 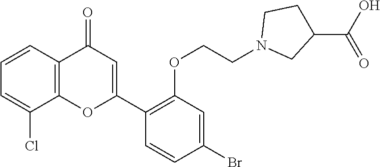 [SMILES: O=C(O)C1CCN(CCOc2cc(Br)ccc2-c2cc(=O)c3cccc(Cl)c3o2)C1] | methyl pyrrolidine-3- carboxylate hydrochloride | 2H), 7.00 (s, 1H), 4.45-4.55 (m, 2H), 3.57-3.69 (m, 2H), 3.03- 3.23 (m, 1H), 1.87-2.28 (m, 4H), 1.12-1.33 (m, 2H). MS obsd. (ESI⁺) [(M + H)⁺]: 492.0. |
| 022 | 1-[2-[5-bromo-2-(8-chloro-4- | CORE: Int-4 | ¹H NMR (DMSO-d₆, 400 MHz) |
| | oxo-chromen-2- | LINKER: 1,2- | δ ppm 7.99 (dd, J = 8.1, 1.5 Hz, |
| | yl)phenoxy]ethyl]piperidine- | dibromoethane | 1H), 7.80-7.87 (m, 2H), 7.34- |
| | 4-carboxylic acid | AMINE: | 7.43 (m, 2H), 7.28-7.34 (m, 1H), |
| | 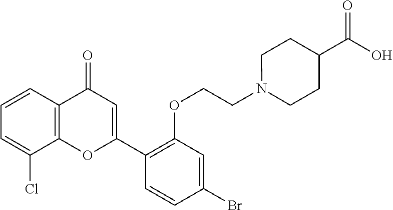 [SMILES: O=C(O)C1CCN(CCOc2cc(Br)ccc2-c2cc(=O)c3cccc(Cl)c3o2)CC1] | methyl piperidine-4- carboxylate hydrochloride | 7.09 (s, 1H), 4.35 (t, J = 5.1 Hz, 2H), 3.09-3.14 (m, 2H), 2.58- 2.70 (m, 2H), 2.18-2.30 (m, 1H), 1.82-1.98 (m, 2H), 1.62-1.78 (m, 4H). MS obsd. (ESI⁺) [(M + H)⁺]: 506.4. |
| 023 | 4-[2-[5-bromo-2-(8-chloro-4- | CORE: Int-4 | ¹H NMR (DMSO-d₆, 400 MHz) |
| | oxo-chromen-2- | LINKER: 1,2- | δ ppm 7.91-7.97 (m, 2H), 7.83- |
| | yl)phenoxy]ethyl]morpholine- | dibromoethane | 7.88 (m, 1H), 7.48 (d, J = 1.6 |
| | 2-carboxylic acid | AMINE: | Hz, 1H), 7.33-7.45 (m, 3H), 4.26 |
| | 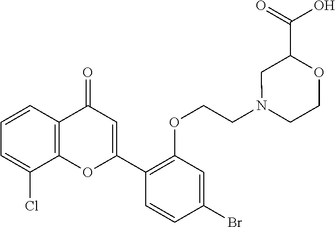 [SMILES: O=C(O)C1CN(CCOc2cc(Br)ccc2-c2cc(=O)c3cccc(Cl)c3o2)CCO1] | methyl morpholine-2- carboxylate hydrochloride | (br t, J = 5.4 Hz, 2H), 4.08 (br dd, J = 8.6, 2.3 Hz, 1H), 3.73- 3.82 (m, 1H), 3.51-3.58 (m, 1H), 2.86-2.92 (m, 1H), 2.72 (br d, J = 6.7 Hz, 2H), 2.63 (br d, J = 12.8 Hz, 1H), 2.15-2.27 (m, 2H). MS obsd. (ESI⁺) [(M + H)⁺]: 506.0. |
| 024 | (3S,4S)-1-[2-[5-bromo-2-(8- | CORE: Int-4 | ¹H NMR (DMSO-d₆, 400 MHz) |
| | chloro-4-oxo-chromen-2- | LINKER: 1,2- | δ ppm 9.96-10.31 (br s, 1H), |
| | yl)phenoxy]ethyl]-4-methyl- | dibromoethane | 8.02 (d, J = 7.9 Hz, 2H), 7.83 (d, |
| | pyrrolidine-3-carboxylic acid | AMINE: | J = 8.4 Hz, 1H), 7.57-7.65 (m, |
| | 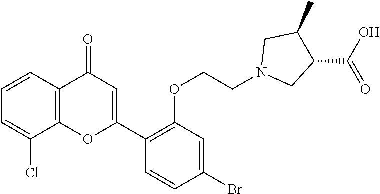 [SMILES: C[C@@H]1CN(CCOc2cc(Br)ccc2-c2cc(=O)c3cccc(Cl)c3o2)C[C@H]1C(=O)O] | methyl (3S,4S)- 4- methyl- pyrrolidine- 3-carboxylate hydrochloride | 1H), 7.42-7.55 (m, 2H), 6.96 (s, 1H), 4.48-4.54 (m, 2H), 3.74- 3.90 (m, 2H), 3.59-3.74 (m, 3H), 2.65-2.98 (m, 2H), 2.25-2.44 (m, 1H), 1.04 (d, J = 6.6 Hz, 3H). MS obsd. (ESI⁺) [(M + H)⁺]: 506.1. |
| 025 | 8-[2-[5-bromo-2-(8-chloro-4- | CORE: Int-4 | ¹H NMR (DMSO-d₆, 400 MHz) |
| | oxo-chromen-2- | LINKER: 1,2- | δ ppm 12.34-12.64 (m, 1H), |
| | yl)phenoxy]ethyl]-8- | dibromoethane | 7.93-8.15 (m, 2H), 7.83 (d, J = |
| | azabicyclo[3.2.1]octane-3- | AMINE: | 8.3 Hz, 1H), 7.38-7.64 (m, 3H), |
| | carboxylic acid | methyl 8- | 6.85-7.04 (m, 1H), 4.47-4.65 (m, |
| | 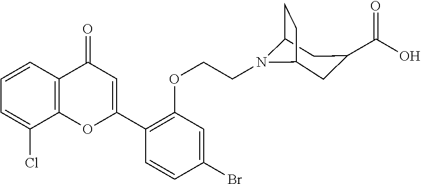 [SMILES: O=C(O)C1CC2CCC(C1)N2CCOc1cc(Br)ccc1-c1cc(=O)c2cccc(Cl)c2o1] | azabicyclo[3.2.1] octane-3- carboxylate hydrochloride | 2H), 3.97-4.15 (m, 3H), 2.63- 2.82 (m, 2H), 2.04-2.24 (m, 2H), 1.93-2.04 (m, 2H), 1.72-1.90 (m, 4H). MS obsd. (ESI⁺) [(M + H)⁺]: 532.2. |
| 026 | 3-[2-[5-bromo-2-(8-chloro-4- | CORE: Int-4 | ¹H NMR (DMSO-d₆, 400 MHz) |
| | oxo-chromen-2- | LINKER: 1,2- | δ ppm 8.03 (d, J = 7.8 Hz, 2H), |
| | yl)phenoxy]ethylamino]bicyclo | dibromoethane | 7.87 (d, J = 8.4 Hz, 1H), 7.44- |
| | [1.1.1]pentane-1-carboxylic | AMINE: | 7.61 (m, 3H), 7.07 (s, 1H), 4.35- |
| | acid | methyl 3- | 4.46 (m, 2H), 2.54-2.64 (m, 2H), |
| | 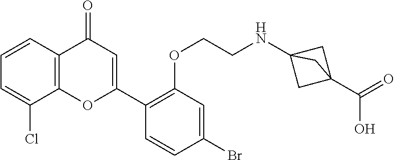 [SMILES: O=C(O)C12CC(NCCOc3cc(Br)ccc3-c3cc(=O)c4cccc(Cl)c4o3)(C1)C2] | aminobicyclo[1. 1.1]pentane-1- carboxylate hydrochloride | 2.11-2.29 (m, 6H). MS obsd. (ESI⁺) [(M + H)⁺]: 504.2. |
| 027 | 1-[2-[5-bromo-2-(8-chloro-4- | CORE: Int-4 | ¹H NMR (DMSO-d₆, 400 MHz) |
| | oxo-chromen-2- | LINKER: 1,2- | δ ppm 8.02 (t, J = 6.6 Hz, 2H), |
| | yl)phenoxy]ethyl]-4,4- | dibromoethane | 7.94 (d, J = 8.6 Hz, 1H), 7.59 (d, |
| | difluoro-pyrrolidine-2- | AMINE: | J = 1.5 Hz, 1H), 7.51 (d, J = 7.8 |
| | carboxylic acid | methyl 4,4- | Hz, 2H), 7.14 (s, 1H), 4.70 (br t, |
| | 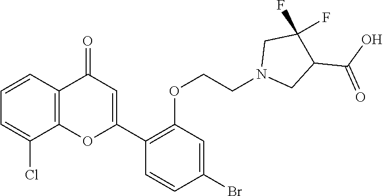 | difluoro- pyrrolidine-3- carboxylate hydrochloride | J = 8.3 Hz, 2H), 4.56-4.63 (m, 2H), 4.46-4.54 (m, 2H), 3.57- 3.66 (m, 1H), 2.77-2.89 (m, 1H), 2.61-2.73 ppm (m, 1H). MS obsd. (ESI⁺) [(M + H)⁺]: 528.0. |
| 029 | 1-[2-[2-(8-chloro-4-oxo- | CORE: Int-3 | ¹H NMR (DMSO-d₆, 400 MHz) |
| | chromen-2- | LINKER: 1,2- | δ ppm 8.00-8.06 (m, 2H), 7.86- |
| | yl)phenoxy]ethyl]piperidine- | dibromoethane | 7.94 (m, 1H), 7.61-7.69 (m, 1H), |
| | 4-carboxylic acid | AMINE: | 7.48-7.55 (m, 1H), 7.31-7.36 (m, |
| | 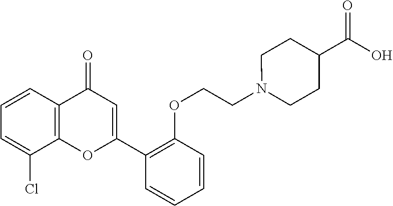 [SMILES: O=C(O)C1CCN(CCOc2ccccc2-c2cc(=O)c3cccc(Cl)c3o2)CC1] | methyl piperidine-4- carboxylate hydrochloride | 1H), 7.22-7.30 (m, 1H), 6.93- 6.99 (m, 1H), 4.50-4.59 (m, 2H), 3.52-3.65 (m, 3H), 3.01-3.16 (m, 2H), 2.36-2.49 (m, 2H), 1.91- 2.03 (m, 2H), 1.63-1.85 (m, 2H). MS obsd. (ESI⁺) [(M + H)⁺]: 428.1. |
| 030 | (2R)-1-[2-[2-(8-chloro-4-oxo- | CORE: Int-3 | ¹H NMR (METHANOL-d₄, |
| | chromen-2- | LINKER: 1,2- | 400 MHz) δ ppm 8.06-8.12 (m, |
| | yl)phenoxy]ethyl]pyrrolidine- | dibromoethane | 1H), 7.96-8.03 (m, 1H), 7.87- |
| | 2-carboxylic acid | AMINE: | 7.94 (m, 1H), 7.55-7.64 (m, 1H), |
| | 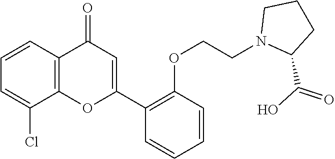 [SMILES: O=C(O)[C@H]1CCCN1CCOc1ccccc1-c1cc(=O)c2cccc(Cl)c2o1] | methyl (2R)- pyrrolidine-2- carboxylate hydrochloride | 7.42-7.51 (m, 1H), 7.18-7.29 (m, 2H), 7.10-7.15 (m, 1H), 4.47- 4.59 (m, 2H), 3.98-4.08 (m, 1H), 3.66-3.89 (m, 3H), 3.16-3.28 (m, 1H), 2.33-2.47 (m, 1H), 1.98- 2.16 (m, 2H), 1.80-1.95 (m, 1H). MS obsd. (ESI⁺) [(M + H)⁺]: 414.1. |
| 031 | 1-[2-[2-(8-chloro-4-oxo- | CORE: Int-3 | ¹H NMR (DMSO-d₆, 400 MHz) |
| | chromen-2- | LINKER: 1,2- | δ ppm 7.95-8.03 (m, 3H), 7.55- |
| | yl)phenoxy]ethyl]pyrrolidine- | dibromoethane | 7.61 (m, 1H), 7.46-7.53 (m, 1H), |
| | 3-carboxylic acid | AMINE: | 7.26-7.33 (m, 2H), 7.14-7.23 (m, |
| | 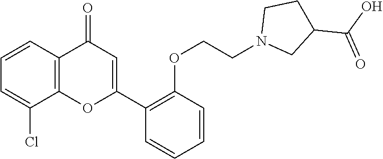 [SMILES: O=C(O)C1CCN(CCOc2ccccc2-c2cc(=O)c3cccc(Cl)c3o2)C1] | methyl pyrrolidine-3- carboxylate hydrochloride | 1H), 4.21-4.28 (m, 2H), 2.77- 2.93 (m, 4H), 2.57-2.73 (m, 3H), 1.82-1.95 (m, 2H). MS obsd. (ESI⁺) [(M + H)⁺]: 414.1. |
| 032 | (2S)-1-[2-[2-(8-chloro-4-oxo- | CORE: Int-3 | ¹H NMR (METHANOL-d₄, |
| | chromen-2- | LINKER: 1,2- | 400 MHz) δ ppm 8.06-8.12 (m, |
| | yl)phenoxy]ethyl]pyrrolidine- | dibromoethane | 1H), 7.96-8.03 (m, 1H), 7.87- |
| | 2-carboxylic acid | AMINE: | 7.94 (m, 1H), 7.55-7.64 (m, 1H), |
| | 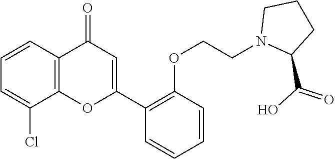 [SMILES: O=C(O)[C@@H]1CCCN1CCOc1ccccc1-c1cc(=O)c2cccc(Cl)c2o1] | methyl (2S)- pyrrolidine-2- carboxylate hydrochloride | 7.42-7.51 (m, 1H), 7.18-7.29 (m, 2H), 7.10-7.15 (m, 1H), 4.47- 4.59 (m, 2H), 3.98-4.08 (m, 1H), 3.66-3.89 (m, 3H), 3.16-3.28 (m, 1H), 2.33-2.47 (m, 1H), 1.98- 2.16 (m, 2H), 1.80-1.95 (m, 1H). MS obsd. (ESI⁺) [(M + H)⁺]: 414.1. |
| 033 | 1-[2-[2-(8-chloro-4-oxo- | CORE: Int-3 | ¹H NMR (DMSO-d₆, 400 MHz) |
| | chromen-2- | LINKER: 1,2- | δ ppm 7.92-8.05 (m, 3H), 7.54- |
| | yl)phenoxy]ethyl]azetidine-3- | dibromoethane | 7.62 (m, 1H), 7.45-7.54 (m, 1H), |
| | carboxylic acid | AMINE: | 7.14-7.29 (m, 3H), 4.07-4.15 (m, |
| | 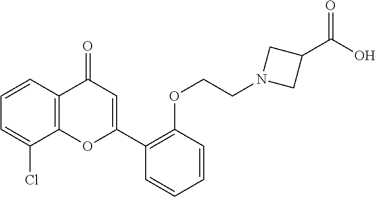 [SMILES: O=C(O)C1CN(CCOc2ccccc2-c2cc(=O)c3cccc(Cl)c3o2)C1] | methyl azetidine-3- carboxylate hydrochloride | 2H), 3.39-3.46 (m, 2H), 3.17- 3.24 (m, 2H), 3.07-3.17 (m, 1H), 2.76-2.86 (m, 2H). MS obsd. (ESI⁺) [(M + H)⁺]: 400.1. |
| 034 | (3S)-1-[2-[2-(8-chloro-4-oxo- | CORE: Int-1 | ¹H NMR (DMSO-d₆, 400 MHz) |
| | chromen-2-yl)-5- | LINKER: 1,2- | δ ppm 8.06-8.13 (m, 1H), 7.99- |
| | (trifluoromethyl)phenoxy]ethyl] | dibromoethane | 8.06 (m, 2H), 7.58-7.67 (m, 2H), |
| | pyrrolidine-3-carboxylic | AMINE: | 7.48-7.56 (m, 1H), 7.06-7.14 (m, |
| | acid | methyl (3S)- | 1H), 4.51-4.60 (m, 2H), 3.03- |
| | 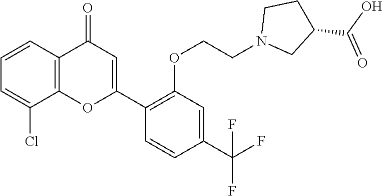 [SMILES: O=C(O)[C@H]1CCN(CCOc2cc(C(F)(F)F)ccc2-c2cc(=O)c3cccc(Cl)c3o2)C1] | pyrrolidine-3- carboxylate hydrochloride | 3.42 (m, 7H), 1.93-2.21 (m, 2H). MS obsd. (ESI⁺) [(M + H)⁺]: 482.1. |
| 035 | 4-[2-[2-(8-chloro-4-oxo- | CORE: Int-1 | ¹H NMR (DMSO-d₆, 400 MHz) |
| | chromen-2-yl)-5- | LINKER: 1,2- | δ ppm 8.03-8.12 (m, 1H), 7.89- |
| | (trifluoromethyl)phenoxy]ethyl] | dibromoethane | 8.00 (m, 2H), 7.50-7.60 (m, 2H), |
| | morpholine-2-carboxylic | AMINE: | 7.32-7.50 (m, 1H), 7.16-7.32 (m, |
| | acid | methyl | 1H), 4.40-4.52 (m, 2H), 4.11- |
| | 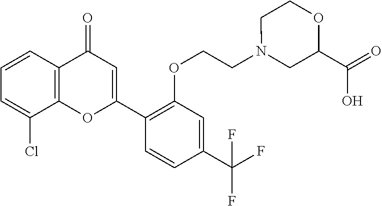 [SMILES: O=C(O)C1CN(CCOc2cc(C(F)(F)F)ccc2-c2cc(=O)c3cccc(Cl)c3o2)CCO1] | morpholine-2- carboxylate hydrochloride | 4.27 (m, 1H), 3.78-3.92 (m, 1H), 3.57-3.66 (m, 2H), 2.87-3.11 (m, 3H), 2.50-2.80 (m, 2H). MS obsd. (ESI⁺) [(M + H)⁺]: 498.1. |
| 036 | (2R)-2-[2-[2-(8-chloro-4-oxo- | CORE: Int-1 | ¹H NMR (DMSO-d₆, 400 MHz) |
| | chromen-2-yl)-5- | LINKER: 1,2- | δ ppm 8.11 (d, J = 8.0 Hz, 1H), |
| | (trifluoromethyl)phenoxy]ethylamino]- | dibromoethane | 8.01 (d, J = 7.9 Hz, 2H), 7.56 (d, |
| | 3-methyl-butanoic | AMINE: | J = 10.1 Hz, 2H), 7.51 (t, J = 7.9 |
| | acid | methyl (2R)-2- | Hz, 1H), 7.16 (s, 1H), 4.35 (t, J = |
| | 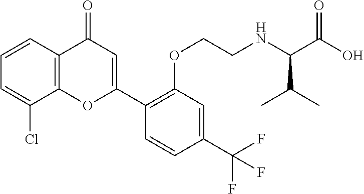 [SMILES: CC(C)[C@@H](NCCOc1cc(C(F)(F)F)ccc1-c1cc(=O)c2cccc(Cl)c2o1)C(=O)O] | amino-3-methyl- butanoate | 5.1 Hz, 2H), 3.16-3.05 (m, 1H), 3.00 (d, J = 5.2 Hz, 1H), 2.95-2.81 (m, 1H), 1.85 (dq, J = 13.2, 6.6 Hz, 1H), 0.83 (t, J = 6.8 Hz, 6H). MS obsd. (ESI⁺) [(M + H)⁺]: 484.1. |
| 037 | (2S)-2-[2-[2-(8-chloro-4-oxo- | CORE: Int-1 | ¹H NMR (DMSO-d₆, 400 MHz) |
| | chromen-2-yl)-5- | LINKER: 1,2- | δ ppm 8.11 (d, J = 8.1 Hz, 1H), |
| | (trifluoromethyl)phenoxy]ethylamino]- | dibromoethane | 8.05-7.97 (m, 2H), 7.57 (d, J = |
| | 3-methyl-butanoic | AMINE: | 7.5 Hz, 2H), 7.51 (t, J = 7.9 Hz, |
| | acid | methyl (2S)-2- | 1H), 7.14 (s, 1H), 4.39 (t, J = 5.2 |
| | 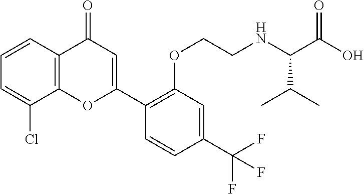 [SMILES: CC(C)[C@H](NCCOc1cc(C(F)(F)F)ccc1-c1cc(=O)c2cccc(Cl)c2o1)C(=O)O] | amino-3-methyl- butanoate | Hz, 2H), 3.21-3.12 (m, 2H), 3.06-2.94 (m, 1H), 1.92 (dq, J = 13.4, 6.6 Hz, 1H), 0.84 (d, J = 6.8 Hz, 6H). MS obsd. (ESI⁺) [(M + H)⁺]: 484.1. |
| 038 | 3-[2-[2-(8-chloro-4-oxo- | CORE: Int-1 | ¹H NMR (DMSO-d₆, 400 MHz) |
| | chromen-2-yl)-5- | LINKER: 1,2- | δ ppm 8.12 (dd, J = 7.7, 5.3 Hz, |
| | (trifluoromethyl)phenoxy] | dibromoethane | 1H), 8.05 (dd, J = 7.9, 1.8 Hz, |
| | ethylamino]cyclobutanecarboxylic | AMINE: | 2H), 7.59-7.68 (m, 2H), 7.54 (td, |
| | acid | methyl 3- | J = 7.9, 1.6 Hz, 1H), 7.10 (d, J = |
| | 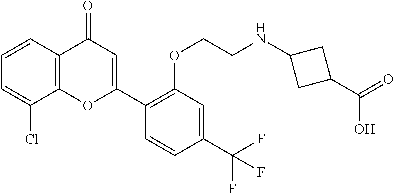 [SMILES: O=C(O)C1CC(NCCOc2cc(C(F)(F)F)ccc2-c2cc(=O)c3cccc(Cl)c3o2)C1] | aminocyclo- butane- carboxylate hydrochloride | 12.7 Hz, 1H), 4.51 (q, J = 4.3 Hz, 2H), 3.84-3.93 (m, 1H), 3.64-3.73 (m, 1H), 2.95-3.09 (m, 1H), 2.78 (br t, J = 8.6 Hz, 1H), 2.18-2.43 ppm (m, 4H). MS obsd. (ESI⁺) [(M + H)⁺]: 482.6. |
| 039 | (3R)-1-[2-[2-(8-chloro-4-oxo- | CORE: Int-14 | ¹H NMR (DMSO-d₆, 400 MHz) |
| | chromen-2-yl)-5-isobutoxy- | LINKER: 1,2- | δ ppm 7.93-8.00 (m, 3H), 7.43- |
| | phenoxy]ethyl]pyrrolidine-3- | dibromoethane | 7.50 (m, 1H), 7.24-7.28 (m, 1H), |
| | carboxylic acid | AMINE: | 6.74-6.83 (m, 2H), 4.23-4.31 (m, |
| | 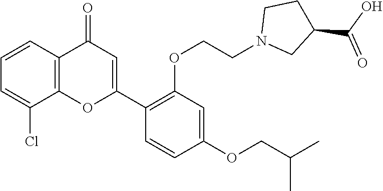 [SMILES: CC(C)COc1ccc(-c2cc(=O)c3cccc(Cl)c3o2)c(OCCN2CC[C@@H](C(=O)O)C2)c1] | methyl (3R)- pyrrolidine-3- carboxylate hydrochloride | 2H), 3.84-3.92 (m, 2H), 2.85- 2.96 (m, 5H), 2.63-2.75 (m, 2H), 2.01-2.12 (m, 1H), 1.88-1.99 (m, 2H), 1.01 (d, J = 6.72 Hz, 6H). MS obsd. (ESI⁺) [(M + H)⁺]: 486.1. |
| 040 | (3S)-1-[2-[2-(8-chloro-4-oxo- | CORE: Int-14 | ¹H NMR (DMSO-d₆, 400 MHz) |
| | chromen-2-yl)-5-isobutoxy- | LINKER: 1,2- | δ ppm 7.97 (d, J = 7.95 Hz, 3H), |
| | phenoxy]ethyl]pyrrolidine-3- | dibromoethane | 7.42-7.50 (m, 1H), 7.26 (s, 1H), |
| | carboxylic acid | AMINE: | 6.80 (s, 2H), 4.21-4.30 (m, 2H), |
| | 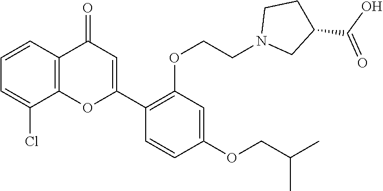 [SMILES: CC(C)COc1ccc(-c2cc(=O)c3cccc(Cl)c3o2)c(OCCN2CC[C@H](C(=O)O)C2)c1] | methyl (3S)- pyrrolidine-3- carboxylate hydrochloride | 3.84-3.91 (m, 2H), 2.81-2.99 (m, 5H), 2.63-2.75 (m, 2H), 1.87- 2.11 (m, 3H), 1.01 (d, J = 6.72 Hz, 6H). MS obsd. (ESI⁺) [(M + H)⁺]: 486.1. |
| 041 | (3R)-1-[2-[5-bromo-2-[8- | CORE: Int-17 | ¹H NMR (DMSO-d₆, 400 MHz) |
| | chloro-4-oxo-5- | LINKER: 1,2- | δ ppm 8.14-8.19 (m, 1H), 7.85- |
| | (trifluoromethyl)chromen-2- | dibromoethane | 7.93 (m, 2H), 7.54-7.60 (m, 1H), |
| | yl]phenoxy]ethyl]pyrrolidine- | AMINE: | 7.43-7.48 (m, 1H), 7.14-7.22 (m, |
| | 3-carboxylic acid | methyl (3R)- | 1H), 4.38-4.47 (m, 2H), 2.87- |
| | 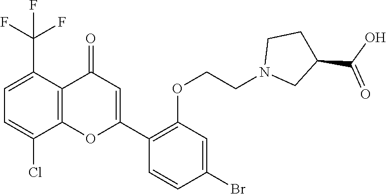 [SMILES: O=C(O)[C@@H]1CCN(CCOc2cc(Br)ccc2-c2cc(=O)c3c(C(F)(F)F)ccc(Cl)c3o2)C1] | pyrrolidine-3- carboxylate hydrochloride | 3.14 (m, 7H), 1.94-2.15 (m, 2H). MS obsd. (ESI⁺) [(M + H)⁺]: 560.1. |
| 042 | (3S)-1-[2-[5-bromo-2-[8- | CORE: Int-17 | ¹H NMR (DMSO-d₆, 400 MHz) |
| | chloro-4-oxo-5- | LINKER: 1,2- | δ ppm 8.13-8.19 (m, 1H), 7.91- |
| | (trifluoromethyl)chromen-2- | dibromoethane | 7.95 (m, 1H), 7.84-7.90 (m, 1H), |
| | yl]phenoxy]ethyl]pyrrolidine- | AMINE: | 7.52-7.58 (m, 1H), 7.41-7.46 (m, |
| | 3-carboxylic acid | methyl (3S)- | 1H), 7.31-7.37 (m, 1H), 4.25- |
| | 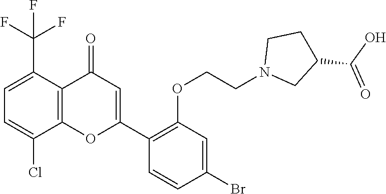 | pyrrolidine-3- carboxylate hydrochloride | 4.34 (m, 2H), 2.78-2.99 (m, 5H), 2.60-2.74 (m, 2H), 1.84-2.03 (m, 2H). MS obsd. (ESI⁺) [(M + H)⁺]: 560.1. |
| 043 | 1-[2-[2-(8-chloro-6-fluoro-4- | CORE: Int-15 | ¹H NMR (DMSO-d₆, 400 MHz) |
| | oxo-chromen-2- | LINKER: 1,2- | δ ppm 8.11 (dd, J = 3.00, 8.13 |
| | yl)phenoxy]ethyl]pyrrolidine- | dibromoethane | Hz, 1H), 7.94-8.00 (m, 1H), |
| | 3-carboxylic acid | AMINE: | 7.68-7.75 (m, 1H), 7.53-7.65 (m, |
| | 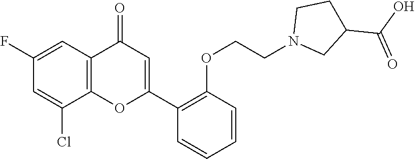 [SMILES: O=C(O)C1CCN(CCOc2ccccc2-c2cc(=O)c3cc(F)cc(Cl)c3o2)C1] | methyl pyrrolidine-3- carboxylate hydrochloride | 1H), 7.30 (s, 2H), 7.15-7.24 (m, 1H), 4.19-4.30 (m, 2H), 2.78- 2.96 (m, 4H), 2.59-2.74 (m, 3H), 1.87-1.99 (m, 2H). MS obsd. (ESI⁺) [(M + H)⁺]: 432.1. |

| | | | |
|---|---|---|---|
| 044 | (2R)-1-[2-[5-bromo-2-(8- | CORE: Int-6 | ¹H NMR (DMSO-d₆, 400 MHz) |
| | chloro-4-oxo-chromen-2-yl)- | LINKER: 1,2- | δ ppm 7.95-8.02 (m, 2H), 7.80- |
| | 4-methyl- | dibromoethane | 7.85 (m, 1H), 7.45-7.53 (m, 2H), |
| | phenoxy]ethyl]pyrrolidine-2- | AMINE: | 7.09-7.13 (m, 1H), 4.26-4.35 (m, |
| | carboxylic acid | methyl (2R)- | 2H), 3.40-3.49 (m, 1H), 3.03- |
| | 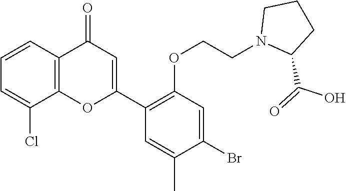 [SMILES: Cc1cc(-c2cc(=O)c3cccc(Cl)c3o2)c(OCCN2CCC[C@@H]2C(=O)O)cc1Br] | pyrrolidine-2- carboxylate hydrochloride | 3.32 (m, 3H), 2.62-2.74 (m, 1H), 2.37 (s, 3H), 2.00-2.14 (m, 1H), 1.61-1.89 (m, 3H). MS obsd. (ESI⁺) [(M + H)⁺]: 506.1. |
| 045 | (2S)-1-[2-[5-bromo-2-(8- | CORE: Int-5 | ¹H NMR (DMSO-d₆, 400 MHz) |
| | chloro-4-oxo-chromen-2-yl)- | LINKER: 1,2- | δ ppm 7.95-8.08 (m, 2H), 7.73- |
| | 4-methyl- | dibromoethane | 7.89 (m, 1H), 7.47-7.53 (m, 1H), |
| | phenoxy]ethyl]pyrrolidine-2- | AMINE: | 7.38-7.42 (m, 1H), 7.02-7.07 (m, |
| | carboxylic acid | methyl (2S)- | 1H), 4.33-4.40 (m, 2H), 3.19- |
| | 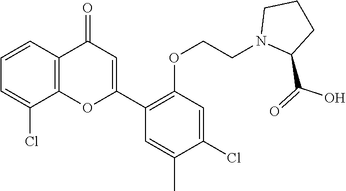 [SMILES: Cc1cc(-c2cc(=O)c3cccc(Cl)c3o2)c(OCCN2CCC[C@H]2C(=O)O)cc1Cl] | pyrrolidine-2- carboxylate hydrochloride | 3.31 (m, 2H), 2.80-2.91 (m, 2H), 2.38 (s, 3H), 2.11-2.22 (m, 1H), 1.67-1.92 (m, 4H). MS obsd. (ESI⁺) [(M + H)⁺]: 462.1 |
| 046 | 3-[3-[5-chloro-2-(8-chloro-4- | CORE: Int-5 | ¹H NMR (DMSO-d₆, 400 MHz) |
| | oxo-chromen-2-yl)-4-methyl- | LINKER: 1,3- | δ ppm 7.97-8.07 (m, 2H), 7.81- |
| | phenoxy]propylamino] | dibromopropane | 7.92 (m, 1H), 7.47-7.56 (m, 1H), |
| | cyclobutanecarboxylic acid | AMINE: | 7.35-7.40 (m, 1H), 6.90-6.96 (m, |
| | 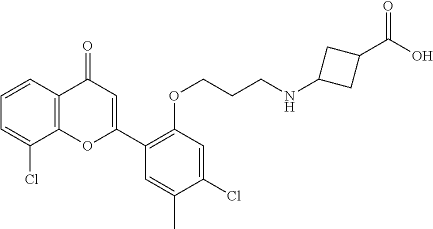 [SMILES: Cc1cc(-c2cc(=O)c3cccc(Cl)c3o2)c(OCCCNC2CC(C(=O)O)C2)cc1Cl] | methyl 3- amino- cyclobutane- carboxylate hydrochloride | 1H), 4.20-4.30 (m, 2H), 3.54- 3.85 (m, 1H), 2.81-3.12 (m, 4H), 2.2-2.37 (m, 5H), 2.18-2.28 (m, 1H), 2.01-2.13 (m, 2H). MS obsd. (ESI⁺) [(M + H)⁺]: 476.1. |
| 047 | (3R)-1-[2-[2-[5-bromo-2-(8- | CORE: Int-4 | ¹H NMR (DMSO-d₆, 400 MHz) |
| | chloro-4-oxo-chromen-2- | LINKER: 1- | δ ppm 9.66-9.92 (m, 1H), 7.90- |
| | yl)phenoxy]ethoxy]ethyl] | bromo-2-(2- | 8.06 (m, 3H), 7.42-7.58 (m, 3H), |
| | pyrrolidine-3-carboxylic acid | bromoethoxy) | 7.29 (s, 1H), 4.35-4.46 (m, 2H), |
| | 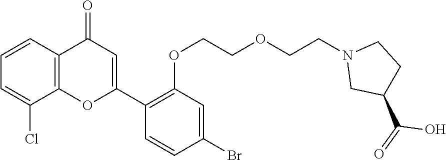 [SMILES: O=C(O)[C@@H]1CCN(CCOCCOc2cc(Br)ccc2-c2cc(=O)c3cccc(Cl)c3o2)C1] | ethane AMINE: methyl (3R)- pyrrolidine-3- carboxylate hydrochloride | 3.86-3.97 (m, 2H), 3.71-3.84 (m, 3H), 3.54-3.66 (m, 2H), 3.24- 3.39 (m, 2H), 3.00-3.24 (m, 2H), 1.91-2.37 ppm (m, 2H). MS obsd. (ESI⁺) [(M + H)⁺]: 536.2. |
| 048 | (2R)-1-[2-[2-[5-bromo-2-(8- | CORE: Int-6 | ¹H NMR (DMSO-d₆, 400 MHz) |
| | chloro-4-oxo-chromen-2-yl)- | LINKER: 1- | δ ppm 7.98-8.07 (m, 2H), 7.90- |
| | 4-methyl- | bromo-2-(2- | 7.96 (m, 1H), 7.45-7.59 (m, 2H), |
| | phenoxy]ethoxy]ethyl] | bromoethoxy) | 7.23-7.30 (m, 1H), 4.24-4.44 (m, |
| | pyrrolidine-2-carboxylic acid | ethane | 2H), 3.51-3.93 (m, 8H), 3.08- |
| | 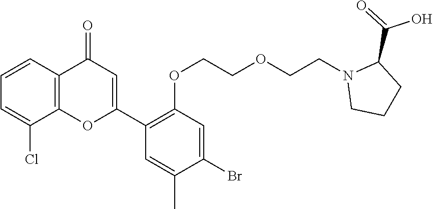 [SMILES: Cc1cc(-c2cc(=O)c3cccc(Cl)c3o2)c(OCCOCCN2CCC[C@@H]2C(=O)O)cc1Br] | AMINE: methyl (2R)- pyrrolidine-2- carboxylate hydrochloride | 3.29 (m, 1H), 2.31-2.41 (m, 4H), 1.68-2.08 (m, 3H). MS obsd. (ESI⁺) [(M + H)⁺]: 550.1. |
| 049 | 4-[2-[2-[5-bromo-2-(8-chloro- | CORE: Int-4 | ¹H NMR (DMSO-d₆, 400 MHz) |
| | 4-oxo-chromen-2- | LINKER: 1- | δ ppm 7.88-8.08 (m, 3H), 7.42- |
| | yl)phenoxy]ethoxy]ethyl] | bromo-2-(2- | 7.59 (m, 3H), 7.30 (s, 1H), 4.30- |
| | morpholine-2-carboxylic acid | bromoethoxy) | 4.50 (m, 3H), 3.98-4.10 (m, 1H), |
| | 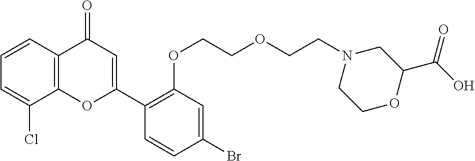 [SMILES: O=C(O)C1CN(CCOCCOc2cc(Br)ccc2-c2cc(=O)c3cccc(Cl)c3o2)CCO1] | ethane AMINE: methyl morpholine-2- carboxylate hydrochloride | 3.81-3.96 (m, 4H), 3.64-3.79 (m, 3H), 3.28 (br d, J = 7.8 Hz, 2H), 2.88-3.22 ppm (m, 2H). MS obsd. (ESI+) [(M + H)+]: 552.1. |
| 050 | (3R)-1-[2-[2-[2-(8-chloro-4- | CORE: Int-9 | ¹H NMR (DMSO-d₆, 400 MHz) |
| | oxo-chromen-2-yl)-5- | LINKER: 1- | δ ppm 7.94-8.03 (m, 2H), 7.76- |
| | methoxy-4-methyl- | bromo-2-(2- | 7.87 (m, 1H), 7.43-7.53 (m, 1H), |
| | phenoxy]ethoxy]ethyl] | bromoethoxy) | 7.27-7.33 (m, 1H), 6.79-6.87 (m, |
| | pyrrolidine-3-carboxylic acid | ethane | 1H), 4.37-4.42 (m, 2H), 3.85- |
| | 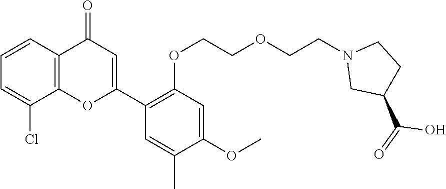 [SMILES: COc1cc(OCCOCCN2CC[C@@H](C(=O)O)C2)c(-c2cc(=O)c3cccc(Cl)c3o2)cc1C] | AMINE: methyl (3R)- pyrrolidine-3- carboxylate hydrochloride | 3.96 (m, 5H), 3.57-3.87 (m, 5H), 3.04-3.40 (m, 3H), 2.18-2.40 (m, 6H). MS obsd. (ESI⁺) [(M + H)⁺]: 502.1. |
| 051 | (3S)-1-[2-[2-[2-(8-chloro-4- | CORE: Int-9 | ¹H NMR (DMSO-d₆, 400 MHz) |
| | oxo-chromen-2-yl)-5- | LINKER: 1- | δ ppm 7.94-8.03 (m, 2H), 7.82 |
| | methoxy-4-methyl- | bromo-2-(2- | (s, 1H), 7.48 (s, 1H), 7.29 (s, |
| | phenoxy]ethoxy]ethyl] | bromoethoxy) | 1H), 6.83 (s, 1H), 4.33-4.44 (m, |
| | pyrrolidine-3-carboxylic acid | ethane | 2H), 3.6-3.93 (m, 10H), 3.06- |
| | 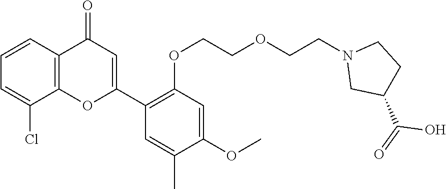 [SMILES: COc1cc(OCCOCCN2CC[C@H](C(=O)O)C2)c(-c2cc(=O)c3cccc(Cl)c3o2)cc1C] | AMINE: methyl (3S)- pyrrolidine-3- carboxylate hydrochloride | 3.39 (m, 3H), 1.95-2.28 (m, 6H). MS obsd. (ESI⁺) [(M + H)⁺]: 502.1. |
| 052 | 4-[2-[5-bromo-2-(8-chloro-4- | CORE: Int-4 | ¹H NMR (DMSO-d₆, 400 MHz) |
| | oxo-chromen-2- | LINKER: 1,2- | δ ppm 8.02 (ddd, J = 9.6, 8.0, |
| | yl)phenoxy]ethyl]morpholine- | dibromoethane | 1.5 Hz, 2H), 7.90-7.98 (m, 1H), |
| | 3-carboxylic acid | AMINE: | 7.60 (d, J = 1.7 Hz, 1H), 7.44- |
| | 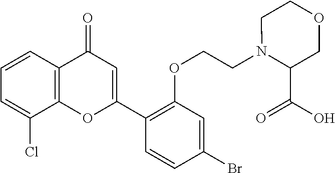 [SMILES: O=C(O)C1COCCN1CCOc1cc(Br)ccc1-c1cc(=O)c2cccc(Cl)c2o1] | methyl morpholine-3- carboxylate hydrochloride | 7.55 (m, 2H), 7.14 (s, 1H), 4.60- 4.68 (m, 2H), 4.47-4.57 (m, 3H), 4.06 (dd, J = 12.5, 3.8 Hz, 1H), 3.76-3.92 (m, 2H), 3.59-3.70 (m, 1H), 3.26-3.31 (m, 1H), 3.06- 3.17 (m, 1H). MS obsd. (ESI⁺) [(M + H)⁺]: 508.1. |
| 053 | 3-[2-[2-(8-chloro-4-oxo- | CORE: Int-2 | ¹H NMR (DMSO-d₆, 400 MHz) |
| | chromen-2-yl)-5-methyl- | LINKER: 1,2- | δ ppm 7.99-8.06 (m, 2H), 7.80- |
| | phenoxy]ethylamino] | dibromoethane | 7.89 (m, 1H), 7.44-7.55 (m, 1H), |
| | cyclobutanecarboxylic acid | AMINE: | 7.12-7.18 (m, 1H), 6.99-7.11 (m, |
| | 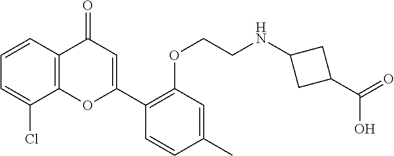 [SMILES: Cc1ccc(-c2cc(=O)c3cccc(Cl)c3o2)c(OCCNC2CC(C(=O)O)C2)c1] | methyl 3- amino- cyclobutane- 1-carboxylate | 2H), 4.34-4.43 (m, 2H), 4.04- 4.15 (m, 1H), 3.86-3.94 (m, 1H), 3.65-3.77 (m, 1H), 3.15-3.19 (m, 2H), 2.98-3.10 (m, 1H), 2.73- 2.88 (m, 1H), 2.42 (s, 3H), 2.21- 2.31 (m, 1H). MS obsd. (ESI⁺) [(M + H)⁺]: 428.1 |
| 054 | 1-[2-[2-(8-chloro-4-oxo- | CORE: Int-2 | ¹H NMR (DMSO-d₆, 400 MHz) |
| | chromen-2-yl)-5-methyl- | LINKER: 1,2- | δ ppm 7.96-8.01 (m, 2H), 7.84- |
| | phenoxy]ethyl]azetidine-3- | dibromoethane | 7.89 (m, 1H), 7.45-7.51 (m, 1H), |
| | carboxylic acid | AMINE: | 7.15-7.19 (m, 1H), 7.07-7.12 (m, |
| | 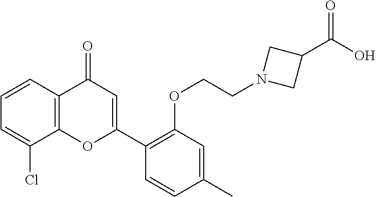 [SMILES: Cc1ccc(-c2cc(=O)c3cccc(Cl)c3o2)c(OCCN2CC(C(=O)O)C2)c1] | methyl azetidine-3- carboxylate hydrochloride | 1H), 6.99-7.04 (m, 1H), 4.07- 4.13 (m, 2H), 3.41-3.46 (m, 2H), 3.19-3.24 (m, 1H), 3.11-3.18 (m, 2H), 2.78-2.84 (m, 2H), 2.42 (s, 3H). MS obsd. (ESI⁺) [(M + H)⁺]: 414.1. |
| 055 | cis-4-[2-[5-bromo-2-(8- | CORE: Int-4 | ¹H NMR (DMSO-d₆, 400 MHz) |
| | chloro-4-oxo-chromen-2- | LINKER: 1,2- | δ ppm 7.90-7.96 (m, 2H), 7.80 |
| | yl)phenoxy]ethylamino]cyclo- | dibromoethane | (d, J = 8.4 Hz, 1H), 7.40-7.48 |
| | hexanecarboxylic acid | AMINE: | (m, 2H), 7.35 (dd, J = 8.3, 1.7 |
| | 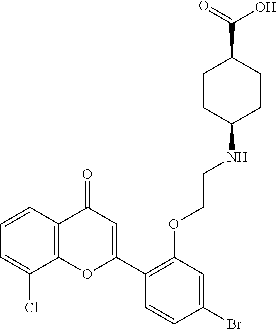 [SMILES: O=c1cc(-c2ccc(Br)cc2OCCN[C@H]2CC[C@@H](C(=O)O)CC2)oc2c(Cl)cccc12] | methyl cis-4- amino- cyclohexane- 1-carboxylate hydrochloride | Hz, 1H), 7.05 (s, 1H), 4.14-4.20 (m, 2H), 2.88 (br t, J = 5.4 Hz, 2H), 2.52-2.57 (m, 2H), 2.20- 2.25 (m, 1H), 1.65-1.78 (m, 2H), 1.42-1.53 (m, 2H), 1.23-1.41 (m, 3H). MS obsd. (ESI⁺) [(M + H)⁺]: 520.1. |
| 056 | trans-4-[2-[5-bromo-2-(8- | CORE: Int-4 | ¹H NMR (DMSO-d₆, 400 MHz) |
| | chloro-4-oxo-chromen-2- | LINKER: 1,2- | δ ppm 7.94 (d, J = 7.9 Hz, 2H), |
| | yl)phenoxy]ethylamino]cyclo- | dibromoethane | 7.81 (d, J = 8.4 Hz, 1H), 7.29- |
| | hexanecarboxylic acid | AMINE: | 7.49 (m, 3H), 7.13 (s, 1H), 4.16 |
| | 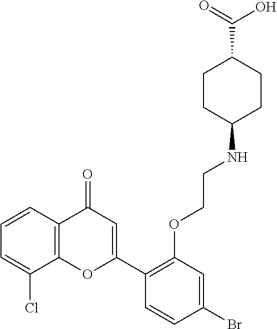 [SMILES: O=c1cc(-c2ccc(Br)cc2OCCN[C@H]2CC[C@H](C(=O)O)CC2)oc2c(Cl)cccc12] | methyl trans-4- amino- cyclohexane- 1-carboxylate hydrochloride | (br t, J = 5.1 Hz, 2H), 2.92 (br t, J = 4.8 Hz, 2H), 2.30-2.37 (m, 1H), 1.95-2.08 (m, 1H), 1.72- 1.87 (m, 4H), 1.11-1.27 (m, 2H), 0.81-1.01 (m, 2H). MS obsd. (ESI⁺) [(M + H)⁺]: 520.1. |
| 057 | 2-[2-[2-(8-chloro-4-oxo- | CORE: Int-1 | ¹H NMR (DMSO-d₆, 400 MHz) |
| | chromen-2-yl)-5- | LINKER: 1,2- | δ ppm 8.11 (d, J = 8.1 Hz, 1H), |
| | (trifluoromethyl)phenoxy] | dibromoethane | 8.03 (d, J = 7.9 Hz, 2H), 7.59- |
| | ethylamino]propanoic acid | AMINE: ethyl | 7.65 (m, 2H), 7.53 (t, J = 7.9 Hz, |
| | 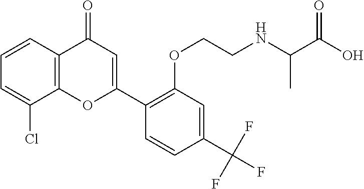 [SMILES: CC(NCCOc1cc(C(F)(F)F)ccc1-c1cc(=O)c2cccc(Cl)c2o1)C(=O)O] | 2- amino- propanoate | 1H), 7.13 (s, 1H), 4.49 (br t, J = 4.6 Hz, 2H), 2.07 (m, 2H), 1.49 (d, J = 6.7 Hz, 1H), 1.32 (br d, J = 7.1 Hz, 3H). MS obsd. (ESI⁺) [(M + H)⁺]: 456.3. |
| 058 | 2-[2-[2-(8-chloro-4-oxo- | CORE: Int-2 | ¹H NMR (DMSO-d₆, 400 MHz) |
| | chromen-2-yl)-5-methyl- | LINKER: 1,2- | δ ppm 7.98-8.03 (m, 2H), 7.84- |
| | phenoxy]ethylamino]-2- | dibromoethane | 7.88 (m, 1H), 7.47-7.52 (m, 1H), |
| | methyl-propanoic acid | AMINE: | 7.13-7.16 (m, 1H), 7.06-7.11 (m, |
| | 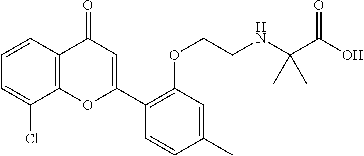 [SMILES: Cc1ccc(-c2cc(=O)c3cccc(Cl)c3o2)c(OCCNC(C)(C)C(=O)O)c1] | methyl 2-amino- 2-methyl- propanoate | 2H), 4.37-4.43 (m, 2H), 2.99- 3.21 (m, 2H), 2.41 (s, 3H), 1.47 (s, 6H). MS obsd. (ESI⁺) [(M + H)⁺]: 416.1. |
| 059 | 3-[3-[2-(8-chloro-4-oxo- | CORE: Int-2 | ¹H NMR (DMSO-d₆, 400 MHz) |
| | chromen-2-yl)-5-methyl- | LINKER: 1,3- | δ ppm 7.98-8.03 (m, 2H), 7.84- |
| | phenyl]propylamino] | dibromoethane | 7.88 (m, 1H), 7.47-7.52 (m, 1H), |
| | cyclobutanecarboxylic | AMINE: | 7.13-7.16 (m, 1H), 7.06-7.11 (m, |
| | acid | methyl 3- | 2H), 4.37-4.43 (m, 2H), 2.99- |
| | 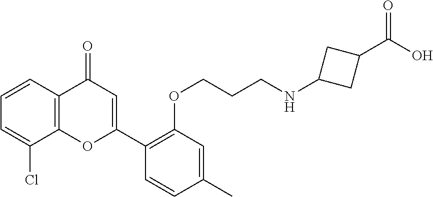 | aminocyclo- butane-1- carboxylate | 3.21 (m, 2H), 2.41 (s, 3H), 1.47 (s, 6H). MS obsd. (ESI⁺) [(M + H)⁺]: 416.1. |
| 060 | cis-4-[3-[5-bromo-2-(8- | CORE: Int-4 | ¹H NMR (DMSO-d₆, 400 MHz) |
| | chloro-4-oxo-chromen-2- | LINKER: 1,3- | δ ppm 7.97-8.04 (m, 2H), 7.83- |
| | yl)phenoxy]propylamino] | dibromopropane | 7.89 (m, 1H), 7.41-7.55 (m, 3H), |
| | tetrahydropyran-2-carboxylic | AMINE: | 6.99 (s, 1H), 4.27-4.36 (m, 2H), |
| | acid | methyl cis-3- | 3.86-4.01 (m, 2H), 3.56-3.81 (m, |
| | 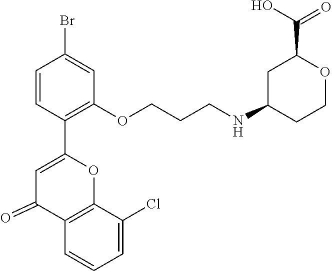 [SMILES: O=C(O)[C@@H]1C[C@H](NCCCOc2cc(Br)ccc2-c2cc(=O)c3cccc(Cl)c3o2)CCO1] | aminocyclo- hexane-1- carboxylate hydrochloride | 1H), 3.38-3.43 (m, 1H), 2.95- 3.12 (m, 2H), 2.08-2.30 (m, 3H), 1.71-1.93 (m, 1H), 1.36-1.57 (m, 2H). MS obsd. (ESI⁺) [(M + H)⁺]: 536.1. |
| 061 | 1-[2-[5-chloro-2-(8-chloro-4- | CORE: Int-5 | ¹H NMR (DMSO-d₆, 400 MHz) |
| | oxo-chromen-2-yl)-4-methyl- | LINKER: 1,2- | δ ppm 7.97-8.05 (m, 2H), 7.80- |
| | phenoxy]ethyl]azetidine-3- | dibromoethane | 7.88 (m, 1H), 7.47-7.56 (m, 1H), |
| | carboxylic acid | AMINE: | 7.39-7.46 (m, 1H), 6.88-6.96 (m, |
| | 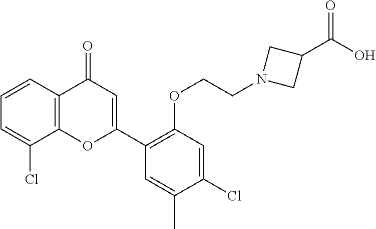 [SMILES: Cc1cc(-c2cc(=O)c3cccc(Cl)c3o2)c(OCCN2CC(C(=O)O)C2)cc1Cl] | methyl azetidine-3- carboxylate hydrochloride | 1H), 4.36-4.46 (m, 2H), 4.15- 4.30 (m, 4H), 3.62-3.70 (m, 2H), 2.34-2.41 (m, 4H). MS obsd. (ESI⁺) [(M + H)⁺]: 448.1. |

### Example 062: 3-[3-[2-(8-chloro-4-oxo-chromen-2-yl)-5-(trifluoromethyl)phenoxy]propylamino]butanoic acid

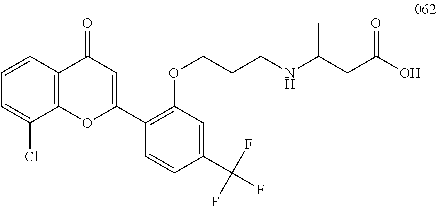 [SMILES: CC(CC(=O)O)NCCCOc1cc(C(F)(F)F)ccc1-c1cc(=O)c2cccc(Cl)c2o1]

### Step 1: Preparation of 2-[2-(3-bromopropoxy)-4-(trifluoromethyl)phenyl]-8-chloro-chromen-4-one

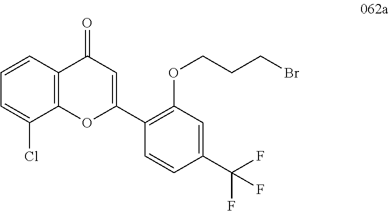 [SMILES: O=c1cc(-c2ccc(C(F)(F)F)cc2OCCCBr)oc2c(Cl)cccc12]

To a solution of 8-chloro-2-[2-hydroxy-4-(trifluoromethyl)phenyl]chromen-4-one (500 mg, 1.47 mmol), 1,3-dibromopropane (1.19 g, 5.87 mmol) in DMF was added K₂CO₃ (203 mg, 1.47 mmol). The reaction mixture was stirred at 50° C. overnight. After the reaction was completed, the reaction mixture was diluted and partitioned between EtOAc (10 mL) and water (30 mL). The organic layer was separated out and the aqueous phase was extracted with EtOAc (30 mL) twice. The combined organic layer was concentrated in vacuo to give the crude product. The crude product was purified by ISCO (eluting with EtOAc:PE=0 to 20%) to give 2-[2-(3-bromopropoxy)-4-(trifluoromethyl)phenyl]-8-chloro-chromen-4-one (680 mg, yield: 100%) as a yellow solid. MS obsd. (ESI⁺) [(M+H)]⁺: 463.0.

### Step 2: Preparation of tert-butyl 3-[3-[2-(8-chloro-4-oxo-chromen-2-yl)-5-(trifluoromethyl)phenoxy]propylamino]butanoate

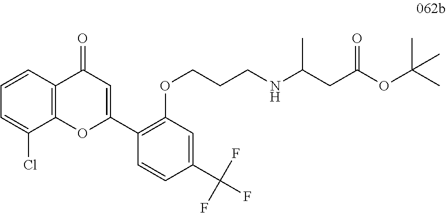 [SMILES: CC(CC(=O)OC(C)(C)C)NCCCOc1cc(C(F)(F)F)ccc1-c1cc(=O)c2cccc(Cl)c2o1]

To a solution of 2-[2-(3-bromopropoxy)-4-(trifluoromethyl)phenyl]-8-chloro-chromen-4-one (94 mg, 204 μmol) and tert-butyl 3-aminobutanoate (64.8 mg, 407 μmol) in DMF was added K₂CO₃ (141 mg, 1.02 mmol). The mixture was stirred at 90° C. overnight. After the reaction was completed, the reaction mixture was diluted with EtOAc and partitioned between EtOAc (30 mL) and water (20 mL). The organic layer was separated out and the aqueous phase was extracted with EtOAc (30 mL) twice. The combined organic layer was concentrated in vacuo to give tert-butyl 3-[3-[2-(8-chloro-4-oxo-chromen-2-yl)-5-(trifluoromethyl)phenoxy]propylamino]butanoate (110 mg, yield: 100%) as a yellow solid. (ESI⁺) [(M+H)]⁺: 540.3.

### Step 3: Preparation of 3-[3-[2-(8-chloro-4-oxo-chromen-2-yl)-5-(trifluoromethyl)phenoxy]propylamino]butanoic acid

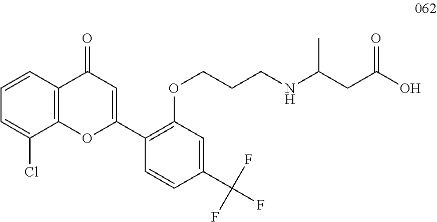 [SMILES: CC(CC(=O)O)NCCCOc1cc(C(F)(F)F)ccc1-c1cc(=O)c2cccc(Cl)c2o1]

To a flask charged with tert-butyl 3-[3-[2-(8-chloro-4-oxo-chromen-2-yl)-5-(trifluoromethyl)phenoxy]propylamino]butanoate (110 mg, 204 μmol) dissolved in DCM (2.04 mL) was added TFA (0.1 mL, 204 μmol). The reaction was stirred at room temperature for 2 hours and then concentrated in vacuo to give yellow oil, which was purified by prep-HPLC to give 3-[3-[2-(8-chloro-4-oxo-chromen-2-yl)-5-(trifluoromethyl)phenoxy]propylamino]butanoic acid (6.5 mg, yield: 6.26%) as a white powder. (ESI⁺) [(M+H)]⁺: 484.2.

Example 062: ¹H NMR (DMSO-d₆, 400 MHz) δ ppm 8.12 (m, 1H), 7.94-8.06 (m, 2H), 7.46-7.69 (m, 3H), 6.95-7.13 (m, 1H), 4.36 (m, 2H), 4.22-4.28 (m, 2H), 2.07 (m, 2H), 1.24 (m, 3H), 1.08 (m, 2H), 0.80-0.92 (m, 1H).

### Example 063

### 2-[3-[2-(8-chloro-4-oxo-chromen-2-yl)-5-(trifluoromethyl)phenoxy]propylamino]-3-cyclohexyl-propanoic acid

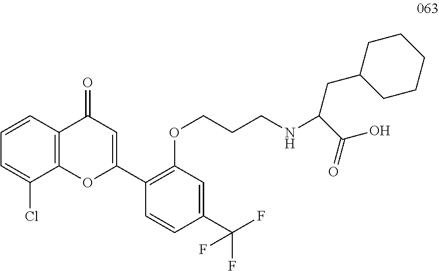 [SMILES: O=C(O)C(CC1CCCCC1)NCCCOc1cc(C(F)(F)F)ccc1-c1cc(=O)c2cccc(Cl)c2o1]

063 was prepared in analogy to the procedure described for the preparation of compound 062 by using tert-butyl 2-amino-3-cyclohexyl-propanoate as the starting material instead of tert-butyl 3-aminobutanoate in Step 2. MS obsd. (ESI⁺) [(M+H)]⁺: 552.6.

Example 063: ¹H NMR (DMSO-d₆, 400 MHz) δ ppm 8.12 (d, J=8.8 Hz, 1H), 7.96-8.07 (m, 2H), 7.41-7.68 (m, 3H), 7.03 (s, 1H), 4.29-4.47 (m, 2H), 2.98-3.10 (m, 2H), 2.12-2.27 (m, 2H), 2.07 (s, 1H), 1.69-1.79 (m, 2H), 1.49-1.65 (m, 4H), 1.40 (m, 1H), 1.04-1.19 (m, 4H), 0.73-0.92 (m, 2H).

### Example 064: (3R)-1-[2-[5-bromo-2-(8-chloro-4-oxo-chromen-2-yl)phenoxy]ethyl]-N-methylsulfonyl-pyrrolidine-3-carboxamide

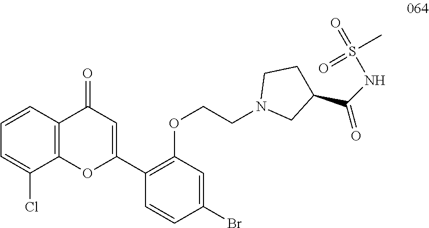 [SMILES: CS(=O)(=O)NC(=O)[C@@H]1CCN(CCOc2cc(Br)ccc2-c2cc(=O)c3cccc(Cl)c3o2)C1]

### Step 1: Preparation of 2-[4-bromo-2-(2-bromoethoxy)phenyl]-8-chloro-chromen-4-one

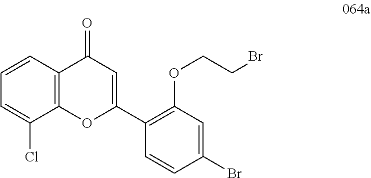 [SMILES: O=c1cc(-c2ccc(Br)cc2OCCBr)oc2c(Cl)cccc12]

To a mixture of 2-(4-bromo-2-hydroxy-phenyl)-8-chloro-chromen-4-one (500 mg, 1.42 mmol, as the “CORE” in Table 2) and 1,2-dibromoethane (1.07 g, 5.69 mmol, as the “LINKER” in Table 2) in DMF (15 mL) was added K₂CO₃ (197 mg, 1.42 mmol). The reaction was stirred at 50° C. overnight. After completion, the reaction mixture was quenched with 4N HCl (20 mL) and extracted with EtOAc (30 mL) three times. The combined organic layer was washed with brine, dried over anhydrous Na₂SO₄ and concentrated in vacuo to give 2-[4-bromo-2-(2-bromoethoxy)phenyl]-8-chloro-chromen-4-one (450 mg, yield: 69%) as a yellow solid. MS obsd. (ESI⁺) [(M+H)⁺]: 459.0.

### Step 2: Preparation of methyl (3R)-1-[2-[5-bromo-2-(8-chloro-4-oxo-chromen-2-yl)phenoxy]ethyl]pyrrolidine-3-carboxylate

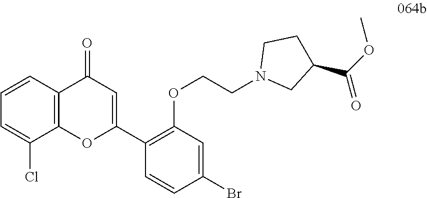 [SMILES: COC(=O)[C@@H]1CCN(CCOc2cc(Br)ccc2-c2cc(=O)c3cccc(Cl)c3o2)C1]

A mixture of 2-[4-bromo-2-(2-bromoethoxy)phenyl]-8-chloro-chromen-4-one (250 mg, 545 μmol), methyl (3R)-pyrrolidine-3-carboxylate hydrochloride (200 mg, 5.49 mmol, as the “CYC” in Table 2) and sodium bicarbonate (275 mg, 3.27 mmol) in ethanol (10 mL) was stirred at 90° C. overnight. After completion, the reaction mixture was diluted with water (20 mL) and extracted with EtOAc (30 mL) three times. The combined organic layer was washed with brine, dried over anhydrous Na₂SO₄ and concentrated in vacuo to give methyl (3R)-1-[2-[5-bromo-2-(8-chloro-4-oxo-chromen-2-yl)phenoxy]ethyl]pyrrolidine-3-carboxylate (200 mg, yield: 72.4%) as a yellow oil. MS obsd. (ESI⁺) [(M+H)⁺]: 508.1.

### Step 3: Preparation of (3R)-1-[2-[5-bromo-2-(8-chloro-4-oxo-chromen-2-yl)phenoxy]ethyl]pyrrolidine-3-carboxylic acid

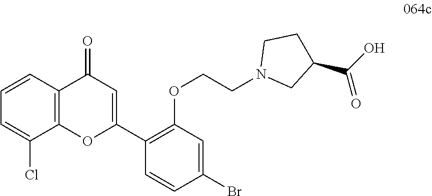 [SMILES: O=C(O)[C@@H]1CCN(CCOc2cc(Br)ccc2-c2cc(=O)c3cccc(Cl)c3o2)C1]

To a mixture of methyl (3R)-1-[2-[5-bromo-2-(8-chloro-4-oxo-chromen-2-yl)phenoxy]ethyl]pyrrolidine-3-carboxylate (200 mg, 395 μmol) in THF (5 mL) and water (5 mL) was added 4M HCl (5 mL, 20 mmol) at room temperature. The reaction mixture was then stirred at 50° C. for 2 hours. After the reaction was completed, the reaction mixture was concentrated in vacuo to give (3R)-1-[2-[5-bromo-2-(8-chloro-4-oxo-chromen-2-yl)phenoxy]ethyl]pyrrolidine-3-carboxylic acid (190 mg, yield: 97.7%) as a white foam .MS obsd. (ESI⁺) [(M+H)⁺]: 492.0.

### Step 4: Preparation of (3R)-1-[2-[5-bromo-2-(8-chloro-4-oxo-chromen-2-yl)phenoxy]ethyl]-N-methylsulfonyl-pyrrolidine-3-carboxamide

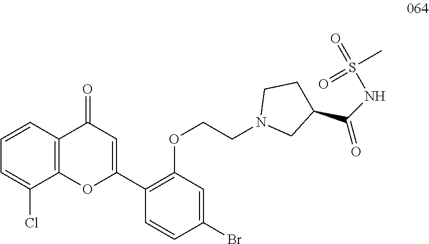 [SMILES: CS(=O)(=O)NC(=O)[C@@H]1CCN(CCOc2cc(Br)ccc2-c2cc(=O)c3cccc(Cl)c3o2)C1]

To a mixture of (3R)-1-[2-[5-bromo-2-(8-chloro-4-oxo-chromen-2-yl)phenoxy]ethyl]-N-methylsulfonyl-pyrrolidine-3-carboxamide (120 mg, 237 μmol), methanesulfonamide (45 mg, 474 μmol, as the “SA” in Table 2), DIPEA (306 mg, 414 μl, 2.37 mmol), DMAP (28.9 mg, 237 μmol) in DCM (5 mL) was added HATU (90.8 mg, 474 μmol). The reaction mixture was then stirred at room temperature for 12 hours. After completion, the reaction mixture was concentrated in vacuo to give the crude product. The crude product was purified by prep-HPLC to give (3R)-1-[2-[5-bromo-2-(8-chloro-4-oxo-chromen-2-yl)phenoxy]ethyl]-N-methylsulfonyl-pyrrolidine-3-carboxamide (13 mg, yield: 9.4%) as a white foam. MS obsd. (ESI⁺) [(M+H)⁺]: 569.1.

Example 064: ¹H NMR (DMSO-d₆, 400 MHz) δ ppm 7.96-8.06 (m, 2H), 7.82-7.90 (m, 1H), 7.55-7.61 (m, 1H), 7.42-7.54 (m, 2H), 7.08-7.17 (m, 1H), 4.34-4.46 (m, 2H), 3.17 (m, 4H), 2.88-3.11 (m, 5H), 1.87-2.15 (m, 3H).

The following compounds 065 to 071 were prepared in analogy to the procedure described for the preparation of Example 064, replacing 2-(4-bromo-2-hydroxy-phenyl)-8-chloro-chromen-4-one with “CORE”, 1,2-dibromoethane with “LINKER” in Step 1, methyl (3R)-pyrrolidine-3-carboxylate hydrochloride with “CYC” in step 2, replacing methanesulfonamide with “SA”, by the reagent indicated in Table 2.

TABLE 2
Compounds synthesis and characterization

| Example | Compounds Name and | CORE, LINKER, | |
|---|---|---|---|
| No. | Structure | CYC and SA | ¹H NMR and (ESI⁺) |
| 065 | (3R)-1-[3-[2-(8-chloro-4-oxo- chromen-2-yl)-5- (trifluoromethyl)phenoxy] propyl]-N-methylsulfonyl- pyrrolidine-3-carboxamide 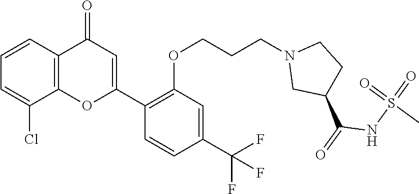 [SMILES: CS(=O)(=O)NC(=O)[C@@H]1CCN(CCCOc2cc(C(F)(F)F)ccc2-c2cc(=O)c3cccc(Cl)c3o2)C1] | CORE: Int-1 LINKER: 1,3- dibromopropane CYC: methyl (3R)- pyrrolidine-3- carboxylatc SA: methanesulfonamide | ¹H NMR (DMSO-d₆, 400 MHz) δ ppm 8.09-8.17 (m, 1H), 7.99-8.08 (m, 2H), 7.45-7.65 (m, 3H), 7.02- 7.08 (m, 1H), 4.28-4.40 (m, 2H), 3.72-3.88 (m, 1H), 3.28-3.45 (m, 8H), 2.35- 2.46 (m, 1H), 2.11-2.25 (m, 3H), 1.85-2.04 (m, 1H). MS obsd. (ESI⁺) [(M + H)⁺]: 573.1. |
| 066 | (3S)-1-[3-[2-(8-chloro-4-oxo- chromen-2-yl)-5- (trifluoromethyl)phenoxy] propyl]-N-methylsulfonyl- pyrrolidine-3-carboxamide 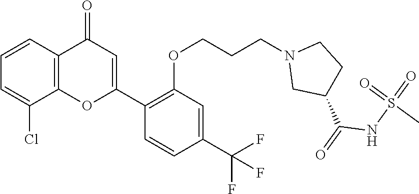 [SMILES: CS(=O)(=O)NC(=O)[C@H]1CCN(CCCOc2cc(C(F)(F)F)ccc2-c2cc(=O)c3cccc(Cl)c3o2)C1] | CORE: Int-1 LINKER: 1,3- dibromopropane CYC: methyl (3S)- pyrrolidine-3- carboxylate SA: methanesulfonamide | ¹H NMR (DMSO-d₆, 400 MHz) δ ppm 8.09-8.17 (m, 1H), 7.98-8.09 (m, 2H), 7.49-7.67 (m, 3H), 7.01- 7.08 (m, 1H), 4.29-4.40 (m, 2H), 3.60-3.85 (m, 2H), 3.20-3.37 (m, 8H), 3.02- 3.14 (m, 1H), 2.10-2.26 (m, 3H). MS obsd. (ESI⁺) [(M + H)⁺]: 573.1. |
| 067 | (3R)-1-[3-[2-(8-chloro-4-oxo- chromen-2-yl)-5- (trifluoromethyl)phenoxy] propyl]-N-cyclopropylsulfonyl- pyrrolidine-3-carboxamide 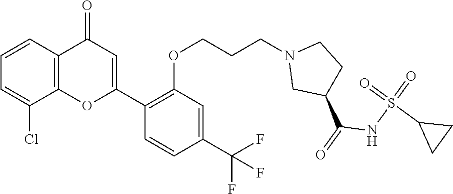 [SMILES: O=C(NS(=O)(=O)C1CC1)[C@@H]1CCN(CCCOc2cc(C(F)(F)F)ccc2-c2cc(=O)c3cccc(Cl)c3o2)C1] | CORE: Int-1 LINKER: 1,3- dibromopropane CYC: methyl (3R)- pyrrolidine-3- carboxylate SA: cyclopropanesulfonamide | ¹H NMR (DMSO-d₆, 400 MHz) δ ppm 8.25 (s, 1H), 8.12 (d, J = 8.4 Hz, 1H), 8.03 (d, J = 7.9 Hz, 2H), 7.48-7.58 (m, 3H), 7.08 (s, 1H), 4.27-4.34 (m, 2H), 2.81-2.88 (m, 2H), 2.72-2.79 (m, 2H), 2.60- 2.72 (m, 6H), 1.77-2.04 (m, 6H). MS obsd. (ESI⁺) [(M + H)⁺]: 599.3 |
| 068 | 4-[2-[2-(8-chloro-4-oxo- chromen-2-yl)-5- (trifluoromethyl)phenoxy] ethyl]-N-methylsulfonyl- morpholine-2-carboxamide 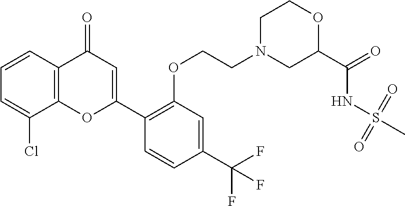 [SMILES: CS(=O)(=O)NC(=O)C1CN(CCOc2cc(C(F)(F)F)ccc2-c2cc(=O)c3cccc(Cl)c3o2)CCO1] | CORE: Int-1 LINKER: 1,2- dibromoethane CYC: methyl morpholine-2- carboxylate hydrochloride SA: methanesulfonamide | ¹H NMR (DMSO-d₆, 400 MHz) δ ppm 11.31- 11.81 (m, 1H), 8.16-8.24 (m, 1H), 7.95-8.06 (m, 2H), 7.45-7.66 (m, 4H), 4.41 (br t, J = 5.2 Hz, 2H), 4.13 (dd, J = 9.8, 2.6 Hz, 1H), 3.86 (br d, J = 11.1 Hz, 1H), 3.63-3.72 (m, 1H), 3.16 (s, 3H), 3.05 (br d, J = 11.0 Hz, 1H), 2.84 (br t, J = 5.2 Hz, 2H), 2.77 (br d, J = 11.5 Hz, 1H), 2.07-2.32 (m, 2H). MS obsd. (ESI⁺) [(M + H)⁺]: 575.2. |
| 069 | (3S)-1-[2-[5-bromo-2-(8- chloro-4-oxo-chromen-2- yl)phenoxylethyl]-N- methylsulfonyl-pyrrolidine-3- carboxamide 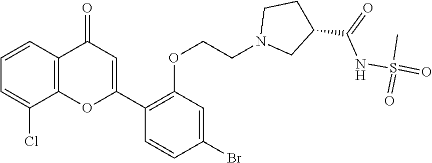 [SMILES: CS(=O)(=O)NC(=O)[C@H]1CCN(CCOc2cc(Br)ccc2-c2cc(=O)c3cccc(Cl)c3o2)C1] | CORE: Int-4 LINKER: 1,2- dibromoethane CYC: methyl (3S)- pyrrolidine-3- carboxylate SA: methanesulfonamide | ¹H NMR (DMSO-d₆, 400 MHz) δ ppm 8.12-8.15 (m, 1H), 7.95-8.04 (m, 2H), 7.80-7.91 (m, 1H), 7.41- 7.58 (m, 3H), 7.11-7.19 (m, 1H), 4.32-4.45 (m, 2H), 3.12-3.20 (m, 6H), 2.81- 3.08 (m, 3H), 1.88-2.12 (m, 3H). MS obsd. (ESI⁺) [(M + H)⁺]: 569.1. |
| 070 | (3R)-1-[2-[2-(8-chloro-4-oxo- chromen-2-yl)-5- (trifluoromethyl)phenoxy] ethyl]-N-methylsulfonyl- pyrrolidine-3-carboxamide 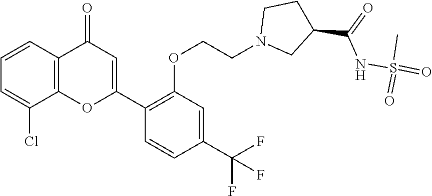 [SMILES: CS(=O)(=O)NC(=O)[C@@H]1CCN(CCOc2cc(C(F)(F)F)ccc2-c2cc(=O)c3cccc(Cl)c3o2)C1] | CORE: Int-1 LINKER: 1,2- dibromoethane CYC: methyl (3R)- pyrrolidine-3- carboxylate SA: methanesulfonamide | ¹H NMR (DMSO-d₆, 400 MHz) δ ppm 12.15 (m, 1H), 7.98-8.14 (m, 3H), 7.61-7.70 (m, 2H), 7.46- 7.59 (m, 1H), 6.99-7.05 (m, 1H), 4.51-4.65 (m, 3H), 3.55-3.90 (m, 4H), 3.13- 3.21 (m, 5H), 1.79-2.24 (m, 2H). MS obsd. (ESI⁺) [(M + H)⁺]: 559.1. |
| 071 | (3S)-1-[2-[2-(8-chloro-4-oxo- chromen-2-yl)-5- (trifluoromethyl)phenoxy] ethyl]-N-methylsulfonyl- pyrrolidine-3-carboxamide 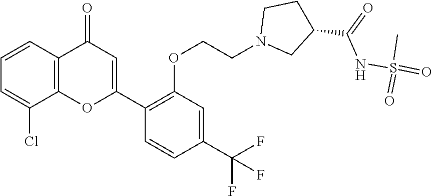 [SMILES: CS(=O)(=O)NC(=O)[C@H]1CCN(CCOc2cc(C(F)(F)F)ccc2-c2cc(=O)c3cccc(Cl)c3o2)C1] | CORE: Int-1 LINKER: 1,2- dibromoethane CYC: methyl (3S)- pyrrolidine-3- carboxylate SA: methanesulfonamide | ¹H NMR (DMSO-d₆, 400 MHz) δ ppm 8.08-8.17 (m, 2H), 7.97-8.06 (m, 2H), 7.57-7.65 (m, 2H), 7.46- 7.56 (m, 1H), 7.15-7.24 (m, 1H), 4.33-4.49 (m, 2H), 3.11-3.18 (m, 5H), 2.80- 3.07 (m, 5H), 1.87-2.11 (m, 2H). MS obsd. (ESI⁺) [(M + H)⁺]: 559.1. |

### Example 072

### 1-[2-[2-(8-chloro-4-oxo-chromen-2-yl)-5-(trifluoromethyl)phenoxy]ethyl]-N-cyclopropylsulfonyl-pyrrolidine-2-carboxamide

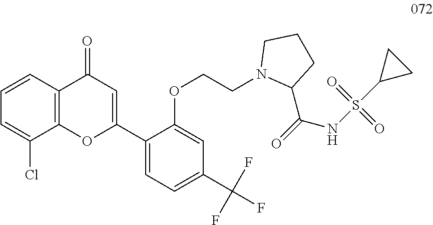 [SMILES: O=C(NS(=O)(=O)C1CC1)C1CCCN1CCOc1cc(C(F)(F)F)ccc1-c1cc(=O)c2cccc(Cl)c2o1]

### Step 1: Preparation of 2-[4-bromo-2-(2-bromoethoxy)phenyl]-8-chloro-chromen-4-one

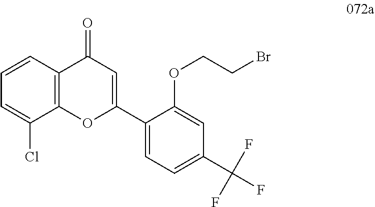 [SMILES: O=c1cc(-c2ccc(C(F)(F)F)cc2OCCBr)oc2c(Cl)cccc12]

To a solution of 8-chloro-2-[2-hydroxy-4-(trifluoromethyl)phenyl]chromen-4-one (680 mg, 2 mmol, as the “CORE” in Table 3) in DMF (20 mL) was added 1,2-dibromoethane (3.75 g, 19.96 mmol, as the “LINKER” in Table 3) and potassium carbonate (827 mg, 5.99 mmol). The reaction was heated at 80° C. and stirred for 5 hours. After the starting material was consumed, the reaction mixture was quenched with water (150 mL) and extracted with EtOAc (50 mL) three times. The combined organic layer was concentrated in vacuo to give 2-[2-(2-bromoethoxy)-4-(trifluoromethyl)phenyl]-8-chloro-chromen-4-one (800 mg, yield: 89.5%) as an off-white solid. MS obsd. (ESI⁺)[(M+H)⁺]: 446.9.

### Step 2: Preparation of tert-butyl 1-[2-[2-(8-chloro-4-oxo-chromen-2-yl)-5-(trifluoromethyl)phenoxy]ethyl]pyrrolidine-2-carboxylate

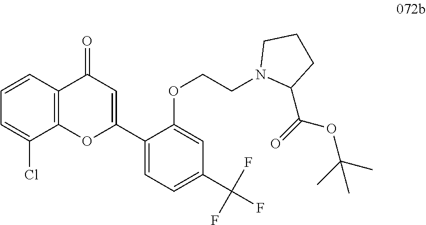 [SMILES: CC(C)(C)OC(=O)C1CCCN1CCOc1cc(C(F)(F)F)ccc1-c1cc(=O)c2cccc(Cl)c2o1]

To a solution of 2-[2-(2-bromoethoxy)-4-(trifluoromethyl)phenyl]-8-chloro-chromen-4-one (700 mg, 1.56 mmol) and tert-butyl pyrrolidine-2-carboxylate (348.12 mg, 2.03 mmol, as the “CYC” in Table 3) in DMF (7 mL) and ethanol (7 mL) were added NaHCO₃ (394.12 mg, 4.69 mmol) and potassium iodide (26 mg, 0.160 mmol). The reaction mixture was stirred at 90° C. for 5 hours. After the reaction was completed, the reaction mixture was concentrated in vacuo to give tert-butyl 1-[2-[2-(8-chloro-4-oxo-chromen-2-yl)-5-(trifluoromethyl)phenoxy]ethyl]pyrrolidine-2-carboxylate (0.5 g, yield: 59.4%) as a light yellow solid. MS obsd. (ESI⁺)[(M+H)⁺]: 538.2.

### Step 3: Preparation of 1-[2-[2-(8-chloro-4-oxo-chromen-2-yl)-5-(trifluoromethyl)phenoxy]ethyl]pyrrolidine-2-carboxylic acid

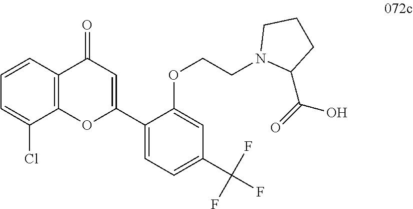 [SMILES: O=C(O)C1CCCN1CCOc1cc(C(F)(F)F)ccc1-c1cc(=O)c2cccc(Cl)c2o1]

To a solution of tert-butyl 1-[2-[2-(8-chloro-4-oxo-chromen-2-yl)-5-(trifluoromethyl)phenoxy]ethyl]pyrrolidine-2-carboxylate (460 mg, 0.860 mmol) in DCM (4 mL) was added trifluoroacetic acid (2 mL, 26 mmol). The reaction was stirred at room temperature for 5 hours. After completion, the reaction mixture was concentrated in vacuo to give 1-[2-[2-(8-chloro-4-oxo-chromen-2-yl)-5-(trifluoromethyl)phenoxy]ethyl]pyrrolidine-2-carboxylic acid (410 mg, yield: 99.5%) as a light yellow solid. MS obsd. (ESI⁺)[(M+H)⁺]: 482.1.

### Step 4: Preparation of 1-[2-[2-(8-chloro-4-oxo-chromen-2-yl)-5-(trifluoromethyl)phenoxy]ethyl]-N-cyclopropylsulfonyl-pyrrolidine-2-carboxamide

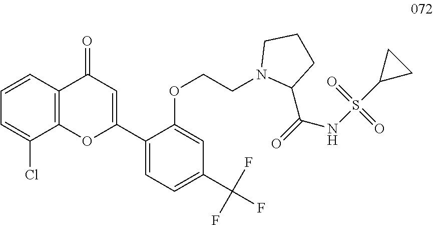 [SMILES: O=C(NS(=O)(=O)C1CC1)C1CCCN1CCOc1cc(C(F)(F)F)ccc1-c1cc(=O)c2cccc(Cl)c2o1]

To a solution 1 of cyclopropanesulfonamide (98 mg, 0.810 mmol, as the “SA” in Table 3) in THF (4 mL) was added sodium hydride (37.36 mg, 0.930 mmol) dropwise at 0° C. and kept stirred at 0° C. for 1 hour. To a solution 2 of 1-[2-[2-(8-chloro-4-oxo-chromen-2-yl)-5-(trifluoromethyl)phenoxy]ethyl]pyrrolidine-2-carboxylic acid (300 mg, 0.620 mmol) in THF (4 mL) were added HATU (473 mg, 1.25 mmol) and DIPEA (0.33 mL, 1.87 mmol), and the solution was stirred at room temperature for 1 hour. Then, the solution 1 was added to the solution 2 and the resulting residue was stirred at room temperature for 16 hours. After completion, the reaction mixture was concentrated in vacuo to give the crude product. The crude product was purified by prep-HPLC to give 1-[2-[2-(8-chloro-4-oxo-chromen-2-yl)-5-(trifluoromethyl)phenoxy]ethyl]-N-cyclopropylsulfonyl-pyrrolidine-2-carboxamide (75 mg, yield: 19.8%) as an off-white solid. MS obsd. (ESI⁺)[(M+H)⁺]: 585.1.

Example 072: ¹H NMR (DMSO-d₆, 400 MHz) δ ppm 7.97-8.14 (m, 3H), 7.47-7.65 (m, 3H), 7.07-7.16 (m, 1H), 4.44-4.57 (m, 2H), 3.06-3.65 (m, 3H), 2.71-2.97 (m, 3H), 2.15-2.31 (m, 1H), 1.66-1.91 (m, 3H), 0.74-1.00 (m, 4H).

The following compounds 073 and 074 were prepared in analogy to the procedure described for the preparation of Example 072, replacing 8-chloro-2-[2-hydroxy-4-(trifluoromethyl)phenyl]chromen-4-one with “CORE”, 1,2-dibromoethane with “LINKER” in Step 1, tert-butyl pyrrolidine-3-carboxylate with “CYC” in Step 2, replacing cyclopropanesulfonamide with “SA”, by the reagent indicated in Table 3.

TABLE 3
Compounds synthesis and characterization

| | | CORE, | |
|---|---|---|---|
| Example | Compounds Name and | LINKER, CYC | |
| No. | Structure | and SA | ¹H NMR and (ESI⁺) |
| 073 | 1-[2-[2-(8-chloro-4-oxo- chromen-2-yl)-5- (trifluoromethyl)phenoxy] ethyl]-N-methylsulfonyl- pyrrolidine-2- carboxamide 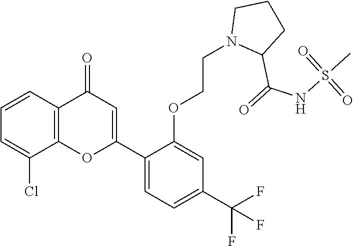 [SMILES: CS(=O)(=O)NC(=O)C1CCCN1CCOc1cc(C(F)(F)F)ccc1-c1cc(=O)c2cccc(Cl)c2o1] | CORE: Int-1 LINKER: 1,2- dibromoethane CYC: tert-butyl pyrrolidine-2- carboxylate SA: methanesulfonamide | ¹H NMR (DMSO-d₆, 400 MHz) δ ppm 8.07-8.12 (m, 1H), 7.99-8.05 (m, 2H), 7.58-7.64 (m, 2H), 7.49-7.56 (m, 1H), 7.06-7.12 (m, 1H), 4.47-4.57 (m, 2H), 3.72-3.81 (m, 1H), 3.37-3.54 (m, 3H), 2.88-2.98 (m, 1H), 2.84 (s, 3H), 2.13-2.26 (m, 1H), 1.64- 1.88 (m, 3H). MS obsd. (ESI⁺) [(M + H)⁺]: 559.1. |
| 074 | 1-[2-[5-bromo-2-(8-chloro-4- oxo-chromen-2- yl)phenoxy]ethyl]-N- methylsulfonyl-pyrrolidine-2- carboxamide 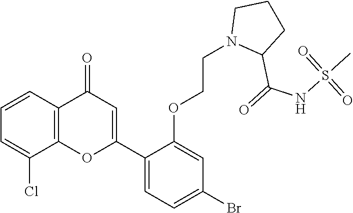 [SMILES: CS(=O)(=O)NC(=O)C1CCCN1CCOc1cc(Br)ccc1-c1cc(=O)c2cccc(Cl)c2o1] | CORE: Int-4 LINKER: 1,2- dibromoethane CYC: tert-butyl pyrrolidine-2- carboxylate SA: methanesulfonamide | ¹H NMR (DMSO-d₆, 400 MHz) δ ppm 7.98-8.04 (m, 2H), 7.81-7.87 (m, 1H), 7.54-7.58 (m, 1H), 7.43-7.54 (m, 2H), 7.02-7.07 (m, 1H), 4.43-4.50 (m, 2H), 3.71-3.80 (m, 1H), 3.41-3.53 (m, 2H), 2.87-2.99 (m, 1H), 2.84 (s, 3H), 2.12-2.25 (m, 1H), 1.66- 1.89 (m, 3H), 1.18-1.29 (m, 1H). MS obsd. (ESI⁺) [(M + H)⁺]: 569.1. |

### Example 075: 1-[2-[5-bromo-2-(8-chloro-4-oxo-chromen-2-yl)phenoxy]ethyl]pyrrolidin-2-one

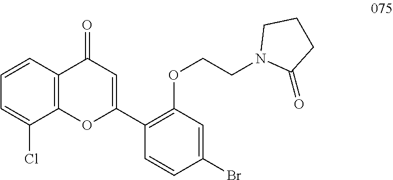 [SMILES: O=C1CCCN1CCOc1cc(Br)ccc1-c1cc(=O)c2cccc(Cl)c2o1]

### Step 1: Preparation of 1-(2-chloroethyl)pyrrolidin-2-one

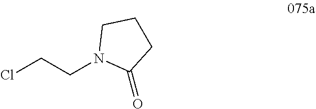

To a solution of 1-(2-hydroxyethyl)pyrrolidin-2-one (200 mg, 1.55 mmol) in DCM (15 mL) was added thionyl chloride (368 mg, 3.1 mmol) at room temperature and the reaction mixture was stirred at 60° C. for 3 hours. After the reaction was completed, the reaction mixture was washed with water and brine. The organic layer was separated out and dried over anhydrous MgSO₄, filtered and concentrated in vacuo to give 1-(2-chloroethyl)pyrrolidin-2-one (200 mg, yield: 65.6%) as brown oil.

### Step 2: Preparation of 1-[2-[5-bromo-2-(8-chloro-4-oxo-chromen-2-yl)phenoxy]ethyl]pyrrolidin-2-one

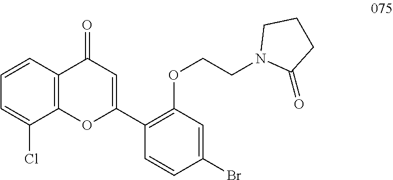 [SMILES: O=C1CCCN1CCOc1cc(Br)ccc1-c1cc(=O)c2cccc(Cl)c2o1]

To a solution of 2-(4-bromo-2-hydroxy-phenyl)-8-chloro-chromen-4-one (200.0 mg, 0.570 mmol) and K₂CO₃ (157.24 mg, 1.14 mmol) in DMF (5 mL) was added 1-(2-chloroethyl)pyrrolidin-2-one (84 mg, 0.570 mmol) and the reaction mixture was stirred at 80° C. for 16 hours. The reaction mixture was filtered and the filtrate was concentrated in vacuo. The residue was purified by prep-HPLC to give 1-[2-[5-bromo-2-(8-chloro-4-oxo-chromen-2-yl)phenoxy]ethyl]pyrrolidin-2-one (42 mg, 15.68% yield) as a yellow solid. MS obsd. (ESI⁺)[(M+H)⁺]: 462.1.

Example 075: ¹H NMR (DMSO-d₆, 400 MHz) δ ppm 8.04-7.95 (m, 2H), 7.86 (d, J=8.4 Hz, 1H), 7.56 (d, J=1.6 Hz, 1H), 7.50 (t, J=7.9 Hz, 1H), 7.43 (dd, J=1.7, 8.4 Hz, 1H), 6.97 (s, 1H), 4.35 (t, J=5.3 Hz, 2H), 3.59 (br t, J=5.3 Hz, 2H), 3.45-3.42 (m, 2H), 2.20 (t, J=8.1 Hz, 2H), 1.86 (m, J=7.5 Hz, 2H).

### Example 076: N-[1-[2-[5-bromo-2-(8-chloro-4-oxo-chromen-2-yl)phenoxy]ethyl]pyrrolidin-3-yl]-N-methyl-methanesulfonamide

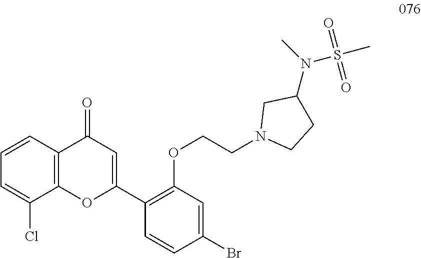 [SMILES: CN(C1CCN(CCOc2cc(Br)ccc2-c2cc(=O)c3cccc(Cl)c3o2)C1)S(C)(=O)=O]

### Step 1: Preparation of 2-[4-bromo-2-(2-bromoethoxy)phenyl]-8-chloro-chromen-4-one

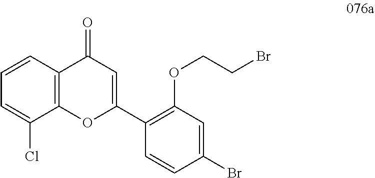 [SMILES: O=c1cc(-c2ccc(Br)cc2OCCBr)oc2c(Cl)cccc12]

To a solution of 2-(4-bromo-2-hydroxy-phenyl)-8-chloro-chromen-4-one (500 mg, 1.42 mmol, as the “CORE” in Table 4) and 1,2-dibromoethane (1.07 g, 5.69 mmol, as the “LINKER” in Table 4) in DMF was added K₂CO₃ (197 mg, 1.42 mmol). The reaction mixture was stirred at 50° C. overnight. After the reaction was completed, the reaction mixture was partitioned between EtOAc (10 mL) and water (30 mL). The organic layer was separated out and the aquatic phase was extracted with EtOAc (30 mL) twice. The combined organic layer was concentrated in vacuo and the residue was purified by ISCO (eluting with EtOAc:PE=0 to 20%) to give 2-[4-bromo-2-(2-bromoethoxy)phenyl]-8-chloro-chromen-4-one (450 mg, yield: 69%) as a yellow solid.

### Step 2: Preparation of N-[1-[2-[5-bromo-2-(8-chloro-4-oxo-chromen-2-yl)phenoxy]ethyl]pyrrolidin-3-yl]-N-methyl-methanesulfonamide

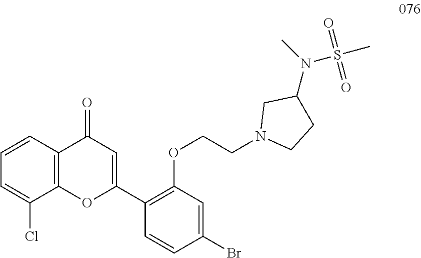 [SMILES: CN(C1CCN(CCOc2cc(Br)ccc2-c2cc(=O)c3cccc(Cl)c3o2)C1)S(C)(=O)=O]

To a mixture of 2-[4-bromo-2-(2-bromoethoxy)phenyl]-8-chloro-chromen-4-one (70 mg, 153 μmol), 3-(methylsulfonyl)pyrrolidine (32.8 mg, 153 μmol, as the “AMINE” in Table 4) in DMF (1 mL) and ethanol (3 mL) was added sodium bicarbonate (51.3 mg, 0.611 mmol) and the reaction mixture was stirred at 50° C. for 5 hours. After the reaction was completed, the mixture was purified by prep-HPLC to give N-[1-[2-[5-bromo-2-(8-chloro-4-oxo-chromen-2-yl)phenoxy]ethyl]pyrrolidin-3-yl]-N-methyl-methanesulfonamide (33.5 mg, yield: 37.5%) as a white solid. MS obsd. (ESI⁺) [(M+2H)⁺]: 557.2.

Example 076: ¹H NMR (DMSO-d₆, 400 MHz) δ ppm 8.03 (d, J=7.9 Hz, 2H), 7.86 (d, J=8.4 Hz, 1H), 7.61 (d, J=1.7 Hz, 1H), 7.40-7.54 (m, 2H), 7.00 (s, 1H), 4.40-4.89 (m, 3H), 3.64-3.85 (m, 2H), 3.03-3.28 (m, 1H), 2.90 (s, 3H), 2.41-2.62 (m, 6H), 1.85-2.29 (m, 2H).

The following compounds 077 to 092 were prepared in analogy to the procedure described for the preparation of Example 076, replacing 2-(4-bromo-2-hydroxy-phenyl)-8-chloro-chromen-4-one with “CORE”, 1,2-dibromoethane with “LINKER” in Step 1, 3-(methylsulfonyl)pyrrolidine with “AMINE” in Step 2, by the reagent indicated in Table 4.

TABLE 4
Compounds synthesis and characterization

| | | CORE, | |
|---|---|---|---|
| Example | Compounds Name and | LINKER and | |
| No. | Structure | AMINE | ¹H NMR and (ESI⁺) |
| 077 | 8-chloro-2-[2-[3-[(3R,4S)-3,4- dihydroxypyrrolidin-1- yl]propoxy]-4- (trifluoromethyl)phenyl] chromen-4-one 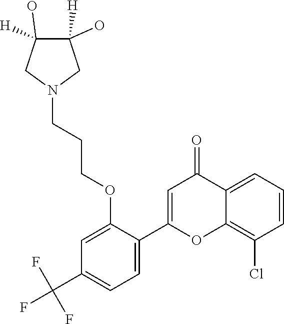 [SMILES: O=c1cc(-c2ccc(C(F)(F)F)cc2OCCCN2C[C@@H](O)[C@@H](O)C2)oc2c(Cl)cccc12] | CORE: Int-1 LINKER: 1,3- dibromopropane AMINE: (3R,4S)- pyrrolidine- 3,4-diol | H NMR (DMSO-d₆, 400 MHz) δ ppm 8.09-8.17 (m, 1H), 7.98- 8.07 (m, 2H), 7.48-7.60 (m, 3H), 7.06 (s, 1H), 4.30 (br t, J = 5.9 Hz, 2H), 4.00 (br s, 2H), 2.97- 3.06 (m, 2H), 2.77 (br d, J = 6.2 Hz, 2H), 2.58 (br d, J = 9.5 Hz, 2H), 1.95-2.04 (m, 2H). MS obsd. (ESI⁺) [(M + H)⁺]: 484.4. |
| 078 | 8-chloro-2-[2-[3-[(1,1- dioxothian-4- yl)amino]propoxy]-4- (trifluoromethyl)phenyl] chromen-4-one 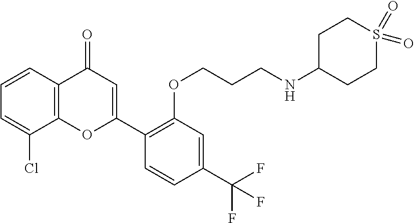 [SMILES: O=c1cc(-c2ccc(C(F)(F)F)cc2OCCCNC2CCS(=O)(=O)CC2)oc2c(Cl)cccc12] | CORE: Int-1 LINKER: 1,3- dibromopropane AMINE: 1,1- dioxothian-4- amine hydrochloride | ¹H NMR (DMSO-d₆, 400 MHz) δ ppm 8.14 (d, J = 7.9 Hz, 1H), 8.00-8.08 (m, 2H), 7.46-7.62 (m, 3H), 7.09 (s, 1H), 4.29-4.40 (m, 2H), 3.01-3.13 (m, 2H), 2.88- 3.00 (m, 2H), 2.61-2.77 (m, 3H), 1.75-2.07 (m, 4H), 1.53-1.66 (m, 2H). MS obsd. (ESI⁺) [(M + H)⁺]: 530.3. |
| 079 | 8-chloro-2-[2-[3-(2- methylsulfonylethylamino) propoxy]-4- (trifluoromethyl)phenyl] chromen-4-one 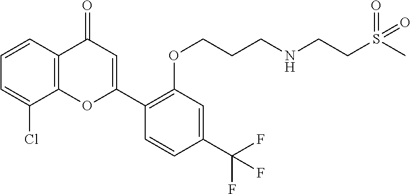 [SMILES: CS(=O)(=O)CCNCCCOc1cc(C(F)(F)F)ccc1-c1cc(=O)c2cccc(Cl)c2o1] | CORE: Int-1 LINKER: 1,3- dibromopropane AMINE: 2 methylsulfonyl ethanamine | ¹H NMR (DMSO-d₆, 400 MHz) δ ppm 8.10-8.17 (m, 1H), 8.00- 8.08 (m, 2H), 7.49-7.65 (m, 3H), 7.05 (s, 1H), 4.31-4.42 (m, 2H), 3.43-3.56 (m, 4H), 3.09-3.19 (m, 5H), 2.09-2.20 (m, 2H). MS obsd. (ESI⁺) [(M + H)⁺]: 503.9. |
| 080 | 2-[3-[2-(8-chloro-4-oxo- chromen-2-yl)-5- (trifluoromethyl)phenoxy] propylamino]ethanesulfonamide 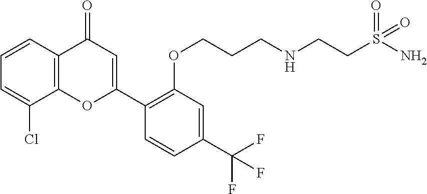 [SMILES: NS(=O)(=O)CCNCCCOc1cc(C(F)(F)F)ccc1-c1cc(=O)c2cccc(Cl)c2o1] | CORE: Int-1 LINKER: 1,3- dibromopropane AMINE: 2 aminoethane- sulfonamidc | ¹H NMR (DMSO-d₆, 400 MHz) δ ppm 8.15 (d, J = 8.4 Hz, 1H), 8.03 (dd, J = 7.8, 3.9 Hz, 2H), 7.46-7.60 (m, 3H), 7.11 (s, 1H), 6.83 (br s, 2H), 4.33 (br t, J = 6.2 Hz, 2H), 3.05-3.15 (m, 2H), 2.89-2.95 (m, 2H), 2.72 (br t, J = 6.7 Hz, 2H), 1.95 (quin, J = 6.2 Hz, 2H). MS obsd. (ESI⁺) [(M + H)⁺]: 505.0. |
| 081 | 8-chloro-2-[2-[3-(4- methylsulfonylpiperazin-1- yl)propoxy]-4- (trifluoromethyl)phenyl] chromen-4-one 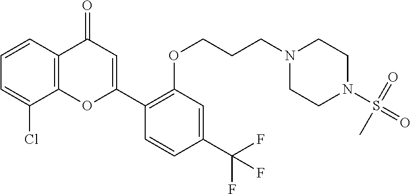 [SMILES: CS(=O)(=O)N1CCN(CCCOc2cc(C(F)(F)F)ccc2-c2cc(=O)c3cccc(Cl)c3o2)CC1] | CORE: Int-1 LINKER: 1,3 dibromopropane AMINE: 1- methylsulfonyl piperazine | ¹H NMR (DMSO-d₆, 400 MHz) δ ppm 8.14 (d, J = 8.2 Hz, 1H), 8.04 (dq, J = 7.9, 1.4 Hz, 2H), 7.61 (d, J = 8.8 Hz, 1H), 7.47- 7.57 (m, 2H), 7.09 (s, 1H), 4.36 (t, J = 6.0 Hz, 2H), 3.53-3.80 (m, 4H), 3.05-3.31 (m, 6H), 3.00 (s, 3H), 2.23 (br s, 2H). MS obsd. (ESI⁺) [(M + H)⁺]: .544.9. |
| 082 | 2-[2-[2-(4-acetylpiperazin-1- yl)ethoxyl-4-bromo-phenyl]- 8-chloro-chromen-4-one 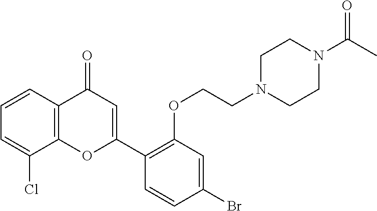 [SMILES: CC(=O)N1CCN(CCOc2cc(Br)ccc2-c2cc(=O)c3cccc(Cl)c3o2)CC1] | CORE: Int-4 LINKER: 1,2- dibromoethane AMINE: 1- piperazin-1- ylethanone | ¹H NMR (DMSO-d₆, 400 MHz) δ ppm 7.97-8.03 (m, 2H), 7.93 (d, J = 8.4 Hz, 1H), 7.55 (d, J = 1.7 Hz, 1H), 7.47-7.53 (m, 2H), 7.43 (dd, J = 8.5, 1.8 Hz, 1H), 4.33 (t, J = 5.3 Hz, 2H), 3.47 (q, J = 4.9 Hz, 4H), 2.77 (t, J = 5.3 Hz, 2H), 2.45-2.49 (m, 2H), 2.42 (t, J = 5.0 Hz, 2H), 1.98 (s, 3H). MS obsd. (ESI⁺) [(M + H)⁺]: 505.0. |
| 083 | 1-[2-[5-bromo-2-(8-chloro-4- oxo-chromen-2- yl)phenoxy]ethyl]pyrrolidine- 3-carboxamide 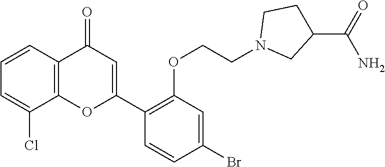 [SMILES: NC(=O)C1CCN(CCOc2cc(Br)ccc2-c2cc(=O)c3cccc(Cl)c3o2)C1] | CORE: Int-4 LINKER: 1,2- dibromoethane AMINE: pyrrolidine-3- carboxamide | ¹H NMR (DMSO-d₆, 400 MHz) δ ppm 8.02 (d, J = 7.9 Hz, 2H), 7.84 (d, J = 8.4 Hz, 1H), 7.57- 7.68 (m, 2H), 7.46-7.56 (m, 2H), 7.07-7.22 (m, 1H), 6.97 (s, 1H), 4.37-4.62 (m, 2H), 3.57-3.87 (m, 4H), 2.95-3.32 (m, 3H), 1.77- 2.35 (m, 2H). MS obsd. (ESI⁺) [(M + H)⁺]: 491.1. |
| 084 | 2-[4-bromo-2-[2-(3- methylsulfonylpyrrolidin-1- yl)ethoxylphenyl]-8-chloro- chromen-4-one 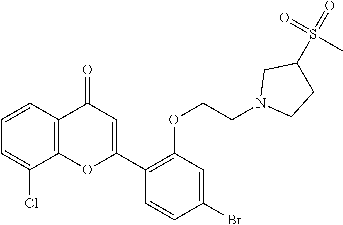 [SMILES: CS(=O)(=O)C1CCN(CCOc2cc(Br)ccc2-c2cc(=O)c3cccc(Cl)c3o2)C1] | CORE: Int-4 LINKER: 1,2- dibromoethane AMINE: 3- methylsulfonyl pyrrolidine | ¹H NMR (DMSO-d₆, 400 MHz) δ ppm 8.02 (d, J = 7.9 Hz, 2H), 7.84 (d, J = 8.4 Hz, 1H), 7.57- 7.68 (m, 2H), 7.46-7.56 (m, 2H), 7.07-7.22 (m, 1H), 6.97 (s, 1H), 4.37-4.62 (m, 2H), 3.57-3.87 (m, 4H), 2.95-3.32 (m, 3H), 1.77- 2.35 (m, 3H). MS obsd. (ESI⁺) [(M + H)⁺]: 526.2. |
| 085 | 4-[2-[5-bromo-2-(8-chloro-4- oxo-chromen-2- yl)phenoxy]ethyl] thiomorpholine-3-carboxamide 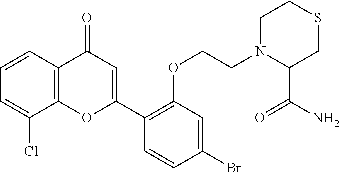 [SMILES: NC(=O)C1CSCCN1CCOc1cc(Br)ccc1-c1cc(=O)c2cccc(Cl)c2o1] | CORE: Int-4 LINKER: 1,2- dibromoethane AMINE: thiomorpholine- 3-carboxamide | ¹H NMR (DMSO-d₆, 400 MHz) δ ppm 7.95 (dd, J = 7.9, 2.3 Hz, 2H), 7.80 (d, J = 8.4 Hz, 1H), 7.35-7.54 (m, 3H), 7.03 (br s, 1H), 4.39 (br s, 2H), 3.45-3.82 (m, 4H), 2.81-2.96 (m, 3H), 2.63-2.78 (m, 1H), 2.49-2.59 (m, 1H). MS obsd. (ESI⁺) [(M + H)⁺]: 523.1. |
| 086 | N-[3-[2-(8-chloro-4-oxo- chromen-2-yl)-5- (trifluoromethyl)phenoxy]-2- hydroxy- propyl] cyclopropanesulfonamide 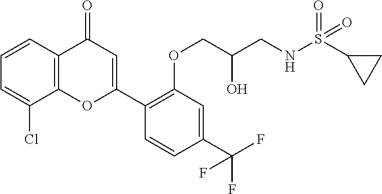 [SMILES: O=c1cc(-c2ccc(C(F)(F)F)cc2OCC(O)CNS(=O)(=O)C2CC2)oc2c(Cl)cccc12] | CORE: Int-1 LINKER: 2- (chloromethyl) oxirane AMINE: cyclopropane- sulfonamide | ¹H NMR (DMSO-d₆, 400 MHz) δ ppm 8.16 (d, J = 7.9 Hz, 1H), 8.03 (dq, J = 7.9, 1.4 Hz, 2H), 7.55-7.64 (m, 2H), 7.52 (t, J = 7.9 Hz, 1H), 7.24 (s, 1H), 7.14- 7.20 (m, 1H), 5.38 (br d, J = 4.4 Hz, 1H), 4.28-4.36 (m, 1H), 4.20-4.25 (m, 1H), 3.97 (br d, J = 4.9 Hz, 1H), 3.08-3.19 (m, 1H), 1.93-2.09 (m, 1H), 0.84- 0.92 (m, 4H). MS obsd. (ESI⁺) [(M + H)⁺]: 518.4. |
| 087 | 8-chloro-2-[2-[2-[4-(1H- tetrazol-5-yl)-1- piperidyl]ethoxy]-4- (trifluoromethyl)phenyl] chromen-4-one 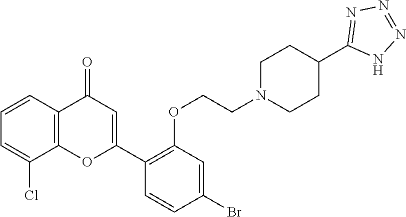 [SMILES: O=c1cc(-c2ccc(Br)cc2OCCN2CCC(c3nnn[nH]3)CC2)oc2c(Cl)cccc12] | CORE: Int-4 LINKER: 1,2- dibromoethane AMINE: 4- (1H-tetrazol-5- yl)piperidine | ¹H NMR (CHLOROFORM-d, 400 MHz) δ ppm 7.97-8.11 (m, 2H), 7.72 (br dd, J = 1.1, 1.6 Hz, 1H), 7.26-7.48 (m, 3H), 7.01 (s, 1H), 4.97-5.15 (m, 2H), 4.60- 4.71 (m, 2H), 4.12 (dt, J = 8.4, 4.1 Hz, 4H), 3.31-3.64 (m, 1H), 3.00-3.31 (m, 2H), 2.26-2.42 (m, 1H), 1.98-2.08 (m, 2H), 1.18 (m, 2H). MS obsd. (ESI⁺) [(M + H)⁺]: 530.0. |
| 088 | 8-chloro-2-[2-[3-(3- methylsulfonylpyrrolidin-1- yl)propoxy]-4- (trifluoromethyl)phenyl] chromen-4-one 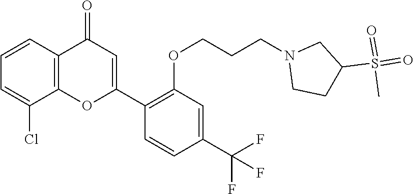 [SMILES: CS(=O)(=O)C1CCN(CCCOc2cc(C(F)(F)F)ccc2-c2cc(=O)c3cccc(Cl)c3o2)C1] | CORE: Int-1 LINKER: 1,3- dibromopropane AMINE: 3- methylsulfonyl pyrrolidine | ¹H NMR (DMSO-d₆, 400 MHz) δ ppm 8.10-8.16 (m, 1H), 8.03 (d, J = 8.1 Hz, 2H), 7.43-7.60 (m, 3H), 7.09 (s, 1H), 4.31 (t, J = 6.1 Hz, 2H), 3.75 (br d, J = 8.8 Hz, 1H), 2.91 (s, 3H), 2.75- 2.85 (m, 2H), 2.52-2.62 (m, 4H), 1.88-2.15 (m,4H). MS obsd. (ESI⁺) [(M + H)⁺]: 529.9. |
| 089 | 2-[4-bromo-2-[2-(1,1-dioxo- 1,4-thiazinan-4- yl)ethoxy]phenyl]-8-chloro- chromen-4-one 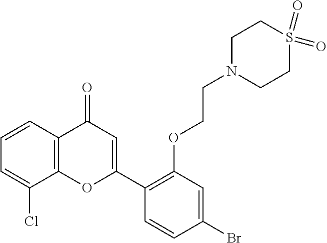 [SMILES: O=c1cc(-c2ccc(Br)cc2OCCN2CCS(=O)(=O)CC2)oc2c(Cl)cccc12] | CORE: Int-4 LINKER: 1,2- dibromoethane AMINE: thiomorpholine 1,1-dioxide | ¹H NMR (DMSO-d₆, 400 MHz) δ ppm 7.99-8.06 (m, 2H), 7.95 (d, J = 8.6 Hz, 1H), 7.38-7.57 (m, 4H), 4.35 (t, J = 5.1 Hz, 2H), 3.21 (br d, J = 4.5 Hz, 4H), 2.95-3.14 (m, 6H). MS obsd. (ESI⁺) [(M + H)⁺]: 511.9. |
| 090 | 2-[4-bromo-2-[2-(2- morpholinoethoxy)ethoxy] phenyl]-8-chloro-chromen-4- one 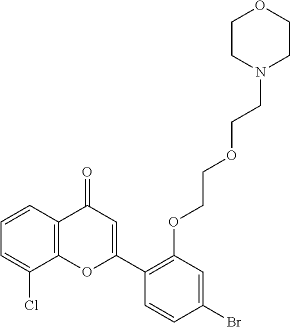 [SMILES: O=c1cc(-c2ccc(Br)cc2OCCOCCN2CCOCC2)oc2c(Cl)cccc12] | CORE: Int-4 LINKER: 1- bromo-2-(2- bromoethoxy) ethane AMINE: morpholine | ¹H NMR (DMSO-d₆, 400 MHz) δ ppm 9.76-10.12 (m, 1H), 7.90- 8.08 (m, 3H), 7.43-7.61 (m, 2H), 7.29 (s, 1H), 4.33-4.48 (m, 2H), 3.79-3.98 (m, 6H), 3.57-3.75 (m, 2H), 3.44-3.53 (m, 4H), 2.87- 3.22 (m, 2H). MS obsd. (ESI⁺) [(M + H)⁺]: 508.1. |
| 091 | 1-[2-[2-(8-chloro-4-oxo- chromen-2-yl)-5-methyl- phenoxy]ethyl]imidazolidin- 2-one 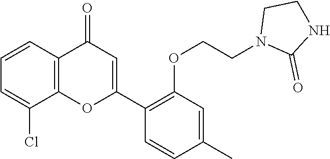 [SMILES: Cc1ccc(-c2cc(=O)c3cccc(Cl)c3o2)c(OCCN2CCNC2=O)c1] | CORE: Int-2 AMINE: tert- butyl N-(3- bromoethyl) carbamate AMINE: imidazolidin- 2-one | ¹H NMR (DMSO-d₆, 400 MHz) δ ppm 7.99 (d, J = 8.1 Hz, 2H), 7.87 (d, J = 8.1 Hz, 1H), 7.49 (t, J = 7.9 Hz, 1H), 7.16 (s, 1H), 7.09 (s, 1H), 7.03 (d, J = 8.1 Hz, 1H), 6.35 (s, 1H), 4.27 (t, J = 5.4 Hz, 2H), 3.25-3.48 (m, 4H), 3.13-3.23 (m, 2H), 2.40 (s, 3H). MS obsd. (ESI⁺) [(M + H)⁺]: 399.0. |
| 092 | 1-[2-[2-(8-chloro-4-oxo- chromen-2-yl)-5-methyl- phenoxy]ethyl]-3-methyl- imidazolidin-2-one 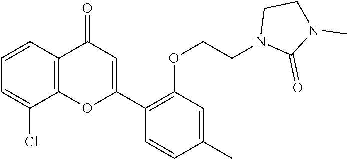 [SMILES: Cc1ccc(-c2cc(=O)c3cccc(Cl)c3o2)c(OCCN2CCN(C)C2=O)c1] | CORE: Int-2 AMINE: tert- butyl N-(3- bromoethyl) carbamate AMINE: 1-methyl- imidazolidin- 2-one | ¹H NMR (HDMSO, 400 MHz) δ ppm 7.99 (d, J = 7.8 Hz, 2H), 7.86 (d, J = 7.9 Hz, 1H), 7.49 (t, J = 7.9 Hz, 1H), 7.16 (s, 1H), 7.06 (m, 2H), 4.28 (s, 2H), 3.49 (t, J = 5.4 Hz, 2H), 3.14-3.22 (m, 4H), 2.60 (s, 3H), 2.40 (s, 3H). MS obsd. (ESI⁺) [(M + H)⁺]: 413.4. |

### Example 093: 8-chloro-2-[2-[2-(4-methylsulfinyl-1-piperidyl)ethoxy]-4-(trifluoromethyl)phenyl]chromen-4-one and Example 094: 8-chloro-2-[2-[2-[4-(methylsulfonimidoyl)-1-piperidyl]ethoxy]-4-(trifluoromethyl)phenyl]chromen-4-one

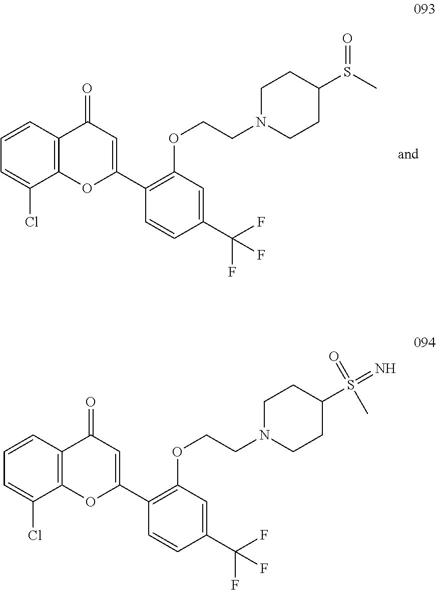 [SMILES: CS(=O)C1CCN(CCOc2cc(C(F)(F)F)ccc2-c2cc(=O)c3cccc(Cl)c3o2)CC1 ; CS(=N)(=O)C1CCN(CCOc2cc(C(F)(F)F)ccc2-c2cc(=O)c3cccc(Cl)c3o2)CC1]

### Step 1: preparation of 8-chloro-2-[2-[2-(4-methylsulfanyl-1-piperidyl)ethoxy]-4-(trifluoromethyl)phenyl]chromen-4-one

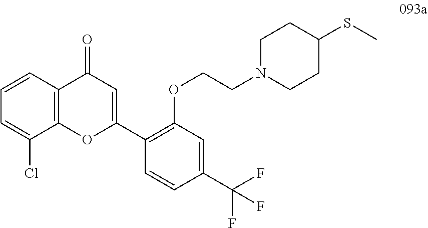 [SMILES: CSC1CCN(CCOc2cc(C(F)(F)F)ccc2-c2cc(=O)c3cccc(Cl)c3o2)CC1]

Compound 093a was prepared in analogy to the procedure described for the preparation of example 076 by using 4-methylsulfanylpiperidine as the starting material instead of 3-(methylsulfonyl)pyrrolidine in Step 2. MS obsd. (ESI⁺) [(M+H)⁺]: 497.9.

### Step 2: preparation of 8-chloro-2-[2-[2-(4-methylsulfinyl-1-piperidyl)ethoxy]-4-(trifluoromethyl)phenyl]chromen-4-one and 8-chloro-2-[2-[2-[4-(methylsulfonimidoyl)-1-piperidyl]ethoxy]-4-(trifluoromethyl)phenyl]chromen-4-one

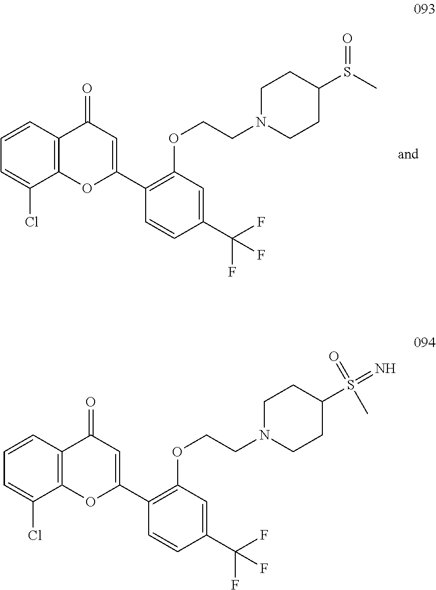 [SMILES: CS(=O)C1CCN(CCOc2cc(C(F)(F)F)ccc2-c2cc(=O)c3cccc(Cl)c3o2)CC1 ; CS(=N)(=O)C1CCN(CCOc2cc(C(F)(F)F)ccc2-c2cc(=O)c3cccc(Cl)c3o2)CC1]

A solution of 8-chloro-2-[2-[2-(4-methylsulfanyl-1-piperidyl)ethoxy]-4-(trifluoromethyl)phenyl]chromen-4-one (40 mg, 80.3 μmol), iodobenzene diacetate (103 mg, 321 μmol) and ammonium carbomate (11.6 mg, 120 μmol) in MeOH (10 mL) was stirred at room temperature for 4 hours. After the reaction was completed, the mixture was purified by prep-HPLC to give 8-chloro-2-[2-[2-(4-methylsulfinyl-1-piperidyl)ethoxy]-4-(trifluoromethyl)phenyl]chromen-4-one (15.2 mg, yield: 35.0%) and 8-chloro-2-[2-[2-[4-(methylsulfonimidoyl)-1-piperidyl]ethoxy]-4-(trifluoromethyl)phenyl]chromen-4-one (2.7 mg, yield: 5.8%) as white solid.

Example 093: MS obsd. (ESI⁺)[(M+H)⁺]: 513.9. ¹H NMR (DMSO-d₆, 400 MHz) δ ppm 8.08-8.13 (m, 1H), 8.05 (dd, J=7.9, 1.3 Hz, 2H), 7.65 (t, J=3.2 Hz, 2H), 7.54 (t, J=7.9 Hz, 1H), 7.06 (s, 1H), 4.62 (br t, J=4.6 Hz, 2H), 3.65 (br s, 2H), 3.57 (br d, J=3.8 Hz, 2H), 3.21-3.31 (m, 1H), 3.04-3.13 (m, 2H), 2.98 (s, 3H), 2.20 (br d, J=1.0 Hz, 2H), 1.78-1.96 (m, 2H).

Example 094: MS obsd. (ESI⁺)[(M+H)⁺]: 528.9. ¹H NMR (DMSO-d₆, 400 MHz) δ ppm 9.46-9.69 (m, 1H), 8.10 (br d, J=8.3 Hz, 1H), 8.00-8.07 (m, 2H), 7.61-7.68 (m, 2H), 7.54 (t, J=7.9 Hz, 1H), 7.03 (br s, 1H), 4.63 (br s, 2H), 3.56-3.77 (m, 4H), 3.06-3.23 (m, 2H), 2.69-2.85 (m, 2H), 2.54-2.58 (m, 2H), 1.94-2.16 (m, 2H), 1.71-1.89 (m, 2H).

### Example 095: ethyl 2-[3-[2-(8-chloro-4-oxo-chromen-2-yl)-5-methyl-phenoxy]propylamino]-2-oxo-acetate

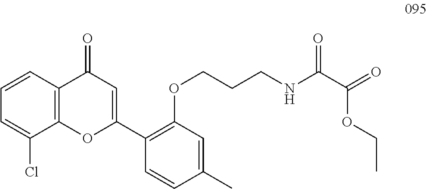 [SMILES: CCOC(=O)C(=O)NCCCOc1cc(C)ccc1-c1cc(=O)c2cccc(Cl)c2o1]

### Step 1: Preparation of tert-butyl N-[3-[2-(8-chloro-4-oxo-chromen-2-yl)-5-methyl-phenoxy]propyl]carbamate

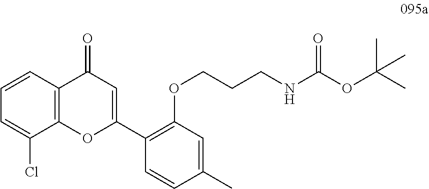 [SMILES: Cc1ccc(-c2cc(=O)c3cccc(Cl)c3o2)c(OCCCNC(=O)OC(C)(C)C)c1]

To a solution of 8-chloro-2-(2-hydroxy-4-methyl-phenyl)chromen-4-one (500 mg, 1.74 mmol, as the “CORE” in Table 5) and tert-butyl (3-bromopropyl)carbamate (415 mg, 1.74 mmol, as the “LINKER” in Table 5) in DMF (18 mL) was added K₂CO₃ (1.21 g, 8.72 mmol) and the mixture was stirred at 50° C. overnight. After the reaction was completed, the reaction mixture was partitioned between EtOAc (20 mL) and water (30 mL). The organic layer was separated out and the aqueous phase was extracted with EtOAc (30 mL) twice. The combined organic layer was concentrated in vacuo and the residue was purified by ISCO (eluting with EtOAc:PE=0 to 30%) to give tert-butyl N-[3-[2-(8-chloro-4-oxo-chromen-2-yl)-5-methyl-phenoxy]propyl]carbamate (530 mg, yield: 68.5%) as a yellow solid. MS obsd. (ESI⁺) [(M+H)⁺]: 444.2.

### Step 2: Preparation of 2-[2-(3-aminopropoxy)-4-methyl-phenyl]-8-chloro-chromen-4-one

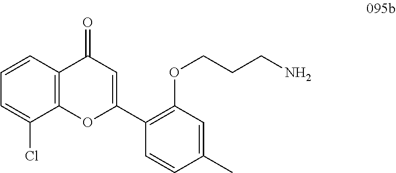 [SMILES: Cc1ccc(-c2cc(=O)c3cccc(Cl)c3o2)c(OCCCN)c1]

Compound tert-butyl N-[3-[2-(8-chloro-4-oxo-chromen-2-yl)-5-methyl-phenoxy]propyl]carbamate (530 mg, 1.06 mmol) was dissolved in the DCM (3 mL), followed by adding TFA (1 mL, 13 mmol) dropwise. The reaction was stirred at room temperature for 2 hours. Then, the reaction mixture was concentrated in vacuo and coevaporated with toluene to give 2-[2-(3-aminopropoxy)-4-methyl-phenyl]-8-chloro-chromen-4-one (400 mg, yield: 97.4%) as yellow oil. MS obsd. (ESI⁺) [(M+H)⁺]: 344.2.

### Step 3: Preparation of tert-butyl 2-[3-[2-(8-chloro-4-oxo-chromen-2-yl)-5-methyl-phenoxy]propylamino]-2-oxo-acetate

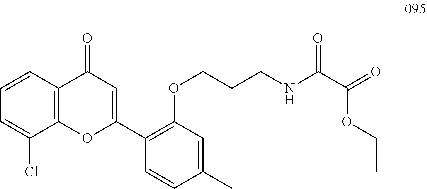 [SMILES: CCOC(=O)C(=O)NCCCOc1cc(C)ccc1-c1cc(=O)c2cccc(Cl)c2o1]

To a mixture solution of 2-[2-(3-aminopropoxy)-4-methyl-phenyl]-8-chloro-chromen-4-one (540 mg, 1.57 mmol) and DIPEA (549 μL, 3.14 mmol) in DCM (15 mL) was added ethyl 2-chloro-2-oxoacetate (197 μL, 1.73 mmol, as the “TAIL” in Table 5) at 0° C. The reaction was stirred at room temperature for 2 hours. After the reaction was completed, the reaction was adjusted to pH-4 by addition of 4N HCl and then extracted with EtOAc (50 mL) three times. The combined organic layer was washed with brine, dried over anhydrous Na₂SO₄ and concentrated in vacuo to give 2-[3-[2-(8-chloro-4-oxo-chromen-2-yl)-5-methyl-phenoxy]propylamino]-2-oxo-acetate (90 mg, yield: 12.3%) as a light yellow foam. MS obsd. (ESI⁺) [(M+H)⁺]: 444.1.

Example 095: ¹H NMR (DMSO-d₆, 400 MHz) δ ppm 8.98-9.07 (i, 1H), 7.95-8.04 (m, 2H), 7.83-7.92 (m, 1H), 7.42-7.55 (2, 1H), 6.97-7.13 (i, 3H), 4.11-4.25 (m, 4H), 3.28-3.34 (m, 2H), 2.40 (s, 3H), 1.94-2.06 (m, 2H), 1.19-1.29 (i, 3H).

The following compounds 096 to 109 were prepared in analogy to the procedure described for the preparation of Example 095, replacing 8-chloro-2-(2-hydroxy-4-methyl-phenyl)chromen-4-one with “CORE”, tert-butyl (3-bromopropyl)carbamate with “LINKER” in Step 1, replacing ethyl 2-chloro-2-oxo acetate with “TAIL” in Step 3, by the reagent indicated in Table 5.

TABLE 5
Compounds synthesis and characterization

| | | CORE, | |
|---|---|---|---|
| Example | | LINKER and | |
| No. | Compounds Name and Structure | TAIL | ¹H NMR and (ESI⁺) |
| 096 | 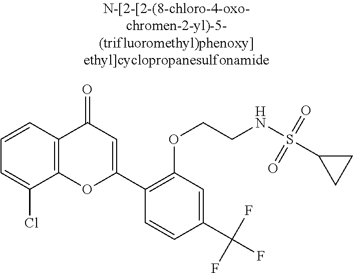 [SMILES: O=c1cc(-c2ccc(C(F)(F)F)cc2OCCNS(=O)(=O)C2CC2)oc2c(Cl)cccc12] | CORE: Int-1 LINKER: tert-butyl N- (2- bromoethyl) carbamate TAIL: cyclopropane- sulfonyl chloride | ¹H NMR (METHANOL-d₄, 400 MHz) δ ppm 8.16 (d, J = 8.7 Hz, 1H), 8.00 (dd, J = 8.0, 1.5 Hz, 1H), 7.83 (dd, J = 7.8, 1.5 Hz, 1H), 7.32-7.52 (m, 3H), 7.25 (s, 1H), 4.51 (m, 1H), 4.27 (t, J = 5.3 Hz, 2H), 3.53 (t, J = 5.3 Hz, 2H), 2.45-2.56 (m, 1H), 0.80-0.97 (m, 4H). MS obsd. (ESI⁺) [(M + H)⁺]: 488.0. |
| 097 | 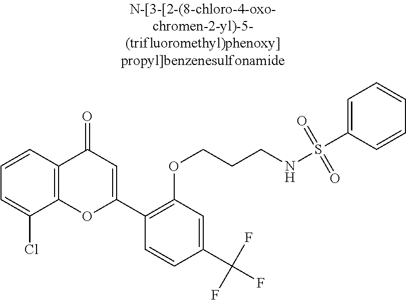 [SMILES: O=c1cc(-c2ccc(C(F)(F)F)cc2OCCCNS(=O)(=O)c2ccccc2)oc2c(Cl)cccc12] | CORE: Int-1 LINKER: tert-butyl N- (3- bromopropyl) carbamate TAIL: benzenesulfonyl chloride | ¹H NMR (DMSO-d₆, 400 MHz) δ ppm 9.39-9.67 (m, 1H), 8.09 (d, J = 8.3 Hz, 1H), 8.03 (dt, J = 7.9, 1.9 Hz, 1H), 7.67-7.75 (m, 3H), 7.39-7.62 (m, 6H), 6.97 (s, 1H), 4.23 (t, J = 6.1 Hz, 2H), 2.93 (d, J = 6.1 Hz, 2H), 1.88 (br t, J = 6.4 Hz, 2H). MS obsd. (ESI⁺) [(M + H)⁺]: 538.2. |
| 098 | 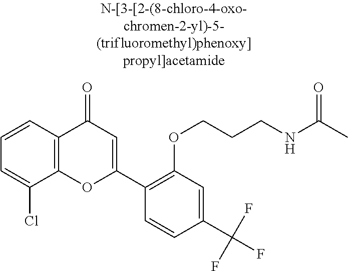 [SMILES: CC(=O)NCCCOc1cc(C(F)(F)F)ccc1-c1cc(=O)c2cccc(Cl)c2o1] | CORE: Int-1 LINKER: tert-butyl N- (3- bromopropyl) carbamate TAIL: acetyl acetate | ¹H NMR (DMSO-J₆, 400 MHz) δ ppm 8.13 (d, J = 8.2 Hz, 1H), 8.02 (d, J = 7.8 Hz, 2H), 7.53 (m, 3H), 7.09 (s, 1H), 4.27 (t, J = 6.3 Hz, 2H), 3.21 (m, 2H), 1.93 (m, 2H), 1.78 (s, 3H). MS obsd. (ESI⁺) [(M + H)⁺]: 440.4. |
| 099 | 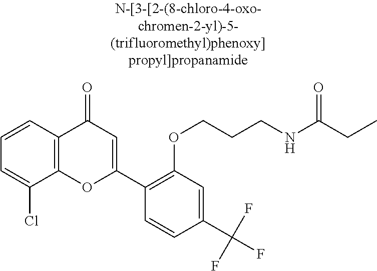 [SMILES: CCC(=O)NCCCOc1cc(C(F)(F)F)ccc1-c1cc(=O)c2cccc(Cl)c2o1] | CORE: Int-1 LINKER: tert-butyl N- (3- bromopropyl) carbamate TAIL: propanoyl propanoate | ¹H NMR (DMSO-d₆, 400 MHz) δ ppm 8.13 (d, J = 8.2 Hz, 1H), 8.03 (dd, J = 7.9, 1.4 Hz, 1H), 7.77-7.92 (m, 1H), 7.44-7.63 (m, 3H), 7.10 (s, 1H), 4.27 (t, J = 6.3 Hz, 2H), 3.21 (br d, J = 6.0 Hz, 2H), 2.04 (d, J = 7.6 Hz, 2H), 1.92 (t, J = 6.5 Hz, 2H), 0.96 (t, J = 7.6 Hz, 3H). MS obsd. (ESI⁺) [(M + H)⁺]: 454.2. |
| 100 | 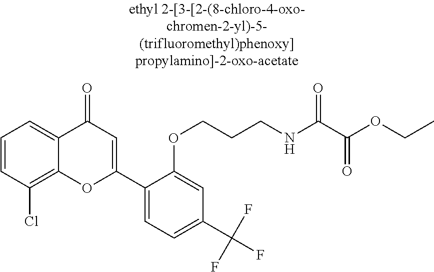 [SMILES: CCOC(=O)C(=O)NCCCOc1cc(C(F)(F)F)ccc1-c1cc(=O)c2cccc(Cl)c2o1] | CORE: Int-1 LINKER: tert-butyl N- (3- bromopropyl) carbamate TAIL: ethyl 2-chloro- 2-oxo-acetate | ¹H NMR (DMSO-d₆, 400 MHz) δ ppm 9.02 (t, J = 5.7 Hz, 1H), 8.12 (d, J = 8.1 Hz, 1H), 8.08- 7.94 (m, 2H), 7.67-7.42 (m, 3H), 7.10 (s, 1H), 4.27 (t, J = 6.1Hz, 2H), 4.17 (q, J = 7.1 Hz, 2H), 3.34-3.30 (m, 2H), 2.07- 1.95 (m, 2H), 1.24 (t, J = 7.1 Hz, 3H). MS obsd. (ESI⁺) [(M + H)⁺]: 498.0. |
| 101 | 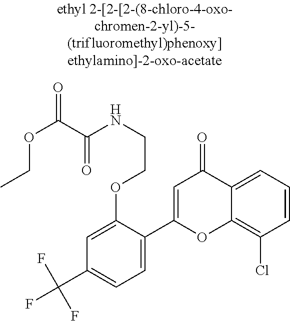 [SMILES: CCOC(=O)C(=O)NCCOc1cc(C(F)(F)F)ccc1-c1cc(=O)c2cccc(Cl)c2o1] | CORE: Int-1 LINKER: tert-butyl N- (3- bromoethyl) carbamate TAIL: ethyl 2-chloro- 2-oxo-acetate | ¹H NMR (DMSO-d₆, 400 MHz) δ ppm 9.07 (br t, J = 5.6 Hz, 1H), 8.11 (d, J = 7.9 Hz, 1H), 8.01 (dq, J = 7.8, 1.7 Hz, 2H), 7.63 (s, 1H), 7.55-7.59 (m, 1H), 7.44-7.54 (m, 1H), 7.02 (s, 1H), 4.42 (t, J = 5.7 Hz, 2H), 4.17 (q, J = 7.1 Hz, 2H), 3.57 (q, J = 5.7 Hz, 2H), 1.21 (t, J = 7.2 Hz, 3H). MS obsd. (ESI⁺) [(M + H)⁺]: 484.1. |
| 102 | 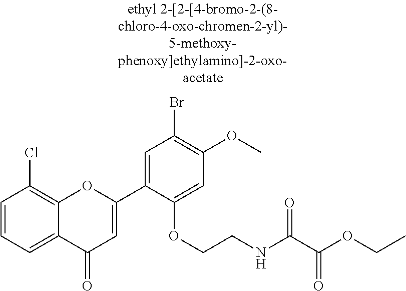 [SMILES: CCOC(=O)C(=O)NCCOc1cc(OC)c(Br)cc1-c1cc(=O)c2cccc(Cl)c2o1] | CORE: Int-11 LINKER: tert-butyl N- (3- bromoethyl) carbamate TAIL: ethyl 2-chloro- 2-oxo-acetate | ¹H NMR (DMSO-d₆, 400 MHz) δ ppm 9.13 (t, J = 5.5 Hz, 1H), 8.11 (s, 1H), 8.04-7.92 (m, 2H), 7.48 (t, J = 7.9 Hz, 1H), 6.99 (d, J = 8.2 Hz, 2H), 4.40 (t, J = 5.8 Hz, 2H), 4.18 (q, J = 7.1 Hz, 2H), 4.00 (s, 3H), 3.59 (q, J = 5.7 Hz, 2H), 1.21 (t, J = 7.1 Hz, 3H). MS obsd. (ESI⁺) [(M + H)⁺]: 524.0. |
| 103 | 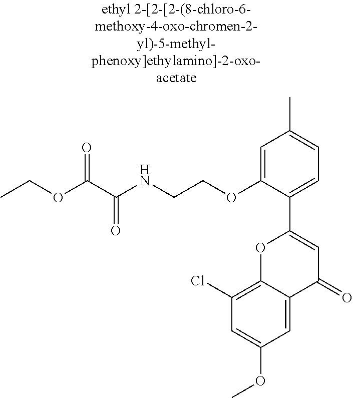 [SMILES: CCOC(=O)C(=O)NCCOc1cc(C)ccc1-c1cc(=O)c2cc(OC)cc(Cl)c2o1] | CORE: Int-16 LINKER: tert-butyl N- (3- bromoethyl) carbamate TAIL: ethyl 2-chloro- 2-oxo-acetate | ¹H NMR (DMSO-d₆, 400 MHz) δ ppm 9.07 (t, J = 5.4 Hz, 1H), 7.83 (d, J = 8.0 Hz, 1H), 7.66 (d, J = 2.9 Hz, 1H), 7.39 (s, 1H), 7.15 (s, 1H), 7.02 (d, J = 8.0 Hz, 1H), 6.96 (s, 1H), 4.29 (t, J = 5.8 Hz, 2H), 4.19 (q, J = 7.1 Hz, 2H), 3.88 (s, 3H), 3.57 (dd, J = 11.6, 5.8 Hz, 2H), 2.40 (s, 3H), 1.22 (t, J = 7.1 Hz, 3H). MS obsd. (ESI⁺) [(M + H)⁺]: 460.0. |
| 104 | 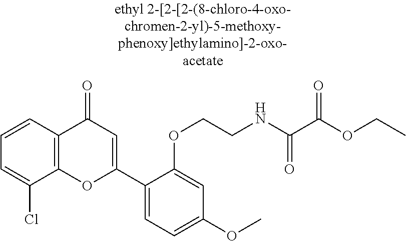 [SMILES: CCOC(=O)C(=O)NCCOc1cc(OC)ccc1-c1cc(=O)c2cccc(Cl)c2o1] | CORE: Int-10 LINKER: tert-butyl N- (3- bromoethyl) carbamate TAIL: ethyl 2-chloro- 2-oxo-acetate | ¹H NMR (DMSO-d₆, 400 MHz) δ ppm 9.09 (s, 1H), 7.96 (dd, J = 10.8, 8.6 Hz, 3H), 7.47 (t, J = 7.9 Hz, 1H), 6.97 (s, 1H), 6.82 (dd, J = 12.1, 3.2 Hz, 2H), 4.32 (t, J = 5.9 Hz, 2H), 4.19 (q, J = 7.1 Hz, 2H), 3.88 (s, 3H), 3.58 (dd, J = 11.5, 5.7 Hz, 2H), 1.22 (t, J = 7.1 Hz, 3H). MS obsd. (ESI⁺) [(M + H)⁺]: 446.1. |
| 105 | 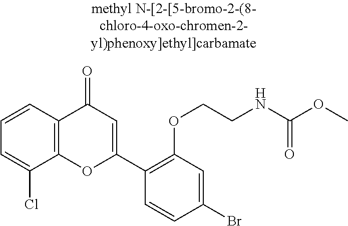 [SMILES: COC(=O)NCCOc1cc(Br)ccc1-c1cc(=O)c2cccc(Cl)c2o1] | CORE: Int-4 LINKER: tert-butyl N- (3- bromopropyl) carbamate TAIL: methyl carbono- chloridate | ¹H NMR (DMSO-d₆, 400 MHz) δ ppm 8.00 (d, J = 7.9 Hz, 2H), 7.86 (d, J = 8.4 Hz, 1H), 7.54- 7.33 (m, 4H), 7.01 (s, 1H), 4.24 (t, J = 5.5 Hz, 2H), 3.50 (s, 3H), 3.44-3.39 (m, 2H). MS obsd. (ESI⁺) [(M + H)⁺]: 452.0. |
| 106 | 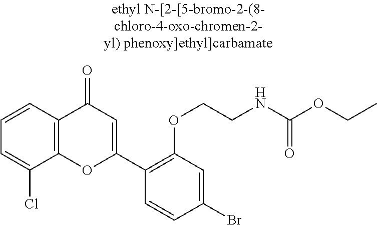 [SMILES: CCOC(=O)NCCOc1cc(Br)ccc1-c1cc(=O)c2cccc(Cl)c2o1] | CORE: Int-4 LINKER: lert-butyl N- (3- bromopropyl) carbamate TAIL: ethyl carbono- chloridate | ¹H NMR (DMSO-d₆, 400 MHz) δ ppm 8.00 (d, J = 7.9 Hz, 2H), 7.86 (d, J = 8.4 Hz, 1H), 7.54- 7.40 (m, 3H), 7.30 (s, 1H), 7.02 (s, 1H), 4.24 (t, J = 5.6 Hz, 2H), 3.94 (q, J = 7.1 Hz, 2H), 3.40 (dd, J = 11.0, 5.5 Hz, 2H), 1.10 (t, J = 7.1 Hz, 3H). MS obsd. (ESI⁺) [(M + H)⁺]: 466.0. |
| 107 | 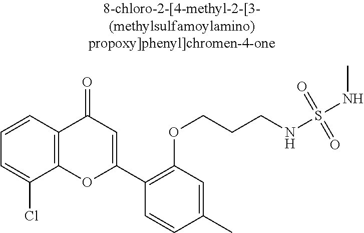 [SMILES: CNS(=O)(=O)NCCCOc1cc(C)ccc1-c1cc(=O)c2cccc(Cl)c2o1] | CORE: Int-2 LINKER: tert-butyl N- (3- bromopropyl) carbamate TAIL: N- methylsulfamoyl chloride | ¹H NMR (DMS0-d₆) δ: 8.00 (d, J = 1.9 Hz, 2H), 7.89 (d, J = 1.9 Hz, 1H), 7.49 (t, J = 1.9 Hz, 1H), 7.13 (s, 1H), 7.07 (s, 1H), 7.03 (d, J = 8.1 Hz, 1H), 6.95 (t, J = 5.7 Hz, 1H), 6.69 (d, J = 5.1 Hz, 1H), 4.23 (t, J = 6.2 Hz, 2H), 3.01 (br d, J = 6.1 Hz, 2H), 2.39- 2.46 (m, 6H), 2.01 (br t, J = 6.4 Hz, 2H). MS obsd. (ESI⁺) [(M + H)⁺]: 437.2. |
| 108 | 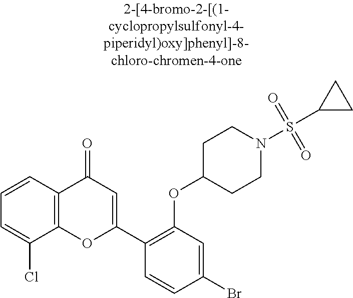 [SMILES: O=c1cc(-c2ccc(Br)cc2OC2CCN(S(=O)(=O)C3CC3)CC2)oc2c(Cl)cccc12] | CORE: Int-4 LINKER: tert-butyl 4- methylsulfonyl oxypiperidine- 1-carboxylate TAIL: cyclopropane- sulfonyl chloride | ¹H NMR (DMSO-d₆, 400 MHz) δ ppm 7.96-8.06 (m, 2H), 7.85 (d, J = 8.4 Hz, 1H), 7.64 (d, J = 1.7 Hz, 1H), 7.50 (t, J = 7.9 Hz, 1H), 7.42 (dd, J = 8.4, 1.8 Hz, 1H), 7.01 (s, 1H), 4.92-5.05 (m, 1H), 3.22-3.31 (m, 4H), 2.43- 2.48 (m, 1H), 1.94-2.07 (m, 2H), 1.71-1.89 (m, 2H), 0.84-0.96 (m, 4H) MS obsd. (ESI⁺) [(M + H)⁺]: 538.2. |
| 109 | 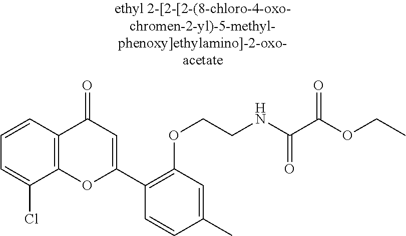 [SMILES: CCOC(=O)C(=O)NCCOc1cc(C)ccc1-c1cc(=O)c2cccc(Cl)c2o1] | CORE: Int-2 LINKER: tert-butyl N- (3- bromoethyl) carbamate TAIL: ethyl 2-chloro- 2-oxo-acetate | ¹H NMR (DMSO-d₆, 400 MHz) δ ppm 9.03-9.12 (m, 1H), 7.94- 8.01 (m, 2H), 7.82-7.88 (m, 1H), 7.44-7.52 (m, 1H), 7.13-7.19 (m, 1H), 7.00-7.06 (m, 1H), 6.96- 7.00 (m, 1H), 4.26-4.34 (m, 2H), 4.14-4.24 (m, 2H), 3.52-3.62 (m, 2H), 2.40 (s, 3H), 1.18-1.27 (m, 3H). MS obsd. (ESI⁺) [(M + H)⁺]: 430.1. |

### Example 110: 2-[[1-[[2-(8-chloro-4-oxo-chromen-2-yl)-5-(trifluoromethyl)phenoxy]methyl]cyclopropyl]amino]-2-oxo-acetic acid

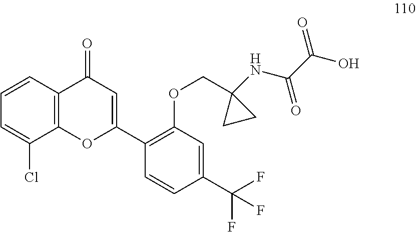 [SMILES: O=C(O)C(=O)NC1(COc2cc(C(F)(F)F)ccc2-c2cc(=O)c3cccc(Cl)c3o2)CC1]

### Step 1: Preparation of tert-butyl N-[1-[[2-(8-chloro-4-oxo-chromen-2-yl)-5-(trifluoromethyl)phenoxy]methyl]cyclopropyl]carbamate

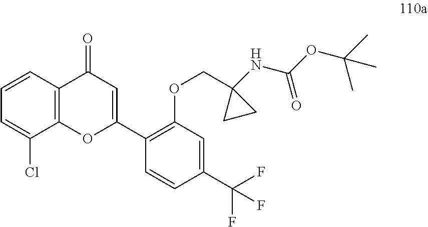 [SMILES: CC(C)(C)OC(=O)NC1(COc2cc(C(F)(F)F)ccc2-c2cc(=O)c3cccc(Cl)c3o2)CC1]

To a solution of 8-chloro-2-[2-hydroxy-4-(trifluoromethyl)phenyl]chromen-4-one (300 mg, 0.88 mmol, as the “CORE” in Table 6) and tert-butyl (1-(bromomethyl)cyclopropyl)carbamate (286 mg, 1.14 mmol, as the “LINKER” in Table 6) in DMF (4 mL) was added K₂CO₃ (609 mg, 4.4 mmol) and the mixture was stirred at 80° C. overnight. After the reaction was completed, the reaction mixture was partitioned between EtOAc (20 mL) and water (30 mL). The organic layer was separated out and the aqueous phase was extracted with EtOAc (30 mL) twice. The combined organic layer was concentrated in vacuo to give tert-butyl N-[1-[[2-(8-chloro-4-oxo-chromen-2-yl)-5-(trifluoromethyl)phenoxy]methyl]cyclopropyl]carbamate (200 mg, yield: 44.5%) as a yellow solid. MS obsd. (ESI⁺) [(M+H)⁺]: 510.1.

### Step 2: Preparation of 2-[2-[(1-aminocyclopropyl)methoxy]-4-(trifluoromethyl)phenyl]-8-chloro-chromen-4-one

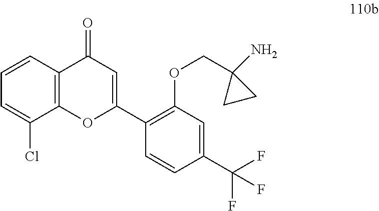 [SMILES: NC1(COc2cc(C(F)(F)F)ccc2-c2cc(=O)c3cccc(Cl)c3o2)CC1]

Compound tert-butyl N-[1-[[2-(8-chloro-4-oxo-chromen-2-yl)-5-(trifluoromethyl)phenoxy]methyl]cyclopropyl]carbamate (200 mg, 0.392 mmol) was dissolved in the DCM (5 mL), followed by adding TFA (1 mL, 13 mmol) dropwise. The reaction was stirred at room temperature for 2 hours. Then, the reaction mixture was concentrated in vacuo to give 2-[2-[(1-aminocyclopropyl)methoxy]-4-(trifluoromethyl)phenyl]-8-chloro-chromen-4-one (130 mg, yield: 80.9%) as a yellow foam. MS obsd. (ESI⁺) [(M+H)⁺]: 410.1.

### Step 3: Preparation of ethyl 2-[[1-[[2-(8-chloro-4-oxo-chromen-2-yl)-5-(trifluoromethyl)phenoxy]methyl]cyclopropyl]amino]-2-oxo-acetate

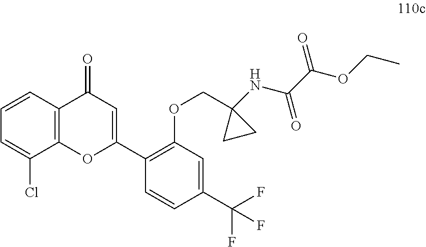 [SMILES: CCOC(=O)C(=O)NC1(COc2cc(C(F)(F)F)ccc2-c2cc(=O)c3cccc(Cl)c3o2)CC1]

To a solution of 2-[2-[(1-aminocyclopropyl)methoxy]-4-(trifluoromethyl)phenyl]-8-chloro-chromen-4-one (150 mg, 0.366 mmol) and DIPEA (128 μL, 0.732 mmol) in DCM (5 mL) was added ethyl 2-chloro-2-oxoacetate (46 μL, 0.403 mmol, as the “TAIL” in Table 6) at 0° C. The reaction was stirred at room temperature for 2 hours. After the reaction was completed, the reaction was adjusted to pH-4 by addition of 4N HCl and then extracted with EtOAc (50 mL) three times. The combined organic layer was washed with brine, dried over anhydrous Na₂SO₄ and concentrated in vacuo to give ethyl 2-[[1-[[2-(8-chloro-4-oxo-chromen-2-yl)-5-(trifluoromethyl)phenoxy]methyl]cyclopropyl]amino]-2-oxo-acetate (142 mg, yield: 76.13%) as a light yellow foam. MS obsd. (ESI⁺) [(M+H)⁺]: 510.1.

### Step 4: Preparation of 2-[[1-[[2-(8-chloro-4-oxo-chromen-2-yl)-5-(trifluoromethyl)phenoxy]methyl]cyclopropyl]amino]-2-oxo-acetic acid

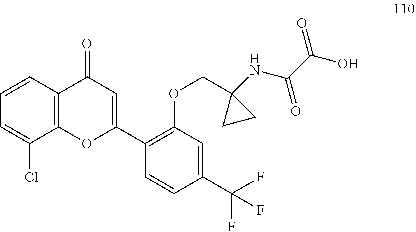 [SMILES: O=C(O)C(=O)NC1(COc2cc(C(F)(F)F)ccc2-c2cc(=O)c3cccc(Cl)c3o2)CC1]

To a solution of ethyl 2-[[1-[[2-(8-chloro-4-oxo-chromen-2-yl)-5-(trifluoromethyl)phenoxy]methyl]cyclopropyl]amino]-2-oxo-acetate (100 mg, 196 μmol) in THF (5 mL) and water (5 mL) was added LiOH (28.2 mg, 1.18 mmol). The mixture was stirred at room temperature for 2 hours. After the reaction was completed, the reaction was adjusted to pH-4 by addition of 4N HCl and extracted with EtOAc (50 mL) three times. The combined organic layer was washed with brine, dried over anhydrous Na₂SO₄ and concentrated in vacuo to give the crude product. The crude product was purified by Pre-HPLC to give 2-[[1-[[2-(8-chloro-4-oxo-chromen-2-yl)-5-(trifluoromethyl)phenoxy]methyl]cyclopropyl]amino]-2-oxo-acetic acid (25 mg, yield: 23.8%) as a yellow foam. MS obsd. (ESI⁺) [(M+H)⁺]: 482.1.

Example 110: ¹H NMR (DMSO-d₆, 400 MHz) δ ppm 9.05-9.12 (m, 1H), 8.07-8.15 (m, 1H), 7.97-8.07 (m, 2H), 7.47-7.63 (m, 3H), 7.05-7.15 (m, 1H), 4.33-4.44 (m, 2H), 0.82-0.99 (m, 4H).

The following compounds 111 to 125 were prepared in analogy to the procedure described for the preparation of Example 110, replacing 8-chloro-2-[2-hydroxy-4-(trifluoromethyl)phenyl]chromen-4-one with “CORE”, tert-butyl (1-(bromomethyl)cyclopropyl)carbamate with “LINKER” in Step 1, replacing ethyl 2-chloro-2-oxoacetate with “TAIL” in Step 3, by the reagent indicated in Table 6.

TABLE 6
Compounds synthesis and characterization

| | | CORE, | |
|---|---|---|---|
| Example | | LINKER and | |
| No. | Compounds Name and Structure | TAIL | ¹H NMR and (ESI⁺) |
| 111 | 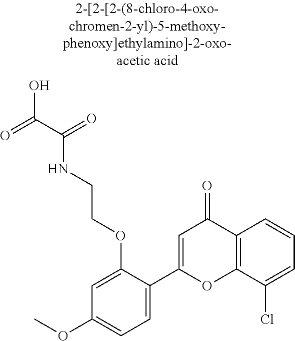 [SMILES: COc1ccc(-c2cc(=O)c3cccc(Cl)c3o2)c(OCCNC(=O)C(=O)O)c1] | CORE: Int-10 LINKER: tert-butyl N- (2- bromoethyl) carbamate TAIL: ethyl 2-chloro- 2-oxo-acetate | ¹H NMR (DMSO-d₆, 400 MHz) δ ppm 13.89 (s, 1H), 9.01 (s, 1H), 8.00-7.93 (m, 3H), 7.47 (t, J = 7.9 Hz, 1H), 7.00 (s, 1H), 6.82 (d, J = 11.9 Hz, 2H), 4.30 (t, J = 6.0 Hz, 2H), 3.88 (s, 3H), 3.58-3.55 (m, 2H). MS obsd. (ESI⁺) [(M + H)⁺]: 418.1. |
| 112 | 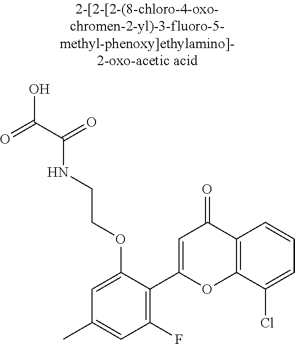 [SMILES: Cc1cc(F)c(-c2cc(=O)c3cccc(Cl)c3o2)c(OCCNC(=O)C(=O)O)c1] | CORE: Int-18 LINKER: tert-butyl N- (2- bromoethyl) carbamate TAIL: ethyl 2-chloro- 2-oxo-acetate | ¹H NMR (DMSO-d₆, 400 MHz) δ ppm 13.64-13.88 (m, 1H), 8.88 (br t, J = 5.6 Hz, 1H), 8.00 (dd, J = 14.9, 7.3 Hz, 2H), 7.51 (t, J = 7.9 Hz, 1H), 6.96-7.02 (m, 1H), 6.82-6.92 (m, 1H), 6.51- 6.60 (m, 1H), 4.13-4.22 (m, 2H), 3.41-3.50 (m, 2H), 2.39 (s, 3H). MS obsd. (ESI⁺) [(M + H)⁺]: 420.0. |
| 113 | 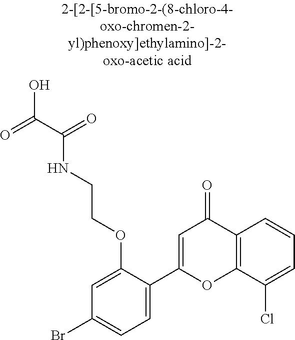 [SMILES: O=C(O)C(=O)NCCOc1cc(Br)ccc1-c1cc(=O)c2cccc(Cl)c2o1] | CORE: Int-4 LINKER: tert-butyl N- (2- bromocthyl) carbamate TAIL: ethyl 2-chloro- 2-oxo-acetate | ¹H NMR (DMSO-d₆, 400 MHz) δ ppm 13.75-13.93 (m, 1H), 9.01 (br t, J = 5.4 Hz, 1H), 7.99 (dd, J = 7.9, 2.4 Hz, 2H), 7.87 (d, J = 8.4 Hz, 1H), 7.56 (d, J = 1.0 Hz, 1H), 7.37-7.53 (m, 2H), 7.01 (s, 1H), 4.34 (br t, J = 5.6 Hz, 2H), 3.56 (q, J = 5.7 Hz, 2H). MS obsd. (ESI⁺) [(M + H)⁺]: 466.0. |
| 114 | 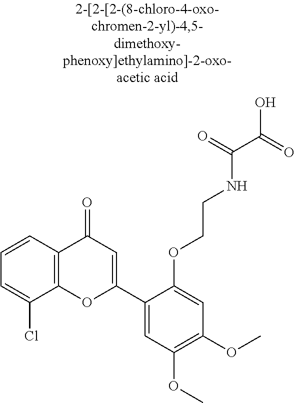 [SMILES: COc1cc(OCCNC(=O)C(=O)O)c(-c2cc(=O)c3cccc(Cl)c3o2)cc1OC] | CORE: Int-8 LINKER: tert-butyl N- (2- bromoethyl) carbamate TAIL: ethyl 2-chloro- 2-oxo-acetate | ¹H NMR (DMSO-d₆, 400 MHz) δ ppm 8.98 (br s, 1H), 7.97 (br d, J = 7.5 Hz, 2H), 7.57 (s, 1H), 7.39-7.50 (m, 1H), 7.06 (s, 1H), 6.92 (s, 1H), 4.30 (br t, J = 5.4 Hz, 2H), 3.91 (s, 3H), 3.81 (s, 3H), 3.57 (t, J = 5.9 Hz, 2H). MS obsd. (ESI⁺) [(M + H)⁺]: 448.0. |
| 115 | 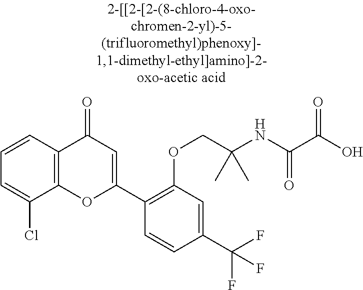 [SMILES: CC(C)(COc1cc(C(F)(F)F)ccc1-c1cc(=O)c2cccc(Cl)c2o1)NC(=O)C(=O)O] | CORE: Int-1 LINKER: tert-butyl N- (2-bromo-1,1- dimethyl- ethyl)carbamate TAIL: ethyl 2-chloro- 2-oxo-acetate | ¹H NMR (DMSO-d₆, 400 MHz) δ ppm 8.24-8.33 (m, 1H), 8.07- 8.15 (m, 1H), 7.96-8.06 (m, 2H), 7.47-7.63 (m, 3H), 6.97-7.08 (m, 1H), 4.32-4.43 (m, 2H), 1.41 (s, 6H). MS obsd. (ESI⁺) [(M + H)⁺]: 484.1. |
| 116 | 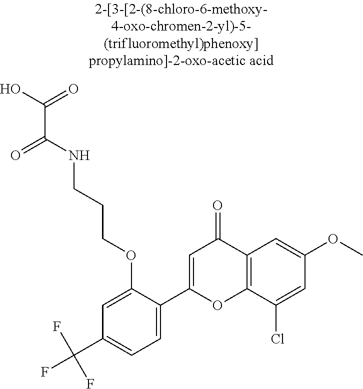 [SMILES: COc1cc(Cl)c2oc(-c3ccc(C(F)(F)F)cc3OCCCNC(=O)C(=O)O)cc(=O)c2c1] | CORE: Int-16 LINKER: tert-bulyl N- (3- bromopropyl) carbamate TAIL: ethyl 2-chloro- 2-oxo-acetate | ¹H NMR (DMSO-d₆, 400 MHz) δ ppm 13.60-13.89 (m, 1H), 8.95 (brs, 1H), 8.10 (d, J = 8.3 Hz, 1H), 7.70 (d, J = 2.9 Hz, 1H), 7.53-7.62 (m, 2H), 7.42 (d, J = 3.1 Hz, 1H), 7.08 (s, 1H), 4.20- 4.31 (m, 2H), 3.82-3.95 (m, 3H), 3.26-3.31 (m, 2H), 1.92-2.02 (m, 2H). MS obsd. (ESI⁺) [(M + H)⁺]: 500.1. |
| 117 | 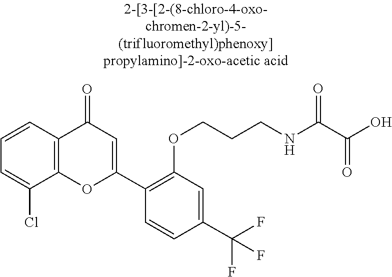 [SMILES: O=C(O)C(=O)NCCCOc1cc(C(F)(F)F)ccc1-c1cc(=O)c2cccc(Cl)c2o1] | CORE: Int-1 LINKER: tert-butyl N- (3- bromopropyl) carbamate TAIL: ethyl 2-chloro- 2-oxo-acetate | ¹H NMR (DMSO-d₆, 400 MHz) δ ppm 8.89 (s, 1H), 8.11 (d, J = 8.1 Hz, 1H), 8.00 (d, J = 7.9 Hz, 2H), 7.64-7.41 (m, 3H),7.13 (s, 1H), 4.25 (t, J = 6.1 Hz, 2H), 3.30 (d, J = 6.2 Hz, 2H), 2.09- 1.89 (m, 2H). MS obsd. (ESI⁺) [(M + H)⁺]: 470.1. |
| 118 | 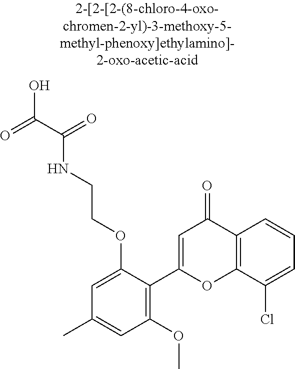 [SMILES: COc1cc(C)cc(OCCNC(=O)C(=O)O)c1-c1cc(=O)c2cccc(Cl)c2o1] | CORE: Int-19 LINKER: tert-butyl N- (2- bromoethyl) carbamate TAIL: ethyl 2-chloro- 2-oxo-acetate | ¹H NMR (DMSO-d₆, 400 MHz) δ ppm 8.70 (br s, 1H), 7.72 (dd, J = 7.9, 1.1 Hz, 1H), 7.55 (dd, J = 7.5, 1.2 Hz, 1H), 7.39 (s, 1H), 7.30 (t, J = 7.8 Hz, 1H), 7.14 (s, 1H), 6.75 (s, 1H), 3.96-4.03 (m, 2H), 3.90 (s, 3H), 3.32-3.45 (m, 2H), 2.46 (s, 3H). MS obsd. (ESI⁺) [(M + H)⁺]: 432.1. |
| 119 | 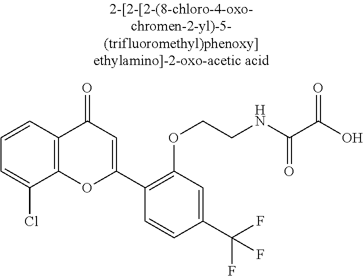 [SMILES: O=C(O)C(=O)NCCOc1cc(C(F)(F)F)ccc1-c1cc(=O)c2cccc(Cl)c2o1] | CORE: Int-1 LINKER: tert-butyl N- (2- bromoethyl) carbamate TAIL: ethyl 2-chloro- 2-oxo-acetate | ¹H NMR (DMSO-d₆, 400 MHz) δ ppm 13.54-13.88 (m, 1H), 9.02 (t, J = 5.6 Hz, 1H), 8.12 (d, J = 7.8 Hz, 1H), 8.01 (dq, J = 7.9, 1.7 Hz, 2H), 7.45-7.66 (m, 3H), 7.05 (s, 1H), 4.41 (t, J = 5.9 Hz, 2H), 3.51-3.63 (m, 2H). MS obsd. (ESI⁺) [(M + H)⁺]: 456.1. |
| 120 | 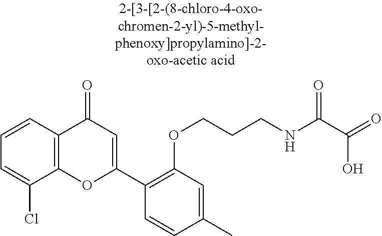 [SMILES: Cc1ccc(-c2cc(=O)c3cccc(Cl)c3o2)c(OCCCNC(=O)C(=O)O)c1] | CORE: Int-2 LINKER: tert-butyl N- (3- bromopropyl) carbamate TAIL: ethyl 2-chloro- 2-oxo-acetate | ¹H NMR (DMSO-d₆, 400 MHz) δ ppm 8.91-9.01 (m, 1H), 7.95- 8.03 (m, 2H), 7.79-7.91 (m, 1H), 7.44-7.53 (m, 1H), 7.06-7.12 (m, 2H), 6.98-7.06 (m, 1H), 4.08- 4.21 (m, 2H), 3.30-3.35 (m, 2H), 2.43 (s, 3H), 1.95-2.07 (m, 2H). MS obsd. (ESI⁺) [(M + H)⁺]: 416.1. |
| 121 | 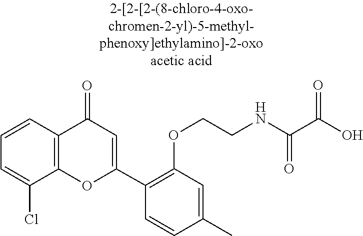 [SMILES: Cc1ccc(-c2cc(=O)c3cccc(Cl)c3o2)c(OCCNC(=O)C(=O)O)c1] | CORE: Int-2 LINKER: tert-butyl N- (2- bromoethyl) carbamate TAIL: ethyl 2-chloro- 2-oxo-acetate | ¹H NMR (DMSO-d₆, 400 MHz) δ ppm 8.94-9.09 (m, 1H), 7.94- 8.04 (m, 2H), 7.83-7.91 (m, 1H), 7.44-7.55 (m, 1H), 7.12-7.20 (m, 1H), 6.96-7.07 (m, 2H), 4.24- 4.33 (m, 2H), 3.55-3.65 (m, 2H), 2.44 (s, 3H). MS obsd. (ESI⁺) [(M + H)⁺]: 402.1. |
| 122 | 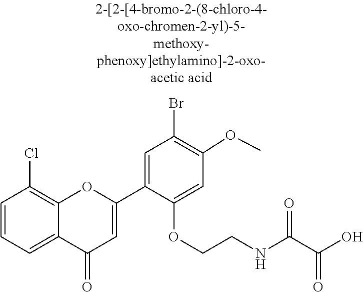 [SMILES: COc1cc(OCCNC(=O)C(=O)O)c(-c2cc(=O)c3cccc(Cl)c3o2)cc1Br] | CORE: Int-11 LINKER: tert-butyl N- (2- bromoethyl) carbamate TAIL: ethyl 2-chloro- 2-oxo-acetate | ¹H NMR (DMSO-d6, 400 MHz) δ ppm 13.88 (s, 1H), 9.08 (s, 1H), 8.12 (s, 1H), 8.03-7.91 (m, 2H), 7.48 (t, J = 7.8 Hz, 1H), 7.00 (d, J = 5.4 Hz, 2H), 4.39 (t, J = 5.8 Hz, 2H), 4.00 (s, 3H), 3.60 (d, J = 5.7 Hz, 2H). MS obsd. (ESI⁺) [(M + H)⁺]: 496.0. |
| 123 | 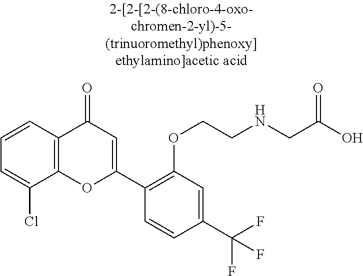 [SMILES: O=C(O)CNCCOc1cc(C(F)(F)F)ccc1-c1cc(=O)c2cccc(Cl)c2o1] | CORE: Int-1 LINKER: tert-butyl N- (2- bromoethyl) carbamate TAIL: methyl 2- bromoacetate | ¹H NMR (DMSO-d₆, 400 MHz) δ ppm 9.17 (br s, 1H), 8.02-8.12 (m, 3H), 7.63 (d, J = 8.2 Hz, 1H), 7.62 (s, 1H), 7.54 (t, J = 7.9 Hz, 1H), 7.07 (s, 1H), 4.57 (t, J = 5.0 Hz, 2H), 3.95 (s, 2H), 3.43- 3.54 (m, 2H). MS obsd. (ESI⁺) [(M + H)⁺]: 442.3. |
| 124 | 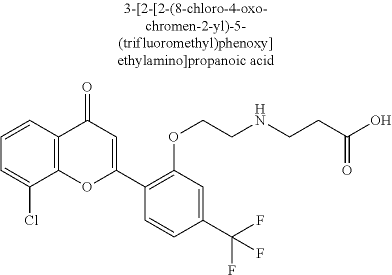 [SMILES: O=C(O)CCNCCOc1cc(C(F)(F)F)ccc1-c1cc(=O)c2cccc(Cl)c2o1] | CORE: Int-1 AMINE: tert- butyl N-(2- bromoethyl) carbamate TAIL: methyl 3- bromo- propanoate | ¹H NMR (DMSO-d₆, 400 MHz) δ ppm 8.10-8.17 (m, 1H), 8.02 (d, J = 8.2 Hz, 2H), 7.49-7.61 (m, 3H), 7.19 (s, 1H), 4.35 (t, J = 5.3 Hz, 2H), 3.04 (m, 2H), 2.83 (t, J = 6.7 Hz, 3H), 2.22- 2.45 (m, 2H). MS obsd. (ESI⁺) [(M + H)⁺]: 456.3. |
| 125 | 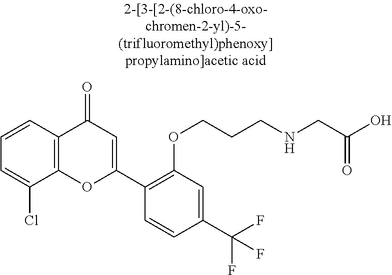 [SMILES: O=C(O)CNCCCOc1cc(C(F)(F)F)ccc1-c1cc(=O)c2cccc(Cl)c2o1] | CORE: Int-1 LINKER: tert-butyl N- (3- bromopropyl) carbamate TAIL: methyl 2- bromoacetate | ¹H NMR (DMSO-d₆, 400 MHz) δ ppm 8.05-8.02 (m, 3H), 7.49- 7.61 (m, 3H), 7.03 (s, 1H), 4.33 (t, J = 6.3 Hz, 2H), 3.13 (m, 2H), 2.95 (t, J = 7.4 Hz, 2H), 2.11 (m, 2H). MS obsd. (ES1⁺) L(M + H)⁺]: 456.3. |

### Example 126

### 2-oxo-2-[(3S)-3-[2-(8-chloro-4-oxo-chromen-2-yl)-5-(trifluoromethyl)phenoxy]pyrrolidin-1-yl]acetic acid

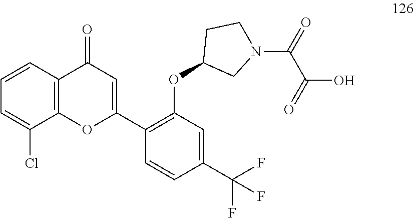 [SMILES: O=C(O)C(=O)N1CC[C@H](Oc2cc(C(F)(F)F)ccc2-c2cc(=O)c3cccc(Cl)c3o2)C1]

### Step 1: Preparation of tert-butyl (3S)-3-[2-(8-chloro-4-oxo-chromen-2-yl)-5-(trifluoromethyl)phenoxy]pyrrolidine-1-carboxylate

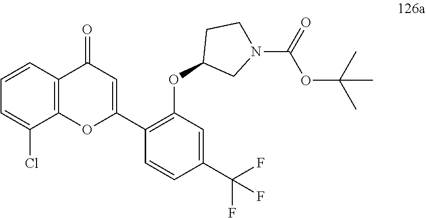 [SMILES: CC(C)(C)OC(=O)N1CC[C@H](Oc2cc(C(F)(F)F)ccc2-c2cc(=O)c3cccc(Cl)c3o2)C1]

To a solution of 8-chloro-2-[2-hydroxy-4-(trifluoromethyl)phenyl]chromen-4-one (300 mg, 881 μmol, as the “CORE” in Table 7) and tert-butyl (S)-3-((methylsulfonyl)oxy)pyrrolidine-1-carboxylate (421 mg, 1.59 mmol, CAS registry number: 127423-61-4, Vendor: Bide Pharmatech, Catalog number: BD265247, as the “CYC” in Table) in DMF (4 mL) was added K₂CO₃ (609 mg, 4.4 mmol). The reaction mixture was stirred at 80° C. overnight. After the reaction went completed, EtOAc and water were poured into the reaction mixture. The aqueous layer was extracted with EtOAc (20 mL) three times, and the organic layer was combined, dried over anhydrous Na₂SO₄, filtered and concentrated in vacuo to afford tert-butyl (3S)-3-[2-(8-chloro-4-oxo-chromen-2-yl)-5-(trifluoromethyl)phenoxy]pyrrolidine-1-carboxylate (450 mg, yield: 100%) as a yellow solid. MS obsd. (ESI⁺) [(M+H)⁺]: 510.1.

### Step 2: Preparation of 8-chloro-2-[2-[(3S)-pyrrolidin-3-yl]oxy-4-(trifluoromethyl)phenyl]chromen-4-one

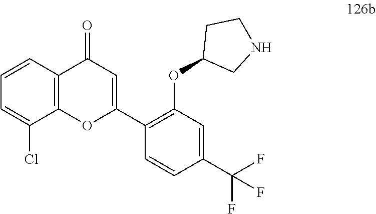 [SMILES: O=c1cc(-c2ccc(C(F)(F)F)cc2O[C@H]2CCNC2)oc2c(Cl)cccc12]

To a solution of tert-butyl (3S)-3-[2-(8-chloro-4-oxo-chromen-2-yl)-5-(trifluoromethyl)phenoxy]pyrrolidine-1-carboxylate (450 mg, 883 μmol) in DCM (5 mL) was added TFA (10 mL, 130 mmol). The reaction was stirred at room temperature for 2 hours. After the reaction was completed, the reaction was adjusted to pH-4 by addition of 4N HCl and then extracted with EtOAc (50 mL) three times. The combined organic layer was washed with brine, dried over anhydrous Na₂SO₄ and concentrated in vacuo to give 8-chloro-2-[2-[(3S)-pyrrolidin-3-yl]oxy-4-(trifluoromethyl)phenyl]chromen-4-one (350 mg, yield: 96.8%) as a light yellow foam. MS obsd. (ESI⁺) [(M+H)⁺]: 410.1.

### Step 3: Preparation of ethyl 2-oxo-2-[(3S)-3-[2-(8-chloro-4-oxo-chromen-2-yl)-5-(trifluoromethyl)phenoxy]pyrrolidin-1-yl]acetate

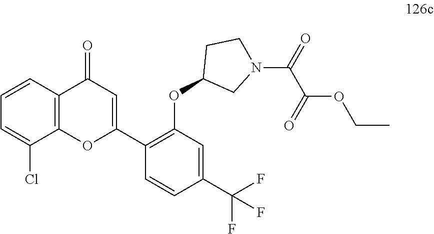 [SMILES: CCOC(=O)C(=O)N1CC[C@H](Oc2cc(C(F)(F)F)ccc2-c2cc(=O)c3cccc(Cl)c3o2)C1]

To a solution of (3S)-3-[2-(8-chloro-4-oxo-chromen-2-yl)-5-(trifluoromethyl)phenoxy]pyrrolidine-1-carboxylate (300 mg, 732 μmol) and DIPEA (256 μL, 1.46 mmol) in DCM (5 mL) was added ethyl 2-chloro-2-oxoacetate (91.6 μL, 805 μmol, as the “TAIL” in Table 7) at 0° C. The reaction was stirred at room temperature for 2 hours. After the reaction was completed, the reaction was adjusted to pH-4 by addition of 4N HCl and then extracted with EtOAc (50 mL) three times. The combined organic layer was washed with brine, dried over anhydrous Na₂SO₄ and concentrated in vacuo to give ethyl 2-oxo-2-[(3S)-3-[2-(8-chloro-4-oxo-chromen-2-yl)-5-(trifluoromethyl)phenoxy]pyrrolidin-1-yl]acetate (300 mg, yield: 80.4%) as a light yellow foam. MS obsd. (ESI⁺) [(M+H)⁺]: 510.1.

### Step 4: Preparation of 2-oxo-2-[(3S)-3-[2-(8-chloro-4-oxo-chromen-2-yl)-5-(trifluoromethyl)phenoxy]pyrrolidin-1-yl]acetic acid

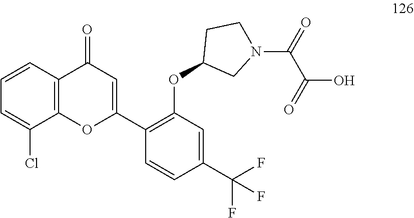 [SMILES: O=C(O)C(=O)N1CC[C@H](Oc2cc(C(F)(F)F)ccc2-c2cc(=O)c3cccc(Cl)c3o2)C1]

To a solution of ethyl 2-oxo-2-[(3S)-3-[2-(8-chloro-4-oxo-chromen-2-yl)-5-(trifluoromethyl)phenoxy]pyrrolidin-1-yl]acetate (300 mg, 588 μmol) in THF (5 mL) and water (5 mL) was added LiOH (84.5 mg, 3.53 mmol). The reaction mixture was stirred at room temperature for 2 hours. After the reaction was completed, the reaction was adjusted to pH-4 by addition of 4N HCl and then extracted with EtOAc (50 mL) three times. The combined organic layer was washed with brine, dried over anhydrous Na₂SO₄ and concentrated in vacuo to give the crude product, which was purified by Pre-HPLC to give 2-oxo-2-[(3S)-3-[2-(8-chloro-4-oxo-chromen-2-yl)-5-(trifluoromethyl)phenoxy]pyrrolidin-1-yl]acetic acid (200 mg, yield: 67%) as a yellow foam. MS obsd. (ESI⁺) [(M+H)⁺]: 482.1.

Example 126: ¹H NMR (DMSO-d₆, 400 MHz) δ ppm 8.06-8.15 (m, 1H), 7.96-8.05 (m, 2H), 7.65-7.70 (m, 1H), 7.57-7.64 (m, 1H), 7.46-7.55 (m, 1H), 6.89-6.97 (m, 1H), 5.42-5.53 (m, 1H), 3.90-4.00 (m, 1H), 3.72-3.83 (m, 1H), 3.60-3.68 (m, 1H), 3.36-3.48 (m, 1H), 2.12-2.36 (m, 2H).

The following compounds 127 to 133 were prepared in analogy to the procedure described for the preparation of Example 126, replacing 8-chloro-2-[2-hydroxy-4-(trifluoromethyl)phenyl]chromen-4-one with “CORE”, and tert-butyl (S)-3-((methylsulfonyl)oxy)pyrrolidine-1-carboxylate with “CYC” in Step 1 and replacing ethyl 2-chloro-2-oxo-acetate with “TAIL” in Step 3, by the reagent indicated in Table 7.

TABLE 7
Compounds synthesis and characterization

| Example | | | |
|---|---|---|---|
| No. | Compounds Name and Structure | CORE, CYC, TAIL | ¹H NMR and (ESI⁺) |
| 127 | 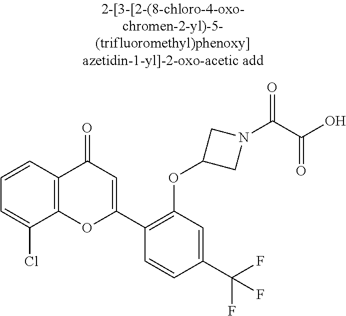 [SMILES: O=C(O)C(=O)N1CC(Oc2cc(C(F)(F)F)ccc2-c2cc(=O)c3cccc(Cl)c3o2)C1] | CORE: Int-1 CYC: tert-butyl 3- methylsulfonyloxy- azetidine-1- carboxylate (CAS registry number: 141699-58-3, Vendor: Bide Pharmatech, Catalog number: BD51861) TAIL: ethyl 2- chloro-2-oxo-acetate | ¹H NMR (DMSO-d₆, 400 MHz) δ ppm 8.11-8.19 (m, 1H), 7.99-8.08 (m, 2H), 7.57-7.66 (m, 1H), 7.46-7.57 (m, 1H), 7.22- 7.28 (m, 1H), 7.07-7.14 (m, 1H), 5.30-5.42 (m, 1H), 4.68-4.81 (m, 1H), 4.32-4.44 (m, 1H), 4.19- 4.28 (m, 1H), 3.78-3.92 (m, 1H). MS obsd. (ESI⁺) [(M + H)⁺]: 468.1. |
| 128 | 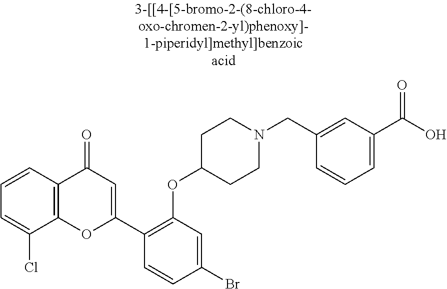 [SMILES: O=C(O)c1cccc(CN2CCC(Oc3cc(Br)ccc3-c3cc(=O)c4cccc(Cl)c4o3)CC2)c1] | CORE: Int-4 CYC: tert-butyl 4- methylsulfonyloxy- piperidine-1- carboxylate (CAS registry number: 141699-59-4, Vendor: Bide Pharmatech, Catalog number: BD13505) TAIL: methyl 3- (bromomethyl) benzoate | ¹H NMR (DMSO-d₆, 400 MHz) δ ppm 12.93- 13.43 (m, 1H), 7.96-8.22 (m, 3H), 7.73-7.95 (m, 2H), 7.61-7.72 (m, 2H), 7.32-7.60 (m, 3H), 6.96 (s, 1H), 4.77-5.11 (m, 1H), 4.29-4.56 (m, 2H), 3.46- 3.64 (m, 2H), 2.88-3.20 (m, 2H), 2.17-2.38 (m, 2H), 1.74-2.15(m,2H). MS obsd. (ESI⁺) [(M + H)⁺]: 568.1. |
| 129 | 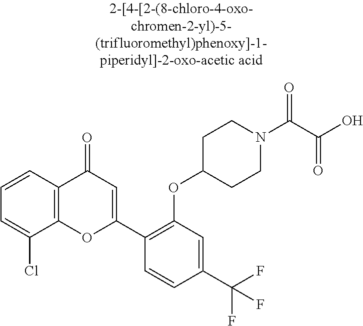 [SMILES: O=C(O)C(=O)N1CCC(Oc2cc(C(F)(F)F)ccc2-c2cc(=O)c3cccc(Cl)c3o2)CC1] | CORE: Int-1 CYC: tert-butyl 4- methylsulfonyloxy- piperidine-1- carboxylate TAIL: ethyl 2- chloro-2-oxo-acetate | ¹H NMR (DMSO-d₆, 400 MHz) δ ppm 8.07-8.14 (m, 1H), 7.98-8.06 (m, 2H), 7.64-7.70 (m, 1H), 7.47-7.58 (m, 2H), 7.01- 7.07 (m, 1H), 5.04-5.15 (m, 1H), 3.41-3.61 (m, 2H), 3.21-3.30 (m, 2H), 1.86-2.03 (m, 2H), 1.51- 1.78 (m, 2H). MS obsd. (ESI⁺) [(M + H)⁺]: 496.1. |
| 130 | 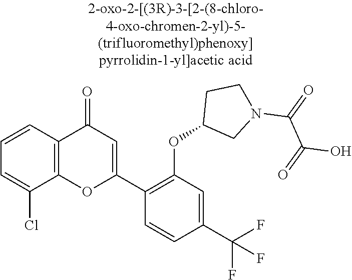 [SMILES: O=C(O)C(=O)N1CC[C@@H](Oc2cc(C(F)(F)F)ccc2-c2cc(=O)c3cccc(Cl)c3o2)C1] | CORE: Int-1 CYC: tert-butyl (R)- 3- ((methylsulfonyl) oxy)pyrrolidine-1- carboxylate TAIL: ethyl 2- chloro-2-oxo-acetate | ¹H NMR (DMSO-d₆, 400 MHz) δ ppm 8.07-8.15 (m, 1H), 7.97-8.05 (m, 2H), 7.65-7.72 (m, 1H), 7.57-7.64 (m, 1H), 7.47- 7.56 (m, 1H), 6.88-7.00 (m, 1H), 5.40-5.55 (m, 1H), 3.69-3.84 (m, 2H), 3.56-3.68 (m, 2H), 2.12- 2.39 (m, 2H). MS obsd. (ESI⁺) [(M + H)⁺]: 482.1. |
| 131 | 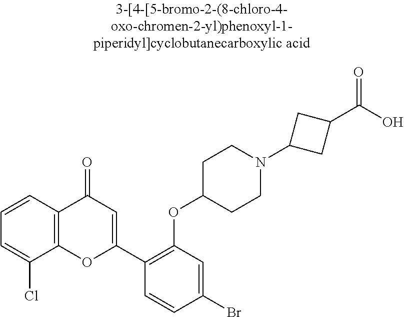 [SMILES: O=C(O)C1CC(N2CCC(Oc3cc(Br)ccc3-c3cc(=O)c4cccc(Cl)c4o3)CC2)C1] | CORE: Int-4 CYC: tert-butyl 4- methylsulfonyloxy- piperidine-1- carboxylate TAIL: methyl 3- chlorocyclobutane- carboxylate | ¹H NMR (DMSO-d₆, 400 MHz) δ ppm 11.57- 12.62 (m, 1H), 7.99-8.13 (m, 2H), 7.79-7.93 (m, 1H), 7.41-7.74 (m, 3H), 6.90-7.02 (m, 1H), 4.78- 5.13 (m, 1H), 3.43-4.02 (m, 3H), 2.74-3.12 (m, 3H), 2.12-2.41 (m, 4H), 1.92-2.11 (m, 1H), 1.66- 1.91 (m, 1H), 1.00-1.51 (m, 2H). MS obsd. (ESI⁺) [(M + H)⁺]: 532.0. |
| 132 | 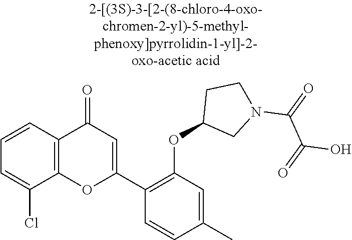 [SMILES: Cc1ccc(-c2cc(=O)c3cccc(Cl)c3o2)c(O[C@H]2CCN(C(=O)C(=O)O)C2)c1] | CORE: Int-2 CYC: tert-butyl (S)- 3- ((methylsulfonyl) oxy)pyrrolidine-1- carboxylate TAIL: ethyl 2- chloro-2-oxo-acetate | ¹H NMR (DMSO-d₆, 400 MHz) δ ppm 7.94-8.02 (m, 2H), 7.79-7.89 (m, 1H), 7.44-7.55 (m, 1H), 7.15-7.23 (m, 1H), 6.97- 7.08 (m, 1H), 6.86-6.94 (m, 1H), 5.22-5.36 (m, 1H), 3.54-3.98 (m, 4H), 2.44 (s, 3H), 2.13-2.36 (m, 2H). MS obsd. (ESI⁺) [(M + H)⁺]: 428.1. |
| 133 | 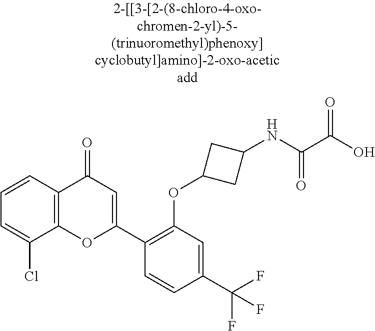 [SMILES: O=C(O)C(=O)NC1CC(Oc2cc(C(F)(F)F)ccc2-c2cc(=O)c3cccc(Cl)c3o2)C1] | CORE: Int-1 CYC: [3-(tert- butoxycarbonyl- amino)cyclobutyl] methanesulfonate TAIL: ethyl 2- chloro-2-oxo-acetate | ¹H NMR (DMSO-d⁶, 400 MHz) δ ppm 7.41 (d, J = 8.2 Hz, 1H), 7.28 (dd, J = 8.1, 1.6 Hz, 1H), 7.06 (dd, J = 7.8, 1.5 Hz, 1H), 6.89 (s, 1H), 6.64 (t, J = 7.9 Hz, 2H), 6.52 (s, 1H), 6.40 (s, 1H), 3.94 (m, 1H), 3.36 (br t, J =7.9 Hz, 1H), 2.25 (m, 2H), 1.49- 1.66 (m, 2H). MS obsd. (ESI⁺) [(M + H)⁺]: 482.0. |

### Example 134: 2-[3-[2-(8-chloro-4-oxo-chromen-2-yl)-5-(trifluoromethyl)phenoxy]propyl-methyl-amino]acetic acid

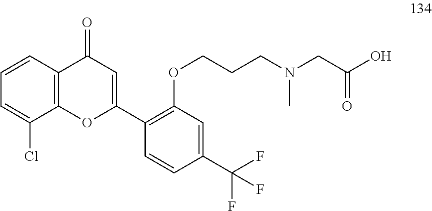 [SMILES: CN(CCCOc1cc(C(F)(F)F)ccc1-c1cc(=O)c2cccc(Cl)c2o1)CC(=O)O]

To a solution of 2-[3-[2-(8-chloro-4-oxo-chromen-2-yl)-5-(trifluoromethyl)phenoxy]propylamino]acetic acid (200 mg, 439 μmol, Example 125) in THF (9 mL) was added formaldehyde (2 mL, 26.9 mmol). The reaction mixture was stirred for 30 minutes and then added sodium cyanoborohydride (138 mg, 2.19 mmol). After stirring for 2 hours, the reaction mixture was diluted with water (30 mL), and the aqueous layer was extracted with EtOAc (10 mL) three times. The combined organic layer was washed with brine, dried over anhydrous Na₂SO₄, filtered and concentrated in vacuo to give the crude product, which was purified by prep-HPLC to afford 2-[3-[2-(8-chloro-4-oxo-chromen-2-yl)-5-(trifluoromethyl)phenoxy]propyl-methyl-amino]acetic acid (66 mg, yield: 30.7%). MS obsd. (ESI⁺) [(M+H)⁺]: 470.4.

Example 134: ¹H NMR (DMSO-d₆, 400 MHz) δ ppm 8.12 (d, J=7.7 Hz, 1H), 8.04 (dd, J=7.9, 1.2 Hz, 1H), 7.38-7.69 (m, 4H), 7.06 (s, 1H), 4.35 (t, J=6.1 Hz, 2H), 3.97 (s, 2H), 3.13-3.25 (m, 2H), 2.79 (s, 3H), 2.15-2.29 (m, 2H), 1.06-1.27 (m, 3H).

### Example 135: 2-[2-[2-(8-chloro-4-oxo-chromen-2-yl)-5-methyl-phenoxy]ethyl-cyclopropyl-amino]-2-oxo-acetic acid

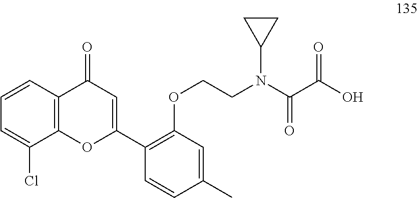 [SMILES: Cc1ccc(-c2cc(=O)c3cccc(Cl)c3o2)c(OCCN(C(=O)C(=O)O)C2CC2)c1]

### Step 1: Preparation of 2-[2-(2-bromoethoxy)-4-methyl-phenyl]-8-chloro-chromen-4-one

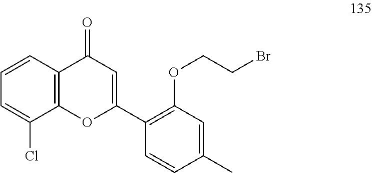 [SMILES: Cc1ccc(-c2cc(=O)c3cccc(Cl)c3o2)c(OCCBr)c1]

To a solution of 8-chloro-2-(2-hydroxy-4-methyl-phenyl)chromen-4-one (500 mg, 1.74 mmol) and 1,2-dibromoethane (750 mg, 3.99 mmol) in DMF (18 ML) was added K₂CO₃ (1.21 g, 8.72 mmol). The reaction mixture was stirred at 80° C. overnight. After the reaction was completed, the reaction mixture was partitioned between EtOAc (10 mL) and water (30 mL). The organic layer was separated out and the aqueous phase was extracted with EtOAc (30 mL) twice. The combined organic layer was concentrated in vacuo and the residue was purified by ISCO (eluting with EtOAc:PE=0 to 30%) to give 2-[2-(2-bromoethoxy)-4-methyl-phenyl]-8-chloro-chromen-4-one (416 mg, yield: 60.6%) as a yellow solid. MS obsd. (ESI⁺) [(M+H)⁺]: 394.0.

### Step 2: Preparation of 8-chloro-2-[2-[2-(cyclopropylamino)ethoxy]-4-methyl-phenyl]chromen-4-one

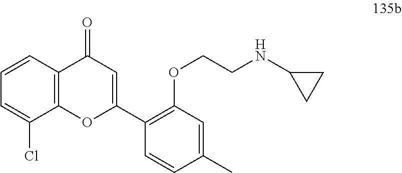 [SMILES: Cc1ccc(-c2cc(=O)c3cccc(Cl)c3o2)c(OCCNC2CC2)c1]

To a solution of 2-[2-(2-bromoethoxy)-4-methyl-phenyl]-8-chloro-chromen-4-one (400 mg, 1.02 mmol) in DMF (10 mL) was added cyclopropanamine (174 mg, 3.05 mmol) and potassium carbonate (702 mg, 5.08 mmol). The reaction was stirred at room temperature overnight. Then, the reaction mixture was quenched by adding water (50 mL), and extracted with EtOAc (30 mL) three times. The combined organic layer was washed with brine, dried over anhydrous Na₂SO₄, filtered and concentrated in vacuo to give 8-chloro-2-[2-[2-(cyclopropylamino)ethoxy]-4-methyl-phenyl]chromen-4-one (376 mg, yield: 100%). MS obsd. (ESI⁺) [(M+H)⁺]: 370.0.

### Step 3: Preparation of 2-[2-[2-(8-chloro-4-oxo-chromen-2-yl)-5-methyl-phenoxy]ethyl-cyclopropyl-amino]-2-oxo-acetic acid

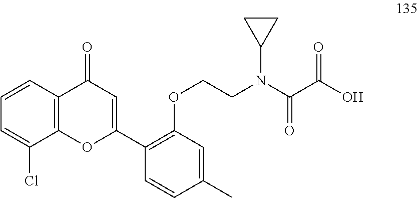 [SMILES: Cc1ccc(-c2cc(=O)c3cccc(Cl)c3o2)c(OCCN(C(=O)C(=O)O)C2CC2)c1]

To a solution of 8-chloro-2-[2-[2-(cyclopropylamino)ethoxy]-4-methyl-phenyl]chromen-4-one (0.175 g, 473 μmol) in DCM (5 mL) cooled in the ice-water bath was added DIPEA (100 μl, 573 μmol). Then, oxalyl chloride (0.5 mL, 5.71 mmol) was slowly injected into the reaction mixture. The reaction was allowed to warm to room temperature and kept stirring overnight. After the starting material was consumed, the reaction mixture was partitioned between EtOAc (10 mL) and water (20 mL). The aqueous layer was extracted with EtOAc (20 mL) twice, and the combined organic layer was washed with brine, dried over Na₂SO₄, filtered and concentrated in vacuo to give the crude product, which was purified by prep-HPLC to give 2-[2-[2-(8-chloro-4-oxo-chromen-2-yl)-5-methyl-phenoxy]ethyl-cyclopropyl-amino]-2-oxo-acetic acid (4.3 mg, yield: 1.75%). MS obsd. (ESI⁺) [(M+H)⁺]: 442.1.

Example 135: ¹H NMR (METHANOL-d₆, 400 MHz) δ ppm 9.10 (s, 1H), 7.76-8.18 (m, 4H), 7.46 (t, J=7.9 Hz, 2H), 7.28 (s, 1H), 4.69-4.80 (m, 2H), 4.45-4.56 (m, 2H), 2.46 (s, 3H), 1.26-1.40 (m, 1H), 0.73-0.97 (m, 4H).

### Example 136: 8-chloro-2-[2-[2-[ethylsulfamoyl(methyl)amino]ethoxy]-4-methyl-phenyl]-4-oxo-chromene

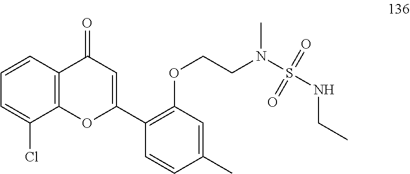 [SMILES: CCNS(=O)(=O)N(C)CCOc1cc(C)ccc1-c1cc(=O)c2cccc(Cl)c2o1]

### Step 1: Preparation of tert-butyl N-[2-[2-(8-chloro-4-oxo-chromen-2-yl)-5-methyl-phenoxy]ethyl]carbamate

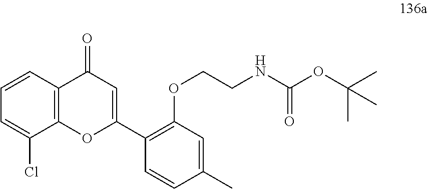 [SMILES: Cc1ccc(-c2cc(=O)c3cccc(Cl)c3o2)c(OCCNC(=O)OC(C)(C)C)c1]

To a solution of 8-chloro-2-(2-hydroxy-4-methyl-phenyl)chromen-4-one (500 mg, 1.74 mmol) and tert-butyl (3-bromopropyl)carbamate (415 mg, 1.74 mmol) in DMF (18 mL) was added K₂CO₃ (1.21 g, 8.72 mmol) and the mixture was stirred at 50° C. overnight. After the reaction was completed, the reaction mixture was partitioned between EtOAc (20 mL) and water (30 mL). The organic layer was separated out and the aqueous phase was extracted with EtOAc (30 mL) twice. The combined organic layer was concentrated in vacuo and the residue was purified by ISCO (eluting with EtOAc:PE=0 to 30%) to give tert-butyl N-[3-[2-(8-chloro-4-oxo-chromen-2-yl)-5-methyl-phenoxy]propyl]carbamate (500 mg, yield: 66.7%) as a yellow solid. MS obsd. (ESI⁺) [(M+H)⁺]: 430.1.

### Step 2: Preparation of tert-butyl N-[2-[2-(8-chloro-4-oxo-chromen-2-yl)-5-methyl-phenoxy]ethyl]-N-methyl-carbamate

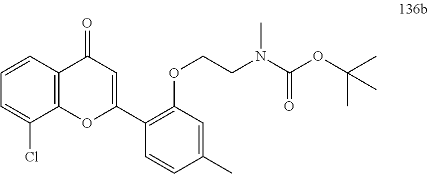 [SMILES: Cc1ccc(-c2cc(=O)c3cccc(Cl)c3o2)c(OCCN(C)C(=O)OC(C)(C)C)c1]

To a solution of N-[2-[2-(8-chloro-4-oxo-chromen-2-yl)-5-methyl-phenoxy]ethyl]carbamate (200 mg, 465 μmol) in DMF (5 mL) was added Mel (116 μl, 1.86 mmol). The reaction was cooled in the ice-water bath, and followed by adding NaH (46.5 mg, 1.16 mmol). The reaction was allowed to warm to room temperature and stirred for 2 hours. Then, the reaction was quenched by adding water (20 mL), and the aqueous layer was extracted with EtOAc (10 mL) three times. The organic layer was combined, washed with brine, dried over anhydrous Na₂SO₄, filtered and concentrated in vacuo to give N-[2-[2-(8-chloro-4-oxo-chromen-2-yl)-5-methyl-phenoxy]ethyl]-N-methyl-carbamate (200 mg, yield: 96.8%).

### Step 3: Preparation of 8-chloro-2-[4-methyl-2-[2-(methylamino)ethoxy]phenyl]chromen-4-one

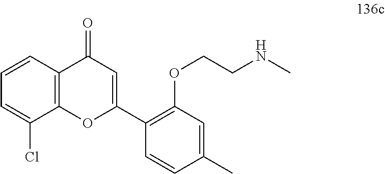 [SMILES: CNCCOc1cc(C)ccc1-c1cc(=O)c2cccc(Cl)c2o1]

To a solution of N-[2-[2-(8-chloro-4-oxo-chromen-2-yl)-5-methyl-phenoxy]ethyl]-N-methyl-carbamate (300 mg, 676 μmol) in DCM (2 mL) was added TFA (2 mL, 26 mmol). The reaction was stirred at room temperature and went complete after 2 hours. Then, the reaction mixture was concentrated in vacuo and azeotroped with toluene twice to give 8-chloro-2-[4-methyl-2-[2-(methylamino)ethoxy]phenyl]chromen-4-one (230 mg, yield: 99%) as a yellow oil. MS obsd. (ESI⁺) [(M+H)⁺]: 344.1.

### Step 4: Preparation of 8-chloro-2-[2-[2-[ethylsulfamoyl(methyl)amino]ethoxy]-4-methyl-phenyl]-4-oxo-chromene

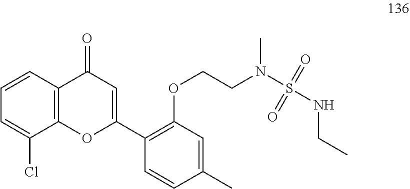 [SMILES: CCNS(=O)(=O)N(C)CCOc1cc(C)ccc1-c1cc(=O)c2cccc(Cl)c2o1]

A solution of 8-chloro-2-[4-methyl-2-[2-(methylamino)ethoxy]phenyl]chromen-4-one (120 mg, 349 μmol), DIPEA (61 μl, 349 μmol) and ethylsulfamoyl chloride (50.1 mg, 349 μmol) in DCM (4 mL) was stirred at room temperature overnight. After completion, the reaction was quenched by adding 1N HCl. The residue was then partitioned between EtOAc (10 mL) and water (20 mL). The organic layer was combined, washed with brine, dried over anhydrous Na₂SO₄, filtered and concentrated in vacuo to give the crude product, which was purified by prep-HPLC to afford 8-chloro-2-[2-[2-[ethylsulfamoyl(methyl)amino]ethoxy]-4-methyl-phenyl]-4-oxo-chromene (3.3 mg, yield: 2%) as a white solid. MS obsd. (ESI⁺) [(M+H)⁺]: 451.7.

Example 136: ¹H NMR (DMSO-d₆, 400 MHz) δ ppm 8.00 (d, J=7.8 Hz, 2H), 7.85 (d, J=7.9 Hz, 1H), 7.49 (t, J=7.9 Hz, 1H), 7.10-7.23 (m, 2H), 7.05 (s, 2H), 4.31 (t, J=5.6 Hz, 2H), 3.50 (t, J=5.7 Hz, 2H), 2.84 (dd, J=7.2, 5.7 Hz, 2H), 2.77 (s, 3H), 2.41 (s, 3H), 0.98 (t, J=7.2 Hz, 3H).

### Example 137: ethyl 2-[2-[2-(8-chloro-4-oxo-chromen-2-yl)-5-methyl-phenoxy]ethyl-methyl-amino]-2-oxo-acetate

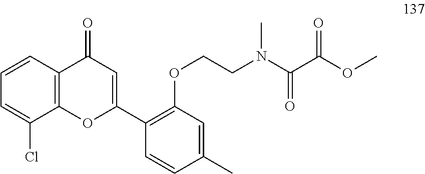 [SMILES: COC(=O)C(=O)N(C)CCOc1cc(C)ccc1-c1cc(=O)c2cccc(Cl)c2o1]

137 was prepared in analogy to the procedure described for the preparation of compound 136 by using ethyl 2-chloro-2-oxo-acetate as the starting material instead of ethylsulfamoyl chloride in Step 4. MS obsd. (ESI⁺) [(M+H)⁺]: 444.0.

Example 137: ¹H NMR (DMSO-d₆, 400 MHz) δ ppm 7.99 (d, J=7.8 Hz, 1H), 7.81 (dd, J=10.0, 8.1 Hz, 1H), 7.49 (t, J=7.9 Hz, 1H), 7.16 (d, J=9.2 Hz, 1H), 7.03 (d, J=7.8 Hz, 1H), 6.84-7.00 (m, 1H), 4.34 (br d, J=4.9 Hz, 2H), 4.04-4.17 (m, 2H), 3.68-3.83 (m, 2H), 3.17 (d, J=4.9 Hz, 3H), 2.40 (s, 3H), 1.06-1.27 (m, 3H).

### Example 138: ethyl 2-[3-[2-(8-chloro-4-oxo-chromen-2-yl)-5-(trifluoromethyl)phenoxy]propyl-ethylsulfonyl-amino]-2-oxo-acetate

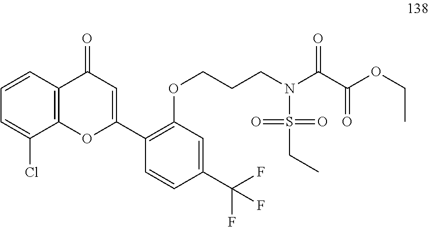 [SMILES: CCOC(=O)C(=O)N(CCCOc1cc(C(F)(F)F)ccc1-c1cc(=O)c2cccc(Cl)c2o1)S(=O)(=O)CC]

### Step 1: Preparation of N-[3-[2-(8-chloro-4-oxo-chromen-2-yl)-5-(trifluoromethyl)phenoxy]propyl]ethanesulfonamide

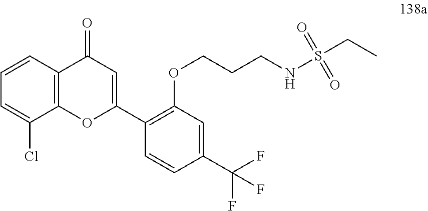 [SMILES: CCS(=O)(=O)NCCCOc1cc(C(F)(F)F)ccc1-c1cc(=O)c2cccc(Cl)c2o1]

To a solution of 2-[2-(3-bromopropoxy)-4-(trifluoromethyl)phenyl]-8-chloro-chromen-4-one (200 mg, 435 μmol, 062a) in DCM was added TEA (303 μl, 2.18 mmol) and ethanesulfonamide (238 mg, 2.18 mmol) dropwise. The reaction was stirred at room temperature overnight. After the reaction was complete, the reaction mixture was quenched by adding 1N HCl, and then partitioned between DCM (10 mL) and water (20 mL). The aqueous layer was extracted with EtOAc (10 mL) twice. The combined organic layer was washed with brine, dried over anhydrous Na₂SO₄, filtered and concentrated in vacuo to give N-[3-[2-(8-chloro-4-oxo-chromen-2-yl)-5-(trifluoromethyl)phenoxy]propyl]ethanesulfonamide (100 mg, yield: 46.9%) as a yellow solid. MS obsd. (ESI⁺) [(M+H)⁺]: 490.5.

### Step 2: Preparation of 2-[3-[2-(8-chloro-4-oxo-chromen-2-yl)-5-(trifluoromethyl)phenoxy]propyl-ethylsulfonyl-amino]-2-oxo-acetate

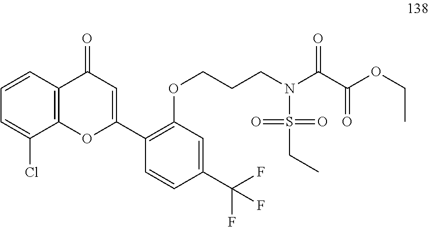 [SMILES: CCOC(=O)C(=O)N(CCCOc1cc(C(F)(F)F)ccc1-c1cc(=O)c2cccc(Cl)c2o1)S(=O)(=O)CC]

Compound N-[3-[2-(8-chloro-4-oxo-chromen-2-yl)-5-(trifluoromethyl)phenoxy]propyl]ethanesulfonamide (100 mg, 204 μmol) was dissolved in the DMF (2 mL), followed by adding ethyl 2-chloro-2-oxo-acetate (36 μL, 320 μmol) and DIPEA (56 μL, 320 μmol). The reaction was stirred at room temperature. After the reaction was completed, the reaction mixture was partitioned between ethyl acetate (10 mL) and water (20 mL). The combined organic layer was washed with brine, dried over anhydrous sodium sulfate, filtered and concentrated in vacuo to give 2-[3-[2-(8-chloro-4-oxo-chromen-2-yl)-5-(trifluoromethyl)phenoxy]propyl-ethylsulfonyl-amino]-2-oxo-acetate (100 mg, yield: 81.5%). MS obsd. (ESI⁺) [(M+H)⁺]: 590.0.

Example 138: ¹H NMR (DMSO-d₆, 400 MHz) δ ppm 7.94-8.17 (m, 3H), 7.44-7.64 (m, 3H), 7.02 (s, 1H), 4.17-4.49 (m, 4H), 3.77-3.97 (m, 2H), 3.59 (d, J=7.3 Hz, 2H), 2.03-2.21 (m, 2H), 1.11-1.40 (m, 6H).

### Example 139: 3-[3-[2-(8-chloro-4-oxo-chromen-2-yl)-5-(trifluoromethyl)phenoxy]propyl-cyclopropylsulfonyl-amino]cyclobutanecarboxylic acid

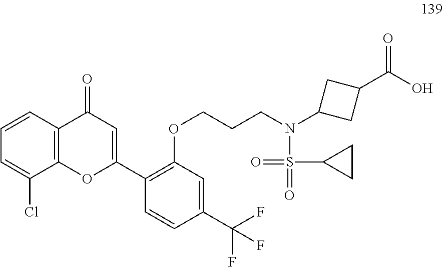 [SMILES: O=C(O)C1CC(N(CCCOc2cc(C(F)(F)F)ccc2-c2cc(=O)c3cccc(Cl)c3o2)S(=O)(=O)C2CC2)C1]

### Step 1: Preparation of methyl 3-[3-[2-(8-chloro-4-oxo-chromen-2-yl)-5-(trifluoromethyl)phenoxy]propylamino]cyclobutanecarboxylate

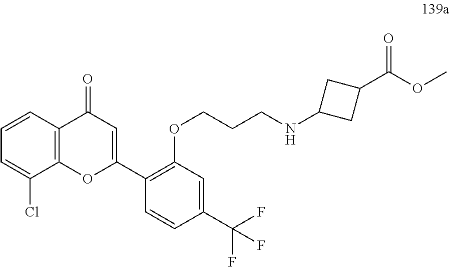 [SMILES: COC(=O)C1CC(NCCCOc2cc(C(F)(F)F)ccc2-c2cc(=O)c3cccc(Cl)c3o2)C1]

To a solution of 2-[2-(3-bromopropoxy)-4-(trifluoromethyl)phenyl]-8-chloro-chromen-4-one (200 mg, 433 μmol) and methyl 3-aminocyclobutane-1-carboxylate (280 mg, 2.17 mmol, as the “TAIL1” in Table 8) in DMF (5 mL) was added NaHCO₃ (364 mg, 4.33 mmol). The mixture was stirred at 50° C. overnight. After the reaction was completed, the reaction mixture was diluted with EtOAc and partitioned between EtOAc (30 mL) and water (30 mL). The organic layer was separated out and the aqueous phase was extracted with EtOAc (30 mL) twice. The combined organic layer was concentrated in vacuo to give methyl 3-[3-[2-(8-chloro-4-oxo-chromen-2-yl)-5-(trifluoromethyl)phenoxy]propylamino]cyclobutanecarboxylate (100 mg, yield: 45.3%) as a yellow solid.

### Preparation of 3-[3-[2-(8-chloro-4-oxo-chromen-2-yl)-5-(trifluoromethyl)phenoxy]propylamino]cyclobutanecarboxylic acid

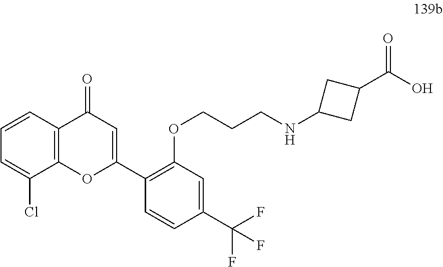 [SMILES: O=C(O)C1CC(NCCCOc2cc(C(F)(F)F)ccc2-c2cc(=O)c3cccc(Cl)c3o2)C1]

To the solution of methyl 3-[3-[2-(8-chloro-4-oxo-chromen-2-yl)-5-(trifluoromethyl)phenoxy]propylamino]cyclobutanecarboxylate (100 mg, 0.196 mmol) in mixed solvent of MeOH (5 mL), THF (5 mL) and water (2 mL) was added LiOH (23.5 mg, 0.981 mmol). The reaction mixture was stirred at room temperature till completion. Then, the reaction mixture was neutralized by acetic acid and then concentrated in vacuo. The crude material was purified by prep-HPLC to give 3-[3-[2-(8-chloro-4-oxo-chromen-2-yl)-5-(trifluoromethyl)phenoxy]propylamino]cyclobutanecarboxylic acid (65 mg, yield: 63.5%) as a white solid. (ESI⁺) [(M+H)]⁺: 495.9.

### Step 3: Preparation of 3-[3-[2-(8-chloro-4-oxo-chromen-2-yl)-5-(trifluoromethyl)phenoxy]propyl-cyclopropylsulfonyl-amino]cyclobutanecarboxylic acid

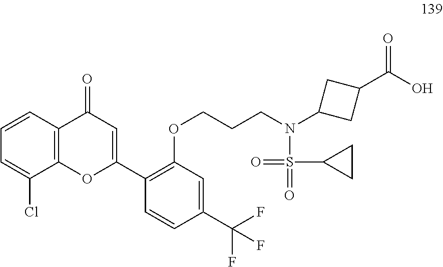 [SMILES: O=C(O)C1CC(N(CCCOc2cc(C(F)(F)F)ccc2-c2cc(=O)c3cccc(Cl)c3o2)S(=O)(=O)C2CC2)C1]

To a solution of 3-[3-[2-(8-chloro-4-oxo-chromen-2-yl)-5-(trifluoromethyl)phenoxy]propylamino]cyclobutanecarboxylic acid (100 mg, 202 μmol, as the “TAIL2” in Table 8) and TEA (84.3 μl, 605 μmol) in DCM (15 mL) was added cyclopropanesulfonyl chloride (85 mg, 605 μmol). The reaction mixture was stirred at room temperature. After the starting material was consumed, the reaction mixture was adjusted to pH=4 with HOAc, and then concentrated in vacuo to give the crude product. The residue was purified by prep-HPLC to give 3-[3-[2-(8-chloro-4-oxo-chromen-2-yl)-5-(trifluoromethyl)phenoxy]propyl-cyclopropylsulfonyl-amino]cyclobutanecarboxylic acid (3.6 mg, yield: 2.83%). MS obsd. (ESI⁺) [(M+H)⁺]: 600.5.

Example 139: 11.79-12.26 (m, 1H), 7.94-8.16 (m, 3H), 7.41-7.66 (m, 3H), 7.07 (s, 1H), 4.26-4.35 (m, 2H), 4.00-4.11 (m, 2H), 2.61-2.73 (m, 3H), 2.13-2.33 (m, 4H), 1.91-2.12 (m, 2H), 0.80-0.95 (m, 4H).

The following compounds 140 to 145 were prepared in analogy to the procedure described for the preparation of Example 139, replacing methyl 3-aminocyclobutane-1-carboxylate with “TAIL1” in Step 1 and replacing cyclopropanesulfonyl chloride with “TAIL2” in Step 3, by the reagent indicated in Table 8.

TABLE 8
Compounds synthesis and characterization

| Example | | TAIL1 and | |
|---|---|---|---|
| No. | Compounds Name and Structure | TAIL2 | ¹H NMR and (ESI⁺) |
| 140 | 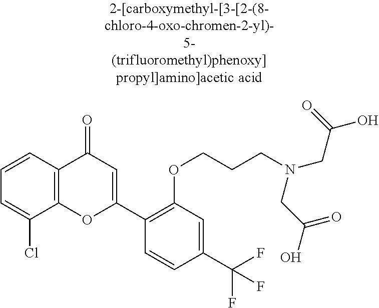 [SMILES: O=C(O)CN(CCCOc1cc(C(F)(F)F)ccc1-c1cc(=O)c2cccc(Cl)c2o1)CC(=O)O] | TAIL1: methyl 2- bromoacetate TAIL2: methyl 2- bromoacetate | ¹H NMR (METHANOL-d₄, 400 MHz) δ ppm 8.26 (d, J = 7.8 Hz, 1H), 8.14 (dd, J = 8.0, 1.5 Hz, 1H), 7.94 (dd, J = 7.8, 1.5 Hz, 1H), 7.42-7.61 (m, 3H), 7.27 (s, 1H), 4.38 (t, J = 5.7 Hz, 2H), 4.28 (s, 4H), 3.57-3.72 (m, 2H), 2.44 (br dd, J = 10.9, 5.5 Hz, 2H). MS obsd. (ESI⁺) [(M + H)⁺]: 514.1. |
| 141 | 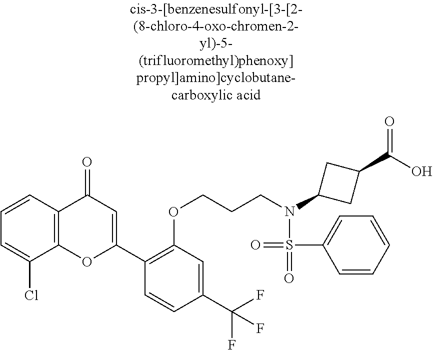 [SMILES: O=c1cc(-c2ccc(C(F)(F)F)cc2OCCCN([C@H]2C[C@@H](C(=O)O)C2)S(=O)(=O)c2ccccc2)oc2c(Cl)cccc12] | TAIL1: methyl 3- chlorocyclo- butane- carboxylate TAIL2: benzene- sulfonyl chloride | ¹H NMR (DMSO-d₆, 400 MHz) δ ppm 11.83-12.21 (m, 1H), 8.11 (d, J = 8.1 Hz, 1H), 8.02 (ddd, J = 11.3, 7.9, 1.5 Hz, 2H), 7.69- 7.77 (m, 2H), 7.60-7.69 (m, 1H), 7.42-7.60 (m, 5H), 7.08 (s, 1H), 4.32 (t, J = 5.8 Hz, 2H), 3.89- 4.05 (m, 1H), 3.14-3.26 (m, 2H), 2.54-2.65 (m, 1H), 1.84-2.35 (m, 4H), 1.14-1.57 (m, 2H). MS obsd. (ESI⁺) [(M + H)⁺]: 636.2. |
| 142 | 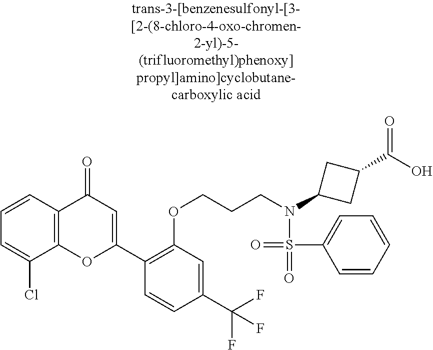 [SMILES: O=c1cc(-c2ccc(C(F)(F)F)cc2OCCCN([C@H]2C[C@H](C(=O)O)C2)S(=O)(=O)c2ccccc2)oc2c(Cl)cccc12] | TAIL1: cis-methyl 3- chlorocyclo- butane- carboxylate TAIL2: benzene- sulfonyl chloride | ¹H NMR (DMS0-d₆, 400 MHz) δ ppm 12.05-12.34 (m, 1H), 8.10 (d, J = 8.1 Hz, 1H), 7.96-8.07 (m, 2H), 7.59-7.73 (m, 3H), 7.46-7.58 (m, 5H), 6.98-7.08 (m, 1H), 4.23-4.42 (m, 3H), 3.20- 3.28 (m, 2H), 2.57-2.66 (m, 1H), 1.95-2.24 (m, 4H), 1.19-1.53 (m, 2H). MS obsd. (ESI⁺) [(M + H)⁺]: 636.2. |
| 143 | 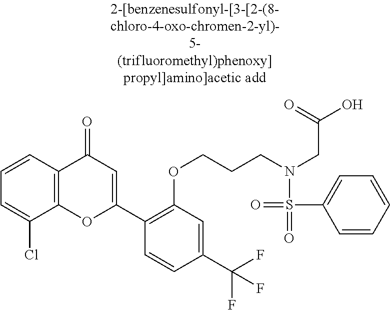 [SMILES: O=C(O)CN(CCCOc1cc(C(F)(F)F)ccc1-c1cc(=O)c2cccc(Cl)c2o1)S(=O)(=O)c1ccccc1] | TAIL1: trans- methyl 2- bromoacetate TAIL2: benzene- sulfonyl chloride | ¹H NMR (DMSO-d₆, 400 MHz) δ ppm 8.01-8.13 (m, 3H), 7.70 (d, J = 7.7 Hz, 2H), 7.54 (q, J = 7.9 Hz, 3H), 7.41-7.47 (m, 3H), 7.06 (s, 1H), 4.24 (t, J = 5.9 Hz, 2H), 3.98 (s, 2H), 3.25-3.33 (m, 2H), 2.00 (m, 2H). MS obsd. (ESI⁺) [(M + H)⁺]: 596.3. |
| 144 | 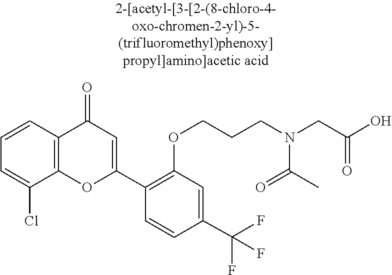 [SMILES: CC(=O)N(CCCOc1cc(C(F)(F)F)ccc1-c1cc(=O)c2cccc(Cl)c2o1)CC(=O)O] | TAIL1: methyl 2- bromoacetate TAIL2: acetyl chloride | ¹H NMR (DMSO-d₆, 400 MHz) δ ppm 8.07-8.22 (m, 1H), 7.95- 8.07 (m, 2H), 7.39-7.67 (m, 3H), 6.97-7.18 (m, 1H), 4.32 (m, 2H), 4.24 (m, 2H), 4.09 (s, 2H), 3.90 (s, 3H), 1.94-2.13 (m, 2H). MS obsd. (ESI⁺) [(M + H)⁺]: 498.4. |
| 145 | 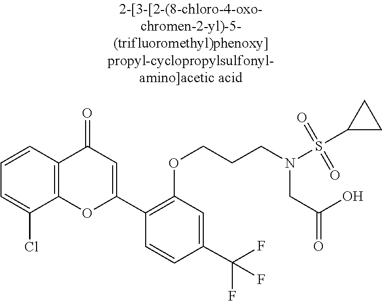 [SMILES: O=C(O)CN(CCCOc1cc(C(F)(F)F)ccc1-c1cc(=O)c2cccc(Cl)c2o1)S(=O)(=O)C1CC1] | TAIL1: methyl 2- bromoacetate TAIL2: cyclopropane- sulfonyl chloride | ¹H NMR (DMSO-d₆, 400 MHz) δ ppm 8.11 (d, J = 8.1 Hz, 1H), 8.03 (d, J = 7.8 Hz, 2H), 7.43- 7.69 (m, 3H), 7.06 (s, 1H), 4.33 (t, J = 5.9 Hz, 2H), 3.99 (s, 2H), 3.38-3.50 (m, 2H), 2.60-2.73 (m, 1H), 2.07 (br t, J = 6.1 Hz, 2H), 0.73-0.94 (m, 4H). MS obsd. (ESI⁺) [(M + H)⁺]: 560.1. |

### Example 146: N′-[2-[2-(8-chloro-4-oxo-chromen-2-yl)-5-(trifluoromethyl) phenoxy]ethyl]oxamide

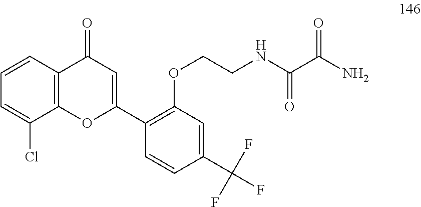 [SMILES: NC(=O)C(=O)NCCOc1cc(C(F)(F)F)ccc1-c1cc(=O)c2cccc(Cl)c2o1]

To a flask charged with 2-[2-[2-(8-chloro-4-oxo-chromen-2-yl)-5-(trifluoromethyl)phenoxy]ethylamino]-2-oxo-acetic acid (200 mg, 0.440 mmol, Example 119) was added NH₃/MeOH (4.0 mL, 0.440 mmol) and then the reaction was stirred at 55° C. for 1 hour. After the starting material was consumed, the reaction mixture was concentrated in vacuo to give the crude compound, which was recrystallized in MeOH to afford N′-[2-[2-(8-chloro-4-oxo-chromen-2-yl)-5-(trifluoromethyl)phenoxy]ethyl]oxamide (116 mg, yield: 57.4%) as a white solid. MS obsd. [(M+H)⁺] (ESI⁺): 455.0.

Example 146: ¹H NMR (400 MHz, DMSO-d₆): δ ppm 8.89 (br t, J=5.8 Hz, 1H), 8.13 (d, J=8.1 Hz, 1H), 7.96-8.05 (m, 3H), 7.77 (br s, 1H), 7.47-7.64 (m, 3H), 7.04 (s, 1H), 4.40 (t, J=5.9 Hz, 2H), 3.58 (q, J=6.0 Hz, 2H).

### Example 147: N′-[2-[2-(8-chloro-4-oxo-chromen-2-yl)-5-methoxy-phenoxy]ethyl]oxamide

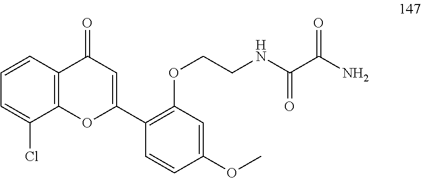 [SMILES: COc1ccc(-c2cc(=O)c3cccc(Cl)c3o2)c(OCCNC(=O)C(N)=O)c1]

The Example 147 was prepare in analogy to the procedure described for the preparation of 146, replacing 2-[2-[2-(8-chloro-4-oxo-chromen-2-yl)-5-(trifluoromethyl)phenoxy]ethylamino]-2-oxo-acetic acid with 2-[2-[2-(8-chloro-4-oxo-chromen-2-yl)-5-methoxy-phenoxy]ethylamino]-2-oxo-acetic acid (111) as starting material. MS obsd. (ESI⁺) [(M+H)⁺]: 417.0.

Example 147: ¹H NMR (400 MHz, DMSO-d6): δ ppm 8.89 (t, J=5.4 Hz, 1H), 8.07 (s, 1H), 7.96 (dd, J=7.8, 5.1 Hz, 3H), 7.79 (s, 1H), 7.47 (t, J=7.8 Hz, 1H), 6.98 (s, 1H), 6.87-6.75 (m, 2H), 4.29 (t, J=5.5 Hz, 2H), 3.88 (s, 3H), 3.59 (dd, J=11.3, 5.4 Hz, 2H).

### Example 148: N-[2-[2-(8-chloro-4-oxo-chromen-2-yl)-5-(trifluoromethyl)phenoxy]ethyl]-N′-cyclopropyl-oxamide

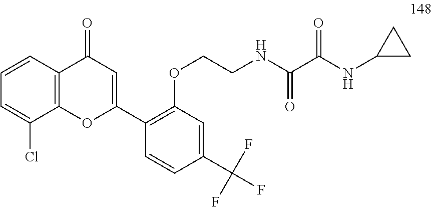 [SMILES: O=C(NCCOc1cc(C(F)(F)F)ccc1-c1cc(=O)c2cccc(Cl)c2o1)C(=O)NC1CC1]

To a solution of ethyl 2-[2-[2-(8-chloro-4-oxo-chromen-2-yl)-5-(trifluoromethyl)phenoxy]ethylamino]-2-oxo-acetate (100 mg, 0.21 mmol, Example 101) in DCM (20 mL) was added cyclopropylamine (0.02 mL, 0.320 mmol) and titanium tetrachloride (80.75 mg, 0.430 mmol) at 0° C. under N₂. Then, the reaction was heated to 110° C. and stirred for 2 hours. After the starting material was consumed, the reaction mixture was cooled to room temperature and then poured into ice-water (20 mL). The aqueous layer was extracted with EtOAc (50 mL) three times. The combined organic layer was washed with brine, dried over anhydrous Na₂SO₄, filtered and concentrated in vacuo to give the crude residue, which was purified by prep-HPLC to afford N-[2-[2-(8-chloro-4-oxo-chromen-2-yl)-5-(trifluoromethyl)phenoxy]ethyl]-N′-cyclopropyl-oxamide (41.3 mg, yield: 39.1%) as a white solid. MS obsd. (ESI⁺)[(M+H)⁺]: 495.1.

Example 148: ¹H NMR (400 MHz, DMSO-d6): δ ppm 8.90 (t, J=5.9 Hz, 1H), 8.66 (d, J=5.3 Hz, 1H), 8.12 (d, J=7.9 Hz, 1H), 8.02 (dq, J=7.9, 1.4 Hz, 2H), 7.62 (s, 1H), 7.46-7.60 (m, 2H), 6.95-7.05 (m, 1H), 4.40 (t, J=5.9 Hz, 2H), 3.58 (q, J=6.0 Hz, 2H), 2.64-2.73 (m, 1H), 0.54-0.64 (m, 4H).

### Example 149: N-[3-[2-(8-chloro-4-oxo-chromen-2-yl)-5-(trifluoromethyl)phenoxy]propyl]-N′-cyclopropylsulfonyl-oxamide

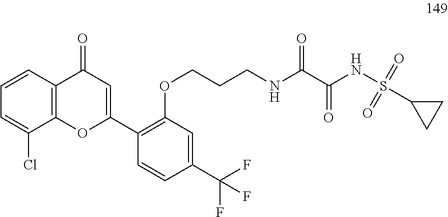 [SMILES: O=C(NCCCOc1cc(C(F)(F)F)ccc1-c1cc(=O)c2cccc(Cl)c2o1)C(=O)NS(=O)(=O)C1CC1]

The Example 149 was prepared in analogy to the procedure described for the preparation of 148, replacing ethyl 2-[2-[2-(8-chloro-4-oxo-chromen-2-yl)-5-(trifluoromethyl)phenoxy]ethylamino]-2-oxo-acetate (Example 101) with ethyl 2-[3-[2-(8-chloro-4-oxo-chromen-2-yl)-5-(trifluoromethyl)phenoxy]propylamino]-2-oxo-acetate (100) and cyclopropanesulfonamide with cyclopropylamine as starting material. MS obsd. (ESI⁺) [(M+H)⁺]: 573.1.

Example 149: ¹H NMR (400 MHz, DMSO-d₆): δ ppm 12.20 (s, 1H), 9.16 (t, J=5.7 Hz, 1H), 8.12 (d, J=8.0 Hz, 1H), 8.03 (ddd, J=8.0, 3.2, 1.5 Hz, 2H), 7.54 (dt, J=15.8, 8.1 Hz, 3H), 7.12 (s, 1H), 4.28 (t, J=6.1 Hz, 2H), 3.38-3.34 (m, 2H), 3.00-2.90 (m, 1H), 2.01 (p, J=6.0 Hz, 2H), 1.16-0.98 (m, 4H).

### Example 150: N-[2-[2-(8-chloro-4-oxo-chromen-2-yl)-5-methyl-phenoxy]ethyl]-2-[(3S)-3-hydroxypyrrolidin-1-yl]-2-oxo-acetamide

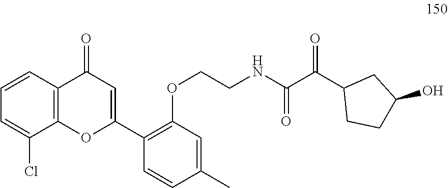 [SMILES: Cc1ccc(-c2cc(=O)c3cccc(Cl)c3o2)c(OCCNC(=O)C(=O)C2CC[C@H](O)C2)c1]

The Example 150 was prepared in analogy to the procedure described for the preparation of 148, replacing ethyl 2-[2-[2-(8-chloro-4-oxo-chromen-2-yl)-5-(trifluoromethyl)phenoxy]ethylamino]-2-oxo-acetate (Example 101) with ethyl 2-[2-[2-(8-chloro-4-oxo-chromen-2-yl)-5-methyl-phenoxy]ethylamino]-2-oxo-acetate (Example 109) and cyclopropanesulfonamide with (3S)-pyrrolidin-3-ol as starting material. MS obsd. (ESI⁺) [(M+H)⁺]: 471.2.

Example 150: ¹H NMR (METHANOL-d₄, 400 MHz) δ ppm 8.80-8.97 (m, 1H), 7.83-8.16 (m, 3H), 7.47 (br t, J=7.6 Hz, 1H), 7.26 (s, 1H), 7.09 (s, 1H), 7.03 (br d, J=7.6 Hz, 1H), 4.31-4.50 (m, 4H), 3.71-3.93 (m, 4H), 3.44-3.64 (m, 2H), 2.45 (s, 3H), 1.82-2.08 (m, 1H).

### Example 151: (2S)-2-[2-[2-(8-chloro-4-oxo-chromen-2-yl)-5-methyl-phenoxy]ethylcarbamoylamino]propanoic acid

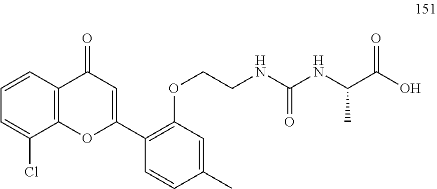 [SMILES: Cc1ccc(-c2cc(=O)c3cccc(Cl)c3o2)c(OCCNC(=O)N[C@@H](C)C(=O)O)c1]

### Step: 1 Preparation of 2-[2-(2-aminoethoxy)-4-methyl-phenyl]-8-chloro-chromen-4-one

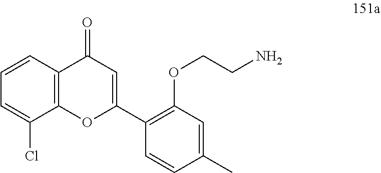 [SMILES: Cc1ccc(-c2cc(=O)c3cccc(Cl)c3o2)c(OCCN)c1]

The compounds 151a was prepared in analogy to the procedure described for the preparation of 095b, using 8-chloro-2-(2-hydroxy-4-methyl-phenyl)chromen-4-one (as the “CORE” in Table 9) and replacing tert-butyl (3-bromopropyl)carbamate with tert-butyl (3-bromoethyl)carbamate as starting material in Step 1.

### Step 2: Preparation of methyl (2S)-2-[2-[2-(8-chloro-4-oxo-chromen-2-yl)-5-methyl-phenoxy]ethylcarbamoylamino]propanoate

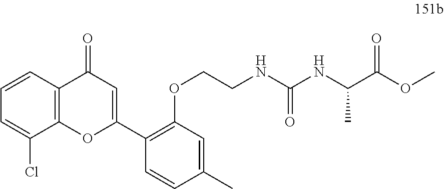 [SMILES: COC(=O)[C@H](C)NC(=O)NCCOc1cc(C)ccc1-c1cc(=O)c2cccc(Cl)c2o1]

To a solution of 2-[2-(2-aminoethoxy)-4-methyl-phenyl]-8-chloro-chromen-4-one (50 mg, 152 μmol) and methyl (S)-2-isocyanatopropanoate (40 mg, 303 μmol, as the “TAIL” in Table 9) in DCM (1.5 mL) was added DIPEA (56 μl, 323 μmol). The reaction was stirred at room temperature for 2 hours. After the reaction was completed, the reaction residue was concentrated in vacuo to afford methyl (2S)-2-[2-[2-(8-chloro-4-oxo-chromen-2-yl)-5-methyl-phenoxy]ethylcarbamoylamino]propanoate (69 mg, yield: 99.2%). MS obsd. (ESI⁺) [(M+H)⁺]: 459.2.

### Step 3: Preparation of (2S)-2-[2-[2-(8-chloro-4-oxo-chromen-2-yl)-5-methyl-phenoxy]ethylcarbamoylamino]propanoic acid

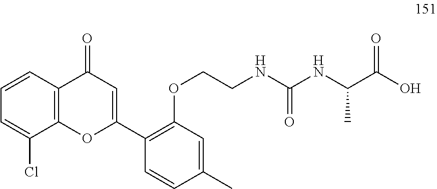 [SMILES: Cc1ccc(-c2cc(=O)c3cccc(Cl)c3o2)c(OCCNC(=O)N[C@@H](C)C(=O)O)c1]

To a solution of methyl (2S)-2-[2-[2-(8-chloro-4-oxo-chromen-2-yl)-5-methyl-phenoxy]ethylcarbamoylamino]propanoate (70 mg, 153 μmol) in THF (1.5 mL) and water (1.5 mL) was added sodium hydroxide (30.5 mg, 763 μmol). After stirring for 2 hours, the reaction mixture was partitioned between EtOAc (10 mL) and water (20 mL). The aqueous layer was extracted with EtOAc (10 mL) and combined organic layer was washed with brine, dried over anhydrous Na₂SO₄, filtered and concentrated in vacuo to give a yellow oil, which was purified by prep-HPLC to afford (2S)-2-[2-[2-(8-chloro-4-oxo-chromen-2-yl)-5-methyl-phenoxy]ethylcarbamoylamino]propanoic acid (7.1 mg, 10.3%).

Example 151: MS obsd. (ESI⁺) [(M+H)⁺]: 445.3. ¹H NMR (DMSO-d₆, 400 MHz) δ ppm 8.00 (dd, J=7.9, 2.3 Hz, 2H), 7.89 (d, J=7.9 Hz, 1H), 7.49 (t, J=7.8 Hz, 1H), 7.14 (s, 1H), 7.08 (s, 1H), 7.02 (d, J=8.7 Hz, 1H), 6.26-6.39 (m, 2H), 4.14 (s, 2H), 4.06 (s, 1H), 2.40 (s, 3H), 1.20 (d, J=7.2 Hz, 3H).

The following compounds 152 to 155 were prepared in analogy to the procedure described for the preparation of Example 151, replacing 8-chloro-2-(2-hydroxy-4-methyl-phenyl)chromen-4-one with “CORE” and replacing methyl (S)-2-isocyanato-3-methylbutanoate with “TAIL” in Step 2, by the reagent indicated in Table 9.

TABLE 9
Compounds synthesis and characterization

| Example | | CORE and | |
|---|---|---|---|
| No. | Compounds Name and Structure | TAIL | ¹H NMR and (ESI⁺) |
| 152 | 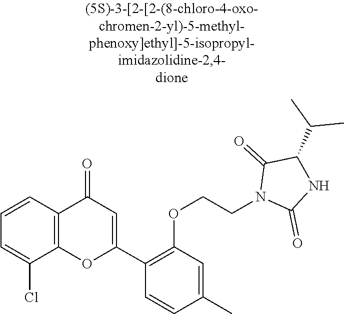 [SMILES: Cc1ccc(-c2cc(=O)c3cccc(Cl)c3o2)c(OCCN2C(=O)N[C@@H](C(C)C)C2=O)c1] | CORE: Int-2 TAIL: methyl (S)-2- isocyanato-3- methylbutanoate | ¹H NMR (DMSO-d₆, 400 MHz) δ ppm 8.22 (s, 1H), 7.92-8.04 (m, 2H), 7.80 (d, J = 7.9 Hz, 1H), 7.48 (t, J = 7.9 Hz, 1H), 7.14 (s, 1H), 7.01 (d, J = 7.9 Hz, 1H), 6.87 (s, 1H), 4.35 (s, 2H), 3.90 (dd, J = 3.7, 1.1 Hz, 1H), 3.79 (s, 2H), 3.17 (s, 3H), 1.84-1.97 (m, 1H), 0.83 (d, J = 7.0 Hz, 3H), 0.64 (d, J = 6.7 Hz, 3H). MS obsd. (ESI⁺) [(M + H)⁺]: 455.3. |
| 153 | 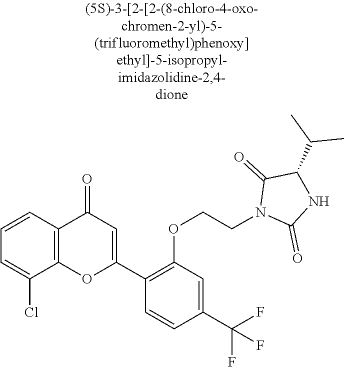 [SMILES: CC(C)[C@@H]1NC(=O)N(CCOc2cc(C(F)(F)F)ccc2-c2cc(=O)c3cccc(Cl)c3o2)C1=O] | CORE: Int-1 TAIL: methyl (S)-2- isocyanato-3- methylbutanoate | ¹H NMR (DMSO-d₆, 400 MHz) δ ppm 8.21 (s, 1H), 7.89-8.11 (m, 3H), 7.61 (s, 1H), 7.38-7.59 (m, 2H), 6.90 (s, 1H), 4.45 (t, J = 5.5 Hz, 2H), 3.84 (dd, J = 3.6, 1.2 Hz, 1H), 3.73-3.83 (m, 2H), 1.76-1.96 (m, 1H), 0.80 (d, J = 7.0 Hz, 3H), 0.60 (d, J = 6.8 Hz, 3H). MS obsd. (ESI⁺) [(M + H)⁺]: 509.1. |
| 154 | 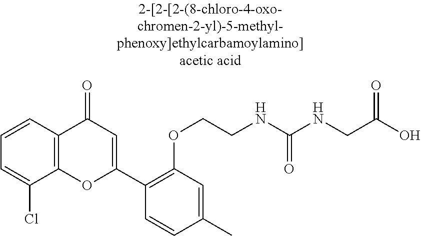 [SMILES: Cc1ccc(-c2cc(=O)c3cccc(Cl)c3o2)c(OCCNC(=O)NCC(=O)O)c1] | CORE: Int-2 TAIL: ethyl 2- isocyanatoacetate | ¹H NMR (DMSO-d₆, 400 MHz) δ ppm 8.00 (m, 2H), 7.88 (m, 1H), 7.42-7.61 (m, 1H), 7.14 (m, 1H), 7.08 (s, 1H), 7.03 (m, 1H), 6.42 (m, 1H), 6.29 (m, 1H), 4.11-4.20 (m, 2H), 3.92 (m, 2H), 3.70 (m, J = 5.5 Hz, 2H), 2.39 (s, 3H). MS obsd. (ESI⁺) [(M + H)⁺]: 431.0. |
| 155 | 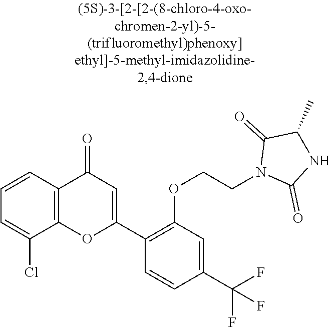 [SMILES: C[C@@H]1NC(=O)N(CCOc2cc(C(F)(F)F)ccc2-c2cc(=O)c3cccc(Cl)c3o2)C1=O] | CORE: Int-1 TAIL: methyl (S)-2- isocyanato- propanoate | ¹H NMR (DMSO-d₆, 400 MHz) δ ppm 8.06-8.22 (m, 2H), 8.02 (d, J = 7.8 Hz, 2H), 7.32-7.66 (m, 3H), 6.92 (s, 1H), 4.47 (t, J = 5.6 Hz, 2H), 4.02 (dd, J = 7.0, 1.1 Hz, 1H), 3.76 (t, J = 5.5 Hz, 2H), 1.11 (d, J = 7.0 Hz, 3H). MS obsd. (ESI⁺) [(M + H)⁺]: 481.0. |

### Example 156: 1-[3-[2-(8-chloro-4-oxo-chromen-2-yl)-5-methyl-phenoxy]propyl]-3-(p-tolylsulfonyl)urea

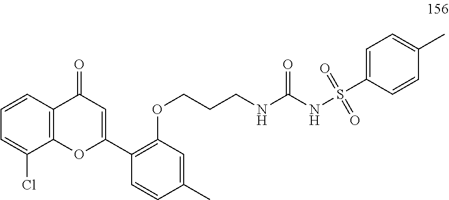 [SMILES: Cc1ccc(S(=O)(=O)NC(=O)NCCCOc2cc(C)ccc2-c2cc(=O)c3cccc(Cl)c3o2)cc1]

A solution of 2-[2-(3-aminopropoxy)-4-methyl-phenyl]-8-chloro-chromen-4-one (100 mg, 291 μmol) in DCM (5.82 mL) was cooled in the ice-water bath. Into the stirring solution was slowly added 4-methylbenzenesulfonyl isocyanate (88.6 μl, 582 μmol). The reaction was allowed to warm to room temperature and stirred overnight. After the reaction went complete, the reaction mixture was concentrated in vacuo to give a yellow solid, which was purified by prep-HPLC and afforded 1-[3-[2-(8-chloro-4-oxo-chromen-2-yl)-5-methyl-phenoxy]propyl]-3-(p-tolylsulfonyl)urea (56 mg, yield: 33.8%) as a light yellow solid. MS obsd. (ESI⁺) [(M+H)⁺]: 541.2.

Example 156: ¹H NMR (DMSO-d₆, 400 MHz) δ ppm 7.98 (dd, J=7.9, 1.4 Hz, 2H), 7.86 (d, J=8.3 Hz, 1H), 7.76 (d, J=8.3 Hz, 2H), 7.48 (t, J=7.8 Hz, 1H), 7.36 (d, J=7.9 Hz, 2H), 7.02 (s, 3H), 6.68 (br t, J=5.6 Hz, 1H), 4.04 (t, J=6.4 Hz, 2H), 3.07-3.21 (m, 2H), 2.39 (s, 3H), 2.35 (s, 3H), 2.07 (s, 1H), 1.88 (br t, J=6.5 Hz, 2H).

### Example 157

### 3-[2-(8-chloro-4-oxo-chromen-2-yl)-5-(trifluoromethyl)phenoxy]propylurea

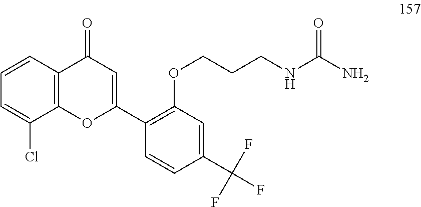 [SMILES: NC(=O)NCCCOc1cc(C(F)(F)F)ccc1-c1cc(=O)c2cccc(Cl)c2o1]

### Step 1: Preparation of 2-[2-(3-aminopropoxy)-4-(trifluoromethyl)phenyl]-8-chloro-chromen-4-one

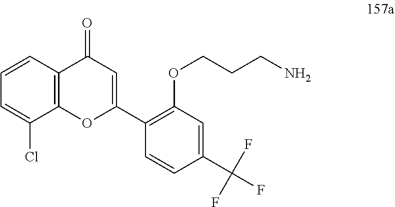 [SMILES: NCCCOc1cc(C(F)(F)F)ccc1-c1cc(=O)c2cccc(Cl)c2o1]

The compound 157a was prepared in analogy to the procedure described for the preparation of 095b, replacing tert-butyl (3-bromoethyl)carbamate with tert-butyl (3-bromopropyl)carbamate and replacing 8-chloro-2-(2-hydroxy-4-methyl-phenyl)chromen-4-one with 8-chloro-2-[2-hydroxy-4-(trifluoromethyl)phenyl]chromen-4-one as starting material in Step 1. (ESI⁺) [(M+H)⁺]: 398.2.

### Step 2: Preparation of 3-[2-(8-chloro-4-oxo-chromen-2-yl)-5-(trifluoromethyl)phenoxy]propylurea

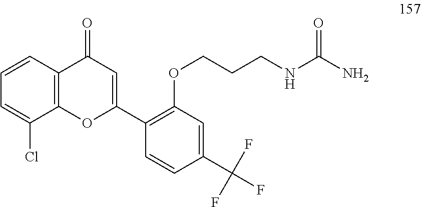 [SMILES: NC(=O)NCCCOc1cc(C(F)(F)F)ccc1-c1cc(=O)c2cccc(Cl)c2o1]

To a solution of 2-[2-(3-aminopropoxy)-4-(trifluoromethyl)phenyl]-8-chloro-chromen-4-one (0.18 g, 453 μmol) in DCM (4 mL) was added isocyanatotrimethylsilane (73.6 μl, 543 μmol). The reaction mixture was stirred at room temperature for 1 day. Then the reaction was concentrated in vacuo to give the crude product, which was purified by prep-HPLC to give 3-[2-(8-chloro-4-oxo-chromen-2-yl)-5-(trifluoromethyl)phenoxy]propylurea (19.7 mg, yield: 9.38%) as a white solid. MS obsd. (ESI⁺) [(M+H)⁺]: 441.5.

Example 157: ¹H NMR (DMSO-d₆, 400 MHz) δ ppm 8.14 (d, J=7.8 Hz, 1H), 8.03 (d, J=7.7 Hz, 2H), 7.45-7.66 (m, 3H), 7.11 (s, 1H), 6.09 (br t, J=5.7 Hz, 1H), 5.40 (s, 2H), 4.18-4.35 (m, 2H), 3.14 (br d, J=6.1 Hz, 2H), 1.90 (m, 2H).

### Example 158: 1-[2-[2-(8-chloro-4-oxo-chromen-2-yl)-5-(trifluoromethyl)phenoxy]ethyl]-3-(p-tolylsulfonyl)urea

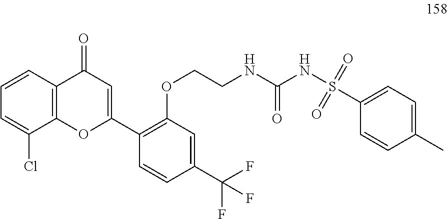 [SMILES: Cc1ccc(S(=O)(=O)NC(=O)NCCOc2cc(C(F)(F)F)ccc2-c2cc(=O)c3cccc(Cl)c3o2)cc1]

### Step 1: Preparation of 2-[2-(2-aminoethoxy)-4-(trifluoromethyl)phenyl]-8-chloro-chromen-4-one

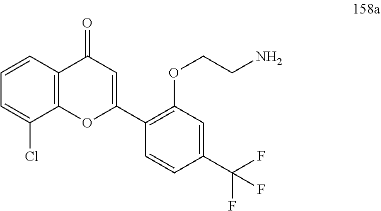 [SMILES: NCCOc1cc(C(F)(F)F)ccc1-c1cc(=O)c2cccc(Cl)c2o1]

The compound 158a was prepared in analogy to the procedure described for the preparation of 157a, replacing tert-butyl (3-bromopropyl)carbamate with tert-butyl (3-bromoethyl)carbamate as starting material. (ESI⁺) [(M+H)⁺]: 384.1.

### Step 2: Preparation of 1-[2-[2-(8-chloro-4-oxo-chromen-2-yl)-5-(trifluoromethyl)phenoxy]ethyl]-3-(p-tolylsulfonyl)urea

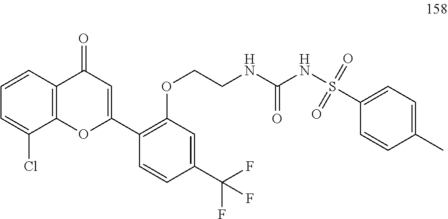 [SMILES: Cc1ccc(S(=O)(=O)NC(=O)NCCOc2cc(C(F)(F)F)ccc2-c2cc(=O)c3cccc(Cl)c3o2)cc1]

The compound 158 was prepared in analogy to the procedure described for the preparation of 158, replacing 2-[2-(3-aminopropoxy)-4-methyl-phenyl]-8-chloro-chromen-4-one with 2-[2-(2-aminoethoxy)-4-(trifluoromethyl)phenyl]-8-chloro-chromen-4-one as starting material. MS obsd. (ESI⁺) [(M+H)⁺]: 581.2.

Example 158: ¹H NMR (DMSO-d₆, 400 MHz) δ ppm 8.12 (d, J=7.9 Hz, 1H), 7.94-8.07 (m, 2H), 7.71 (d, J=8.2 Hz, 1H), 7.45-7.67 (m, 3H), 7.29 (d, J=7.9 Hz, 2H), 7.03 (s, 1H), 6.67 (br t, J=5.4 Hz, 1H), 4.24 (t, J=5.6 Hz, 2H), 3.44 (br d, J=5.6 Hz, 2H), 2.32 (s, 3H).

### Example 159: 8-chloro-4-oxo-2-[2-[2-(sulfamoylamino)ethoxy]-4-(trifluoromethyl)phenyl]chromene

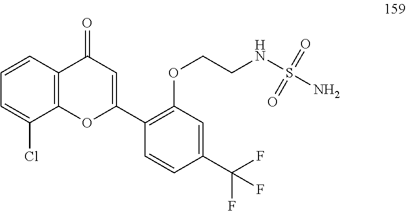 [SMILES: NS(=O)(=O)NCCOc1cc(C(F)(F)F)ccc1-c1cc(=O)c2cccc(Cl)c2o1]

### Step 1: Preparation of tert-butyl N-[2-[2-(8-chloro-4-oxo-chromen-2-yl)-5-(trifluoromethyl)phenoxy]ethylsulfamoyl]carbamate

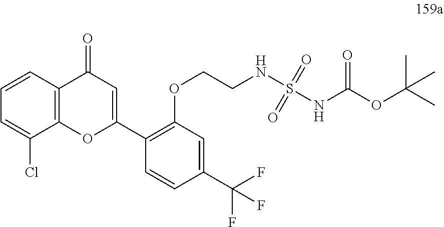 [SMILES: CC(C)(C)OC(=O)NS(=O)(=O)NCCOc1cc(C(F)(F)F)ccc1-c1cc(=O)c2cccc(Cl)c2o1]

To a solution of 2-[2-(2-aminoethoxy)-4-(trifluoromethyl)phenyl]-8-chloro-chromen-4-one (200 mg, 447 μmol) in DMF (5 mL) was added K₂CO₃ (309 mg, 2.23 mmol) and tert-butyl N-chlorosulfonylcarbamate (193 mg, 983 μmol). The reaction was stirred at 70° C. overnight After the starting material was consumed, the reaction was cooled to room temperature and quenched by water (20 mL). The aqueous layer was extracted with EtOAc (10 mL) twice, and the organic layer was combined, washed with water and brine, dried over anhydrous Na₂SO₄, filtered and concentrated in vacuo to give tert-butyl N-[2-[2-(8-chloro-4-oxo-chromen-2-yl)-5-(trifluoromethyl)phenoxy]ethylsulfamoyl]carbamate (252 mg, 100%) as brown oil. MS obsd. (ESI⁺) [(M+H)⁺]: 563.2.

### Step 2: Preparation of 8-chloro-4-oxo-2-[2-[2-(sulfamoylamino)ethoxy]-4-(trifluoromethyl)phenyl]chromene

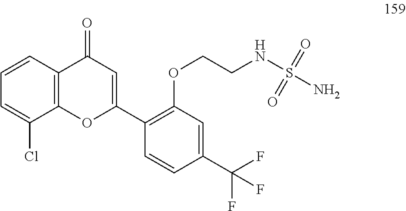 [SMILES: NS(=O)(=O)NCCOc1cc(C(F)(F)F)ccc1-c1cc(=O)c2cccc(Cl)c2o1]

To a solution of tert-butyl N-[2-[2-(8-chloro-4-oxo-chromen-2-yl)-5-(trifluoromethyl)phenoxy]ethylsulfamoyl]carbamate (275 mg, 489 μmol) in DCM (6 mL) was added TFA (3 mL, 489 μmol) dropwise. The reaction was stirred for 2 hours and then the reaction was concentrated in vacuo to give the crude product, which was purified by prep-HPLC to afford 8-chloro-4-oxo-2-[2-[2-(sulfamoylamino)ethoxy]-4-(trifluoromethyl)phenyl]chromene (12.4 mg, yield: 5.37%) as a white solid. MS obsd. (ESI⁺) [(M+H)⁺]: 462.9.

Example 159: ¹H NMR (DMSO-d₆, 400 MHz) δ ppm 8.17 (d, J=7.9 Hz, 1H), 8.04 (dd, J=7.9, 0.9 Hz, 2H), 7.45-7.70 (m, 3H), 7.28 (s, 1H), 6.84-6.94 (m, 1H), 6.67 (s, 2H), 4.37 (t, J=5.7 Hz, 2H), 3.46-3.55 (m, 2H).

### Example 160: 8-chloro-4-oxo-2-[2-[3-(sulfamoylamino)propoxy]-4-(trifluoromethyl)phenyl]chromene

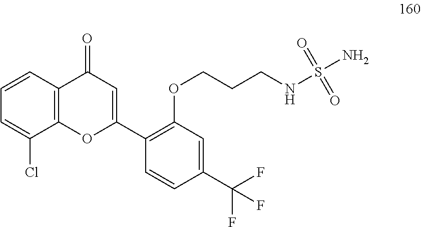 [SMILES: NS(=O)(=O)NCCCOc1cc(C(F)(F)F)ccc1-c1cc(=O)c2cccc(Cl)c2o1]

The Example 160 was prepared in analogy to the procedure described for the preparation of 159, replacing 2-[2-(2-aminoethoxy)-4-(trifluoromethyl)phenyl]-8-chloro-chromen-4-one with 2-[2-(3-aminopropoxy)-4-(trifluoromethyl)phenyl]-8-chloro-chromen-4-one as starting material. MS obsd. (ESI⁺) [(M+H)⁺]: 476.9.

Example 160: ¹H NMR (DMSO-d₆, 400 MHz) δ ppm 8.15 (d, J=7.8 Hz, 1H), 8.03 (dd, J=7.9, 1.8 Hz, 2H), 7.54 (d, J=16.3 Hz, 3H), 7.12 (s, 1H), 6.62-6.74 (m, 1H), 6.53 (s, 2H), 4.33 (s, 2H), 3.08 (d, J=6.2 Hz, 2H), 1.98-2.08 (m, 2H).

### Example 161: 8-chloro-2-[4-methyl-2-[2-(sulfamoylamino)ethoxy]phenyl]-4-oxo-chromene

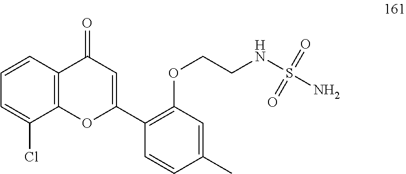 [SMILES: Cc1ccc(-c2cc(=O)c3cccc(Cl)c3o2)c(OCCNS(N)(=O)=O)c1]

The compound 161 was prepared in analogy to the procedure described for the preparation of 159, replacing 2-[2-(2-aminoethoxy)-4-(trifluoromethyl)phenyl]-8-chloro-chromen-4-one with 2-[2-(2-aminoethoxy)-4-methyl-phenyl]-8-chloro-chromen-4-one as starting material. MS obsd. (ESI⁺) [(M+H)⁺]: 410.1.

Example 161: ¹H NMR (DMSO-d₆, 400 MHz) δ ppm 7.95-8.10 (m, 4H), 7.51 (t, J=7.9 Hz, 1H), 7.36 (s, 1H), 7.03 (s, 1H), 4.35 (br t, J=4.8 Hz, 2H), 3.27 (m, 2H), 2.45 (s, 3H).

### Example 162 2-[3-[2-[2-(8-chloro-4-oxo-chromen-2-yl)-5-(trifluoromethyl)phenoxy]ethyl]-2-oxo-imidazolidin-1-yl]-2-oxo-acetic acid

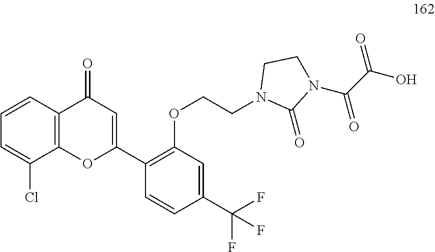 [SMILES: O=C(O)C(=O)N1CCN(CCOc2cc(C(F)(F)F)ccc2-c2cc(=O)c3cccc(Cl)c3o2)C1=O]

### Step 1: Preparation of 1-(2-chloroethyl)-3-[2-[2-(8-chloro-4-oxo-chromen-2-yl)-5-(trifluoromethyl)phenoxy]ethyl]urea

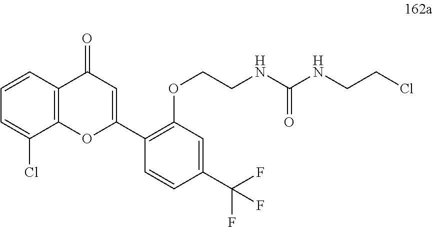 [SMILES: O=C(NCCCl)NCCOc1cc(C(F)(F)F)ccc1-c1cc(=O)c2cccc(Cl)c2o1]

To a solution of 2-[2-(2-aminoethoxy)-4-(trifluoromethyl)phenyl]-8-chloro-chromen-4-one (110 mg, 287 μmol) in DCM (5 ml) was added 1-chloro-2-isocyanatoethane (36.7 μL, 430 μmol) and DIPEA (50.1 μL, 287 μmol). The mixture was stirred at room temperature overnight. After stirring for 2 hours, the reaction mixture was concentrated in vacuo to give 1-(2-chloroethyl)-3-[2-[2-(8-chloro-4-oxo-chromen-2-yl)-5-(trifluoromethyl)phenoxy]ethyl]urea (140 mg, yield: 100%). MS obsd. (ESI⁺) [(M+H)⁺]: 489.2.

### Step 2: Preparation of 1-[2-[2-(8-chloro-4-oxo-chromen-2-yl)-5-(trifluoromethyl)phenoxy]ethyl]imidazolidin-2-one

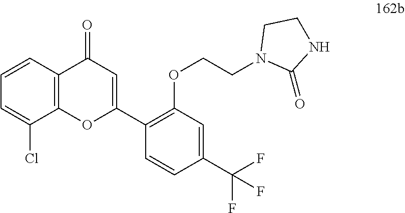 [SMILES: O=C1NCCN1CCOc1cc(C(F)(F)F)ccc1-c1cc(=O)c2cccc(Cl)c2o1]

To a solution of 1-(2-chloroethyl)-3-[2-[2-(8-chloro-4-oxo-chromen-2-yl)-5-(trifluoromethyl)phenoxy]ethyl]urea (140 mg, 287 μmol) in THF (5 mL) was added KOH (80.4 mg, 1.43 mmol). The reaction was stirred at 80° C. overnight. After the starting material was consumed, the reaction was quenched by adding water (20 mL). The aqueous layer was extracted with EtOAc (10 mL) three times. The organic layer was combined, washed with brine, dried over anhydrous sodium sulfate, filtered and concentrated in vacuo to give 1-[2-[2-(8-chloro-4-oxo-chromen-2-yl)-5-(trifluoromethyl)phenoxy]ethyl]imidazolidin-2-one (0.126 g, yield: 97.1%) as a light yellow solid. MS obsd. (ESI⁺) [(M+H)⁺]: 453.0.

### Step 3: Preparation of 2-[3-[2-[2-(8-chloro-4-oxo-chromen-2-yl)-5-(trifluoromethyl)phenoxy]ethyl]-2-oxo-imidazolidin-1-yl]-2-oxo-acetic acid

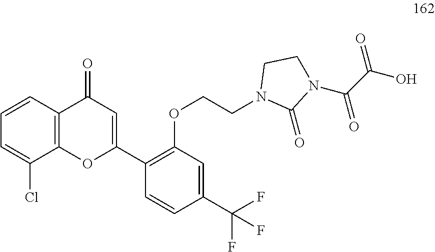 [SMILES: O=C(O)C(=O)N1CCN(CCOc2cc(C(F)(F)F)ccc2-c2cc(=O)c3cccc(Cl)c3o2)C1=O]

To a solution of 1-[2-[2-(8-chloro-4-oxo-chromen-2-yl)-5-(trifluoromethyl)phenoxy]ethyl]imidazolidin-2-one (100 mg, 221 μmol) in DCM (5 mL) was added oxalyl dichloride (56.1 mg, 442 μmol) and DIPEA (38.6 μL, 221 μmol). The reaction was stirred at room temperature for 1 hour and the starting material was completely converted. Then, into the reaction was added water and the reaction mixture was stirred for another 1 hour. The reaction was partitioned between DCM (10 mL) and water (20 mL). The organic layer was combined, washed with brine, dried over anhydrous sodium sulfate, filtered and concentrated in vacuo to give yellow oil crude. The crude product was purified by prep-HPLC to give 2-[3-[2-[2-(8-chloro-4-oxo-chromen-2-yl)-5-(trifluoromethyl)phenoxy]ethyl]-2-oxo-imidazolidin-1-yl]-2-oxo-acetic acid (11.5 mg, yield: 8.93%) as a white solid. MS obsd. (ESI⁺) [(M+H)⁺]: 525.0.

Example 162: ¹H NMR (DMSO-d₆, 400 MHz) δ ppm 8.12 (d, J=8.1 Hz, 1H), 8.02 (d, J=8.1 Hz, 2H), 7.30-7.71 (m, 3H), 7.07 (s, 1H), 4.47 (t, J=5.2 Hz, 2H), 3.63-3.72 (m, 4H), 3.54-3.62 (m, 2H).

### Example 163: (3S)-1-[3-[2-(8-chloro-4-oxo-chromen-2-yl)-5-(trifluoromethyl)phenoxy]-2-hydroxy-propyl]pyrrolidine-3-carboxylic acid

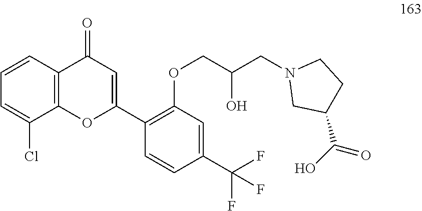 [SMILES: O=C(O)[C@H]1CCN(CC(O)COc2cc(C(F)(F)F)ccc2-c2cc(=O)c3cccc(Cl)c3o2)C1]

### Step 1: Preparation of 8-chloro-2-[2-(oxiran-2-ylmethoxy)-4-(trifluoromethyl)phenyl]chromen-4-one

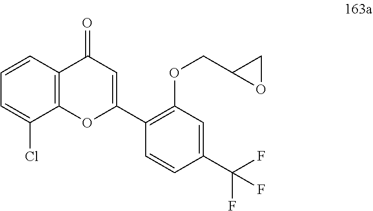 [SMILES: O=c1cc(-c2ccc(C(F)(F)F)cc2OCC2CO2)oc2c(Cl)cccc12]

To a solution of 8-chloro-2-[2-hydroxy-4-(trifluoromethyl)phenyl]chromen-4-one (400 mg, 1.17 mmol) and 2-(chloromethyl)oxirane (435 mg, 4.7 mmol) in DMF was added K₂CO₃ (162 mg, 1.17 mmol). The reaction mixture was stirred at 50° C. overnight. After the reaction was complete, the mixture was partitioned between EtOAc (10 mL) and water (30 mL). The organic layer was concentrated in vacuo to give 8-chloro-2-[2-(oxiran-2-ylmethoxy)-4-(trifluoromethyl)phenyl]chromen-4-one (460 mg, yield: 98.7%) as a white solid. MS obsd. (ESI⁺) [(M+H)⁺]: 397.1.

### Step 2: Preparation of methyl (3S)-1-[3-[2-(8-chloro-4-oxo-chromen-2-yl)-5-(trifluoromethyl)phenoxy]-2-hydroxy-propyl]pyrrolidine-3-carboxylate

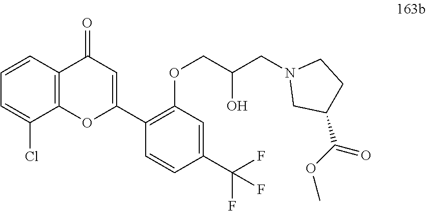 [SMILES: COC(=O)[C@H]1CCN(CC(O)COc2cc(C(F)(F)F)ccc2-c2cc(=O)c3cccc(Cl)c3o2)C1]

A mixture of 8-chloro-2-[2-(oxiran-2-ylmethoxy)-4-(trifluoromethyl)phenyl]chromen-4-one (80 mg, 202 μmol), methyl (S)-pyrrolidine-3-carboxylate (104 mg, 807 μmol) and K₂CO₃ (27.9 mg, 202 μmol) was stirred at 50° C. After the reaction was complete, the reaction mixture was concentrated in vacuo to give methyl (3S)-1-[3-[2-(8-chloro-4-oxo-chromen-2-yl)-5-(trifluoromethyl)phenoxy]-2-hydroxy-propyl]pyrrolidine-3-carboxylate (80 mg, yield 75.4%). MS obsd. (ESI⁺) [(M+H)⁺]: 526.1.

### Step 3: Preparation of (3S)-1-[3-[2-(8-chloro-4-oxo-chromen-2-yl)-5-(trifluoromethyl)phenoxy]-2-hydroxy-propyl]pyrrolidine-3-carboxylic acid

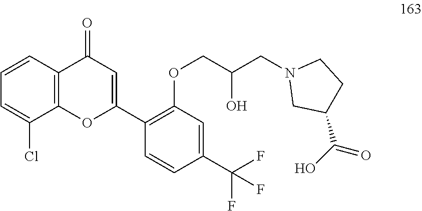 [SMILES: O=C(O)[C@H]1CCN(CC(O)COc2cc(C(F)(F)F)ccc2-c2cc(=O)c3cccc(Cl)c3o2)C1]

To a solution of methyl (3S)-1-[3-[2-(8-chloro-4-oxo-chromen-2-yl)-5-(trifluoromethyl)phenoxy]-2-hydroxy-propyl]pyrrolidine-3-carboxylate (60 mg, 114 μmol) in the mixed solvent of DMF (5 mL) and MeOH (2 mL) was added LiOH (2.73 mg, 114 μmol). The reaction mixture was stirred at room temperature overnight. After the reaction was complete, the reaction mixture was acidified with HOAc and then the resulting solution was purified by prep-HPLC to afford (3S)-1-[3-[2-(8-chloro-4-oxo-chromen-2-yl)-5-(trifluoromethyl)phenoxy]-2-hydroxy-propyl]pyrrolidine-3-carboxylic acid (13 mg, yield: 21.1%) as a white powder. MS obsd. (ESI⁺) [(M+H)⁺]: 511.8.

Example 163: ¹H NMR (DMSO-d₆, 400 MHz) δ ppm 8.14 (d, J=8.1 Hz, 1H), 8.04 (d, J=7.9 Hz, 2H), 7.58-7.67 (m, 2H), 7.47-7.56 (m, 1H), 7.18 (d, J=4.6 Hz, 1H), 6.07 (br dd, J=10.0, 4.1 Hz, 1H), 4.25-4.42 (m, 3H), 3.75-3.88 (m, 1H), 3.54-3.70 (m, 1H), 3.38-3.47 (m, 1H), 3.27 (br d, J=3.3 Hz, 2H), 3.08-3.20 (m, 1H), 2.76-2.83 (m, 1H), 1.96-2.29 (m, 2H).

### Example 164

### (3S)-1-[3-[5-bromo-2-(8-chloro-4-oxo-chromen-2-yl)phenoxy]-2-hydroxy-propyl]pyrrolidine-3-carboxylic acid

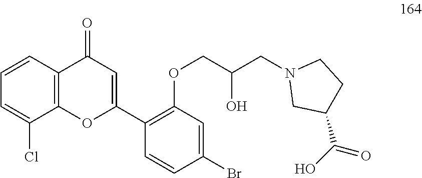 [SMILES: O=C(O)[C@H]1CCN(CC(O)COc2cc(Br)ccc2-c2cc(=O)c3cccc(Cl)c3o2)C1]

The compound 164 was prepared in analogy to the procedure described for the preparation of 163, replacing 8-chloro-2-[2-hydroxy-4-(trifluoromethyl)phenyl]chromen-4-one with 2-(4-bromo-2-hydroxy-phenyl)-8-chloro-chromen-4-one as starting material in Step 1. MS obsd. (ESI⁺) [(M+H)⁺]: 522.1.

Example 164: ¹H NMR (DMSO-d₆, 400 MHz) δ ppm 12.56-13.11 (m, 1H), 8.02 (d, J=7.8 Hz, 2H), 7.88 (d, J=8.4 Hz, 1H), 7.37-7.64 (m, 3H), 7.12 (d, J=3.1 Hz, 1H), 5.90-6.15 (m, 1H), 4.13-4.39 (m, 4H), 3.72-3.93 (m, 2H), 3.56-3.70 (m, 2H), 3.10-3.21 (m, 1H), 1.85-2.27 (m, 3H).

### Example 165: (3R)-1-[3-[5-bromo-2-(8-chloro-4-oxo-chromen-2-yl)phenoxy]-2-hydroxy-propyl]pyrrolidine-3-carboxylic acid

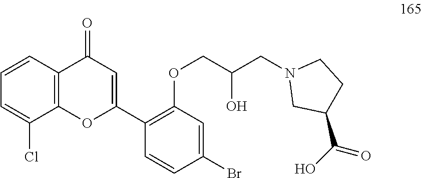 [SMILES: O=C(O)[C@@H]1CCN(CC(O)COc2cc(Br)ccc2-c2cc(=O)c3cccc(Cl)c3o2)C1]

The compound 165 was prepared in analogy to the procedure described for the preparation of 164, replacing 8-chloro-2-[2-hydroxy-4-(trifluoromethyl)phenyl]chromen-4-one with 2-(4-bromo-2-hydroxy-phenyl)-8-chloro-chromen-4-one in Step 1 and methyl (R)-pyrrolidine-3-carboxylate with methyl (S)-pyrrolidine-3-carboxylate in Step 2 as starting material. MS obsd. (ESI⁺) [(M+H)⁺]: 522.0.

Example 165: ¹H NMR (DMSO-d₆, 400 MHz) δ ppm 12.73-13.22 (m, 1H), 8.03 (dd, J=7.8, 1.2 Hz, 2H), 7.89 (d, J=8.4 Hz, 1H), 7.37-7.62 (m, 3H), 7.13 (dd, J=3.0, 1.3 Hz, 1H), 5.97-6.14 (m, 1H), 4.13-4.43 (m, 4H), 3.75-3.96 (m, 2H), 3.59-3.72 (m, 2H), 3.06-3.23 (m, 1H), 1.95-2.41 (m, 3H).

### Example 166: 2-[[3-[2-(8-chloro-4-oxo-chromen-2-yl)-5-(trifluoromethyl)phenoxy]-2-hydroxy-propyl]amino]-2-oxo-acetic acid

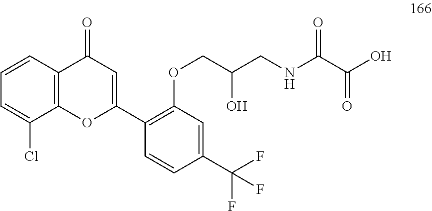 [SMILES: O=C(O)C(=O)NCC(O)COc1cc(C(F)(F)F)ccc1-c1cc(=O)c2cccc(Cl)c2o1]

### Step 1: Preparation of 8-chloro-2-[2-[2-hydroxy-3-[(4-methoxyphenyl)methylamino]propoxy]-4-(trifluoromethyl)phenyl]chromen-4-one

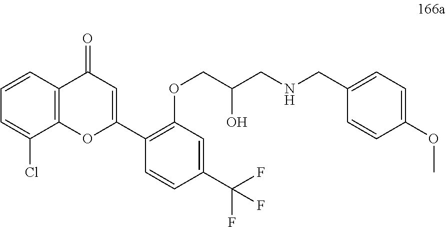 [SMILES: COc1ccc(CNCC(O)COc2cc(C(F)(F)F)ccc2-c2cc(=O)c3cccc(Cl)c3o2)cc1]

To a solution of 4-aminomethyl-anisole (0.26 mL, 2.02 mmol) in ethanol (8.86 mL) was added K₂CO₃ (1.38 g, 10.1 mmol) and 8-chloro-2-[2-(oxiran-2-ylmethoxy)-4-(trifluoromethyl)phenyl]chromen-4-one (800 mg, 2.02 mmol) under N₂. Then, the reaction was heated and to 50° C. and kept stirring at this temperature for 16 hours. After the starting material was consumed, the reaction mixture was concentrated in vacuo. The crude product was purified by flash column (eluting with EtOAc:Isohexane=60%) to give 8-chloro-2-[2-[2-hydroxy-3-[(4-methoxyphenyl)methylamino]propoxy]-4-(trifluoromethyl)phenyl]chromen-4-one (800 mg, yield: 74.3%) as a white solid. MS obsd. (ESI⁺)[(M+H)⁺]: 534.0.

### Step 2: Preparation of 2-[2-(3-amino-2-hydroxy-propoxy)-4-(trifluoromethyl)phenyl]-8-chloro-chromen-4-one

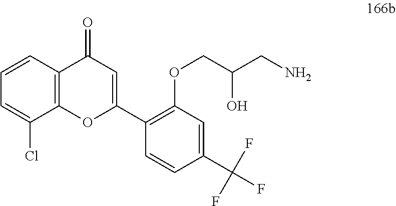 [SMILES: NCC(O)COc1cc(C(F)(F)F)ccc1-c1cc(=O)c2cccc(Cl)c2o1]

A solution of 8-chloro-2-[2-[2-hydroxy-3-[(4-methoxyphenyl)methylamino]propoxy]-4-(trifluoromethyl)phenyl]chromen-4-one (800 mg, 1.5 mmol) and TFA (10.0 mL, 1.5 mmol) in DCM was stirred at 100° C. under N₂ for 8 hours. After the reaction was consumed, the reaction mixture was concentrated in vacuo to give 2-[2-(3-amino-2-hydroxy-propoxy)-4-(trifluoromethyl)phenyl]-8-chloro-chromen-4-one (300 mg, yield: 48.4%) as colorless oil. MS obsd. (ESI⁺)[(M+H)⁺]: 414.0.

### Step 3: Preparation of ethyl 2-[[3-[2-(8-chloro-4-oxo-chromen-2-yl)-5-(trifluoromethyl)phenoxy]-2-hydroxy-propyl]amino]-2-oxo-acetate

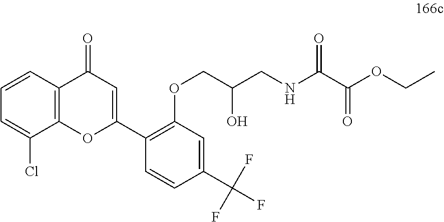 [SMILES: CCOC(=O)C(=O)NCC(O)COc1cc(C(F)(F)F)ccc1-c1cc(=O)c2cccc(Cl)c2o1]

To a solution of 2-[2-(3-amino-2-hydroxy-propoxy)-4-(trifluoromethyl)phenyl]-8-chloro-chromen-4-one (110 mg, 0.270 mmol) in DCM (5.5 mL) was added TEA (0.07 mL, 0.530 mmol) and ethyl oxalyl chloride (43.56 mg, 0.320 mmol) at 0° C. Then, the reaction was stirred at room temperature for 16 hours. After the reaction was completed, the reaction mixture was quenched with water (100 mL) and the aqueous layer was extracted with DCM (50 mL) three times. The combined organic layer was washed with brine (50 mL) twice, dried over anhydrous MgSO₄, filtered and concentrated in vacuo to give the crude product, which was purified by flash column chromatography (EtOAc:PE=50%) to afford ethyl 2-[[3-[2-(8-chloro-4-oxo-chromen-2-yl)-5-(trifluoromethyl)phenoxy]-2-hydroxy-propyl]amino]-2-oxo-acetate (80 mg, yield: 58.6%) as a light yellow solid. MS obsd. (ESI⁺)[(M+H)⁺]: 514.0.

### Step 4: Preparation of 2-[[3-[2-(8-chloro-4-oxo-chromen-2-yl)-5-(trifluoromethyl)phenoxy]-2-hydroxy-propyl]amino]-2-oxo-acetic acid

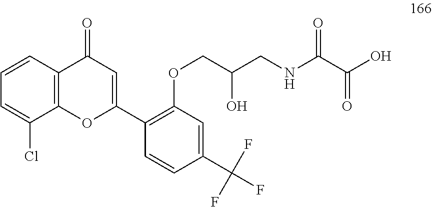 [SMILES: O=C(O)C(=O)NCC(O)COc1cc(C(F)(F)F)ccc1-c1cc(=O)c2cccc(Cl)c2o1]

To a solution of ethyl 2-[[3-[2-(8-chloro-4-oxo-chromen-2-yl)-5-(trifluoromethyl)phenoxy]-2-hydroxy-propyl]amino]-2-oxo-acetate (116.81 mg, 0.230 mmol) in THF (1.2 mL) and water (1.2 mL) was added lithium hydroxide (19 mg, 0.450 mmol). Then the mixture was stirred at room temperature for 2 hours. After the reaction was completed, the reaction mixture was adjusted to pH 6 with 1N HCl. The resulting residue was concentrated in vacuo to give the crude product, which was purified by prep-HPLC to afford 2-[[3-[2-(8-chloro-4-oxo-chromen-2-yl)-5-(trifluoromethyl)phenoxy]-2-hydroxy-propyl]amino]-2-oxo-acetic acid (51 mg, yield: 43.9%) as a white solid. MS obsd. (ESI⁺)[(M+H)⁺]: 486.0.

Example 166: 13.69-13.92 (m, 1H), 8.82 (br t, J=5.9 Hz, 1H), 8.16 (d, J=8.1 Hz, 1H), 8.02 (d, J=7.7 Hz, 2H), 7.46-7.65 (m, 3H), 7.29 (s, 1H), 5.31-5.50 (m, 1H), 4.22-4.31 (m, 1H), 4.10-4.19 (m, 1H), 3.97-4.07 (m, 1H), 3.40-3.52 (m, 2H).

### Example 167: 2-[2-[2-(8-chloro-4-oxo-chromen-2-yl)-5-(trifluoromethyl)phenoxy]ethylamino]acetamide

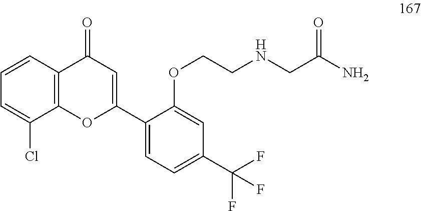 [SMILES: NC(=O)CNCCOc1cc(C(F)(F)F)ccc1-c1cc(=O)c2cccc(Cl)c2o1]

### Step 1: Preparation of ethyl 2-[2-[2-(8-chloro-4-oxo-chromen-2-yl)-5-(trifluoromethyl)phenoxy]ethylamino]acetate

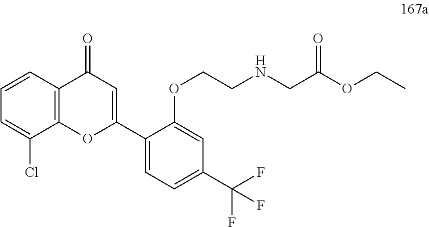 [SMILES: CCOC(=O)CNCCOc1cc(C(F)(F)F)ccc1-c1cc(=O)c2cccc(Cl)c2o1]

To a solution of 2-[2-(2-aminoethoxy)-4-(trifluoromethyl)phenyl]-8-chloro-chromen-4-one (400 mg, 1.04 mmol) and ethyl glyoxalate (213 mg, 1.04 mmol) in methanol (16 mL) was added sodium cyanoborohydride (196.5 mg, 3.13 mmol). The reaction mixture was stirred at 30° C. for 6 hours and then quenched with water (2 mL). The resulting mixture was concentrated in vacuo to give the crude product, which was purified by flash chromatography (eluting with EtOAc:PE=20%) to give ethyl 2-[2-[2-(8-chloro-4-oxo-chromen-2-yl)-5-(trifluoromethyl)phenoxy]ethylamino]acetate (203 mg, yield: 41.5%) as a light yellow solid. MS obsd. (ESI⁺)[(M+H)⁺]: 470.1.

### Step 2: Preparation of 2-[2-[2-(8-chloro-4-oxo-chromen-2-yl)-5-(trifluoromethyl)phenoxy]ethylamino]acetic acid

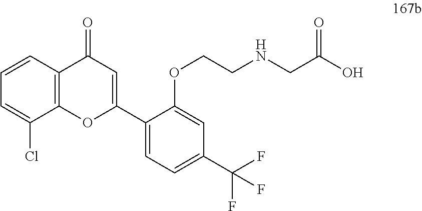 [SMILES: O=C(O)CNCCOc1cc(C(F)(F)F)ccc1-c1cc(=O)c2cccc(Cl)c2o1]

To a solution of ethyl 2-[2-[2-(8-chloro-4-oxo-chromen-2-yl)-5-(trifluoromethyl)phenoxy]ethylamino]acetate (170 mg, 0.360 mmol) in methanol was added hydrochloric acid (15 mL, 180 mmol). The reaction mixture was stirred at 100° C. for 6 hours and then concentrated in vacuo. The resulting residue was triturated with EtOAc (10 mL) and filtered to afford 2-[2-[2-(8-chloro-4-oxo-chromen-2-yl)-5-(trifluoromethyl)phenoxy]ethylamino]acetic acid (150 mg, yield: 93.8%) as a white solid. MS obsd. (ESI⁺)[(M+H)⁺]: 442.1.

### Step 3: Preparation of 2-[2-[2-(8-chloro-4-oxo-chromen-2-yl)-5-(trifluoromethyl)phenoxy]ethylamino]acetamide

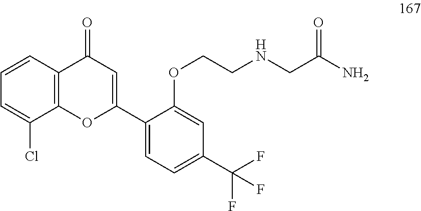 [SMILES: NC(=O)CNCCOc1cc(C(F)(F)F)ccc1-c1cc(=O)c2cccc(Cl)c2o1]

To a solution of 2-[2-[2-(8-chloro-4-oxo-chromen-2-yl)-5-(trifluoromethyl)phenoxy]ethylamino]acetic acid (150 mg, 0.340 mmol), DIPEA (0.24 mL, 1.36 mmol), and HATU (258.2 mg, 0.680 mmol) in DCM (5 mL) was added ammonium chloride (36 mg, 0.680 mmol). The reaction mixture was stirred at 30° C. for 12 hours. After the reaction was completed, the reaction mixture was quenched with water (1 ML) and concentrated in vacuo to give the crude product, which was purified by prep-HPLC to afford 2-[2-[2-(8-chloro-4-oxo-chromen-2-yl)-5-(trifluoromethyl)phenoxy]ethylamino]acetamide (12.5 mg, yield: 8.1%) as a white solid. MS obsd. (ESI⁺): 441.1

Example 167: ¹H NMR (400 MHz, DMSO-d₆): δ ppm 8.15 (d, J=7.9 Hz, 1H), 7.96-8.07 (m, 2H), 7.45-7.63 (m, 3H), 7.24 (s, 2H), 7.01 (br s, 1H), 4.33 (t, J=5.4 Hz, 2H), 3.04-3.16 (m, 2H), 2.95 (t, J=5.3 Hz, 2H).

### Example 168: 2-[2-[2-(8-chloro-4-oxo-chromen-2-yl)-5-(trifluoromethyl)phenoxy]ethylamino]propanamide

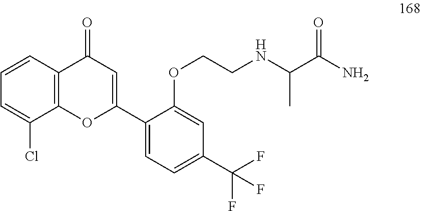 [SMILES: CC(NCCOc1cc(C(F)(F)F)ccc1-c1cc(=O)c2cccc(Cl)c2o1)C(N)=O]

The compound 168 was prepared in analogy to the procedure described for the preparation of 168, replacing ethyl glyoxalate with ethyl pyruvate as starting material in Step 1. MS obsd. (ESI⁺) [(M+H)⁺]: 455.1.

Example 168: ¹H NMR (DMSO-d₆, 400 MHz) δ ppm 8.13-8.19 (m, 1H), 7.97-8.05 (m, 2H), 7.48-7.61 (m, 3H), 7.26-7.33 (m, 2H), 6.95 (br s, 1H), 4.29-4.37 (m, 2H), 3.03-3.14 (m, 1H), 2.77-2.99 (m, 2H), 1.13 (d, J=6.7 Hz, 3H).

### Example 169: 1-[3-[5-bromo-2-(8-chloro-4-oxo-chromen-2-yl)phenoxy]propyl]-5-oxo-pyrrolidine-3-carboxylic acid

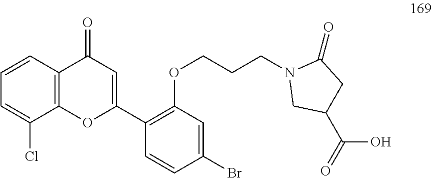 [SMILES: O=C(O)C1CC(=O)N(CCCOc2cc(Br)ccc2-c2cc(=O)c3cccc(Cl)c3o2)C1]

### Step 1: Preparation of methyl 1-[3-[5-bromo-2-(8-chloro-4-oxo-chromen-2-yl)phenoxy]propyl]-5-oxo-pyrrolidine-3-carboxylate

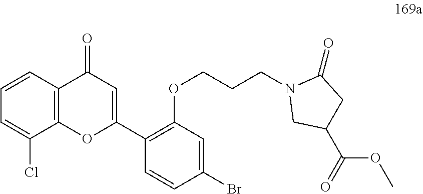 [SMILES: COC(=O)C1CC(=O)N(CCCOc2cc(Br)ccc2-c2cc(=O)c3cccc(Cl)c3o2)C1]

To a solution of 2-[2-(3-aminopropoxy)-4-bromo-phenyl]-8-chloro-chromen-4-one (300 mg, 0.670 mmol) in methanol (6 mL) were added TEA (0.09 mL, 0.670 mmol) and dimethyl itaconate (106.59 mg, 0.670 mmol). The reaction mixture was stirred at 60° C. for 20 hours and then quenched with water (50 mL). The aqueous layer was extracted with EtOAc (50 mL) three times. The organic layer was combined, washed with brine, dried over anhydrous sodium sulfate and concentrated in vacuo. The resulting residue was purified by flash chromatography (EtOAc:PE=50%) to give methyl 1-[3-[5-bromo-2-(8-chloro-4-oxo-chromen-2-yl)phenoxy]propyl]-5-oxo-pyrrolidine-3-carboxylate (270 mg, yield: 68.9%) as a yellow solid. MS obsd. (ESI⁺) [(M+H)⁺]: 534.0.

### Step 2: Preparation of 1-[3-[5-bromo-2-(8-chloro-4-oxo-chromen-2-yl)phenoxy]propyl]-5-oxo-pyrrolidine-3-carboxylic acid

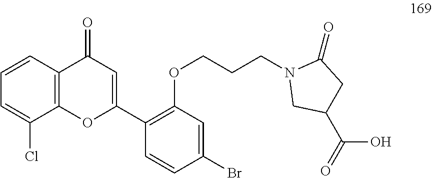 [SMILES: O=C(O)C1CC(=O)N(CCCOc2cc(Br)ccc2-c2cc(=O)c3cccc(Cl)c3o2)C1]

To a solution of methyl 1-[3-[5-bromo-2-(8-chloro-4-oxo-chromen-2-yl)phenoxy]propyl]-5-oxo-pyrrolidine-3-carboxylate (270 mg, 0.500 mmol) in THF (2 mL) and water (2 mL) was added lithium hydroxide (18.14 mg, 0.760 mmol). The mixture was stirred at room temperature for 2 hours. After the reaction was completed, the reaction mixture was added water (20 mL) and EtOAc (20 mL) to form a suspension. The suspension was filtered and the filter cake was washed with EtOAc (50 mL) to afford 1-[3-[5-bromo-2-(8-chloro-4-oxo-chromen-2-yl)phenoxy]propyl]-5-oxo-pyrrolidine-3-carboxylic acid (86 mg, yield: 32.4%) as a yellow solid. MS obsd. (ESI⁺) [(M+H)⁺]: 520.1.

Example 169: ¹H NMR (400 MHz, DMSO) δ ppm: 12.61 (br s, 1H), 8.00 (d, J=7.8 Hz, 2H), 7.87 (d, J=8.3 Hz, 1H), 7.38-7.54 (m, 3H), 7.05-7.15 (m, 1H), 4.17 (t, J=6.2 Hz, 2H), 3.53-3.62 (m, 1H), 3.45-3.52 (m, 1H), 3.34-3.38 (m, 2H), 3.09-3.23 (m, 1H), 2.33-2.47 (m, 2H), 1.91-2.08 (m, 2H).

Similar flavone compounds, 7-hydroxy-2-(2-hydroxyphenyl)chromen-4-one (compound F-1) in patent WO2015061294 for treating HBV infection as STING agonist and 6-methoxy-2-(2-methoxyphenyl)chromen-4-one (compound F-2) disclosed in patent WO 2001003681 for treating infections, were chosen as reference compounds in present invention.

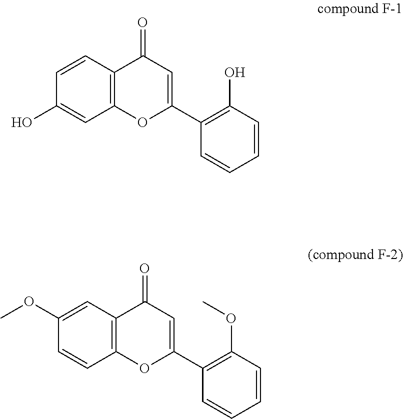 [SMILES: O=c1cc(-c2ccccc2O)oc2cc(O)ccc12 ; COc1ccc2oc(-c3ccccc3OC)cc(=O)c2c1]

### BIOLOGICAL EXAMPLES

### BIO-Example 1: Engineered HepDES19 Primary Screen Assay

The assay was employed to screen for cccDNA inhibitors. HepDES19 is a cccDNA-producing cell line. In this cell line, HBeAg in the cell culture supernatant as surrogate marker, as HBeAg production depends on cccDNA level and activity. HepDES19 is an engineered cell line which contains a 1.1 unit length HBV genome, and pgRNA transcription from the transgene is controlled by Tetracycline (Tet). In the absence of Tet, pgRNA transcription will be induced, but HBV e antigen (HBeAg) could not be produced from this pgRNA due to very short leader sequence before the HBeAg start codon and the start codon is disrupted. Only after cccDNA is formed, the missing leader sequence and start codon mutation would be restored from the 3′-terminal redundancy of pgRNA, and then HBeAg could be synthesized. Therefore, HBeAg could be used as a surrogate marker for cccDNA (Zhou, T. et al., Antiviral Res. (2006), 72(2), 116-124; Guo, H. et al., J. Virol. (2007), 81(22), 12472-12484).

HepDES19 cells were seeded at 2×10⁶ cells per T150 flask and cultured with the culture medium (Dulbecco's Modified Eagle Medium: Nutrient Mixture F-12 [DMEM-F12, Gibco Cat. 11320-82], 10% Fetal Bovine Serum [FBS, Clontech Cat. 631101], 0.1 mM Non-Essential Amino Acids Solution [NEAA, Gibco Cat. 11140-050], 50 μg/mL Penicillin-Streptomycin [PS, Invitrogen Cat. 15140-163], 500 μg/mL Geneticin [G418, Invitrogen Cat. 10131-027]) containing 3 μg/mL Tet (Sigma, Cat. 87128) for 5 days. Cells were then seeded at 4×10⁶ cells per T150 in the same culture medium as described above in the absence of Tet for 8 days. Cells were then harvested and frozen at density of 2×10⁶ cells per mL. For compound testing, the frozen cells were thawed and seeded into 96-well plates at a density of 6×10⁴ cells per well. At 24 hours after seeding, half log serial dilutions of compounds made with Dimethyl sulfoxide (DMSO, Sigma, Cat. D2650) were further diluted with the same culture medium as described above before they were added to the cells to reach desired final compound concentrations and 1% DMSO concentration. Plates were then incubated at 37° C. for another 5 days before measurement of HBeAg level and cell viability. Intracellular HBeAg level were measured with enzyme-linked immunosorbent assay (ELISA) kit (Shanghai Kehua Diagnostic Medical Products Co., Ltd). Cell viability was assessed using Cell Counting Kit-8 (DonJindo, Cat. CK04-20). IC₅₀ values were derived from the dose-response curve using 4 parameter logistic curve fit method.

The compounds of the present invention were tested for their capacity to inhibit extracellular HBeAg level as described herein. The compounds of this invention were found to have IC₅₀ below 50 μM. Particular compounds of formula (I) were found to have IC₅₀ below 5.0 μM. Results of HepDES19 primary screen assay are given in Table BIO1.

TABLE BIO1
Activity data in HepDES19 primary screen assay

| Example | IC₅₀ | Example | IC₅₀ | Example | IC₅₀ |
|---|---|---|---|---|---|
| No. | (μM) | No. | (μM) | No. | (μM) |
| 001 | 0.35 | 002 | 0.25 | 003 | 2.41 |
| 004 | 2.02 | 005 | 8.93 | 006 | 4.68 |
| 007 | 9.50 | 008 | 0.17 | 009 | 7.47 |
| 010 | 0.22 | 011 | 3.61 | 012 | 0.11 |
| 013 | 4.23 | 014 | 0.37 | 015 | 7.85 |
| 016 | 0.89 | 017 | 0.34 | 018 | 0.41 |
| 019 | 0.57 | 020 | 0.87 | 021 | 0.18 |
| 022 | 1.31 | 023 | 0.13 | 024 | 1.04 |
| 025 | 0.79 | 026 | 0.46 | 027 | 0.09 |
| 029 | 1.85 | 030 | 1.33 | 031 | 0.17 |
| 032 | 0.65 | 033 | 1.09 | 034 | 1.12 |
| 035 | 2.38 | 036 | 2.03 | 037 | 4.12 |
| 038 | 7.24 | 039 | 3.48 | 040 | 9.06 |
| 041 | 0.03 | 042 | 0.05 | 043 | 1.42 |
| 044 | 4.75 | 045 | 6.50 | 046 | 4.37 |
| 047 | 0.99 | 048 | 6.68 | 049 | 2.81 |
| 050 | 6.93 | 051 | 7.86 | 052 | 0.08 |
| 053 | 0.10 | 054 | 0.24 | 055 | 0.98 |
| 056 | 1.00 | 057 | 1.50 | 058 | 3.41 |
| 059 | 4.68 | 060 | 4.87 | 061 | 5.71 |
| 062 | 2.02 | 063 | 4.32 | 064 | 0.08 |
| 065 | 7.21 | 066 | 2.79 | 067 | 8.40 |
| 068 | 5.51 | 069 | 1.77 | 070 | 1.01 |
| 071 | 1.03 | 072 | 2.89 | 073 | 0.14 |
| 074 | 1.03 | 075 | 0.11 | 076 | 1.64 |
| 077 | 0.05 | 078 | 7.80 | 079 | 0.78 |
| 080 | 3.14 | 081 | 7.11 | 082 | 0.24 |
| 083 | 0.56 | 084 | 0.59 | 085 | 1.08 |
| 086 | 3.22 | 087 | 5.24 | 088 | 0.27 |
| 089 | 1.94 | 090 | 1.30 | 091 | 2.83 |
| 092 | 0.22 | 093 | 5.98 | 094 | 9.79 |
| 095 | 9.30 | 096 | 0.13 | 097 | 1.16 |
| 098 | 4.34 | 099 | 6.47 | 100 | 3.84 |
| 101 | 1.08 | 102 | 2.91 | 103 | 3.66 |
| 104 | 9.65 | 105 | 1.90 | 106 | 1.10 |
| 107 | 2.71 | 108 | 1.14 | 109 | 4.11 |
| 110 | 3.76 | 111 | 0.18 | 112 | 0.49 |
| 113 | 0.49 | 114 | 0.87 | 115 | 1.04 |
| 116 | 1.80 | 117 | 2.16 | 118 | 1.98 |
| 119 | 3.43 | 120 | 2.96 | 121 | 4.25 |
| 122 | 7.72 | 123 | 5.11 | 124 | 5.88 |
| 125 | 2.03 | 126 | 0.91 | 127 | 0.08 |
| 128 | 0.27 | 129 | 4.75 | 130 | 4.82 |
| 131 | 5.86 | 132 | 0.35 | 133 | 4.57 |
| 134 | 8.17 | 135 | 0.21 | 136 | 0.58 |
| 137 | 3.40 | 138 | 4.56 | 139 | 10.20 |
| 140 | 0.19 | 141 | 0.35 | 142 | 0.74 |
| 143 | 2.42 | 144 | 11.40 | 145 | 14.30 |
| 146 | 0.33 | 147 | 1.20 | 148 | 1.21 |
| 149 | 8.79 | 150 | 12.20 | 151 | 0.63 |
| 152 | 0.48 | 153 | 0.83 | 154 | 1.43 |
| 155 | 5.11 | 156 | 4.14 | 157 | 4.15 |
| 158 | 8.35 | 159 | 0.83 | 160 | 2.43 |
| 161 | 4.74 | 162 | 0.08 | 163 | 2.76 |
| 164 | 2.96 | 165 | 3.19 | 166 | 1.57 |
| 167 | 3.30 | 168 | 1.14 | 169 | 0.99 |
| F-1 | >50 | F-2 | >50 | | |

### BIO-Example 2: Cryopreserved Primary Human Hepatocytes (PHH) Assay

This assay is used to confirm the anti-HBV effect of the compounds in HBV PHH infection assay. Cryopreserved PHH (BioreclamationIVT, Lot YJM) was thawed at 37° C. and gently transferred into pre-warmed InVitroGRO HT medium (BioreclamationIVT, Cat. S03317). The mixture was centrifuged at 70 relative centrifugal force (RCF) for 3 minutes at RT, and the supernatant was discarded. Pre-warmed InVitroGRO CP medium (BioreclamationIVT, Cat #S03316) was added to the cell pellet to gently re-suspend cells. The cells were seeded at the density of 5.8×10⁴ cells per well to collagen I coated 96-well plate (Gibco, Cat. A1142803) with the InVitroGRO CP medium. All plates were incubated at 37° C. with 5% CO₂ and 85% humidity.

At 20 hours after plating, the medium was changed to PHH culture medium (Dulbecco's Modified Eagle Medium (DMEM)/F12 (1:1) (Gibco, Cat. 11320-033), 10% fetal bovine serum (Gibco Cat. 10099141), 100 U/mL penicillin, 100 μg/mL streptomycin (Gibco, Cat. 151401-122), 5 ng/mL human epidermal growth factor (Invitrogen Cat. PHG0311L), 20 ng/mL dexamethasone (Sigma, Cat. D4902) and 250 ng/mL human recombinant insulin (Gibco, Cat. 12585-014)). And the cells were incubated at 37° C. with 5% CO₂ and 85% humidity for 4 hours. The medium was then changed to pre-warmed PHH culture medium containing 4% polyethylene glycol (PEG) MW8000 (Sigma, Cat. P1458-50ML) and 1% DMSO (Sigma, Cat. D2650). 5.8×10⁶ genomic equivalents of HBV were added into the medium.

At 24 hours post-infection, the cells were gently washed with PBS and refreshed with PHH culture medium supplemented with 1% DMSO, and 0.25 mg/mL Matrix gel (Corning, Cat. 356237) at 200 μL per well. All plates were immediately placed in at 37° C. CO₂ incubator. 24 hours later, serial dilutions of compounds made with DMSO were further diluted with the same culture medium (PHH culture medium supplemented with 1% DMSO and 0.25 mg/mL Matrix gel as described above) before they were added to the cells to reach desired final compound concentrations and 1% DMSO concentration. The medium containing the compounds were refreshed every three days.

At 9 days post-compound treatment, extracellular HBsAg level were measured with Chemiluminescence Immuno Assay (CLIA) kit (Autobio, HBsAg Quantitative CLIA). Extracellular HBV DNA was extracted by MagNA Pure 96 system (Roche) and then determined

HBV-Forward Primer (SEQ ID NO: 1):
AAGAAAAACCCCGCCTGTAA (5' to 3');
HBV-Reverse Primer (SEQ ID NO: 2):
CCTGTTCTGACTACTGCCTCTCC (5' to 3');
HBV-Probe:
5′ + tetramethylrhodamine + SEQ ID NO: 3 + black
hole quencher 2-3′, wherein SEQ ID NO: 3 is
CCTGATGTGATGTTCTCCATGTTCAGC.

HBsAg IC₅₀ and HBV DNA IC₅₀ values were derived from the dose-response curve using 4 parameter logistic curve fit method. The compounds of formula (I) have HBsAg IC₅₀<20 μM, particularly <1 μM; and HBV DNA IC₅₀<50 μM. Results of Cryopreserved PHH assay are given in TableBIO2.

TABLE BIO2
HBsAg IC₅₀ data in Cryopreserved PHH assay

| | HBsAg | | HBsAg | | HBsAg |
|---|---|---|---|---|---|
| Example | IC50 | Example | IC50 | Example | IC50 |
| No. | (μM) | No. | (μM) | No. | (μM) |
| 001 | 5.43 | 002 | 4.01 | 005 | 8.34 |
| 006 | 3.82 | 007 | 4.95 | 013 | 7.35 |
| 014 | 5.08 | 015 | 9.75 | 016 | 7.64 |
| 017 | 3.42 | 018 | 5.13 | 019 | 7.13 |
| 020 | 8.36 | 022 | 6.80 | 023 | 4.95 |
| 025 | 7.63 | 027 | 5.85 | 041 | 5.48 |
| 042 | 7.30 | 047 | 2.83 | 049 | 7.81 |
| 052 | 5.82 | 064 | 6.41 | 066 | 9.03 |
| 073 | 7.47 | 075 | 3.57 | 076 | 7.95 |
| 077 | 2.18 | 080 | 4.02 | 082 | 7.56 |
| 084 | 3.48 | 087 | 7.84 | 088 | 7.80 |
| 089 | 5.81 | 093 | 6.13 | 098 | 6.34 |
| 101 | 1.41 | 116 | 3.41 | 117 | 1.90 |
| 119 | 0.67 | 125 | 3.19 | 126 | 4.21 |
| 131 | 5.05 | 133 | 2.80 | 146 | 7.08 |
| 157 | 3.40 | 160 | 10.00 | 164 | 9.66 |
| 165 | 9.98 | 166 | 3.03 | 167 | 7.94 |

1. A compound of formula (I), 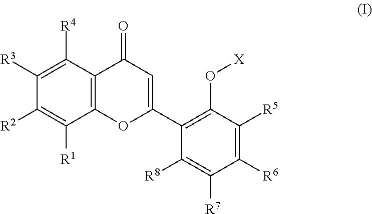
wherein:
R¹ is halogen;
R² is selected from H, OH, halogen, C₁₋₆alkyl, haloC₁₋₆alkyl and C₁₋₆alkoxy;
R³ is selected from H, OH, halogen, C₁₋₆alkyl, haloC₁₋₆alkyl and C₁₋₆alkoxy;
R⁴ is selected from H, OH, halogen, C₁₋₆alkyl, haloC₁₋₆alkyl and C₁₋₆alkoxy;
R⁵ is selected from H, OH, halogen, C₁₋₆alkyl, haloC₁₋₆alkyl and C₁₋₆alkoxy;
R⁶ is selected from H, OH, halogen, C₁₋₆alkyl, haloC₁₋₆alkyl and C₁₋₆alkoxy;
R⁷ is selected from H, OH, halogen, C₁₋₆alkyl, haloC₁₋₆alkyl and C₁₋₆alkoxy;
R⁸ is selected from H, OH, halogen, C₁₋₆alkyl, haloC₁₋₆alkyl and C₁₋₆alkoxy;
X is 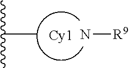
or -L-Y,
wherein:
Cy1 is an N-containing heterocyclyl;
R⁹ is selected from C₃₋₇cycloalkylsulfonyl, carboxycarbonyl, carboxyphenylC₁₋₆alkyl and carboxyC₃₋₇cycloalkyl;
L is selected from C₁₋₆alkyl, C₁₋₆alkoxyC₁₋₆alkyl, C₃₋₇cycloalkyl, C₃₋₇cycloalkylC₁₋₆alkyl and C₁₋₆alkylheterocyclylC₁₋₆alkyl; wherein C₁₋₆alkyl is unsubstituted or substituted by OH;
and
Y is —NR¹⁰R¹¹ or 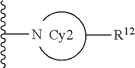
wherein:
Cy2 is N-containing heterocyclyl; wherein N-containing heterocyclyl is unsubstituted or substituted by one or two or three substituents independently selected from halogen, C₁₋₆alkyl and OH;
R¹⁰ is selected from H, C₁₋₆alkyl, C₃₋₇cycloalkyl, C₁₋₆alkylsulfonyl, C₃₋₇cycloalkylsulfonyl, carboxyC₁₋₆alkyl, phenylsulfonyl and C₁₋₆alkylcarbonyl;
R¹¹ is selected from carboxyC₃₋₇cycloalkyl, carboxyC₁₋₆alkyl, carboxycarbonyl, carboxyheterocyclyl, carboxyC₃₋₇cycloalkylC₁₋₆alkyl, heterocyclyl, C₁₋₆alkylsulfonylC₁₋₆alkyl, aminosulfonylC₁₋₆alkyl, C₃₋₇cycloalkylsulfonyl, C₁₋₆alkoxycarbonylcarbonyl, phenylsulfonyl, C₁₋₆alkylcarbonyl, C₁₋₆alkoxycarbonyl, C₁₋₆alkylaminosulfonyl, aminocarbonylcarbonyl, C₃₋₇cycloalkylaminocarbonylcarbonyl, C₃₋₇cycloalkylsulfonylaminocarbonylcarbonyl, hydroxyheterocyclylcarbonylcarbonyl, carboxyC₁₋₆alkylaminocarbonyl, C₁₋₆alkylphenylsulfonylaminocarbonyl, aminocarbonyl, aminosulfonyl and aminocarbonylC₁₋₆alkyl; and
R¹² is selected from H, carboxy, carboxyC₁₋₆alkyl, C₁₋₆alkylsulfonylaminocarbonyl, C₃₋₇cycloalkylsulfonylaminocarbonyl, C₁₋₆alkyl(C₁₋₆alkylsulfonyl)amino, C₁₋₆alkylsulfonyl, C₁₋₆alkylcarbonyl, C₁₋₆alkylsulfinyl, C₁₋₆alkylsulfonimidoyl, aminocarbonyl, carboxycarbonyl and heterocyclyl;
or a pharmaceutically acceptable salt thereof.

2. A compound according to claim 1, wherein:
R² is H;
R³ is selected from H, halogen and C₁₋₆alkoxy;
R⁴ is selected from H and haloC₁₋₆alkyl;
R⁵ is H;
R⁶ is selected from H, halogen, C₁₋₆alkyl, haloC₁₋₆alkyl and C₁₋₆alkoxy;
R⁷ is selected from H, halogen, C₁₋₆alkyl and C₁₋₆alkoxy;
R⁸ is selected from H, halogen and C₁₋₆alkoxy;
X is 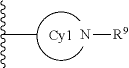
or -L-Y, wherein Cy1 is selected from piperidyl, pyrrolidinyl and azetidinyl;
R⁹ is selected from C₃₋₇cycloalkylsulfonyl, carboxycarbonyl, carboxyphenylC₁₋₆alkyl and carboxyC₃₋₇cycloalkyl;
L is selected from C₁₋₆alkyl, C₁₋₆alkoxyC₁₋₆alkyl, C₃₋₇cycloalkyl, C₃₋₇cycloalkylC₁₋₆alkyl and C₁₋₆alkyloxetanylC₁₋₆alkyl; wherein C₁₋₆alkyl is unsubstituted or substituted by OH;
and
Y is —NR¹⁰R¹¹ or 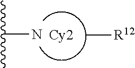
wherein:
Cy2 is selected from pyrrolidinyl, morpholinyl, azetidinyl, piperidyl, azabicyclo[3.2.1]octanyl, oxopyrrolidinyl, piperazinyl, thiomorpholinyl, dioxothiazinanyl, oxoimidazolidinyl and dioxoimidazolidinyl; wherein pyrrolidinyl is unsubstituted or substituted by one or two substituents independently selected from halogen, C₁₋₆alkyl and OH;
R¹⁰ is selected from H, C₁₋₆alkyl, C₃₋₇cycloalkyl, C₁₋₆alkylsulfonyl, C₃₋₇cycloalkylsulfonyl, carboxyC₁₋₆alkyl, phenylsulfonyl and C₁₋₆alkylcarbonyl;
R¹¹ is selected from carboxyC₃₋₇cycloalkyl, carboxyC₁₋₆alkyl, carboxycarbonyl, carboxytetrahydropyranyl, carboxyC₃₋₇cycloalkylC₁₋₆alkyl, dioxothiazinanyl, C₁₋₆alkylsulfonylC₁₋₆alkyl, aminosulfonylC₁₋₆alkyl, C₃₋₇cycloalkylsulfonyl, C₁₋₆alkoxycarbonylcarbonyl, phenylsulfonyl, C₁₋₆alkylcarbonyl, C₁₋₆alkoxycarbonyl, C₁₋₆alkylaminosulfonyl, aminocarbonylcarbonyl, C₃₋₇cycloalkylaminocarbonylcarbonyl, C₃₋₇cycloalkylsulfonylaminocarbonylcarbonyl, hydroxypyrrolidinylcarbonylcarbonyl, carboxyC₁₋₆alkylaminocarbonyl, C₁₋₆alkylphenylsulfonylaminocarbonyl, aminocarbonyl, aminosulfonyl and aminocarbonylC₁₋₆alkyl; and
R¹² is selected from H, carboxy, carboxyC₁₋₆alkyl, C₁₋₆alkylsulfonylaminocarbonyl, C₃₋₇cycloalkylsulfonylaminocarbonyl, C₁₋₆alkyl(C₁₋₆alkylsulfonyl)amino, C₁₋₆alkylsulfonyl, C₁₋₆alkylcarbonyl, C₁₋₆alkylsulfinyl, C₁₋₆alkylsulfonimidoyl, aminocarbonyl, carboxycarbonyl and 2H-tetrazolyl;
or a pharmaceutically acceptable salt thereof.

3. A compound according to claim 2, wherein:
R¹ is Cl;
R² is H;
R³ is selected from H, F and methoxy;
R⁴ is selected from H and CF₃;
R⁵ is H;
R⁶ is selected from H, Cl, Br, methyl, CF₃, methoxy, ethoxy and isobutoxy;
R⁷ is selected from H, Br, methyl and methoxy;
R⁸ is selected from H, F and methoxy;
X is 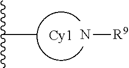
or -L-Y;
wherein:
Cy1 is selected from piperidyl, pyrrolidinyl and azetidinyl;
R⁹ is selected from cyclopropylsulfonyl, carboxycarbonyl, carboxyphenylmethyl and carboxycyclobutyl; and
L is selected from ethyl, propyl, isobutyl, ethoxyethyl, cyclobutyl, cyclopropylmethyl and methyloxetanylmethyl; wherein propyl is unsubstituted or substituted one time by OH;
and
Y is —NR¹⁰R¹¹ or 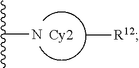
wherein:
Cy2 is selected from pyrrolidinyl, morpholinyl, azetidinyl, piperidyl, azabicyclo[3.2.1]octanyl, oxopyrrolidinyl, piperazinyl, thiomorpholinyl, dioxothiazinanyl, oxoimidazolidinyl and dioxoimidazolidinyl; wherein pyrrolidinyl is unsubstituted or substituted by one or two substituents independently selected from F, methyl, isopropyl, and OH;
R¹⁰ is selected from H, methyl, cyclopropyl, ethylsulfonyl, cyclopropylsulfonyl, carboxymethyl, phenylsulfonyl and methylcarbonyl;
R¹¹ is selected from carboxycyclobutyl, carboxycyclopentyl, carboxyisobutyl, carboxycarbonyl, carboxydimethylbutyl, carboxybicyclo[1.1.1]pentanyl, carboxycyclohexyl, carboxyethyl, carboxyisopropyl, carboxytetrahydropyranyl, carboxycyclohexylethyl, dioxothiazinanyl, methylsulfonylethyl, aminosulfonylethyl, cyclopropylsulfonyl, ethoxycarbonylcarbonyl, phenylsulfonyl, methylcarbonyl, ethylcarbonyl, methoxycarbonyl, ethoxycarbonyl, methylaminosulfonyl, carboxymethyl, ethylaminosulfonyl, aminocarbonylcarbonyl, cyclopropylaminocarbonylcarbonyl, cyclopropylsulfonylaminocarbonylcarbonyl, hydroxypyrrolidinylcarbonylcarbonyl, carboxyethylaminocarbonyl, carboxymethylaminocarbonyl, methylphenylsulfonylaminocarbonyl, aminocarbonyl, aminosulfonyl, aminocarbonylmethyl and aminocarbonylethyl; and
R¹² is selected from H, carboxy, carboxymethyl, methylsulfonylaminocarbonyl, cyclopropylsulfonylaminocarbonyl, methyl(methylsulfonyl)amino, methylsulfonyl, methylcarbonyl, methylsulfinyl, methylsulfonimidoyl, aminocarbonyl, carboxycarbonyl and 2H-tetrazolyl;
or a pharmaceutically acceptable salt thereof.

4. A compound according to claim 1, or a pharmaceutically acceptable salt thereof, wherein R³ is selected from H.

5. A compound according to claim 1, or a pharmaceutically acceptable salt thereof, wherein R⁶ is selected from halogen, C₁₋₆alkyl and haloC₁₋₆alkyl.

6. A compound according to claim 5, or a pharmaceutically acceptable salt thereof, wherein R⁶ is selected from Br, methyl and CF₃.

7. A compound according to claim 1, or a pharmaceutically acceptable salt thereof, R⁷ is selected from H and C₁₋₆alkoxy.

8. A compound according to claim 7, or a pharmaceutically acceptable salt thereof, wherein R⁷ is selected from H and methoxy.

9. A compound according to claim 1, or a pharmaceutically acceptable salt thereof, wherein X is -L-Y.

10. A compound according to a claim 1, or a pharmaceutically acceptable salt thereof, wherein L is selected from C₁₋₆alkyl and C₁₋₆alkoxyC₁₋₆alkyl, wherein C₁₋₆alkyl is unsubstituted or substituted by OH.

11. A compound according to claim 10, or a pharmaceutically acceptable salt thereof, wherein L is selected from ethyl, propyl and ethoxyethyl, wherein propyl is unsubstituted or substituted one time by OH.

12. A compound according to claim 1, or a pharmaceutically acceptable salt thereof, wherein Y is 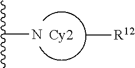
and wherein Cy2 is selected from pyrrolidinyl and morpholinyl.

13. A compound according to claim 1, or a pharmaceutically acceptable salt thereof, wherein R¹² is selected from carboxy, C₁₋₆alkylsulfonylaminocarbonyl and C₁₋₆alkylsulfonyl.

14. A compound according to claim 13, or a pharmaceutically acceptable salt thereof, wherein R¹² is selected from carboxy, methylsulfonylaminocarbonyl and methylsulfonyl.

15. A compound according to claim 1, or a pharmaceutically acceptable salt thereof, wherein Y is —NHR¹¹.

16. A compound according to claim 1, or a pharmaceutically acceptable salt thereof, wherein R¹¹ is selected from carboxyC₃₋₇cycloalkyl, carboxycarbonyl, carboxyC₁₋₆alkylaminocarbonyl, aminocarbonyl and aminosulfonyl.

17. A compound according to claim 16, or a pharmaceutically acceptable salt thereof, wherein R¹¹ is selected from carboxycyclobutyl, carboxycyclopentyl, carboxycarbonyl, carboxyethylaminocarbonyl, aminocarbonyl and aminosulfonyl.

18. A compound according to claim 1 having the formula (II), 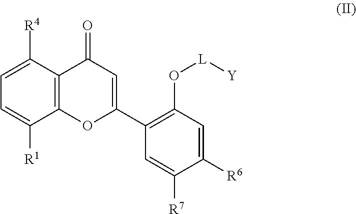
wherein:
R¹ is halogen;
R⁴ is selected from H and haloC₁₋₆alkyl;
R⁶ is selected from halogen, C₁₋₆alkyl and haloC₁₋₆alkyl;
R⁷ is selected from H and C₁₋₆alkoxy;
L is selected from C₁₋₆alkyl and C₁₋₆alkoxyC₁₋₆alkyl; wherein C₁₋₆alkyl is unsubstituted or substituted by OH; and
Y is —NHR¹¹ or 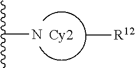
wherein:
Cy2 is selected from pyrrolidinyl and morpholinyl;
R¹¹ is selected from carboxyC₃₋₇cycloalkyl, carboxycarbonyl, carboxyC₁₋₆alkylaminocarbonyl, aminocarbonyl and aminosulfonyl; and
R¹² is selected from carboxy, C₁₋₆alkylsulfonylaminocarbonyl and C₁₋₆alkylsulfonyl;
or a pharmaceutically acceptable salt thereof.

19. A compound according to claim 18, wherein:
R¹ is Cl;
R⁴ is selected from H and CF₃;
R⁶ is selected from Br, methyl and CF₃;
R⁷ is selected from H and methoxy;
L is selected from ethyl, propyl and ethoxyethyl; wherein propyl is unsubstituted or substituted one time by OH; and
Y is —NHR¹¹ or 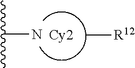
wherein:
Cy2 is selected from pyrrolidinyl and morpholinyl;
R¹¹ is selected from carboxycyclobutyl, carboxycyclopentyl, carboxycarbonyl, carboxyethylaminocarbonyl, aminocarbonyl and aminosulfonyl; and
R¹² is selected from carboxy, methylsulfonylaminocarbonyl and methylsulfonyl;
or a pharmaceutically acceptable salt thereof.

20. A compound according to claim 1, selected from:
1-[3-[5-bromo-2-(8-chloro-4-oxo-chromen-2-yl)phenoxy]propyl]pyrrolidine-3-carboxylic acid;
4-[3-[5-bromo-2-(8-chloro-4-oxo-chromen-2-yl)phenoxy]propyl]morpholine-2-carboxylic acid;
(2R)-1-[3-[5-bromo-2-(8-chloro-4-oxo-chromen-2-yl)phenoxy]propyl]pyrrolidine-2-carboxylic acid;
(2S)-1-[3-[5-bromo-2-(8-chloro-4-oxo-chromen-2-yl)phenoxy]propyl]pyrrolidine-2-carboxylic acid;
(3R)-1-[3-[5-bromo-2-(8-chloro-4-oxo-chromen-2-yl)phenoxy]propyl]pyrrolidine-3-carboxylic acid;
3-[3-[2-(8-chloro-4-oxo-chromen-2-yl)-5-(trifluoromethyl)phenoxy]propylamino]cyclobutanecarboxylic acid;
3-[3-[2-(8-chloro-4-oxo-chromen-2-yl)-5-(trifluoromethyl)phenoxy]propylamino]cyclopentanecarboxylic acid;
2-[1-[3-[2-(8-chloro-4-oxo-chromen-2-yl)-5-(trifluoromethyl)phenoxy]propyl]pyrrolidin-3-yl]acetic acid;
(2R)-2-[3-[2-(8-chloro-4-oxo-chromen-2-yl)-5-(trifluoromethyl)phenoxy]propylamino]-3-methyl-butanoic acid;
2-[[3-[[2-(8-chloro-4-oxo-chromen-2-yl)-5-(trifluoromethyl)phenoxy]methyl]oxetan-3-yl]methylamino]-2-oxo-acetic acid;
2-[3-[2-(8-chloro-4-oxo-chromen-2-yl)-5-(trifluoromethyl)phenoxy]propylamino]-4,4-dimethyl-pentanoic acid;
1-[3-[2-(8-chloro-4-oxo-chromen-2-yl)-5-(trifluoromethyl)phenoxy]propyl]pyrrolidine-3-carboxylic acid;
(3R)-1-[3-[2-(8-chloro-4-oxo-chromen-2-yl)-5-(trifluoromethyl)phenoxy]propyl]pyrrolidine-3-carboxylic acid;
(3S)-1-[3-[2-(8-chloro-4-oxo-chromen-2-yl)-5-(trifluoromethyl)phenoxy]propyl]pyrrolidine-3-carboxylic acid;
(3S)-1-[3-[2-(8-chloro-4-oxo-chromen-2-yl)-5-ethoxy-phenoxy]propyl]pyrrolidine-3-carboxylic acid;
(3R)-1-[3-[5-bromo-2-(8-chloro-4-oxo-chromen-2-yl)-4-methoxy-phenoxy]propyl]-pyrrolidine-3-carboxylic acid;
(3S)-1-[3-[5-bromo-2-(8-chloro-4-oxo-chromen-2-yl)-4-methoxy-phenoxy]propyl]-pyrrolidine-3-carboxylic acid;
4-[3-[2-(8-chloro-4-oxo-chromen-2-yl)-5-(trifluoromethyl)phenoxy]propyl]morpholine-2-carboxylic acid;
1-[2-[5-bromo-2-(8-chloro-4-oxo-chromen-2-yl)phenoxy]ethyl]azetidine-3-carboxylic acid;
1-[2-[5-bromo-2-(8-chloro-4-oxo-chromen-2-yl)phenoxy]ethyl]pyrrolidine-2-carboxylic acid;
1-[2-[5-bromo-2-(8-chloro-4-oxo-chromen-2-yl)phenoxy]ethyl]pyrrolidine-3-carboxylic acid;
1-[2-[5-bromo-2-(8-chloro-4-oxo-chromen-2-yl)phenoxy]ethyl]piperidine-4-carboxylic acid;
4-[2-[5-bromo-2-(8-chloro-4-oxo-chromen-2-yl)phenoxy]ethyl]morpholine-2-carboxylic acid;
(3S,4S)-1-[2-[5-bromo-2-(8-chloro-4-oxo-chromen-2-yl)phenoxy]ethyl]-4-methyl-pyrrolidine-3-carboxylic acid;
8-[2-[5-bromo-2-(8-chloro-4-oxo-chromen-2-yl)phenoxy]ethyl]-8-azabicyclo[3.2.1]octane-3-carboxylic acid;
3-[2-[5-bromo-2-(8-chloro-4-oxo-chromen-2-yl)phenoxy]ethylamino]bicyclo[1.1.1]pentane-1-carboxylic acid;
1-[2-[5-bromo-2-(8-chloro-4-oxo-chromen-2-yl)phenoxy]ethyl]-4,4-difluoro-pyrrolidine-2-carboxylic acid;
1-[2-[2-(8-chloro-4-oxo-chromen-2-yl)phenoxy]ethyl]piperidine-4-carboxylic acid;
(2R)-1-[2-[2-(8-chloro-4-oxo-chromen-2-yl)phenoxy]ethyl]pyrrolidine-2-carboxylic acid;
1-[2-[2-(8-chloro-4-oxo-chromen-2-yl)phenoxy]ethyl]pyrrolidine-3-carboxylic acid;
(2S)-1-[2-[2-(8-chloro-4-oxo-chromen-2-yl)phenoxy]ethyl]pyrrolidine-2-carboxylic acid;
1-[2-[2-(8-chloro-4-oxo-chromen-2-yl)phenoxy]ethyl]azetidine-3-carboxylic acid;
(3S)-1-[2-[2-(8-chloro-4-oxo-chromen-2-yl)-5-(trifluoromethyl)phenoxy]ethyl]pyrrolidine-3-carboxylic acid;
4-[2-[2-(8-chloro-4-oxo-chromen-2-yl)-5-(trifluoromethyl)phenoxy]ethyl]morpholine-2-carboxylic acid;
(2R)-2-[2-[2-(8-chloro-4-oxo-chromen-2-yl)-5-(trifluoromethyl)phenoxy]ethylamino]-3-methyl-butanoic acid;
(2S)-2-[2-[2-(8-chloro-4-oxo-chromen-2-yl)-5-(trifluoromethyl)phenoxy]ethylamino]-3-methyl-butanoic acid;
3-[2-[2-(8-chloro-4-oxo-chromen-2-yl)-5-(trifluoromethyl)phenoxy]ethylamino]cyclobutanecarboxylic acid;
(3R)-1-[2-[2-(8-chloro-4-oxo-chromen-2-yl)-5-isobutoxy-phenoxy]ethyl]pyrrolidine-3-carboxylic acid;
(3S)-1-[2-[2-(8-chloro-4-oxo-chromen-2-yl)-5-isobutoxy-phenoxy]ethyl]pyrrolidine-3-carboxylic acid;
(3R)-1-[2-[5-bromo-2-[8-chloro-4-oxo-5-(trifluoromethyl)chromen-2-yl]phenoxy]ethyl]-pyrrolidine-3-carboxylic acid;
(3S)-1-[2-[5-bromo-2-[8-chloro-4-oxo-5-(trifluoromethyl)chromen-2-yl]phenoxy]ethyl]-pyrrolidine-3-carboxylic acid;
1-[2-[2-(8-chloro-6-fluoro-4-oxo-chromen-2-yl)phenoxy]ethyl]pyrrolidine-3-carboxylic acid;
(2R)-1-[2-[5-bromo-2-(8-chloro-4-oxo-chromen-2-yl)-4-methyl-phenoxy]ethyl]pyrrolidine-2-carboxylic acid;
(2S)-1-[2-[5-chloro-2-(8-chloro-4-oxo-chromen-2-yl)-4-methyl-phenoxy]ethyl]pyrrolidine-2-carboxylic acid;
3-[3-[5-chloro-2-(8-chloro-4-oxo-chromen-2-yl)-4-methyl-phenoxy]propylamino]-cyclobutanecarboxylic acid;
(3R)-1-[2-[2-[5-bromo-2-(8-chloro-4-oxo-chromen-2-yl)phenoxy]ethoxy]ethyl]pyrrolidine-3-carboxylic acid;
(2R)-1-[2-[2-[5-bromo-2-(8-chloro-4-oxo-chromen-2-yl)-4-methyl-phenoxy]ethoxy]ethyl]-pyrrolidine-2-carboxylic acid;
4-[2-[2-[5-bromo-2-(8-chloro-4-oxo-chromen-2-yl)phenoxy]ethoxy]ethyl]morpholine-2-carboxylic acid;
(3R)-1-[2-[2-[2-(8-chloro-4-oxo-chromen-2-yl)-5-methoxy-4-methyl-phenoxy]ethoxy]ethyl]-pyrrolidine-3-carboxylic acid;
(3S)-1-[2-[2-[2-(8-chloro-4-oxo-chromen-2-yl)-5-methoxy-4-methyl-phenoxy]ethoxy]ethyl]-pyrrolidine-3-carboxylic acid;
4-[2-[5-bromo-2-(8-chloro-4-oxo-chromen-2-yl)phenoxy]ethyl]morpholine-3-carboxylic acid;
3-[2-[2-(8-chloro-4-oxo-chromen-2-yl)-5-methyl-phenoxy]ethylamino]cyclobutanecarboxylic acid;
1-[2-[2-(8-chloro-4-oxo-chromen-2-yl)-5-methyl-phenoxy]ethyl]azetidine-3-carboxylic acid;
cis-4-[2-[5-bromo-2-(8-chloro-4-oxo-chromen-2-yl)phenoxy]ethylamino]-cyclohexanecarboxylic acid;
trans-4-[2-[5-bromo-2-(8-chloro-4-oxo-chromen-2-yl)phenoxy]ethylamino]-cyclohexanecarboxylic acid;
2-[2-[2-(8-chloro-4-oxo-chromen-2-yl)-5-(trifluoromethyl)phenoxy]ethylamino]propanoic acid;
2-[2-[2-(8-chloro-4-oxo-chromen-2-yl)-5-methyl-phenoxy]ethylamino]-2-methyl-propanoic acid;
3-[3-[2-(8-chloro-4-oxo-chromen-2-yl)-5-methyl-phenoxy]propylamino]-cyclobutanecarboxylic acid;
cis-4-[3-[5-bromo-2-(8-chloro-4-oxo-chromen-2-yl)phenoxy]propylamino]tetrahydropyran-2-carboxylic acid;
1-[2-[5-chloro-2-(8-chloro-4-oxo-chromen-2-yl)-4-methyl-phenoxy]ethyl]azetidine-3-carboxylic acid;
3-[3-[2-(8-chloro-4-oxo-chromen-2-yl)-5-(trifluoromethyl)phenoxy]propylamino]butanoic acid;
2-[3-[2-(8-chloro-4-oxo-chromen-2-yl)-5-(trifluoromethyl)phenoxy]propylamino]-3-cyclohexyl-propanoic acid;
(3R)-1-[2-[5-bromo-2-(8-chloro-4-oxo-chromen-2-yl)phenoxy]ethyl]-N-methylsulfonyl-pyrrolidine-3-carboxamide;
(3R)-1-[3-[2-(8-chloro-4-oxo-chromen-2-yl)-5-(trifluoromethyl)phenoxy]propyl]-N-methylsulfonyl-pyrrolidine-3-carboxamide;
(3S)-1-[3-[2-(8-chloro-4-oxo-chromen-2-yl)-5-(trifluoromethyl)phenoxy]propyl]-N-methylsulfonyl-pyrrolidine-3-carboxamide;
(3R)-1-[3-[2-(8-chloro-4-oxo-chromen-2-yl)-5-(trifluoromethyl)phenoxy]propyl]-N-cyclopropylsulfonyl-pyrrolidine-3-carboxamide;
4-[2-[2-(8-chloro-4-oxo-chromen-2-yl)-5-(trifluoromethyl)phenoxy]ethyl]-N-methylsulfonyl-morpholine-2-carboxamide;
(3S)-1-[2-[5-bromo-2-(8-chloro-4-oxo-chromen-2-yl)phenoxy]ethyl]-N-methylsulfonyl-pyrrolidine-3-carboxamide;
(3R)-1-[2-[2-(8-chloro-4-oxo-chromen-2-yl)-5-(trifluoromethyl)phenoxy]ethyl]-N-methylsulfonyl-pyrrolidine-3-carboxamide;
(3S)-1-[2-[2-(8-chloro-4-oxo-chromen-2-yl)-5-(trifluoromethyl)phenoxy]ethyl]-N-methylsulfonyl-pyrrolidine-3-carboxamide;
1-[2-[2-(8-chloro-4-oxo-chromen-2-yl)-5-(trifluoromethyl)phenoxy]ethyl]-N-cyclopropylsulfonyl-pyrrolidine-2-carboxamide;
1-[2-[2-(8-chloro-4-oxo-chromen-2-yl)-5-(trifluoromethyl)phenoxy]ethyl]-N-methylsulfonyl-pyrrolidine-2-carboxamide;
1-[2-[5-bromo-2-(8-chloro-4-oxo-chromen-2-yl)phenoxy]ethyl]-N-methylsulfonyl-pyrrolidine-2-carboxamide;
1-[2-[5-bromo-2-(8-chloro-4-oxo-chromen-2-yl)phenoxy]ethyl]pyrrolidin-2-one;
N-[1-[2-[5-bromo-2-(8-chloro-4-oxo-chromen-2-yl)phenoxy]ethyl]pyrrolidin-3-yl]-N-methyl-methanesulfonamide;
8-chloro-2-[2-[3-[(3R,4S)-3,4-dihydroxypyrrolidin-1-yl]propoxy]-4-(trifluoromethyl)phenyl]chromen-4-one;
8-chloro-2-[2-[3-[(1,1-dioxothian-4-yl)amino]propoxy]-4-(trifluoromethyl)phenyl]chromen-4-one;
8-chloro-2-[2-[3-(2-methylsulfonylethylamino)propoxy]-4-(trifluoromethyl)phenyl]chromen-4-one;
2-[3-[2-(8-chloro-4-oxo-chromen-2-yl)-5-(trifluoromethyl)phenoxy]propylamino]ethanesulfonamide;
8-chloro-2-[2-[3-(4-methylsulfonylpiperazin-1-yl)propoxy]-4-(trifluoromethyl)phenyl]-chromen-4-one;
2-[2-[2-(4-acetylpiperazin-1-yl)ethoxy]-4-bromo-phenyl]-8-chloro-chromen-4-one;
1-[2-[5-bromo-2-(8-chloro-4-oxo-chromen-2-yl)phenoxy]ethyl]pyrrolidine-3-carboxamide;
2-[4-bromo-2-[2-(3-methylsulfonylpyrrolidin-1-yl)ethoxy]phenyl]-8-chloro-chromen-4-one;
4-[2-[5-bromo-2-(8-chloro-4-oxo-chromen-2-yl)phenoxy]ethyl]thiomorpholine-3-carboxamide;
N-[3-[2-(8-chloro-4-oxo-chromen-2-yl)-5-(trifluoromethyl)phenoxy]-2-hydroxy-propyl]cyclopropanesulfonamide;
8-chloro-2-[2-[2-[4-(1H-tetrazol-5-yl)-1-piperidyl]ethoxy]-4-(trifluoromethyl)phenyl]chromen-4-one;
8-chloro-2-[2-[3-(3-methylsulfonylpyrrolidin-1-yl)propoxy]-4-(trifluoromethyl)phenyl]chromen-4-one;
2-[4-bromo-2-[2-(1,1-dioxo-1,4-thiazinan-4-yl)ethoxy]phenyl]-8-chloro-chromen-4-one;
2-[4-bromo-2-[2-(2-morpholinoethoxy)ethoxy]phenyl]-8-chloro-chromen-4-one;
1-[2-[2-(8-chloro-4-oxo-chromen-2-yl)-5-methyl-phenoxy]ethyl]imidazolidin-2-one;
1-[2-[2-(8-chloro-4-oxo-chromen-2-yl)-5-methyl-phenoxy]ethyl]-3-methyl-imidazolidin-2-one;
8-chloro-2-[2-[2-(4-methylsulfinyl-1-piperidyl)ethoxy]-4-(trifluoromethyl)phenyl]chromen-4-one;
8-chloro-2-[2-[2-[4-(methylsulfonimidoyl)-1-piperidyl]ethoxy]-4-(trifluoromethyl)phenyl]chromen-4-one;
ethyl 2-[3-[2-(8-chloro-4-oxo-chromen-2-yl)-5-methyl-phenoxy]propylamino]-2-oxo-acetate;
N-[2-[2-(8-chloro-4-oxo-chromen-2-yl)-5-(trifluoromethyl)phenoxy]ethyl]cyclopropanesulfonamide;
N-[3-[2-(8-chloro-4-oxo-chromen-2-yl)-5-(trifluoromethyl)phenoxy]propyl]benzenesulfonamide;
N-[3-[2-(8-chloro-4-oxo-chromen-2-yl)-5-(trifluoromethyl)phenoxy]propyl]acetamide;
N-[3-[2-(8-chloro-4-oxo-chromen-2-yl)-5-(trifluoromethyl)phenoxy]propyl]propanamide;
ethyl 2-[3-[2-(8-chloro-4-oxo-chromen-2-yl)-5-(trifluoromethyl)phenoxy]propylamino]-2-oxo-acetate;
ethyl 2-[2-[2-(8-chloro-4-oxo-chromen-2-yl)-5-(trifluoromethyl)phenoxy]ethylamino]-2-oxo-acetate;
ethyl 2-[2-[4-bromo-2-(8-chloro-4-oxo-chromen-2-yl)-5-methoxy-phenoxy]ethylamino]-2-oxo-acetate;
ethyl 2-[2-[2-(8-chloro-6-methoxy-4-oxo-chromen-2-yl)-5-methyl-phenoxy]ethylamino]-2-oxo-acetate;
ethyl 2-[2-[2-(8-chloro-4-oxo-chromen-2-yl)-5-methoxy-phenoxy]ethylamino]-2-oxo-acetate;
methyl N-[2-[5-bromo-2-(8-chloro-4-oxo-chromen-2-yl)phenoxy]ethyl]carbamate;
ethyl N-[2-[5-bromo-2-(8-chloro-4-oxo-chromen-2-yl)phenoxy]ethyl]carbamate;
8-chloro-2-[4-methyl-2-[3-(methylsulfamoylamino)propoxy]phenyl]chromen-4-one;
2-[4-bromo-2-[(1-cyclopropylsulfonyl-4-piperidyl)oxy]phenyl]-8-chloro-chromen-4-one;
ethyl 2-[2-[2-(8-chloro-4-oxo-chromen-2-yl)-5-methyl-phenoxy]ethylamino]-2-oxo-acetate;
2-[[1-[[2-(8-chloro-4-oxo-chromen-2-yl)-5-(trifluoromethyl)phenoxy]methyl]cyclopropyl]amino]-2-oxo-acetic acid;
2-[2-[2-(8-chloro-4-oxo-chromen-2-yl)-5-methoxy-phenoxy]ethylamino]-2-oxo-acetic acid;
2-[2-[2-(8-chloro-4-oxo-chromen-2-yl)-3-fluoro-5-methyl-phenoxy]ethylamino]-2-oxo-acetic acid;
2-[2-[5-bromo-2-(8-chloro-4-oxo-chromen-2-yl)phenoxy]ethylamino]-2-oxo-acetic acid;
2-[2-[2-(8-chloro-4-oxo-chromen-2-yl)-4,5-dimethoxy-phenoxy]ethylamino]-2-oxo-acetic acid;
2-[[2-[2-(8-chloro-4-oxo-chromen-2-yl)-5-(trifluoromethyl)phenoxy]-1,1-dimethyl-ethyl]amino]-2-oxo-acetic acid;
2-[3-[2-(8-chloro-6-methoxy-4-oxo-chromen-2-yl)-5-(trifluoromethyl)phenoxy]-propylamino]-2-oxo-acetic acid;
2-[3-[2-(8-chloro-4-oxo-chromen-2-yl)-5-(trifluoromethyl)phenoxy]propylamino]-2-oxo-acetic acid;
2-[2-[2-(8-chloro-4-oxo-chromen-2-yl)-3-methoxy-5-methyl-phenoxy]ethylamino]-2-oxo-acetic acid;
2-[2-[2-(8-chloro-4-oxo-chromen-2-yl)-5-(trifluoromethyl)phenoxy]ethylamino]-2-oxo-acetic acid;
2-[3-[2-(8-chloro-4-oxo-chromen-2-yl)-5-methyl-phenoxy]propylamino]-2-oxo-acetic acid;
2-[2-[2-(8-chloro-4-oxo-chromen-2-yl)-5-methyl-phenoxy]ethylamino]-2-oxo-acetic acid;
2-[2-[4-bromo-2-(8-chloro-4-oxo-chromen-2-yl)-5-methoxy-phenoxy]ethylamino]-2-oxo-acetic acid;
2-[2-[2-(8-chloro-4-oxo-chromen-2-yl)-5-(trifluoromethyl)phenoxy]ethylamino]acetic acid;
3-[2-[2-(8-chloro-4-oxo-chromen-2-yl)-5-(trifluoromethyl)phenoxy]ethylamino]propanoic acid;
2-[3-[2-(8-chloro-4-oxo-chromen-2-yl)-5-(trifluoromethyl)phenoxy]propylamino]acetic acid;
2-oxo-2-[(3S)-3-[2-(8-chloro-4-oxo-chromen-2-yl)-5-(trifluoromethyl)phenoxy]pyrrolidin-1-yl]acetic acid;
2-[3-[2-(8-chloro-4-oxo-chromen-2-yl)-5-(trifluoromethyl)phenoxy]azetidin-1-yl]-2-oxo-acetic acid;
3-[[4-[5-bromo-2-(8-chloro-4-oxo-chromen-2-yl)phenoxy]-1-piperidyl]methyl]benzoic acid;
2-[4-[2-(8-chloro-4-oxo-chromen-2-yl)-5-(trifluoromethyl)phenoxy]-1-piperidyl]-2-oxo-acetic acid;
2-oxo-2-[(3R)-3-[2-(8-chloro-4-oxo-chromen-2-yl)-5-(trifluoromethyl)phenoxy]pyrrolidin-1-yl]acetic acid;
3-[4-[5-bromo-2-(8-chloro-4-oxo-chromen-2-yl)phenoxy]-1-piperidyl]cyclobutanecarboxylic acid;
2-[(3S)-3-[2-(8-chloro-4-oxo-chromen-2-yl)-5-methyl-phenoxy]pyrrolidin-1-yl]-2-oxo-acetic acid;
2-[[3-[2-(8-chloro-4-oxo-chromen-2-yl)-5-(trifluoromethyl)phenoxy]cyclobutyl]amino]-2-oxo-acetic acid;
2-[3-[2-(8-chloro-4-oxo-chromen-2-yl)-5-(trifluoromethyl)phenoxy]propyl-methyl-amino]acetic acid;
2-[2-[2-(8-chloro-4-oxo-chromen-2-yl)-5-methyl-phenoxy]ethyl-cyclopropyl-amino]-2-oxo-acetic acid;
8-chloro-2-[2-[2-[ethylsulfamoyl(methyl)amino]ethoxy]-4-methyl-phenyl]-4-oxo-chromene;
ethyl 2-[2-[2-(8-chloro-4-oxo-chromen-2-yl)-5-methyl-phenoxy]ethyl-methyl-amino]-2-oxo-acetate;
ethyl 2-[3-[2-(8-chloro-4-oxo-chromen-2-yl)-5-(trifluoromethyl)phenoxy]propyl-ethylsulfonyl-amino]-2-oxo-acetate;
3-[3-[2-(8-chloro-4-oxo-chromen-2-yl)-5-(trifluoromethyl)phenoxy]propyl-cyclopropylsulfonyl-amino]cyclobutanecarboxylic acid;
2-[carboxymethyl-[3-[2-(8-chloro-4-oxo-chromen-2-yl)-5-(trifluoromethyl)phenoxy]propyl]-amino]acetic acid;
cis-3-[benzenesulfonyl-[3-[2-(8-chloro-4-oxo-chromen-2-yl)-5-(trifluoromethyl)-phenoxy]propyl]amino]cyclobutanecarboxylic acid;
trans-3-[benzenesulfonyl-[3-[2-(8-chloro-4-oxo-chromen-2-yl)-5-(trifluoromethyl)phenoxy]-propyl]amino]cyclobutanecarboxylic acid;
2-[benzenesulfonyl-[3-[2-(8-chloro-4-oxo-chromen-2-yl)-5-(trifluoromethyl)phenoxy]-propyl]amino]acetic acid;
2-[acetyl-[3-[2-(8-chloro-4-oxo-chromen-2-yl)-5-(trifluoromethyl)phenoxy]propyl]amino]-acetic acid;
2-[3-[2-(8-chloro-4-oxo-chromen-2-yl)-5-(trifluoromethyl)phenoxy]propyl-cyclopropylsulfonyl-amino]acetic acid;
N′-[2-[2-(8-chloro-4-oxo-chromen-2-yl)-5-(trifluoromethyl)phenoxy]ethyl]oxamide;
N′-[2-[2-(8-chloro-4-oxo-chromen-2-yl)-5-methoxy-phenoxy]ethyl]oxamide;
N-[2-[2-(8-chloro-4-oxo-chromen-2-yl)-5-(trifluoromethyl)phenoxy]ethyl]-N′-cyclopropyl-oxamide;
N-[3-[2-(8-chloro-4-oxo-chromen-2-yl)-5-(trifluoromethyl)phenoxy]propyl]-N′-cyclopropyl-sulfonyl-oxamide;
N-[2-[2-(8-chloro-4-oxo-chromen-2-yl)-5-methyl-phenoxy]ethyl]-2-[(3S)-3-hydroxy-pyrrolidin-1-yl]-2-oxo-acetamide;
(2S)-2-[2-[2-(8-chloro-4-oxo-chromen-2-yl)-5-methyl-phenoxy]ethylcarbamoylamino]propanoic acid;
(5S)-3-[2-[2-(8-chloro-4-oxo-chromen-2-yl)-5-methyl-phenoxy]ethyl]-5-isopropyl-imidazolidine-2,4-dione;
(5S)-3-[2-[2-(8-chloro-4-oxo-chromen-2-yl)-5-(trifluoromethyl)phenoxy]ethyl]-5-isopropyl-imidazolidine-2,4-dione;
2-[2-[2-(8-chloro-4-oxo-chromen-2-yl)-5-methyl-phenoxy]ethylcarbamoylamino]acetic acid;
(5S)-3-[2-[2-(8-chloro-4-oxo-chromen-2-yl)-5-(trifluoromethyl)phenoxy]ethyl]-5-methyl-imidazolidine-2,4-dione;
1-[3-[2-(8-chloro-4-oxo-chromen-2-yl)-5-methyl-phenoxy]propyl]-3-(p-tolylsulfonyl)urea;
3-[2-(8-chloro-4-oxo-chromen-2-yl)-5-(trifluoromethyl)phenoxy]propylurea;
1-[2-[2-(8-chloro-4-oxo-chromen-2-yl)-5-(trifluoromethyl)phenoxy]ethyl]-3-(p-tolylsulfonyl)urea;
8-chloro-4-oxo-2-[2-[2-(sulfamoylamino)ethoxy]-4-(trifluoromethyl)phenyl]chromene;
8-chloro-4-oxo-2-[2-[3-(sulfamoylamino)propoxy]-4-(trifluoromethyl)phenyl]chromene;
8-chloro-2-[4-methyl-2-[2-(sulfamoylamino)ethoxy]phenyl]-4-oxo-chromene;
2-[3-[2-[2-(8-chloro-4-oxo-chromen-2-yl)-5-(trifluoromethyl)phenoxy]ethyl]-2-oxo-imidazolidin-1-yl]-2-oxo-acetic acid;
(3S)-1-[3-[2-(8-chloro-4-oxo-chromen-2-yl)-5-(trifluoromethyl)phenoxy]-2-hydroxy-propyl]pyrrolidine-3-carboxylic acid;
(3S)-1-[3-[5-bromo-2-(8-chloro-4-oxo-chromen-2-yl)phenoxy]-2-hydroxy-propyl]pyrrolidine-3-carboxylic acid;
(3R)-1-[3-[5-bromo-2-(8-chloro-4-oxo-chromen-2-yl)phenoxy]-2-hydroxy-propyl]pyrrolidine-3-carboxylic acid;
2-[[3-[2-(8-chloro-4-oxo-chromen-2-yl)-5-(trifluoromethyl)phenoxy]-2-hydroxy-propyl]amino]-2-oxo-acetic acid;
2-[2-[2-(8-chloro-4-oxo-chromen-2-yl)-5-(trifluoromethyl)phenoxy]ethylamino]acetamide;
2-[2-[2-(8-chloro-4-oxo-chromen-2-yl)-5-(trifluoromethyl)phenoxy]ethylamino]propanamide; and
1-[3-[5-bromo-2-(8-chloro-4-oxo-chromen-2-yl)phenoxy]propyl]-5-oxo-pyrrolidine-3-carboxylic acid;
or a pharmaceutically acceptable salt thereof.

21. A compound according to claim 1, selected from:
1-[3-[5-bromo-2-(8-chloro-4-oxo-chromen-2-yl)phenoxy]propyl]pyrrolidine-3-carboxylic acid;
4-[3-[5-bromo-2-(8-chloro-4-oxo-chromen-2-yl)phenoxy]propyl]morpholine-2-carboxylic acid;
3-[3-[2-(8-chloro-4-oxo-chromen-2-yl)-5-(trifluoromethyl)phenoxy]propylamino]cyclobutanecarboxylic acid;
3-[3-[2-(8-chloro-4-oxo-chromen-2-yl)-5-(trifluoromethyl)phenoxy]propylamino]cyclopentanecarboxylic acid;
(3R)-1-[3-[2-(8-chloro-4-oxo-chromen-2-yl)-5-(trifluoromethyl)phenoxy]propyl]pyrrolidine-3-carboxylic acid;
(3S)-1-[3-[2-(8-chloro-4-oxo-chromen-2-yl)-5-(trifluoromethyl)phenoxy]propyl]pyrrolidine-3-carboxylic acid;
(3S)-1-[3-[5-bromo-2-(8-chloro-4-oxo-chromen-2-yl)-4-methoxy-phenoxy]propyl]pyrrolidine-3-carboxylic acid;
4-[3-[2-(8-chloro-4-oxo-chromen-2-yl)-5-(trifluoromethyl)phenoxy]propyl]morpholine-2-carboxylic acid;
1-[2-[5-bromo-2-(8-chloro-4-oxo-chromen-2-yl)phenoxy]ethyl]pyrrolidine-2-carboxylic acid;
4-[2-[5-bromo-2-(8-chloro-4-oxo-chromen-2-yl)phenoxy]ethyl]morpholine-2-carboxylic acid;
(3R)-1-[2-[5-bromo-2-[8-chloro-4-oxo-5-(trifluoromethyl)chromen-2-yl]phenoxy]ethyl]pyrrolidine-3-carboxylic acid;
(3R)-1-[2-[2-[5-bromo-2-(8-chloro-4-oxo-chromen-2-yl)phenoxy]ethoxy]ethyl]pyrrolidine-3-carboxylic acid;
4-[2-[5-bromo-2-(8-chloro-4-oxo-chromen-2-yl)phenoxy]ethyl]morpholine-3-carboxylic acid;
(3R)-1-[2-[5-bromo-2-(8-chloro-4-oxo-chromen-2-yl)phenoxy]ethyl]-N-methylsulfonyl-pyrrolidine-3-carboxamide;
1-[2-[2-(8-chloro-4-oxo-chromen-2-yl)-5-(trifluoromethyl)phenoxy]ethyl]-N-methylsulfonyl-pyrrolidine-2-carboxamide;
2-[4-bromo-2-[2-(3-methylsulfonylpyrrolidin-1-yl)ethoxy]phenyl]-8-chloro-chromen-4-one;
(2S)-2-[2-[2-(8-chloro-4-oxo-chromen-2-yl)-5-methyl-phenoxy]ethylcarbamoylamino]propanoic acid;
3-[2-(8-chloro-4-oxo-chromen-2-yl)-5-(trifluoromethyl)phenoxy]propylurea;
8-chloro-4-oxo-2-[2-[3-(sulfamoylamino)propoxy]-4-(trifluoromethyl)phenyl]chromene;
(3R)-1-[3-[5-bromo-2-(8-chloro-4-oxo-chromen-2-yl)phenoxy]-2-hydroxy-propyl]pyrrolidine-3-carboxylic acid; and
2-[[3-[2-(8-chloro-4-oxo-chromen-2-yl)-5-(trifluoromethyl)phenoxy]-2-hydroxy-propyl]amino]-2-oxo-acetic acid;
or a pharmaceutically acceptable salt thereof.

22. A process for preparing a compound according to claim 1, the process comprising at least one of the following steps:
(a) substituting a compound of formula (XI), 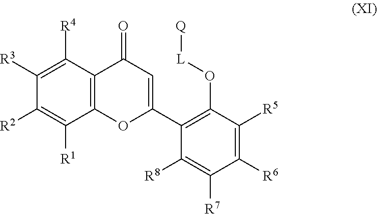
with a compound of formula (X-1), 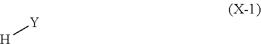
in the presence of a base;
(b) substituting a compound of formula (XI) with amine (X-2), 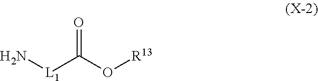
in the presence of a base;
(c) hydrolyzing a compound of formula (I-2), 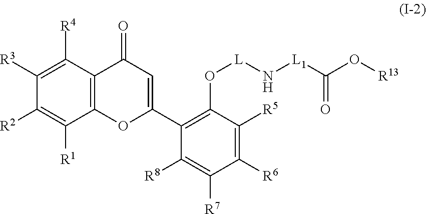
in the presence of a base;
(d) substituting a compound of formula (I-2) with a compound of formula (XVII-1), 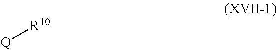
in the presence of a base;
(e) hydrolyzing a compound of formula (I-4), 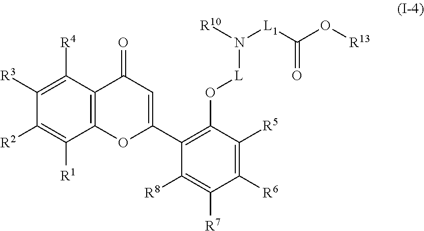
in the presence of a base or a Lewis acid;
(f) treating a compound of formula (I-3), 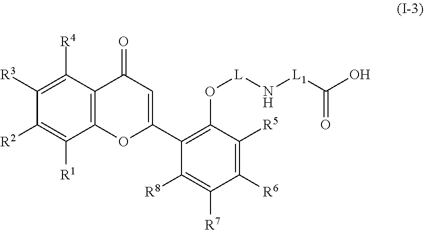
with a compound of formula (XVII-1) in the presence of a base;
(g) Condensation of a compound of formula (I-3), with a compound of formula (X-3), 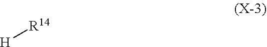
in the presence of a condensation reagent;
(h) substituting a compound of formula (XIX), 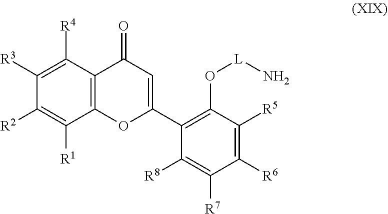
with a compound of formula (XVII-3), 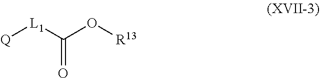
in the presence of abase;
(i) substituting a compound of formula (XIX), with a compound of formula (XVII-5)
Q-R¹¹ (XVII-5)
in the presence of a base;
(j) deprotecting a compound of formula (XVI), 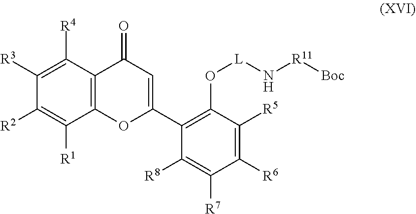
in the presence of a Lewis acid;
(k) hydrolyzing a compound of formula (XX), 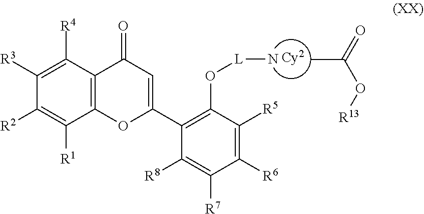
in the presence of a base or a Lewis acid;
(l) Condensation of a compound of formula (I-8), 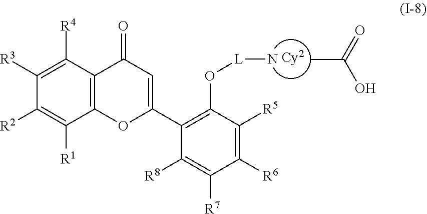
with a compound of formula (X-3), in the presence of a condensation reagent and a base;
(m) treating a compound of formula (XXII), 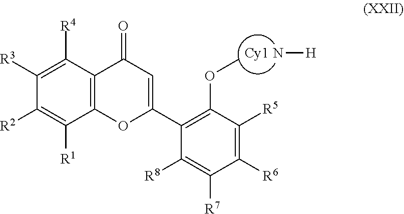
with a compound of formula (XVII-7); 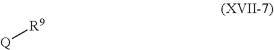
in the presence of a base;
(n) hydrolyzing a compound of formula (XXIII), 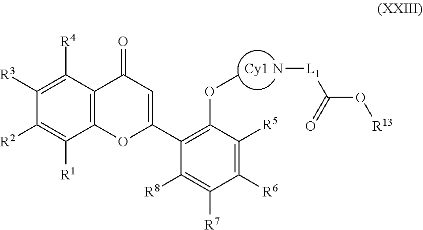
in the presence of a base;
(o) treating a compound of formula (XXXI), 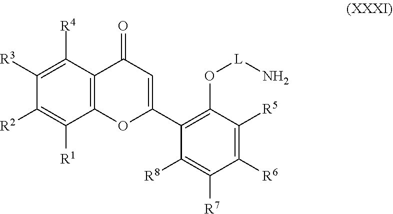
with isocyanato(trimethyl)silane in the presence of a base;
(p) treating a compound of formula (XXXI) with a compound of formula (XXXII-1) 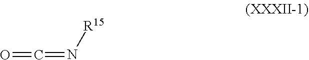
in the presence of a base;
(q) deprotecting a compound of formula (XXXIII), 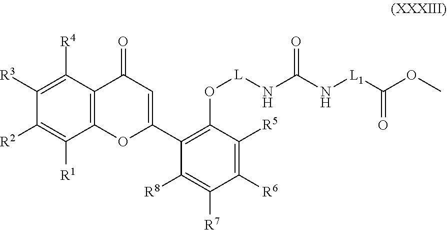
in the presence of a base;
(r) hydrolyzing a compound of formula (XXXV), 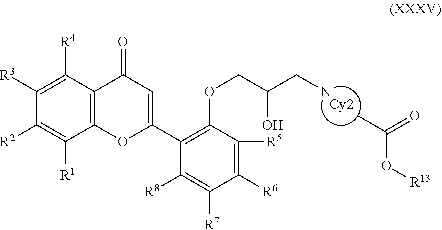
in the presence of a base;
(s) hydrolyzing a compound of formula (XXXVIII), 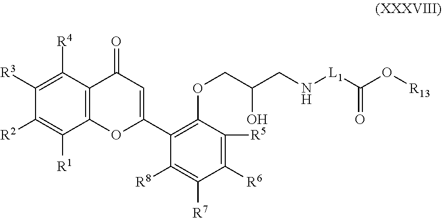
in the presence of a base;
wherein:
Q is halogen or Omns;
L₁ is C₁₋₆alkyl, carbonyl or C₃₋₇cycloalkyl;
R¹³ is C₁₋₆alkyl;
R¹⁴ is C₁₋₆alkylsulfonylamino, C₃₋₇cycloalkylsulfonylamino, C₃₋₇cycloalkylamino or hydroxyheterocyclyl; and
R¹⁵ is C₁₋₆alkylphenylsulfonyl.

23. (canceled)

24. A pharmaceutical composition comprising a compound in accordance with claim 1 and a therapeutically inert carrier.

25. (canceled)

26. (canceled)

27. (canceled)

28. (canceled)

29. (canceled)

30. (canceled)

31. (canceled)

32. A compound manufactured according to the process of claim 22.

33. A method for the treatment or prophylaxis of HBV infection, which method comprises administering to a patient in need thereof an effective amount of a compound according to claim 1.

34. A method for the treatment or prophylaxis of HBV infection, which method comprises administering to a patient in need thereof an effective amount of a compound of claim 20.

35. A method of inhibiting a target selected from HiBsAg, HBeAg, cccDNA, HBV DNA, the method comprising administering to a patient an effective amount of a compound of claim 1.

36. A method of inhibiting a target selected from HiBsAg, HBeAg, cccDNA, HBV DNA, the method comprising administering to a patient an effective amount of a compound of claim 20.

37. A pharmaceutical composition comprising a compound in accordance with claim 20 and a therapeutically inert carrier.


SEQUENCE LISTING (3 sequences)

SEQ ID NO: 1 - DNA, 20 bp, Artificial Sequence
  Description of Artificial Sequence Synthetic primer
aagaaaaaccccgcctgtaa

SEQ ID NO: 2 - DNA, 23 bp, Artificial Sequence
  Description of Artificial Sequence Synthetic primer
cctgttctgactactgcctctcc

SEQ ID NO: 3 - DNA, 27 bp, Artificial Sequence
  Description of Artificial Sequence Synthetic probe
cctgatgtgatgttctccatgttcagc